(19) United States
(12) Patent Application Publication
(10) Pub. No.: US 2007/0039069 A1
(43) Pub. Date: Feb. 15, 2007

(54) Nucleic acid molecules associated with oil in plants

(76) Inventors: James A. Rogers, Ivoryton, CT (US); Monica Ravanello, Vacaville, CA (US); Thomas Savage, Sacramento, CA (US); Cathy Laurie, Santa Clara, CA (US); John LeDeaux, Creve Coeur, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/812,829

(22) Filed: Mar. 29, 2004

Related U.S. Application Data
(63) Continuation-in-part of application No. 10/806,075, filed on Mar. 22, 2004, now abandoned.

(51) Int. Cl. A01H 1/00; C12N 15/82; A01H 5/00

(52) U.S. Cl. 800/281; 800/320.1

(57) ABSTRACT

Polynucleotides that encode proteins associated with oil content in plants are useful in constructs to make transgenic plants, e.g., maize or soybean, with desirable oil content phenotype and progeny of any generation derived from the fertile transgenic plants. Markers associated with oil content QTL are useful in breeding for plants with desired oil content.

### REFERENCE TO RELATED APPLICATIONS

This application is a continuation in part of Ser. No. 10/806,075 which claims priority to Ser. No. 10/613,520 is also a continuation in part of Ser. No. 10/389,566 which claims priority to U.S. Provisional Applications 60/365,301 filed Mar. 15, 2002, 60/391,786 filed Jun. 25, 2002 and 60/392,018 filed Jun. 26, 2002, each of which is incorporated herein by reference in its entirety.

### INCORPORATION OF SEQUENCE LISTING

Two copies of the sequence listing (Seq. Listing Copy 1 and Seq. Listing Copy 2) and a computer-readable form of the sequence listing, all on CD-ROMs, each containing the file named “pa_{—}00678.rpt”, which is 7,821 kilobytes (measured in MS-Windows) and was created on Mar. 18, 2004, are herein incorporated by reference.

### INCORPORATION OF TABLES

Two copies of Tables 1-5 (Tables 1-5, Copy 1 and Tables 1-5, Copy 2) all on CD-ROMs, each containing the file named “pa_{—}00678.txt”, which is 192 kilobytes (measured in MS-Windows) and was created on Mar. 29, 2004, are herein incorporated by reference.

TABLES FILED ON CD
The patent application contains tables filed on compact disc. These tables have been included at the end of the specification

### FIELD OF THE INVENTION

Disclosed herein are inventions in the field of plant molecular biology, plant genetics and plant breeding. More specifically disclosed are nucleic acid and amino acid molecules associated with oil in plants, particularly oil in maize. Also disclosed are genetic markers for such nucleic acid molecules and genes and QTLs associated with oil in maize. Such markers are useful for discovery and isolation of genes useful in enhancing the level of oil in plants and for molecular breeding of maize with enhanced levels of oil. Also disclosed are transgenic plants with over expression of one or more genes associated with oil.

### BACKGROUND OF THE INVENTION

Maize, Zea mays L., is one of the major crops grown worldwide as a primary source for animal feed, human food and industrial purposes. Maize plants with improved agronomic traits, such as yield or pest resistance, improved quality traits such as oil, protein or starch quality or quantity, or improved processing characteristics, such as extractability of desirable compounds, are desirable for both the farmer and consumer of maize and maize derived products. The ability to breed or develop transgenic plants with improved traits depends in part on identification of genes associated with a trait. The unique maize sequences disclosed herein may be useful as mapping tools to assist in plant breeding and in designing transgenic plants. Homologous sequences in plant species other than maize and in fungi, algae and bacteria may be useful to confer novel phenotypes in transgenic maize and other oil-producing plants.

Increases in the oil content of maize seeds can be achieved by altering the expression of one or more genes that encode a protein that functionally increases oil production or storage. Effective changes in expression may include constitutive increases, constitutive decreases or alterations in the tissue-specific pattern of expression. See, for instance, U.S. Pat. No. 6,268,550, which discloses that a higher oil content soybean is associated with a twofold increase in acetyl CoA carboxylase (ACCase) activity during early to mid stages of development when compared with a low oil content soybean. In view of a correlation of increased expression of the ACCase gene with an increase in the oil content of the seed, it is predicted that over expression of the ACCase enzyme is likely to lead to an increase in the oil content of the plants and seeds. Since metabolic pathways affecting oil production and storage are complex and controlled by a large number of enzymes and transcription factors, there is a need to discover and modulate the expression of other genes associated with oil.

Polymorphisms are useful as genetic markers for genotyping applications in the agriculture field, e.g., in plant genetic studies and commercial breeding. See for instance U.S. Pat. Nos. 5,385,835; 5,492,547 and 5,981,832, the disclosures of all of which are incorporated herein by reference. The highly conserved nature of DNA combined with the rare occurrences of stable polymorphisms provide genetic markers that are both predictable and discerning of different genotypes. Among the classes of existing genetic markers are a variety of polymorphisms indicating genetic variation including restriction-fragment-length polymorphisms (RFLPs), amplified fragment-length polymorphisms (AFLPs), simple sequence repeats (SSRs), single nucleotide polymorphisms (SNPs), and insertion/deletion polymorphisms (Indels). Because the number of genetic markers for a plant species is limited, the discovery of additional genetic markers associated with a trait will facilitate genotyping applications including marker-trait association studies, gene mapping, gene discovery, marker-assisted selection, and marker-assisted breeding. Evolving technologies make certain genetic markers more amenable for rapid, large scale use. For instance, technologies for SNP detection indicate that SNPs may be preferred genetic markers.

### SUMMARY OF THE INVENTION

This invention provides genes that have been identified as being associated with high oil in maize. An aspect of this invention provides homologs of such genes from a variety of other plant species and other organisms, e.g. fungi, algae and bacteria. Nucleic acid molecules derived from such genes and homologous genes which encode proteins that are effective in the production and/or storage of oil in plant seeds are useful in other aspects of this invention, e.g. DNA constructs for producing transgenic plants and seed with higher or lower oil. Thus, a particular aspect of this invention is transgenic plant seed having in its genome a recombinant DNA construct comprising at least one oil-associated gene of this invention operably linked to a promoter which is functional in the plant to transcribe the oil-associated gene. In one preferred aspects of this invention such transgenic plant seeds can grow into plants having enhanced seed oil as compared to wild type. Conversely, an alternative aspect of this invention employs gene suppression technology, e.g. RNAi gene suppression, to provide transgenic plant seeds having a recombinant DNA construct which includes DNA effective for suppression of an oil-associated gene. Such seed can be grown into plants having reduced seed oil as compared to wild type. Alternatively, the suppression of the oil-associated gene could lead to plants with increased seed oil compared to wild type, depending on the action of the gene.

Another aspect of this invention provides hybrid maize seed that is produced by crossing two parental maize lines where at least one of the parental maize lines is a transgenic maize line which has in its genome a recombinant DNA construct for producing transgenic maize with enhanced seed oil as compared to its parents, e.g. its non-transgenic ancestors. Such hybrid maize seed will have a recombinant DNA construct comprising at least one oil-associated gene of this invention operably linked to a promoter which is functional in maize to transcribe the oil-associated gene. Still another aspect of this invention provides hybrid maize seed that can produce maize plants characterized by agronomic traits of seed oil level, yield and standability. Preferably, seed oil level is greater than seed oil level in said closest non-transgenic parental lines and, even more preferably, there is essentially no reduction in yield and standability traits in said maize plants as compared to yield and standability traits for said closest non-transgenic parental lines.

Still another aspect of this invention provides methods of producing hybrid maize plants having enhanced levels of seed oil production and/or seed oil storage as compared to the closest non-transgenic ancestor maize lines. Such methods comprise producing a transgenic maize plant having in its genome a recombinant DNA construct comprising at least one oil-associated gene of this invention operably linked to a promoter which is functional in maize to transcribe the oil-associated gene. Such methods further comprise crossing transgenic progeny of transgenic maize plants with at least one other maize plant to produce hybrid maize plants having enhanced levels of seed oil production.

Yet another aspect of this invention relates to a method for producing vegetable oil by growing and harvesting oil from plants of this invention.

This invention also provides maize oil markers that have been identified as statistically significant in associating with high oil in maize. Such markers are especially useful in methods of this invention relating to breeding maize for high oil. More particularly, this invention provides a method of breeding maize comprising selecting from a breeding population of maize plants a selected maize plant with higher oil than other maize plants in the breeding population based on allelic polymorphisms associated by linkage disequilibrium to a higher seed oil-related trait, where the selected maize plant has 1 or more higher oil alleles linked to a maize oil marker of this invention. The maize oil markers are also useful in a method of breeding maize comprising selecting a maize line having a haplotype characterized by the maize oil markers. The maize oil markers are also useful in methods of this invention for identifying other polymorphic maize DNA loci, which are useful for genotyping between at least two varieties of maize. More particularly such a method comprises identifying a locus comprising at least 20 consecutive nucleotides which are linked to a maize oil marker locus of this invention. Thus, a further aspect of this invention provides methods of breeding maize comprising selecting a maize line having a polymorphism associated by linkage disequilibrium to a seed oil-related trait locus where such polymorphism is linked to a maize oil marker of this invention.

Aspects of this invention related to maize oil markers are isolated nucleic acid molecules that are useful for detecting a polymorphism associated with oil in maize, e.g. molecules that are known in the art as PCR primers and hybridization probes for using the markers in genotyping.

### DETAILED DESCRIPTION OF PREFERRED EMBODIMENTS

In the sequence listing:

SEQ ID NOs 1-73 are DNA sequences of amplicons for oil-assoicated markers,

SEQ ID NOs 74-146 are DNA sequences for oil-associated genes,

SEQ ID NOs 147-219 are amino acid sequences for proteins encoded by oil-associated genes, and

SEQ ID NOs 220-2337 are amino acid sequences for proteins encoded by homologs of oil-associated genes.

In Tables 1-5:

Table 5 identifies polymorphic markers, i.e. SNPs and Indels, in each of the 73 oil-assoicated marker amplicons sequences, i.e. SEQ ID NO:1-73,

Table 2 identifies each of the 73 DNA sequences for oil-associated genes by arbitrary name of the gene and the encoded protein, i.e. SEQ ID NO:74-146,

Table 3 identifies each of the 73 amino acid sequences for proteins encoded by an oil-associated gene by annotated function, i.e. SEQ ID NO:147-219,

Table 4 identifies homologs of oil-associated genes by reference to a name assigned to a sequence in a protein database for SEQ ID NO:147-219, and

Table 5 identifies each of the amino acid sequences of proteins encoded by homologs of oil-associated genes, i.e SEQ ID NO:220-2337, by reference to the name assigned in Table 4 and indication of source organism.

As used herein certain terms are defined as follows.

An “oil-associated gene” means a nucleic acid molecule comprising at least a functional part of the open reading frame of a gene (or a homolog thereof) that either overlaps with, or is associated by linkage disequilibrium with, any one or more of the 73 genomic amplicons of SEQ ID NO:1 through SEQ ID NO:73, which contain markers having a statistically significant association with an oil trait. More particularly, oil-associated genes are found in the group consisting of:
(a) on maize chromosome 1 the genes characterized by nucleic acid sequences of SEQ ID NO: 140, 128, 108, 111, 123, 105, 131, 100, 78, 101, and 146; genes encoding proteins having an amino acid sequence selected from the group consisting of SEQ ID NO: 213, 201, 181, 184, 196, 178, 204, 173, 151, 174, and 219; and homologs thereof selected from plants, fungi, algae and bacteria;
(b) on maize chromosome 2 the genes characterized by nucleic acid sequences of SEQ ID NO: 95, 126, 82, 74, 89, 113, and 116; genes encoding proteins having an amino acid sequence selected from the group consisting of SEQ ID NO: 168, 199, 155, 147, 162, 186, and 189; and homologs thereof selected from plants, fungi, algae and bacteria;
(c) on maize chromosome 3 the genes characterized by nucleic acid sequences of SEQ ID NO: 80, 98, 94, 87, 99, 79, and 135; genes encoding proteins having an amino acid sequence selected from the group consisting of SEQ ID NO: 153, 171, 167, 160, 172, 152, and 208; and homologs thereof selected from plants, fungi, algae and bacteria;
(d) on maize chromosome 4 the genes characterized by nucleic acid sequences of SEQ ID NO: 134, 130, 110, 91, 77, 86, 97, 85, and 102; genes encoding proteins having an amino acid sequence selected from the group consisting of SEQ ID NO: 207, 203, 183, 164, 150, 159, 170, 158, and 175; and homologs thereof selected from plants, fungi, algae and bacteria;
(e) on maize chromosome 5 the genes characterized by nucleic acid sequences of SEQ ID NO: 133, 118, 117, 144, 141, 93, 139, 129, 103, and 119; genes encoding proteins having an amino acid sequence selected from the group consisting of SEQ ID NO: 206, 191, 190, 217, 214, 166, 212, 202, 176, and 192; and homologs thereof selected from plants, fungi, algae and bacteria;
(f) on maize chromosome 6 the genes characterized by nucleic acid sequences of SEQ ID NO: 75, 122, 121, 145, 84, 96, and 107; genes encoding proteins having an amino acid sequence selected from the group consisting of SEQ ID NO: 148, 195, 194, 218, 157, 169, and 180; and homologs thereof selected from plants, fungi, algae and bacteria;
(g) on maize chromosome 7 the genes characterized by nucleic acid sequences of SEQ ID NO: 114, 115, 104, 109, 143, 83, and 106; genes encoding proteins having an amino acid sequence selected from the group consisting of SEQ ID NO: 187, 188, 177, 182, 216, 156, and 179; and homologs thereof selected from plants, fungi, algae and bacteria;
(h) on maize chromosome 8 the genes characterized by nucleic acid sequences of SEQ ID NO: 112, 132, 142, 90, 124, 127, and 81; genes encoding proteins having an amino acid sequence selected from the group consisting of SEQ ID NO: 185, 205, 215, 163, 197, 200, and 154; and homologs thereof selected from plants, fungi, algae and bacteria;
(i) on maize chromosome 9 the genes characterized by nucleic acid sequences of SEQ ID NO: 120, 137, 76, 125, and 136; genes encoding proteins having an amino acid sequence selected from the group consisting of SEQ ID NO: 193, 210, 149, 198, and 209; and homologs thereof selected from plants, fungi, algae and bacteria;
(j) on maize chromosome 10 the genes characterized by nucleic acid sequences of SEQ ID NO: 138, 88, and 92; genes encoding proteins having an amino acid sequence selected from the group consisting of SEQ ID NO: 211, 161, and 165; and homologs thereof selected from plants, fungi, algae and bacteria;
(k) nucleic acid molecules comprising oligonucleotides of at least 40 consecutive nucleic acid residues of a gene in sections (a) through (j) and having at least 60%, more preferably at least 70%, even more preferably at least 80%, and most preferably at least 90% identity with a same length fragment of said gene; and
(l) nucleic acid molecules encoding proteins having amino acid sequence which has at least 80% identity, preferably at least 90% identity, to an amino acid sequence of a protein in sections (a) through (j) over a window of alignment.

An “allele” means an alternative sequence at a particular locus; the length of an allele can be as small as 1 nucleotide base but is typically larger. Allelic sequence can be amino acid sequence or nucleic acid sequence.

A “locus” is a short sequence that is usually unique and usually found at one particular location by a point of reference, e.g., a short DNA sequence that is a gene, or part of a gene or intergenic region. A locus of this invention can be a unique PCR product. The loci of this invention are polymorphic between certain individuals.

“Genotype” means the specification of an allelic composition at one or more loci within an individual organism. In the case of diploid organisms, there are two alleles at each locus; a diploid genotype is said to be homozygous when the alleles are the same, and heterozygous when the alleles are different.

“Consensus sequence” means
(a) a constructed DNA sequence that identifies SNP and Indel polymorphisms in alleles at a locus. Consensus sequence of a polymorphic locus can be based on either strand of DNA at the locus and states the nucleotide base of either one of each SNP in the locus and the nucleotide bases of all Indels in the locus. Thus, although a consensus sequence of a polymorphic locus may not be a copy of an actual DNA sequence, a consensus sequence is useful for precisely designing primers and probes for actual polymorphisms in the locus.
(b) a conserved amino acid sequence of part or all of the proteins encoded by homologous genes.

“Homolog” of an oil-associated gene as used herein means a gene from a the same or a different organism that performs the same biological function as the oil-associated gene. An orthologous relation between two organisms is not necessarily manifest as a one-to-one correspondence between two genes, because a gene can be duplicated or deleted after organism phylogenetic separation, such as speciation. So for a given gene, there may be no ortholog or more than one ortholog or the function may be performed by an alternatively spliced gene. Other complicating factors include limited gene identification, redundant copies of the same gene with different sequence lengths or corrected sequence. A local sequence alignment program, e.g. BLAST, can be used to search a database of sequences to find similar sequences, and the summary Expectation value (E-value) can be used to measure the sequence base similarity. Because query results with the best E-value for a particular organism may not necessarily be an ortholog or the only ortholog, it is necessary to use a reciprocal BLAST search to filter the hit sequences with significant E-values before calling them orthologs. The reciprocal BLAST entails search of the significant hits against a database of genes from the base organism that are similar to the query gene. A hit is a likely ortholog when the reciprocal BLAST's best hit is the query gene itself or is one of the duplicated genes of the query gene after speciation. Some skilled in the art may argue that what is called a homolog is in fact an ortholog or a paralog. Regardless, the term homolog is used herein to describe genes which are assumed to have functional similarity by inference from sequence base similarity.

“Phenotype” means the detectable characteristics of a cell or organism that are a manifestation of gene expression.

“Marker” means a polymorphic sequence. A “polymorphism” is a variation among individuals in sequence, particularly in DNA sequence. Useful polymorphisms include a single nucleotide polymorphisms (SNPs) and insertions or deletions in DNA sequence (Indels).

“Maize oil marker” means a marker in any one of the genomic amplicons of SEQ ID NO:1 through SEQ ID NO:73 and markers in linkage disequilibrium with a marker in said amplicons.

“Marker assay” means a method for detecting a polymorphism at a particular locus using a particular method, e.g., phenotype (such as seed color, flower color, or other visually detectable trait), restriction fragment length polymorphism (RFLP), single base extension, electrophoresis, sequence alignment, allelic specific oligonucleotide hybridization (ASO), RAPID, etc. Preferred marker assays include single base extension as disclosed in U.S. Pat. No. 6,013,431 and allelic discrimination where endonuclease activity releases a reporter dye from a hybridization probe as disclosed in U.S. Pat. No. 5,538,848, the disclosures of both of which are incorporated herein by reference.

“Linkage” refers to relative frequency at which types of gametes are produced in a cross. For example, if locus A has alleles “A” or “a” and locus B has alleles “B” or “b,” a cross between parent 1 with AABB genotype and parent II with aabb genotype will produce four possible gametes where the haploid genotypes are segregated into AB, Ab, aB and ab. The null expectation is that there will be independent and equal segregation into each of the four possible genotypes, i.e., with no linkage, ¼ of the gametes will be of each genotype. Segregation of gametes into a genotypes differing from ¼ are attributed to linkage. Two loci are said to be “genetically linked” when they show this deviation from the expected equal frequency of ¼.

“Linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and ab is p′q′. Any deviation from the expected frequency is called linkage disequilibrium.

“Quantitative Trait Locus (QTL)” means a locus that controls to some degree numerically representable traits that are usually continuously distributed.

“Haplotype” means the genotype for multiple loci or genetic markers in a haploid gamete. Generally, these loci or markers reside within a relatively small and defined region of a chromosome. A preferred haplotype comprises the 10 cM region or the 5 cM region or the 2 cM region surrounding an informative marker having a significant association with oil.

“Hybridizing” means the capacity of two nucleic acid molecules or fragments thereof to form anti-parallel, double-stranded nucleotide structure. The nucleic acid molecules of this invention are capable of hybridizing to other nucleic acid molecules under certain circumstances. A nucleic acid molecule is said to be the “complement” of another nucleic acid molecule if the molecules exhibit “complete complementarity,” i.e., each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules that hybridize to other nucleic acid molecules, e.g., at least under low stringency conditions are said to be “hybridizable cognates” of the other nucleic acid molecules. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985), each of which is incorporated herein by reference. Departures from complete complementarity are therefore permissible, as long as such departures do not completely preclude the capacity of the molecules to form a double-stranded structure. Thus, in order for a nucleic acid molecule to serve as a primer or probe, it need only be sufficiently complementary in sequence to be able to form a stable double-stranded structure under the particular solvent and salt concentrations employed. Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6, incorporated herein by reference. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

“Sequence identity” refers to the extent to which two optimally aligned DNA or amino acid sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc. Burlington, Mass.). Polynucleotides of the present invention that are variants of the polynucleotides provided herein will generally demonstrate significant identity with the polynucleotides provided herein. Of particular interest are DNA homologs having at least about 70% sequence identity, at least about 80% sequence identity, at least about 90% sequence identity, and more preferably even greater, such as 98% or 99% sequence identity with DNA sequences of an oil-associated gene described herein. Homologous DNA can be characterized by the cognate encoded protein and will have at least 80%, preferably at least 90% identity with amino acid sequence of a protein encoded by an oil-associated gene.

“Genetic transformation” means a process of introducing a DNA construct (e.g., a vector or expression cassette) into a cell or protoplast in which that exogenous DNA is incorporated into a chromosome or is capable of autonomous replication.

“Exogenous gene” means a gene or partial gene that is not normally present in a given host genome in the exogenous gene's present form. In this respect, the gene itself may be native to the host genome; however, the exogenous gene will comprise the native gene altered by the addition or deletion of one or more different regulatory elements.

“Expression” means the combination of intracellular processes, including transcription and translation undergone by a coding DNA molecule such as a structural gene to produce a polypeptide.

“Progeny” means any subsequent generation, including the seeds and plants therefrom, that is derived from a particular parental plant or set of parental plants.

“Promoter” means a recognition site on a DNA sequence or group of DNA sequences that provides an expression control element for a structural gene and to which RNA polymerase specifically binds and initiates RNA synthesis (transcription) of that gene.

“R₀ transgenic plant” means a plant that has been directly transformed with a selected DNA or has been regenerated from a cell or cell cluster that has been transformed with a selected DNA.

“Regeneration” means the process of growing a plant from a plant cell (e.g., plant protoplast, callus or explant).

“DNA construct” means a chimeric DNA molecule that is designed for introduction into a host genome by genetic transformation. Preferred DNA constructs will comprise all of the genetic elements necessary to direct the expression of one or more exogenous genes. In particular embodiments of the instant invention, it may be desirable to introduce a DNA construct into a host cell in the form of an expression cassette.

“Transformed cell” means a cell the DNA complement of which has been altered by the introduction of an exogenous DNA molecule into that cell.

“Transgene” means a segment of DNA that has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation or may be inherited from a plant of any previous generation that was transformed with the DNA segment.

“Transgenic plant” means a plant or progeny plant of any subsequent generation derived therefrom, wherein the DNA of the plant or progeny thereof contains an introduced exogenous DNA segment not originally present in a non-transgenic plant of the same strain. The transgenic plant may additionally contain sequences that are native to the plant being transformed, but wherein the “exogenous” gene has been altered in order to alter the level or pattern of expression of the gene.

“Transit peptide” means a polypeptide sequence that is capable of directing a polypeptide to a particular organelle or other location within a cell.

“Vector” means a DNA molecule capable of replication in a host cell and/or to which another DNA segment can be operatively linked so as to bring about replication of the attached segment. A plasmid is an exemplary vector.

“Purified” refers to a nucleic acid molecule or polypeptide separated from substantially all other molecules normally associated with it in its native state. More preferably, a substantially purified molecule is the predominant species present in a preparation. A substantially purified molecule may be greater than 60% free or 75% free or 90% free or 95% free from the other molecules (exclusive of solvent) present in the natural mixture. The terms “isolated and purified” and “substantially purified” are not intended to encompass molecules present in their native state.

As used herein “yield” means the production of a crop, e.g. shelled corn kernels or soybean or cotton fiber, per unit of production area, e.g. in bushels per acre or metric tons per hectare, often reported on a moisture adjusted basis, e.g. corn is typically reported at 15.5% moisture. Moreover a bushel of corn is defined by law in the State of Iowa as 56 pounds by weight, a useful conversion factor for corn yield is: 100 bushels per acre is equivalent to 6.272 metric tons per hectare. Other measurements for yield are in common practice.

The molecules and organisms of the invention may also be “recombinant,” which describes (a) nucleic acid molecules that are constructed or modified outside of cells and that can replicate or function in a living cell, (b) molecules that result from the transcription, replication or translation of recombinant nucleic acid molecules, or (c) organisms that contain recombinant nucleic acid molecules or are modified using recombinant nucleic acid molecules.

As used herein a “transgenic” organism, e.g. plant or seed, is one whose genome has been altered by the incorporation of exogenous genetic material or additional copies of native genetic material, e.g. by transformation or recombination of the organism or an ancestor organism. Transgenic plants include progeny plants of an original plant derived from a transformation process including progeny of breeding transgenic plants with wild type plants or other transgenic plants. Crop plants of interest in the present invention include, but are not limited to maize, soybean, cotton, canola (rape), sunflower, safflower and flax.

“Enhanced oil” in a transgenic cell or organism having recombinant DNA comprising an oil-associated gene is determined by reference to cell or organism without that recombinant DNA, e.g. a wild-type plant, a non-recombinant ancestor plant line or a negative segregant progeny from a hemizygous transgenic plant. Enhanced oil can be determined by direct or indirect measurement. Enhanced oil activity can be achieved by linking a constitutive promoter to an oil-associated gene. Reduced oil can also be achieved through genetic engineering of oil-associated genes, e.g. by a variety of mechanisms including anti-sense, co-suppression, double stranded RNA (dsRNA), mutation or knockout.

As used herein “gene suppression” means any of the well-known methods for suppressing expression of protein. Posttranscriptional gene suppression is mediated by transcription of integrated recombinant DNA to form double-stranded RNA (dsRNA) having homology to a gene targeted for suppression. This formation of dsRNA most commonly results from transcription of an integrated inverted repeat of the target gene, and is a common feature of gene suppression methods known as anti-sense suppression, co-suppression and RNA interference (RNAi). See Redenbaugh et al. in “Safety Assessment of Genetically Engineered Flavr Savr™ Tomato, CRC Press, Inc. (1992); Jorgensen et al., Mol. Gen. Genet., 207:471-477 (1987); and Stam et al., The Plant Journal, 12(1), 63-82 (1997). Methods for such gene suppression are disclosed in U.S. Pat. No. 5,107,065 (Shewmaker et al.); U.S. Pat. No. 5,283,184 (Jorgensen et al.); U.S. Pat. No. 6,326,193 U.S. Pat. No. 6,506,559 (Fire et al.); U.S. 2002/0048814 A1 (Oeller); U.S. 2003/0018993 A1 (Gutterson et al.); U.S. 2003/0175965 A1 (Lowe et al.); U.S. 2003/0036197 A1 (Glassman et al.); U.S. patent application Ser. No. 10/465,800 (Fillatti), and U.S. application Ser. No. 10/393,347 (Shewmaker et al.), incorporated herein by reference. Transcriptional suppression can be mediated by a transcribed dsRNA having homology to a promoter DNA sequence to effect what is called promoter trans suppression. Constructs useful for such gene suppression mediated by promoter trans suppression are disclosed by Mette et al., The EMBO Journal, Vol. 18, No. 1, pp. 241-148, 1999 and by Mette et al., The EMBO Journal, Vol. 19, No. 19, pp. 5194-5201-148, 2000. Suppression of an oil-associated gene by RNAi can be achieved using a recombinant DNA construct having a promoter operably linked to a DNA element comprising a sense and anti-sense element of a segment of genomic DNA of the oil-associated gene, e.g. a segment of at least about 23 nucleotides, more preferably about 50 to 200 nucleotides where the sense and anti-sense DNA components can be directly linked or joined by an intron or artificial DNA segment that can form a loop when the transcribed RNA hybridizes to form a hairpin structure. For example, genomic DNA from a polymorphic locus of SEQ ID NO:1 through SEQ ID NO:73 can be used in a recombinant construct for suppression of a cognate oil-associated gene by RNAi suppression.

Characteristics of Oil-Associated Genes

This invention provides nucleic acid molecules comprising DNA sequence representing oil-associated genes having a nucleic acid sequence of SEQ ID NO:74 through SEQ ID NO:146 or fragments of such oil-associated genes such as substantial parts of oil-associated genes providing the protein coding sequence part of the oil-associated gene. The oil-associated genes of this invention have been identified by marker trait association.

Homologous oil-associated genes have been identified in other plants and in other organisms such as fungi, algae and bacteria using the nucleic acid sequence of a known oil-associated gene or the amino acid sequence of a protein encoded by an oil-associated gene in any of a variety of search algorithms, e.g. the BLAST search algorithm, in public or proprietary DNA and protein databases. Existence of a gene is inferred if significant sequence similarity extends over the sequence of the target gene. Because homology-based methods may overlook genes unique to the source organism, for which homologous nucleic acid molecules have not yet been identified in databases, gene prediction programs are also used. Gene prediction programs generally use “signals” in the sequence, such as splice sites or “content” statistics, such as codon bias; to predict gene structures (Stormo, Genome Research 10: 394-397, 2000). Proteins encoded by homologs of oil-associated genes are identified by reference to Tables 4 and 5 have amino acid sequences of SEQ IS NO:220 through SEQ ID NO:2337.

With respect to nucleotide sequences, degeneracy of the genetic code provides the possibility to substitute at least one base of the base sequence of a gene with a different base without causing the amino acid sequence of the polypeptide produced from the gene to be changed. Hence, the DNA of the present invention may also have any codon changed in a sequence of SEQ ID NO: 1 through SEQ ID NO: 146 by substitution in accordance with degeneracy of genetic code. See U.S. Pat. No. 5,500,365, incorporated herein by reference.

More particularly, the homologous oil-associated genes can be characterized by reference to an artificial consensus sequence of conserved amino acids determined from an alignment of protein sequence encoded by such homologs.

Characteristics of Maize Oil Markers

The maize loci of this invention comprise a DNA sequence that comprises at least 20 consecutive nucleotides and includes or is adjacent to one or more polymorphisms identified in Table 1. Such maize loci have a nucleic acid sequence having at least 90% sequence identity or at least 95% or for some alleles at least 98% and in many cases at least 99% sequence identity, to the sequence of the same number of nucleotides in either strand of a segment of maize DNA that includes or is adjacent to the polymorphism. The nucleotide sequence of one strand of such a segment of maize DNA may be found in a polymorphic locus with a sequence in the group consisting of SEQ ID NO:1 through SEQ ID NO:73. It is understood by the very nature of polymorphisms that for at least some alleles there will be no identity to the polymorphism, per se. Thus, sequence identity can be determined for sequence that is exclusive of the polymorphism sequence. The polymorphisms in each locus are identified more particularly in Table 1.

For many genotyping applications it is useful to employ as markers polymorphisms from more than one locus. Thus, aspects of the invention use a collection of different loci. The number of loci in such a collection can vary but will be a finite number, e.g., as few as 2 or 5 or 10 or 25 loci or more, for instance up to 40 or 75 or 100 or more loci.

Another aspect of the invention provides nucleic acid molecules that are capable of hybridizing to the polymorphic maize loci of this invention, e.g. PCR primers and hybridization probes. In certain embodiments of the invention, e.g., which provide PCR primers, such molecules comprise at least 15 nucleotide bases. Molecules useful as primers can hybridize under high stringency conditions to one of the strands of a segment of DNA in a polymorphic locus of this invention. Primers for amplifying DNA are provided in pairs, i.e., a forward primer and a reverse primer. One primer will be complementary to one strand of DNA in the locus and the other primer will be complementary to the other strand of DNA in the locus, i.e., the sequence of a primer is at least 90% or at least 95% identical to a sequence of the same number of nucleotides in one of the strands. It is understood that such primers can hybridize to a sequence in the locus that is distant from the polymorphism, e.g., at least 5, 10, 20, 50 or up to about 100 nucleotide bases away from the polymorphism. Design of a primer of this invention will depend on factors well known in the art, e.g., avoidance of repetitive sequence.

Another aspect of the nucleic acid molecules of this invention are hybridization probes for polymorphism assays. In one aspect of the invention such probes are oligonucleotides comprising at least 12 nucleotide bases and a detectable label. The purpose of such a molecule is to hybridize, e.g., under high stringency conditions, to one strand of DNA in a segment of nucleotide bases that includes or is adjacent to the polymorphism of interest in an amplified part of a polymorphic locus. Such oligonucleotides are at least 90% or at least 95% identical to the sequence of a segment of the same number of nucleotides in one strand of maize DNA in a polymorphic locus. The detectable label can be a radioactive element or a dye. In preferred aspects of the invention, the hybridization probe further comprises a fluorescent label and a quencher, e.g., for use in hybridization probe assays of the type known as Taqman assays, available from Applied Biosystems of Foster City, Calif.

For assays where the molecule is designed to hybridize adjacent to a polymorphism that is detected by single base extension, e.g., of a labeled dideoxynucleotide, such molecules can comprise at least 15 or at least 16 or 17 nucleotide bases in a sequence that is at least 90% or at least 95% identical to a sequence of the same number of consecutive nucleotides in either strand of a segment of polymorphic maize DNA. Oligonucleotides for single base extension assays are available from Orchid Biosystems.

Such primer and probe molecules are generally provided in groups of two primers and one or more probes for use in genotyping assays. Moreover, it is often desirable to conduct a plurality of genotyping assays for a plurality of polymorphisms. Thus, this invention also provides collections of nucleic acid molecules, e.g., in sets that characterize a plurality of polymorphisms.

Characteristics of Protein and Polypeptide Molecules

The nucleic acid molecules of this invention encode certain protein or smaller polypeptide molecules including those having an amino acid sequence of SEQ ID NO: 147 through SEQ ID NO: 219. Homologs of the polypeptides of the present invention may be identified by comparison of the amino acid sequence of the polypeptide to amino acid sequences of polypeptides from the same or different plant sources, e.g. manually or by using known homology-based search algorithms such as those commonly known and referred to as BLAST, FASTA, and Smith-Waterman.

A further aspect of the invention comprises functional homolog proteins which differ in one or more amino acids from those of a polypeptide provided herein as the result of one or more of the well-known conservative amino acid substitutions, e.g. valine is a conservative substitute for alanine and threonine is a conservative substitute for serine. Conservative substitutions for an amino acid within the native polypeptide sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the invention comprises polypeptides which differ in one or more amino acids from those of a described protein sequence as the result of deletion or insertion of one or more amino acids in a native sequence.

Recombinant DNA Constructs for Plant Transformation

The present invention contemplates the use of polynucleotides which encode a protein effective for imparting altered oil levels in plants. Such polynucleotides are assembled in recombinant DNA constructs using methods known to those of ordinary skill in the art. A useful technology for building DNA constructs and vectors for transformation is the GATEWAY™ cloning technology (available from Invitrogen Life Technologies, Carlsbad, Calif.) uses the site specific recombinase LR cloning reaction of the Integrase/att system from bacteriophage lambda vector construction, instead of restriction endonucleases and ligases. The LR cloning reaction is disclosed in U.S. Pat. Nos. 5,888,732 and 6,277,608, U.S. Patent Application Publications 2001283529, 2001282319 and 20020007051, all of which are incorporated herein by reference. The GATEWAY™ Cloning Technology Instruction Manual which is also supplied by Invitrogen also provides concise directions for routine cloning of any desired DNA into a vector comprising operable plant expression elements.

Transgenic DNA constructs used for transforming plant cells will comprise the heterologous DNA which one desires to introduced into and a promoter to express the heterologous DNA in the host maize cells. As is well known in the art such constructs typically also comprise a promoter and other regulatory elements, 3′ untranslated regions (such as polyadenylation sites), transit or signal peptides and marker genes elements as desired. For instance, see U.S. Pat. Nos. 5,858,642 and 5,322,938 which disclose versions of the constitutive promoter derived from cauliflower mosaic virus (CaMV35S), U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. Patent Application Publication 2002/0192813A1 which discloses 5′, 3′ and intron elements useful in the design of effective plant expression vectors, U.S. patent application Ser. No. 09/078,972 which discloses a coixin promoter, U.S. patent application Ser. No. 09/757,089 which discloses a maize chloroplast aldolase promoter, all of which are incorporated herein by reference.

In many aspects of the invention it is preferred that the promoter element in the DNA construct should be seed or kernel tissue specific. Such promoters can be identified and isolated by those skilled in the art from the regulatory region of plant genes which are over expressed in seed tissue, e.g. embryo or endosperm. For example, specific seed tissue-specific promoters for use in this invention include an L3 oleosin promoter as disclosed in U.S. Pat. No. 6,433,252, a gamma coixin promoter as disclosed in U.S. patent application Ser. No. 09/078,972, and emb5 promoter as disclosed in U.S. provisional application Ser. No. 60/434,242, all of which are incorporated herein by reference.

In general, it is preferred to introduce heterologous DNA randomly, i.e. at a non-specific location, in the plant genome. In special cases, it may be useful to target heterologous DNA insertion in order to achieve site specific integration, e.g. to replace an existing gene in the genome. In some other cases it may be useful to target a heterologous DNA integration into the genome at a predetermined site from which it is known that gene expression occurs. Several site specific recombination systems exist which are known to function in plants and include cre-lox as disclosed in U.S. Pat. No. 4,959,317 and FLP-FRT as disclosed in U.S. Pat. No. 5,527,695, both incorporated herein by reference.

Constructs and vectors may also include a transit peptide for targeting of a gene target to a plant organelle, particularly to a chloroplast, leucoplast or other plastid organelle. For a description of the use of a chloroplast transit peptide see U.S. Pat. No. 5,188,642, incorporated herein by reference.

In practice, DNA is introduced into only a small percentage of target cells in any one experiment. Selectable marker genes are used to provide an efficient system for identification of those cells that are stably transformed by receiving and integrating a transgenic DNA construct into their genomes. Preferred selectable marker genes confer resistance to a selective agent, such as an antibiotic or herbicide. Potentially transformed cells are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Useful selectable marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS). Examples of such selectable marker genes are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Exogenous Oil-Associated Genes for Modification of Plant Phenotypes

A particularly important advance of the present invention is that it provides DNA sequences useful for producing desirable oil-related phenotypes in plants, preferably in crop plants such as soybean, cotton, canola, sunflower, safflower, flax and most preferably in maize.

The choice of a selected DNA sequence for expression in a plant host cell in accordance with the invention will depend on the purpose of gene expression, e.g., expression of a native gene or homolog by a constitutive promoter, over expression of a native gene or homolog, suppression of a native gene, or altered tissue- or stage-specific expression of a native gene or homolog by a tissue- or stage-specific promoter.

In certain embodiments of the invention, transformation of a recipient cell may be carried out with more than one exogenous DNA coding region. As used herein, an “exogenous coding region” or “selected coding region” is a coding region not normally found in the host genome in an identical context. By this, it is meant that the coding region may be isolated from a different species than that of the host genome, or alternatively, isolated from the host genome, but it is operably linked to one or more regulatory regions that differ from those found in the unaltered, native gene. Two or more exogenous coding regions also can be supplied in a single transformation event using either distinct transgene-encoding vectors, or using a single vector incorporating two or more coding sequences.

Enhancement of an oil-related trait can also be effected by suppression of one or more genes that express proteins that divert oil producing materials into competing products or that degrade oil products. Site-directed inactivation of a gene, while possible, is typically difficult to achieve. Other more effective methods of gene suppression include the use anti-sense RNA, co-suppression, interfering RNA, processing defective RNA, transposon tagging, backcrossing or homologous recombination. Post transcriptional gene suppression by RNA interference is a superior and preferred method of gene suppression. In a preferred embodiment gene suppression may complement over expression of an oil-associated gene.

Transformation Methods and Transgenic Plants

Methods and compositions for transforming plants by introducing a transgenic DNA construct into a plant genome in the practice of this invention can include any of the well-known and demonstrated methods. Preferred methods of plant transformation are microprojectile bombardment as illustrated in U.S. Pat. Nos. 5,015,580; 5,550,318; 5,538,880; 6,160,208: 6,194,636 and 6,399,861 and Agrobacterium-mediated transformation as illustrated in U.S. Pat. Nos. 5,824,877; 5,591,616; 5,981,840 and 6,384,301, all of which are incorporated herein by reference. See also U.S. application Ser. No. 09/823,676, incorporated herein by reference, for a description of vectors, transformation methods, and production of transformed Arabidopsis thaliana plants where genes in a recombinant DNA construct are constitutively expressed by a CaMV35S promoter.

Transformation methods of this invention to provide plants with enhanced environmental stress tolerance are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. No. 6,194,636 and U.S. patent application Ser. No. 09/757,089, which are incorporated herein by reference.

Regeneration and Seed Production

Cells that survive the exposure to the selective agent, or cells that have been scored positive in a screening assay, may be cultured in media that supports regeneration of plants. Such media is well-known to one of skill in the art.

The transformed cells, identified by selection or screening and cultured in an appropriate medium that supports regeneration, will then be allowed to mature into plants. Developing plantlets are transferred to soil-less plant growth mix, and hardened off, e.g., in an environmentally controlled chamber at about 85% relative humidity, 600 ppm CO₂, and 25-250 microeinsteins m⁻²s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Regenerating plants are preferably grown at about 19° C. to 28° C. After the regenerating plants have reached the stage of shoot and root development, they may be transferred to a greenhouse for further growth and testing. Plants may be pollinated using conventional plant breeding methods known to those of skill in the art and seed produced.

Progeny may be recovered from transformed plants and tested for expression of the exogenous expressible gene. The transgenic seeds of this invention can be harvested from fertile transgenic plants and be used to grow progeny generations of transformed plants of this invention, including hybrid plants; said progeny generations will contain the DNA construct expressing an oil-associated gene which provides the benefits of enhanced oil production and/or storage.

Seeds of R₀ transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

Characterization of Transgenic Plants for Presence of Exogenous DNA

To confirm the presence of the exogenous DNA in regenerating plants, a variety of assays may be performed. Such assays include, for example, “molecular biological” assays, such as Southern and Northern blotting and PCR; “biochemical” assays, such as detecting the presence of RNA, e.g., double-stranded RNA, or a protein product, e.g., by immunological means (ELISAs and Western blots) or by enzymatic function; plant part assays, such as leaf or root assays; and also, by analyzing the phenotype of the whole regenerated plant. Genomic DNA may be isolated from callus cell lines or any plant parts to determine the presence of the exogenous gene through the use of techniques well known to those skilled in the art.

The presence of DNA elements introduced through the methods of this invention may be determined by polymerase chain reaction (PCR). Using this technique, discreet fragments of DNA are amplified and detected by gel electrophoresis. This type of analysis permits one to determine whether a gene is present in a stable transformant, but it does not necessarily prove integration of the introduced gene into the host cell genome. Typically, DNA has been integrated into the genome of all transformants that demonstrate the presence of the gene through PCR analysis. In addition, it is not possible using PCR techniques to determine whether transformants have exogenous genes introduced into different sites in the genome, i.e., whether transformants are of independent origin. Using PCR techniques it is possible to clone fragments of the host genomic DNA adjacent to an introduced gene.

Positive proof of DNA integration into the host genome and the independent identities of transformants may be determined using the technique of Southern hybridization. Using this technique, specific DNA sequences that were introduced into the host genome and flanking host DNA sequences can be identified. Hence the Southern hybridization pattern of a given transformant serves as an identifying characteristic of that transformant. In addition, it is possible through Southern hybridization to demonstrate the presence of introduced genes in high molecular weight DNA, i.e., confirm that the introduced gene has been integrated into the host cell genome. The technique of Southern hybridization provides information that can be obtained using PCR, e.g., the presence of a gene, but also demonstrates integration into the genome and characterizes each individual transformant. It is contemplated that using the techniques of dot or slot blot hybridization, which are modifications of Southern hybridization techniques, one could obtain the same information that is derived from PCR, e.g., the presence of a gene.

Both PCR and Southern hybridization techniques can be used to demonstrate transmission of a transgene to progeny. In most instances the characteristic Southern hybridization pattern for a given transformant will segregate in progeny as one or more Mendelian genes, indicating stable inheritance of the transgene.

Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species. It is further contemplated that TAQMAN® technology (Applied Biosystems, Foster City, Calif.) may be used to quantitate both DNA and RNA in a transgenic cell.

Although Southern blotting and PCR may be used to detect the gene(s) in question, they do not provide information as to whether the gene is being expressed. Expression may be evaluated by specifically identifying the protein products of the introduced genes or evaluating the phenotypic changes brought about by their expression. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the product of interest such as evaluation by amino acid sequencing following purification.

Event-Specific Transgene Assays

Southern blotting, PCR and RT-PCR techniques can be used to identify the presence or absence of a given transgene but, depending upon experimental design, may not specifically and uniquely identify identical or related transgene constructs located at different insertion points within the recipient genome. To more precisely characterize the presence of transgenic material in a transformed plant, one skilled in the art could identify the point of insertion of the transgene and, using the sequence of the recipient genome flanking the transgene, develop an assay that specifically and uniquely identifies a particular insertion event. Many methods can be used to determine the point of insertion such as, but not limited to, Genome Walker™ technology (CLONTECH, Palo Alto, Calif.), Vectorette™ technology (Sigma, St. Louis, Mo.), restriction site oligonucleotide PCR, uneven PCR, and generation of genomic DNA clones containing the transgene of interest in a vector such as, but not limited to, lambda phage.

Once the sequence of the genomic DNA directly adjacent to the transgenic insert on either or both sides has been determined, one skilled in the art can develop an assay to specifically and uniquely identify the insertion event. For example, two oligonucleotide primers can be designed, one wholly contained within the transgene and one wholly contained within the flanking sequence, that can be used together with the PCR technique to generate a PCR product unique to the inserted transgene. In one embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the transgene. In another embodiment, the two oligonucleotide primers for use in PCR could be designed such that one primer is complementary to sequences in both the transgene and adjacent flanking sequence such that the primer spans the junction of the insertion site while the second primer could be homologous to sequences contained wholly within the genomic sequence adjacent to the insertion site. Confirmation of the PCR reaction may be monitored by, but not limited to, size analysis on gel electrophoresis, sequence analysis, hybridization of the PCR product to a specific radiolabeled DNA or RNA probe or to a molecular beacon, or use of the primers in conjugation with a TAQMAN™ probe and technology (Applied Biosystems, Foster City, Calif.)

Site-Specific Integration or Excision of Transgenes

It is specifically contemplated by the inventors that one could employ techniques for the site-specific integration or excision of transformation constructs prepared in accordance with the instant invention. An advantage of site-specific integration or excision is that it can be used to overcome problems associated with conventional transformation techniques, in which transformation constructs typically randomly integrate into a host genome and multiple copies of a construct may integrate. Site-specific integration can be achieved in plants by means of homologous recombination as disclosed, for example, in U.S. Pat. Nos. 5,527,695 and 5,658,772, incorporated herein by reference.

Deletion of Sequences Located within the Transgenic Insert

During the transformation process it is often necessary to include ancillary sequences, such as selectable marker or reporter genes, for tracking the presence or absence of a desired trait gene transformed into the plant on the DNA construct. Such ancillary sequences often do not contribute to the desired trait or characteristic conferred by the phenotypic trait gene. Homologous recombination is a method by which introduced sequences may be selectively deleted in transgenic plants.

Deletion of sequences by homologous recombination relies upon directly repeated DNA sequences positioned about the region to be excised, so that the repeated DNA sequences direct excision utilizing native cellular recombination mechanisms. The first fertile transgenic plants are crossed to produce either hybrid or inbred progeny plants, and from those progeny plants, one or more second fertile transgenic plants are selected that contain a second DNA sequence that has been altered by recombination, preferably resulting in the deletion of the ancillary sequence. The first fertile plant can be either hemizygous or homozygous for the DNA sequence containing the directly repeated DNA that will drive the recombination event as disclosed in U.S. application Ser. No. 09/521,557, incorporated herein by reference.

Detecting Polymorphisms

Polymorphisms in DNA sequences can be detected by a variety of effective methods well known in the art including those methods disclosed in U.S. Pat. Nos. 5,468,613 and 5,217,863 by hybridization to allele-specific oligonucleotides; in U.S. Pat. Nos. 5,468,613 and 5,800,944 by probe ligation; in U.S. Pat. No. 5,616,464 by probe linking; and in U.S. Pat. Nos. 6,004,744; 6,013,431; 5,595,890; 5,762,876; and 5,945,283 by labeled base extension, all of which are incorporated herein by reference.

In another preferred method for detecting polymorphisms, SNPs and Indels can be detected by methods disclosed in U.S. Pat. Nos. 5,210,015; 5,876,930; and 6,030,787 in which an oligonucleotide probe having a 5′fluorescent reporter dye and a 3′quencher dye covalently linked to the 5′ and 3′ ends of the probe. When the probe is intact, the proximity of the reporter dye to the quencher dye results in the suppression of the reporter fluorescence, e.g., by Forster-type energy transfer. A PCR reaction is designed such that forward and reverse primers hybridize to specific sequences of the target DNA flanking a polymorphism. The hybridization probe hybridizes to polymorphism-containing sequence within the amplified PCR product. In the subsequent PCR cycle, DNA polymerase with 5′→3′ exonuclease activity cleaves the probe and separates the reporter dye from the quencher dye resulting in increased fluorescence of the reporter. A useful assay is available from AB Biosystems as the Taqman® assay, which employs four synthetic oligonucleotides in a single reaction that concurrently amplifies the maize genomic DNA, discriminates between the alleles present, and directly provides a signal for discrimination and detection. Two of the four oligonucleotides serve as PCR primers and generate a PCR product encompassing the polymorphism to be detected. Two others are allele-specific fluorescence-resonance-energy-transfer (FRET) probes. FRET probes incorporate a fluorophore and a quencher molecule in close proximity so that the fluorescence of the fluorophore is quenched. The signal from a FRET probe is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), VIC (a dye from Applied Biosystems Foster City, Calif.), and 6-carboxyfluorescein phosphoramidite (FAM). A useful quencher is 6-carboxy-N,N,N′,N′-tetramethylrhodamine (TAMRA). Additionally, the 3′end of each FRET probe is chemically blocked so that it cannot act as a PCR primer. During the assay, maize genomic DNA is added to a buffer containing the two PCR primers and two FRET probes. Also present is a third fluorophore used as a passive reference, e.g., rhodamine X (ROX), to aid in later normalization of the relevant fluorescence values (correcting for volumetric errors in reaction assembly). Amplification of the genomic DNA is initiated. During each cycle of the PCR, the FRET probes anneal in an allele-specific manner to the template DNA molecules. Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred.

To design primers and probes for the assay the locus sequence is first masked to prevent design of any of the three primers to sites that match known maize repetitive elements (e.g., transposons) or are of very low sequence complexity (di- or tri-nucleotide repeat sequences). Design of primers to such repetitive elements will result in assays of low specificity, through amplification of multiple loci or annealing of the FRET probes to multiple sites.

PCR primers are designed (a) to have a length in the size range of 18 to 25 bases and matching sequences in the polymorphic locus, (b) to have a calculated melting temperature in the range of 57° C. to 60° C., e.g., corresponding to an optimal PCR annealing temperature of 52° C. to 55° C., (c) to produce a product that includes the polymorphic site and has a length in the size range of 75 to 250 base pairs. The PCR primers are preferably located on the locus so that the polymorphic site is at least one base away from the 3′ end of each PCR primer. The PCR primers must not contain regions that are extensively self- or inter-complementary.

FRET probes are designed to span the sequence of the polymorphic site, preferably with the polymorphism located in the 3′ most ⅔ of the oligonucleotide. In the preferred embodiment, the FRET probes will have incorporated at their 3′end a chemical moiety that, when the probe is annealed to the template DNA, binds to the minor groove of the DNA, thus enhancing the stability of the probe-template complex. The probes should have a length in the range of 12 to 17 bases and, with the 3′MGB, have a calculated melting temperature of 5° C. to 7° C. above that of the PCR primers. Probe design is disclosed in U.S. Pat. Nos. 5,538,848; 6,084,102; and 6,127,121.

Use of Polymorphisms to Establish Marker/Trait Associations

The polymorphisms in the loci of this invention can be used in marker/trait associations that are inferred from statistical analysis of genotypes and phenotypes of the members of a population. These members may be individual organisms of, e.g., maize, families of closely related individuals, inbred lines, dihaploids or other groups of closely related individuals. Such maize groups are referred to as “lines”, indicating line of descent. The population may be descended from a single cross between two individuals or two lines (e.g., a mapping population) or it may consist of individuals with many lines of descent. Each individual or line is characterized by a single or average trait phenotype and by the genotypes at one or more marker loci.

Several types of statistical analysis can be used to infer marker/trait association from the phenotype/genotype data, but a basic idea is to detect markers, i.e., polymorphisms, for which alternative genotypes have significantly different average phenotypes. For example, if a given marker locus A has three alternative genotypes (AA, Aa and aa), and if those three classes of individuals have significantly different phenotypes, then one infers that locus A is associated with the trait. The significance of differences in phenotype may be tested by several types of standard statistical tests such as linear regression of marker genotypes on phenotype or analysis of variance (ANOVA). Commercially available, statistical software packages commonly used to do this type of analysis include SAS Enterprise Miner (SAS Institute Inc., Cary, N.C.) and Splus (Insightful Corporation. Cambridge, Mass.).

Often the goal of an association study is not simply to detect marker/trait associations, but to estimate the location of genes affecting the trait directly (i.e., QTLs) relative to the marker locations. In a simple approach to this goal, one makes a comparison among marker loci of the magnitude of difference among alternative genotypes or the level of significance of that difference. Trait genes are inferred to be located nearest the marker(s) that have the greatest associated genotypic difference. In a more complex analysis, such as interval mapping (Lander and Botstein, Genetics 121:185-199, 1989), each of many positions along the genetic map (say at 1 cM intervals) is tested for the likelihood that a QTL is located at that position. The genotype/phenotype data are used to calculate for each test position a LOD score (log of likelihood ratio). When the LOD score exceeds a critical threshold value, there is significant evidence for the location of a QTL at that position on the genetic map (which will fall between two particular marker loci).

1. Linkage Disequilibrium Mapping and Association Studies

Another approach to determining trait gene location is to analyze trait-marker associations in a population within which individuals differ at both trait and marker loci. Certain marker alleles may be associated with certain trait locus alleles in this population due to population genetic process such as the unique origin of mutations, founder events, random drift and population structure. This association is referred to as linkage disequilibrium. In linkage disequilibrium mapping, one compares the trait values of individuals with different genotypes at a marker locus. Typically, a significant trait difference indicates close proximity between marker locus and one or more trait loci. If the marker density is appropriately high and the linkage disequilibrium occurs only between very closely linked sites on a chromosome, the location of trait loci can be very precise.

A specific type of linkage disequilibrium mapping is known as association studies. This approach makes use of markers within candidate genes, which are genes that are thought to be functionally involved in development of the trait because of information such as biochemistry, physiology, transcriptional profiling and reverse genetic experiments in model organisms. In association studies, markers within candidate genes are tested for association with trait variation. If linkage disequilibrium in the study population is restricted to very closely linked sites (i.e., within a gene or between adjacent genes), a positive association provides nearly conclusive evidence that the candidate gene is a trait gene.

2. Positional Cloning and Transgenic Applications

Traditional linkage mapping typically localizes a trait gene to an interval between two genetic markers (referred to as flanking markers). When this interval is relatively small (say less than 1 Mb), it becomes feasible to precisely identify the trait gene by a positional cloning procedure. A high marker density is required to narrow down the interval length sufficiently. This procedure requires a library of large insert genomic clones (such as a BAC library), where the inserts are pieces (usually 100-150 kb in length) of genomic DNA from the species of interest. The library is screened by probe hybridization or PCR to identify clones that contain the flanking marker sequences. Then a series of partially overlapping clones that connects the two flanking clones (a “contig”) is built up through physical mapping procedures. These procedures include fingerprinting, STS content mapping and sequence-tagged connector methodologies. Once the physical contig is constructed and sequenced, the sequence is searched for all transcriptional units. The transcriptional unit that corresponds to the trait gene can be determined by comparing sequences between mutant and wild type strains, by additional fine-scale genetic mapping, and/or by functional testing through plant transformation. Trait genes identified in this way become leads for transgenic product development. Similarly, trait genes identified by association studies with candidate genes become leads for transgenic product development.

3. Marker-Aided Breeding and Marker-Assisted Selection

When a trait gene has been localized in the vicinity of genetic markers, those markers can be used to select for improved values of the trait without the need for phenotypic analysis at each cycle of selection. In marker-aided breeding and marker-assisted selection, associations between trait genes and markers are established initially through genetic mapping analysis (as in sections 1 or 2 above). In the same process, one determines which marker alleles are linked to favorable trait gene alleles. Subsequently, marker alleles associated with favorable trait gene alleles are selected in the population. This procedure will improve the value of the trait provided that there is sufficiently close linkage between markers and trait genes. The degree of linkage required depends upon the number of generations of selection because, at each generation, there is opportunity for breakdown of the association through recombination.

4. Prediction of Crosses for New Inbred Line Development

The associations between specific marker alleles and favorable trait gene alleles also can be used to predict what types of progeny may segregate from a given cross. This prediction may allow selection of appropriate parents to generation populations from which new combinations of favorable trait gene alleles are assembled to produce a new inbred line. For example, if line A has marker alleles previously known to be associated with favorable trait alleles at loci 1, 20 and 31, while line B has marker alleles associated with favorable effects at loci 15, 27 and 29, then a new line could be developed by crossing A×B and selecting progeny that have favorable alleles at all 6 trait loci.

5. Hybrid Prediction

Commercial corn seed is produced by making hybrids between two elite inbred lines that belong to different “heterotic groups”. These groups are sufficiently distinct genetically that hybrids between them show high levels of heterosis or hybrid vigor (i.e., increased performance relative to the parental lines). By analyzing the marker constitution of good hybrids, one can identify sets of alleles at different loci in both male and female lines that combine well to produce heterosis. Understanding these patterns, and knowing the marker constitution of different inbred lines, can allow prediction of the level of heterosis between different pairs of lines. These predictions can narrow down the possibilities of which line(s) of opposite heterotic group should be used to test the performance of a new inbred line.

6. Identity by Descent

One theory of heterosis predicts that regions of identity by descent (IBD) between the male and female lines used to produce a hybrid will reduce hybrid performance. Identity by descent can be inferred from patterns of marker alleles in different lines. An identical string of markers at a series of adjacent loci may be considered identical by descent if it is unlikely to occur independently by chance. Analysis of marker fingerprints in male and female lines can identify regions of IBD. Knowledge of these regions can inform the choice of hybrid parents, because avoiding IBD in hybrids is likely to improve performance. This knowledge may also inform breeding programs in that crosses could be designed to produce pairs of inbred lines (one male and one female) that show little or no IBD.

A fingerprint of an inbred line is the combination of alleles at a set of marker loci. High density fingerprints can be used to establish and trace the identity of germplasm, which has utility in germplasm ownership protection.

Genetic markers are used to accelerate introgression of transgenes into new genetic backgrounds (i.e., into a diverse range of germplasm). Simple introgression involves crossing a transgenic line to an elite inbred line and then backcrossing the hybrid repeatedly to the elite (recurrent) parent, while selecting for maintenance of the transgene. Over multiple backcross generations, the genetic background of the original transgenic line is replaced gradually by the genetic background of the elite inbred through recombination and segregation. This process can be accelerated by selection on marker alleles that derive from the recurrent parent.

Use of Polymorphism Assay for Mapping a Library of DNA Clones

The polymorphisms and loci of this invention are useful for identifying and mapping DNA sequence of QTLs and genes linked to the polymorphisms. For instance, BAC or YAC clone libraries can be queried using polymorphisms linked to a trait to find a clone containing specific QTLs and genes associated with the trait. For instance, QTLs and genes in a plurality, e.g., hundreds or thousands, of large, multi-gene sequences can be identified by hybridization with an oligonucleotide probe that hybridizes to a mapped and/or linked polymorphism. Such hybridization screening can be improved by providing clone sequence in a high density array. The screening method is more preferably enhanced by employing a pooling strategy to significantly reduce the number of hybridizations required to identify a clone containing the polymorphism. When the polymorphisms are mapped, the screening effectively maps the clones.

For instance, in a case where thousands of clones are arranged in a defined array, e.g., in 96-well plates, the plates can be arbitrarily arranged in three-dimensionally, arrayed stacks of wells each comprising a unique DNA clone. The wells in each stack can be represented as discrete elements in a three dimensional array of rows, columns and plates. In one aspect of the invention the number of stacks and plates in a stack are about equal to minimize the number of assays. The stacks of plates allow the construction of pools of cloned DNA.

For a three-dimensionally arrayed stack, pools of cloned DNA can be created for (a) all of the elements in each row, (b) all of the elements of each column, and (c) all of the elements of each plate. Hybridization screening of the pools with an oligonucleotide probe that hybridizes to a polymorphism unique to one of the clones will provide a positive indication for one column pool, one row pool and one plate pool, thereby indicating the well element containing the target clone.

In the case of multiple stacks, additional pools of all of the clone DNA in each stack allows indication of the stack having the row-column-plate coordinates of the target clone. For instance, a 4608 clone set can be disposed in 48 96-well plates. The 48 plates can be arranged in 8 sets of 6-plate stacks providing 6×12×8 three-dimensional arrays of elements, i.e., each stack comprises 6 stacks of 8 rows and 12 columns. For the entire clone set there are 36 pools, i.e., 6 stack pools, 8 row pools, 12 column pools and 8 stack pools. Thus, a maximum of 36 hybridization reactions is required to find the clone harboring QTLs or genes associated or linked to each mapped polymorphism.

Once a clone is identified, genes within that clone can be tested for whether they affect the trait by analysis of recombinants in a mapping population, further linkage disequilibrium analysis, and ultimately transgenic testing. Additional genes can be identified by finding additional clones overlapping the one containing the original polymorphism through contig building, as described above.

Breeding Plants of the Invention

In addition to direct transformation of a particular plant genotype with a construct prepared according to the current invention, transgenic plants may be made by crossing a plant having a construct of the invention to a second plant lacking the construct. For example, a selected coding region operably linked to a promoter can be introduced into a particular plant variety by crossing, without the need for ever directly transforming a plant of that given variety. Therefore, the current invention not only encompasses a plant directly regenerated from cells that have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:
(a) plant seeds of the first (starting line) and second (donor plant line that comprises a transgene of the invention) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.
   Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plants containing the desired gene, DNA sequence or element;
(c) crossing the progeny plant to a plant of the second genotype; and
(d) repeating steps (b) and (c) for the purpose of transferring the desired gene, DNA sequence or element from a plant of a first genotype to a plant of a second genotype.

Plant Breeding

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

Backcrossing can be used to improve a starting plant. Backcrossing transfers a specific desirable trait from one source to an inbred or other plant that lacks that trait. This can be accomplished, for example, by first crossing a superior inbred (A) (recurrent parent) to a donor inbred (non-recurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred but are like the superior parent for most or almost all other genes. The last backcross generation would be selfed to give progeny that are pure breeding for the gene(s) being transferred, i.e., one or more transformation events.

Therefore, through a series a breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants that are true breeding for one or more transgenes. By crossing different inbred plants, one may produce a large number of different hybrids with different combinations of transgenes. In this way, plants may be produced that have the desirable agronomic properties frequently associated with hybrids (“hybrid vigor”), as well as the desirable characteristics imparted by one or more transgene(s).

It is desirable to introgress the genes of the present invention into maize hybrids for characterization of the phenotype conferred by each gene in a transformed plant. The host genotype into which the transgene was introduced, preferably LH59, is an elite inbred and therefore only limited breeding is necessary in order to produce high yielding maize hybrids. The transformed plant, regenerated from callus is crossed, to the same genotype, e.g., LH59. The progeny are self-pollinated twice, and plants homozygous for the transgene are identified. Homozygous transgenic plants are crossed to a testcross parent in order to produce hybrids. The test cross parent is an inbred belonging to a heterotic group that is different from that of the transgenic parent and for which it is known that high yielding hybrids can be generated, for example hybrids are produced from crosses of LH59 to either LH195 or LH200.

The following examples illustrate the identification of polymorphic markers useful for mapping and isolating genes of this invention and as markers of QTLs and genes associated with an oil-related trait. Other examples illustrate the identification of oil-related genes and partial genes. Still other examples illustrate methods for inserting genes of this invention into a plant expression vector, i.e., operably linked to a promoter and other regulatory elements, to confer an oil-related trait to a transgenic plant.

### EXAMPLE 1

This example illustrates the identification of oil-associated genes and maize oil markers.

a. Candidate Oil Genes

A set of more than 800 candidate oil genes was identified (a) as homologs of plant genes that are believed to be in an oil-related metabolic pathway of a model plant such as Arabidopsis thaliana; (b) by comparing transcription profiling results for high oil and low oil maize lines; and (c) by subtractive hybridization between endosperm tissues of high oil and low oil maize lines. The sequences of the candidate oil genes were queried against a proprietary collection of maize genes and partial maize genes, e.g., genomic sequence or ESTs, to identify a set of more than 800 candidate maize oil genes.

b. Maize Polymorphisms

Maize polymorphisms were identified by comparing alignments of DNA sequences from separate maize lines. Candidate polymorphisms were qualified by the following parameters:
(a) The minimum length of sequence for a synthetic reference sequence is 200 bases.
(b) The percentage identity of observed bases in a region of 15 bases on each side of a candidate SNP, is 75%.
(c) The minimum phred quality in each of the various sequences at a polymorphism site is 35.
(d) The minimum phred quality in a region of 15 bases on each side of the polymorphism site is 20.
   c. Oil Informative Markers

The SNP and Indel polymorphisms in each locus were qualified for detection by development of an assay, e.g., Taqman® assay (Applied Biosystems, Foster City, Calif.). Assay qualified polymorphisms are evaluated for oil informativeness by comparing allelic frequencies in the two parental lines of an association study population. The parent lines were representatives of an oil rich maize population and an oil poor maize population, i.e., the University of Illinois High Oil and Low Oil maize lines as described by Dudley and Lambert (1992, Maydica 37: 81-87). Informativeness is reported as an allelic frequency difference between parental populations, i.e. the high oil line and the low oil line. When one of the parents, e.g., the high oil line, is fixed, its allelic frequency is 1. Markers were qualified if they had an allelic frequency difference of at least 0.6. If the marker was fixed in either parent with a frequency of 0 or 1, a marker could be selected at a lower allelic frequency difference of at least 0.4. The informative markers were viewed on a genetic map to identify marker-deficient regions of chromosomes. Markers with lower allelic frequency difference, e.g., as low as 0.15, were selected to fill in the marker-deficient regions of chromosomes. A set of informative markers were used in a marker-trait association study to verify oil-associated genes from the set of candidate oil genes.

d. Labeled Probe Degradation Assay for SNP Detection

A quantity of maize genomic template DNA (e.g., about 2-20 ng) is mixed in 5 μL total volume with four oligonucleotides, which can be designed by Applied Biosystems, i.e., a forward primer, a reverse primer, a hybridization probe having a VIC reporter attached to the 5′ end, and a hybridization probe having a FAM reporter attached to the 5′end as well as PCR reaction buffer containing the passive reference dye ROX. The PCR reaction is conducted for 35 cycles using a 60° C. annealing-extension temperature. Following the reaction, the fluorescence of each fluorophore as well as that of the passive reference is determined in a fluorimeter. The fluorescence value for each fluorophore is normalized to the fluorescence value of the passive reference. The normalized values are plotted against each other for each sample. The data points should fall into clearly separable clusters.

To confirm that an assay produces accurate results, each new assay is performed on a number of replicates of samples of known genotypic identity representing each of the three possible genotypes, i.e., two homozygous alleles and a heterozygous sample. To be a valid and useful assay, it must produce clearly separable clusters of data points, such that one of the three genotypes can be assigned for at least 90% of the data points, and the assignment is observed to be correct for at least 98% of the data points. Subsequent to this validation step, the assay is applied to progeny of a cross between two highly inbred individuals to obtain segregation data, which are then used to calculate a genetic map position for the polymorphic locus.

e. Marker Mapping

The maize markers were genetically mapped based on the genotypes of certain SNPs. The genotypes were combined with genotypes for public core SSR and RFLP markers scored on recombinant inbred lines. Before mapping, any loci showing distorted segregation (P<0.01 for a Chi-square test of a 1:1 segregation ratio) were removed. These loci could be added to the map later but without allowing them to change marker order.

A map was constructed using the JoinMap version 2.0 software, which is described by Stam (“Construction of integrated genetic linkage maps by means of a new computer package: JoinMap, The Plant Journal, 3: 739-744 (1993); Stam, P. and van Ooijen, J. W. “JoinMap version 2.0: Software for the calculation of genetic linkage maps (1995) CPRO-DLO, Wageningen). JoinMap implements a weighted-least squares approach to multipoint mapping in which information from all pairs of linked loci (adjacent or not) is incorporated. Linkage groups were formed using a LOD threshold of 5.0. The SSR and RFLP public markers were used to assign linkage groups to chromosomes. Linkage groups were merged within chromosomes before map construction.

Haldane's mapping function was used to convert recombination fractions to map distances. Lenient criteria was applied for excluding pairwise linkage data; only data with a LOD not greater than 0.001 or a recombination fraction not less than 0.499 are excluded. Parameters for ordering loci were a jump threshold of 5.0, a triplet threshold of 7.0 and a ripple value of 3. About 38% of the loci were ordered in two rounds of map construction with a jump threshold of 5.0, which prevents the addition of a locus to the map if such addition results in a jump of more than 5.0 to a goodness-of-fit criterion. The remaining loci were added to the map without application of such a jump threshold. Addition of these loci had a negligible effect on the map order and distances for the initial loci. Mapped SNP polymorphisms are identified in Table 6.

f. Marker Trait Association

The informative maize markers were used in an association study to identify which of the candidate genes were more significantly associated with oil level in corn (Zea mays).

The University of Illinois has corn lines differing in seed oil that have been developed by long-term selection. A high oil line (IHO) produces about 18% seed oil and a low oil line (ILO) produces about 1.5% seed oil. The IHO and ILO lines are available from the University of Illinois for research. A random mated population (RMn) was produced from random mating offspring of a cross between IHO and ILO by chain crossing for 10 generations to produce an RM10 population. From the RM10 population 504 S1-derived lines were developed by selfing and these lines constitute an association study population. This population along with 72 control samples were genotyped using oil informative SNPs.

Phenotypes were measured on 504 association population lines in replicated field trials with an alpha(0,1) incomplete block design. The field trials comprised the 504 lines grown in each of two years at each of 3 locations with 2 replicates per location. The lines were blocked within each replicate. These field trials were performed on the 504 RM10:S1 lines, per se, and on hybrids made by crossing each line to a tester line, i.e., line (7051), but detailed marker genotyping information was obtained for only 499 of the lines.

Analysis of Variance

One approach to detecting marker-trait associations is to do analysis of variance (ANOVA) of each marker separately (i.e. single marker ANOVA with a model of trait=marker−x). When 488 markers were analyzed in this way for both per se and hybrid data, 186 markers were identified as having a significant effect on oil % at the alpha=0.05 level. See prior U.S. application Ser. No. 10/389,566.

Multiple Regression Analysis

An alternative statistical approach is to use multiple regression to determine which of a set of markers are simultaneously significantly associated with a trait of interest. First, it was established that a simple additive model is appropriate for these data. An analysis of variance of the raw observations was used to estimate variance components for environment (location×year combination), genotype (RM10:S1 line) and the genotype×environment interaction. The genotype×environment interaction variance component is < 1/0th the component for genotype. Similarly, ANOVAs of the line means show little or no dominance. In 488 tests of dominance (one per marker), only 27 have a p-value <0.05, which is close to the number expected by chance (24). All pairwise interactions between markers were tested also and we observed just 5.7% of the tests significant at the 5% level. Therefore, in subsequent analyses the genotypes were coded as −1, 0, 1 (for AA, Aa, aa) and multiple regression models without interaction terms were used.

One reason for using a multiple regression approach is that it is expected to be more sensitive in detecting trait effects in the presence of multiple QTLs. The reason is that, with single marker regression, nearly all the variance is in the error term. With multiple regression, if some of the markers account for variation in the trait, that variation is removed from the error term, thus providing greater statistical power. Of two new multiple regression methods that were evaluated along with single marker ANOVA, stepwise multiple regression was found to perform best in simulations. For details of the simulation results, see Laurie et al, in preparation.

Stepwise multiple regression was done with the “maxr” option of “PROC REG” of SAS software. “The MAXR method begins by finding the one-variable model producing the highest R². Then another variable, the one that yields the greatest increase in R², is added. Once the two-variable model is obtained, each of the variables in the model is compared to each variable not in the model. For each comparison, the MAXR method determines if removing one variable and replacing it with the other variable increases R². After comparing all possible switches, the MAXR method makes the switch that produces the largest increase in R². Comparisons begin again, and the process continues until the MAXR method finds that no switch could increase R². Thus, the two-variable model achieved is considered the “best” two-variable model the technique can find. Another variable is then added to the model, and the comparing-and-switching process is repeated to find the “best” three-variable model, and so forth. “(SAS Online Documentation, 1999 SAS Institute, Inc., Version 8). The “best” model (in terms of maximizing R²) was identified by MAXR for each model size in the range of 1 to 120 markers.

The “best” subset size was selected by minimizing a criterion that is equivalent to maximum likelihood with a penalty on model complexity. In general, the criterion=−2 log likelihood of the model−pk, where p is the number of parameters in the model (the number of markers plus one for the intercept) and k is a penalty factor. The Schwarz Bayesian Criterion (BIC, Rawlings, J. O., S. G. Pantula and D. A. Dickey, 1998, Applied Regression Analysis. Springer-Verlag, New York.) was used, for which k=ln(n), in this case, ln(499)=6.2). The “best” model dimension is taken as the minimum value of SBC, evaluated from 1 to 120 regressors.

Analyzing the RM10:S1 per se data by maxr/bic, 50 markers are selected. One disadvantage of the maxr/bic procedure is that it is difficult to assess statistical significance in a rigorous way. Although one gets probability values from tests of the partial regression coefficients, those values are not easily interpreted because the data were used to select markers that maximize the R² of regression. The p-values of the single-marker regressions are straightforward probabilities. If the 50 markers having lowest single marker p-values are selected, the greatest p-value is 0.0097. Since these markers are highly significant and the simulations show that maxr/bic essentially always does better than single marker regressions, it is assumed that the maxr/bic selected markers are at least as “significant” as those selected by single marker regression. Analyzing the hybrid data by maxr/bic, 39 markers are selected. If the 39 markers with lowest p-values of single marker regression from hybrids are selected, the largest p-value in the set is 0.0029.

There are 73 markers that are selected in either the per se and/or hybrid data sets (16 of these are selected in both). These 73 markers are significantly associated with oil in maize, which means it is very likely that they either directly cause variation in oil or they are closely linked to QTL that cause such variation. These 73 significant markers which are very likely to either reside within an oil gene or to be closely linked to an oil gene are in the 73 polymorphic loci of SEQ ID NO: 1 through SEQ ID NO:73 and identified more particularly in Table 1. A set of 73 of the candidate genes having sequence that overlaps with any one or more of the 73 genomic amplicons of SEQ ID NO:1 through SEQ ID NO:73 were identified and designated as oil-associated genes and are identified as having a cDNA sequence of SEQ ID NO:74 through SEQ ID NO:146. Because these oil-associated genes contain or are associated by linkage disequilibrium to a statistically significant maize oil marker, these oil-associated genes are most likely to be oil genes.

Tables 1-5 provides a description of 73 genomic amplicons defining polymorphic loci of the maize oil markers of this invention, 73 oil-associated genes and the cognate proteins and homologous proteins. These particular aspects of the invention are identified by:

“seq_num”, which refers to the sequence number of the nucleic acid sequence or amino acid sequence, e.g., a SEQ ID NO.; and

“seq_id”, which refers to an arbitrary identifying name for an amplicon, e.g. “Amplicon nnn”, for an oil-associated gene, e.g., “MRT4577_nnnnC”, for a cognate protein of an oil-associated gene, e.g. “MRT4577_nnnnP”, of for a cognate protein of a homolog to an oil-associated gene, e.g. “MRT4577_nnnnP” or a name from a database such as GenBank, e.g. “gi:6539874”.

“organism_name” which refers to the source organism for the gene or protein.

More particularly, the maize oil markers in the 73 genomic amplicons are described by:

MUTATION_ID, which refers to one or more arbitrary identifying names for each polymorphism;

START_POS which refers to the position in the nucleotide sequence of the polymorphic maize DNA locus where the polymorphism begins;

END_POS which refers to the position in the nucleotide sequence of the polymorphic maize DNA locus where the polymorphism ends; for SNPs the START_POS and END_POS are common;

TYPE which refers to the identification of the polymorphism as an SNP or IND (Indel);

ALLELEn and STRAINn which refer to the nucleotide sequence of a polymorphism in a specific allelic maize variety; and

GENE_ID refers to the SEQ_ID of the oil-associated gene identified later in Table 1.

More particularly, the oil-associated genes and their cognate proteins are described by:

DESCRIPTION, which refers to a functional description of an oil-associated gene, e.g., “gene encoding MRT4577_nnnnP” or a functional description of a cognate protein, e.g., a GenBank annotation or “long ORF” indicating no known protein function for an amino acid sequence that is translated from a longest available ORF.

Table 6 provides genetic map positions of maize oil markers and linked oil-associated genes; a description of the probability of significance of the marker/trait association (as determined from per se or hybrid association analysis for the marker); and the identification and sequence number of the oil-associated gene and their translated proteins. More particularly, Table 6 identifies maize oil markers, oil-associated genes and proteins by:

“Map Position” which identifies the distance measured in cM from the 5′ end of a maize chromosome for the SNP identified by “Mutation ID”, which refers to an arbitrary identifying name for each polymorphism;

Seq Num, which refers to the sequence number of a genomic amplicon containing the maize oil marker;

Protein Seq Num, which refers to the sequence number of the amino acid sequence, e.g., a SEQ ID NO, for the cognate protein encoded by a linked oil-associated gene.

TABLE 6

| Map Position | Mutation ID | Seq Num | Protein Seq Num |
|---|---|---|---|
| 1-30.4 | 144506 | 67 | 213 |
| 1-44 | 104827 | 55 | 201 |
| 1-46.8 | 37716 | 35 | 181 |
| 1-60.6 | 40189 | 38 | 184 |
| 1-85.9 | 69188 | 50 | 196 |
| 1-86.3 | 36286 | 32 | 178 |
| 1-99 | 107077 | 58 | 204 |
| 1-124.6 | 33373 | 27 | 173 |
| 1-129.5 | 9626 | 5 | 151 |
| 1-132.1 | 34903 | 28 | 174 |
| 1-178.6 | 151382 | 73 | 219 |
| 2-5.8 | 31064 | 22 | 168 |
| 2-19.5 | 82235 | 53 | 199 |
| 2-35.9 | 13691 | 9 | 155 |
| 2-92.5 | 551 | 1 | 147 |
| 2-114.9 | 22775 | 16 | 162 |
| 2-127 | 41850 | 40 | 186 |
| 2-152.4 | 43579 | 43 | 189 |
| 3-9.1 | 10667 | 7 | 153 |
| 3-19.7 | 32137 | 25 | 171 |
| 3-58.6 | 29867 | 21 | 167 |
| 3-59.3 | 21190 | 14 | 160 |
| 3-61.7 | 32247 | 26 | 172 |
| 3-62.7 | 9739 | 6 | 152 |
| 3-111.4 | 110780 | 62 | 208 |
| 4-38.7 | 110069 | 61 | 207 |
| 4-80 | 106845 | 57 | 203 |
| 4-108.2 | 39511 | 37 | 183 |
| 4-109.2 | 23289 | 18 | 164 |
| 4-110.3 | 8979 | 4 | 150 |
| 4-119.2 | 18439 | 13 | 159 |
| 4-128.1 | 32049 | 24 | 170 |
| 4-135.8 | 17900 | 12 | 158 |
| 4-144.8 | 35338 | 29 | 175 |
| 5-39.9 | 109403 | 60 | 206 |
| 5-57.7 | 52081 | 45 | 191 |
| 5-62.3 | 51419 | 44 | 190 |
| 5-66.9 | 146415 | 71 | 217 |
| 5-69.6 | 144731 | 68 | 214 |
| 5-76.4 | 29820 | 20 | 166 |
| 5-80.9 | 143418 | 66 | 212 |
| 5-83 | 104850 | 56 | 202 |
| 5-100.9 | 35377 | 30 | 176 |
| 5-104.5 | 58375 | 46 | 192 |
| 6-52.8 | 4463 | 2 | 148 |
| 6-53.1 | 60751 | 49 | 195 |
| 6-58.1 | 59008 | 48 | 194 |
| 6-61.5 | 148039 | 72 | 218 |
| 6-67.5 | 14694 | 11 | 157 |
| 6-110.4 | 31684 | 23 | 169 |
| 6-121 | 37634 | 34 | 180 |
| 7-62 | 42164 | 41 | 187 |
| 7-72.8 | 42930 | 42 | 188 |
| 7-99.8 | 35408 | 31 | 177 |
| 7-107.5 | 38914 | 36 | 182 |
| 7-122.2 | 145260 | 70 | 216 |
| 7-124.5 | 15184 | 10 | 156 |
| 7-186.5 | 36490 | 33 | 179 |
| 8-16.4 | 40320 | 39 | 185 |
| 8-40.9 | 107937 | 59 | 205 |
| 8-53.9 | 145200 | 69 | 215 |
| 8-55.7 | 23091 | 17 | 163 |
| 8-59.3 | 77568 | 51 | 197 |
| 8-65.8 | 104389 | 54 | 200 |
| 8-106.8 | 13100 | 8 | 154 |
| 9-20.5 | 58904 | 47 | 193 |
| 9-94.6 | 112139 | 64 | 210 |
| 9-110.3 | 8937 | 3 | 149 |
| 9-110.3 | 78438 | 52 | 198 |
| 9-165.8 | 110886 | 63 | 209 |
| 10-50.5 | 143408 | 65 | 211 |
| 10-56.7 | 22717 | 15 | 161 |
| 10-73.6 | 27447 | 19 | 165 |

### EXAMPLE 2

This example illustrates transgenic corn with altered oil level using recombinant DNA from an oil-associated gene.

GATEWAY™ destination vectors (available from Invitrogen Life Technologies, Carlsbad, Calif.) are constructed for insertion of recombinant DNA from oil-associated genes for corn transformation. The elements of each destination vector are summarized in Table 7 below and include a selectable marker transcription region and a DNA insertion transcription region. The selectable marker transcription region comprises a Cauliflower Mosaic Virus 35S promoter operably linked to a gene encoding neomycin phosphotransferase II (nptII) followed by both the 3′ region of the Agrobacterium tumefaciens nopaline synthase gene (nos) and the 3′ region of the potato proteinase inhibitor II (pinII) gene. The DNA insertion transcription region comprises a rice actin 1 promoter, a rice actin 1 exon 1 intron1 enhancer, an att-flanked insertion site and the 3′ region of the potato pinII gene. Following standard procedures provided by Invitrogen the att-flanked insertion region is replaced by recombination with DNA from an oil-associated gene, in a sense orientation for expression of the cognate protein from an oil-associated gene and in a gene suppression orientation (i.e. either anti-sense orientation or in a sense- and anti-sense orientation) for a suppression of an oil associated gene. Although the vector with DNA from an oil-associated gene inserted at the att-flanked insertion region is useful for plant transformation by direct DNA delivery, such as microprojectile bombardment, it is preferable to bombard target plant tissue with tandem transcription units that have been cut from the vector. For Agrobacterium-mediated transformation of plants the vector also comprises T-DNA borders from Agrobacterium flanking the transcription units.

Vectors for Agrobacterium-mediated transformation are prepared with recombinant DNA from each of the oil-associated genes having a sequence of SEQ ID NO: 74 through SEQ ID NO: 146 and for each of the homologous oil-associated genes encoding a protein having an amino acid sequence of SEQ ID NO: 220 through SEQ ID NO: 2337 with the DNA solely in sense orientation for expression of the oil-associated protein. Each vector is transformed into corn callus which is propagated into a plant that is grown to produce transgenic seed. Progeny plants are self-pollinated to produce seed which is selected for homozygous seed. Homozygous seed is used for producing inbred plants, for introgressing the trait into elite lines, and for crossing to make hybrid seed. Progeny transgenic plants (both inbreds of the transgenic plant and hybrids with other corn lines) comprise the recombinant DNA from an oil-associated gene and have enhanced oil in seed. Transgenic corn including inbred and hybrids with enhanced oil are also produced with recombinant DNA from each of the homologous genes of an oil-associated gene that encode a protein having an amino acid sequence of SEQ ID NO:220 through SEQ ID NO:2337. Transgenic corn plants with recombinant DNA from each oil-associated gene and each homolog of an oil-associated gene are also produced where the rice actin 1 promoter and enhancer are replaced with each of the promoters in the group consisting of a maize globulin 1 promoter, a maize L3 oleosin promoter, a maize emb5 promoter, a zein Z27 promoter, a gamma coixin promoter, and a CaMV 35S promoter. Seed produced by the plants is provided to growers to enable production of corn crops with enhanced oil.

Vectors for Agrobacterium-mediated transformation are also prepared with recombinant DNA from each of the oil-associated genes having a sequence of SEQ ID NO: 74 through SEQ ID NO: 146 in a gene suppression orientation for suppression of the maize endogenous oil-associated gene. Each vector is transformed into corn callus which is propagated into a plant that is grown to produce transgenic seed. Progeny plants are self-pollinated to produce seed which is selected for homozygous seed. Homozygous seed is used for producing inbred plants, for introgressing the trait into elite lines, and for crossing to make hybrid seed. Progeny transgenic plants (both inbreds of the transgenic plant and hybrids with other corn lines) comprise the recombinant DNA from an oil-associated gene and have reduced oil in seed. Transgenic corn plants with recombinant DNA for suppressing each oil-associated gene are also produced where the rice actin 1 promoter and enhancer are replaced with each of the promoters in the group consisting of a maize globulin 1 promoter, a maize L3 oleosin promoter, a maize emb5 promoter, a zein Z27 promoter, a gamma coixin promoter, and a CaMV 35S promoter. Seed produced by the plants is provided to growers to enable production of corn crops with reduced oil.

TABLE 7
Elements of an exemplary corn transformation vector

| FUNCTION | ELEMENT | REFERENCE |
|---|---|---|
| | Rice actin 1 | U.S. Pat. No. 5,641,876 |
| | promoter | |
| DNA insertion | Rice actin 1 | U.S. Pat. No. 5,641,876 |
| transcription region | promoter | |
| DNA insertion | [symbol] actin 1 | [symbol] g Technology |
| transcription region | exon 1, intron 1 | Instruction Manual |
| (att-flanked | enhancer | |
| insertion region) | | |
| | CmR gene | GATEWAY ™Cloning |
| | | Technology Instruction |
| | | Manual |
| | ccdA, ccdB genes | GATEWAY ™Cloning |
| | | Technology Instruction |
| | | Manual |
| | attR2 | GATEWAY ™Cloning |
| | | Technology Instruction |
| | | Manual |
| DNA insertion | Potato pinII | An et al. (1989) Plant |
| transcription region | 3′ region | Cell 1: 115-122 |
| selectable marker | CaMV 35S promoter | U.S. Pat. No. 5,858,742 |
| transcription region | | |
| | nptII selectable | U.S. Pat. No. 5,858,742 |
| | marker | |
| | nos 3region | U.S. Pat. No. 5,858,742 |
| | PinII 3′ region | An et al. (1989) Plant |
| | | Cell 1: 115-122 |
| | ColE1 origin of | |
| | replication | |
| | F1 origin of | |
| | replication | |
| | Bla ampicillin | |
| | resistance | |

### EXAMPLE 3

This example illustrates transgenic soybean with altered oil level using recombinant DNA from an oil-associated gene.

GATEWAY™ destination vectors (available from Invitrogen Life Technologies, Carlsbad, Calif.) are constructed for insertion of recombinant DNA from oil-associated genes for soybean transformation. Constructs for use in transformation of soybean are prepared by restriction enzyme based cloning into a common expression vector. Elements of an exemplary common expression vector are shown in Table 8 below and include a selectable marker expression cassette and a gene of interest expression cassette. The selectable marker expression cassette comprises Arabidopsis act 7 gene (AtAct7) promoter with intron and 5′UTR, the transit peptide of Arabidopsis EPSPS, the synthetic CP4 coding region with dicot preferred codon usage and a 3′ UTR of the nopaline synthase gene. The gene of interest expression cassette comprises a Cauliflower Mosaic Virus 35S promoter operably linked to an oil-associated gene in a sense orientation for expression of an oil-enhancing protein and in a gene suppression orientation (i.e. either anti-sense orientation or in a sense- and anti-sense orientation for suppression of an oil-associated gene.

Vectors similar to that described above are be constructed for use in Agrobacterium mediated soybean transformation systems, with recombinant DNA from each of the oil-associated genes having a sequence of SEQ ID NO:74 though SEQ ID NO:146 and homologous genes which encode proteins with an amino acid sequence of SEQ ID NO:220 through SEQ ID NO:2337 with the DNA in sense orientation for expression of the cognate protein. Transgenic soybean plants are produced using vectors for each oil-associated gene and homolog; the transgenic soybean plants have enhanced oil in the seed. Transgenic soybean plants are also produced for recombinant DNA from each of the oil-associated genes and homologs is transcribed by each of the promoters in the group consisting of a maize globulin 1 promoter, a maize L3 oleosin promoter, a maize emb5 promoter, a zein Z27 promoter, a gamma coixin promoter, and a CaMV 35S promoter. Seed produced by the plants is provided to growers to enable production of soybean crops with enhanced oil.

Vectors for Agrobacterium-mediated transformation are also prepared with recombinant DNA from each of the homologs of oil-associated genes from Glycine max, e.g. DNA encoding the protein with the amino acid sequence of SEQ ID NO:244, 318, 318, 353 and each of the others listed in Table 5, in a gene suppression orientation for suppression of the endogenous soybean homolog. Each vector is transformed into corn callus which is propagated into a plant that is grown to produce transgenic seed. Progeny plants are self-pollinated to produce seed which is selected for homozygous seed. Homozygous seed is used for producing inbred plants, for introgressing the trait into elite lines, and for crossing to make hybrid seed. Progeny transgenic plants (both inbreds of the transgenic plant and hybrids with other corn lines) comprise the recombinant DNA from an oil-associated gene and have reduced oil in seed. Transgenic corn plants with recombinant DNA for suppressing each oil-associated gene are also produced where the rice actin 1 promoter and enhancer are replaced with each of the promoters in the group consisting of a maize globulin 1 promoter, a maize L3 oleosin promoter, a maize emb5 promoter, a zein Z27 promoter, a gamma coixin promoter, and a CaMV 35S promoter. Seed produced by the plants is provided to growers to enable production of corn crops with reduced oil.

TABLE 8
Elements of an exemplary soybean transformation construct

| Function | Element | Reference |
|---|---|---|
| Agro transformation | B-ARGtu.right border | Depicker, A. et |
| | | al (1982) Mol Appl |
| | | Genet 1: 561-573 |
| Antibiotic resistance | CR-Ec.aadA-SPC/STR | |
| Represser of primers | CR-Ec.rop | |
| from the ColE1 plasmid | | |
| Origin of replication | OR-Ec.oriV-RK2 | |
| Agro transformation | B-ARGtu.left border | Barker, R. F. et |
| | | al (1983) Plant |
| | | Mol Biol 2: |
| | | 335-350 |
| Plant selectable | Arabidopsis act 7 | McDowell et al. |
| marker expression | gene (AtAct7) | (1996) Plant |
| cassette | promoter with | Physiol. 111: |
| | intron and 5′UTR | 699-711. |
| | 5′ UTR of | |
| | Arabidopsis act 7 | |
| | gene | |
| | Intron in 5′UTR | |
| | of AtAct7 | |
| | Transit peptide | Klee, H. J. et al |
| | region of | (1987) MGG 210: |
| | Arabidopsis EPSPS | 437-442 |
| | Synthetic CP4 | |
| | coding region with | |
| | dicot preferred | |
| | codon usage | |
| | A 3′ UTR of the | U.S. Pat. No. |
| | nopaline synthase | 5,858,742 |
| | gene of | |
| | Agrobacterium | |
| | tumefaciens Ti | |
| | plasmid | |
| Plant gene of | Promoter for 35S | U.S. Pat. No. |
| interest expression | RNA from CaMV | 5,322,938 |
| cassette | containing a | |
| | duplication of | |
| | the −90 to −350 | |
| | region | |
| | Gene of interest | |
| | insertion site | |
| | Cotton E6 3′ | GenBank accession |
| | end | U30508 |

TABLE 1

| | | | | | | ALLELE1 | ALLELE2 | ALLELE3 | ALLELE4 | |
|---|---|---|---|---|---|---|---|---|---|---|
| SEQ_NUM | SEQ_ID | MUTATION_ID | START_POS | END_POS | TYPE | STRAINS1 | STRAINS2 | STRAINS3 | STRAINS4 | CANDIDATE_ID |
| 1 | Amplicon150 | 548 | 85 | 85 | SNP | A | | C | | MRT4577_407583C |
| 1 | Amplicon150 | 549 | 108 | 108 | SNP | C | | T | | MRT4577_407583C |
| 1 | Amplicon150 | 550 | 158 | 158 | SNP | A | | T | | MRT4577_407583C |
| 1 | Amplicon150 | 551 | 175 | 175 | SNP | G | | T | | MRT4577_407583C |
| 2 | Amplicon50699 | 4463 | 282 | 282 | SNP | C | b73 | T | mo17 | MRT4577_37957C |
| 3 | Amplicon174322 | 8937 | 152 | 152 | SNP | A | mo17 | T | b73 | MRT4577_306229C |
| 4 | Amplicon174423 | 8979 | 197 | 197 | SNP | A | mo17 | T | b73 | MRT4577_305583C |
| 5 | Amplicon175589 | 9626 | 239 | 239 | SNP | C | mo17 | G | b73 | MRT4577_189292C |
| 5 | Amplicon175589 | 9627 | 261 | 261 | SNP | A | b73 | C | mo17 | MRT4577_189292C |
| 6 | Amplicon175758 | 9739 | 291 | 291 | SNP | A | b73 | G | mo17 | MRT4577_409052C |
| 7 | Amplicon176352 | 9927 | 41 | 41 | SNP | A | mo17 | T | b73 | MRT4577_371170C |
| 7 | Amplicon176352 | 10667 | 309 | 309 | SNP | A | mo17 | G | b73 | MRT4577_371170C |
| 8 | Amplicon176822 | 11713 | 301 | 301 | SNP | C | mo17 | G | b73 | MRT4577_169297C |
| 8 | Amplicon176822 | 13100 | 287 | 287 | SNP | A | b73 | C | mo17 | MRT4577_169297C |
| 9 | Amplicon177147 | 13685 | 231 | 231 | SNP | A | b73 | G | mo17 | MRT4577_273665C |
| 9 | Amplicon177147 | 13687 | 246 | 246 | SNP | C | b73 | T | mo17 | MRT4577_273665C |
| 9 | Amplicon177147 | 13688 | 301 | 301 | SNP | A | b73 | C | mo17 | MRT4577_273665C |
| 9 | Amplicon177147 | 13689 | 393 | 393 | SNP | A | b73 | C | mo17 | MRT4577_273665C |
| 9 | Amplicon177147 | 13691 | 490 | 490 | SNP | C | mo17 | T | b73 | MRT4577_273665C |
| 10 | Amplicon177165 | 13783 | 67 | 67 | SNP | A | b73 | G | mo17 | MRT4577_285101C |
| 10 | Amplicon177165 | 13785 | 102 | 102 | SNP | C | mo17 | T | b73 | MRT4577_285101C |
| 10 | Amplicon177165 | 13787 | 112 | 112 | IND | * | mo17 | T | b73 | MRT4577_285101C |
| 10 | Amplicon177165 | 13791 | 144 | 144 | SNP | C | mo17 | T | b73 | MRT4577_285101C |
| 10 | Amplicon177165 | 13793 | 145 | 145 | SNP | A | mo17 | T | b73 | MRT4577_285101C |
| 10 | Amplicon177165 | 13795 | 191 | 191 | SNP | A | mo17 | T | b73 | MRT4577_285101C |
| 10 | Amplicon177165 | 13797 | 192 | 192 | SNP | A | b73 | C | mo17 | MRT4577_285101C |
| 10 | Amplicon177165 | 13799 | 194 | 194 | SNP | C | mo17 | G | b73 | MRT4577_285101C |
| 10 | Amplicon177165 | 13801 | 230 | 230 | SNP | A | b73 | G | mo17 | MRT4577_285101C |
| 10 | Amplicon177165 | 13803 | 242 | 244 | IND | *** | b73 | TAC | mo17 | MRT4577_285101C |
| 10 | Amplicon177165 | 13805 | 275 | 275 | SNP | A | b73 | G | mo17 | MRT4577_285101C |
| 10 | Amplicon177165 | 13807 | 335 | 335 | SNP | A | mo17 | C | b73 | MRT4577_285101C |
| 10 | Amplicon177165 | 13811 | 568 | 568 | SNP | C | b73 | T | mo17 | MRT4577_285101C |
| 10 | Amplicon177165 | 15184 | 391 | 391 | SNP | C | b73 | T | mo17 | MRT4577_285101C |
| 11 | Amplicon177361 | 14692 | 75 | 75 | SNP | C | b73 | G | mo17 | MRT4577_284415C |
| 11 | Amplicon177361 | 14694 | 105 | 105 | SNP | A | mo17 | C | b73 | MRT4577_284415C |
| 11 | Amplicon177361 | 14697 | 529 | 529 | SNP | C | b73 | T | mo17 | MRT4577_284415C |
| 11 | Amplicon177361 | 14698 | 557 | 557 | SNP | C | b73 | T | mo17 | MRT4577_284415C |
| 11 | Amplicon177361 | 14700 | 561 | 561 | SNP | G | mo17 | T | b73 | MRT4577_284415C |
| 12 | Amplicon177729 | 16576 | 64 | 64 | SNP | C | mo17 | T | b73 | MRT4577_38704C |
| 12 | Amplicon177729 | 16578 | 84 | 84 | SNP | A | mo17 | T | b73 | MRT4577_38704C |
| 12 | Amplicon177729 | 16582 | 209 | 209 | SNP | G | mo17 | T | b73 | MRT4577_38704C |
| 12 | Amplicon177729 | 16584 | 249 | 249 | SNP | C | mo17 | T | b73 | MRT4577_38704C |
| 12 | Amplicon177729 | 16585 | 251 | 254 | IND | **** | b73 | GGAC | mo17 | MRT4577_38704C |
| 12 | Amplicon177729 | 16588 | 332 | 332 | SNP | G | mo17 | T | b73 | MRT4577_38704C |
| 12 | Amplicon177729 | 16589 | 378 | 378 | SNP | G | mo17 | T | b73 | MRT4577_38704C |
| 12 | Amplicon177729 | 16591 | 392 | 392 | SNP | A | b73 | T | mo17 | MRT4577_38704C |
| 12 | Amplicon177729 | 16593 | 398 | 398 | SNP | C | b73 | T | mo17 | MRT4577_38704C |
| 12 | Amplicon177729 | 16595 | 399 | 399 | IND | * | b73 | T | mo17 | MRT4577_38704C |
| 12 | Amplicon177729 | 17900 | 156 | 156 | SNP | A | mo17 | G | b73 | MRT4577_38704C |
| 12 | Amplicon177729 | 17908 | 257 | 260 | IND | **** | b73 | CTGG | mo17 | MRT4577_38704C |
| 13 | Amplicon177848 | 17120 | 151 | 151 | SNP | A | mo17 | G | b73 | MRT4577_47332C |
| 13 | Amplicon177848 | 18439 | 172 | 172 | SNP | A | b73 | G | mo17 | MRT4577_47332C |
| 14 | Amplicon178666 | 21190 | 286 | 286 | SNP | A | b73 | G | mo17 | MRT4577_386264C |
| 14 | Amplicon178666 | 21192 | 499 | 499 | SNP | C | mo17 | T | b73 | MRT4577_386264C |
| 15 | Amplicon178700 | 22717 | 64 | 64 | SNP | A | mo17 | T | b73 | MRT4577_25879C |
| 16 | Amplicon178723 | 21524 | 116 | 116 | IND | * | mo17 | T | b73 | MRT4577_419574C |
| 16 | Amplicon178723 | 21526 | 118 | 118 | IND | * | mo17 | A | b73 | MRT4577_419574C |
| 16 | Amplicon178723 | 21528 | 210 | 216 | IND | ******* | b73 | AGCTAGC | mo17 | MRT4577_419574C |
| 16 | Amplicon178723 | 21530 | 218 | 218 | IND | * | b73 | T | mo17 | MRT4577_419574C |
| 16 | Amplicon178723 | 21532 | 482 | 482 | SNP | C | mo17 | T | b73 | MRT4577_419574C |
| 16 | Amplicon178723 | 21533 | 486 | 486 | SNP | C | mo17 | G | b73 | MRT4577_419574C |
| 16 | Amplicon178723 | 21535 | 488 | 488 | SNP | C | b73 | G | mo17 | MRT4577_419574C |
| 16 | Amplicon178723 | 21536 | 489 | 489 | IND | * | mo17 | T | b73 | MRT4577_419574C |
| 16 | Amplicon178723 | 21539 | 491 | 491 | SNP | C | b73 | T | mo17 | MRT4577_419574C |
| 16 | Amplicon178723 | 21541 | 497 | 497 | SNP | A | mo17 | T | b73 | MRT4577_419574C |
| 16 | Amplicon178723 | 21543 | 501 | 502 | IND | ** | mo17 | GC | b73 | MRT4577_419574C |
| 16 | Amplicon178723 | 21545 | 504 | 504 | SNP | A | b73 | G | mo17 | MRT4577_419574C |
| 16 | Amplicon178723 | 22775 | 527 | 527 | SNP | A | mo17 | G | b73 | MRT4577_419574C |
| 17 | Amplicon178785 | 23091 | 170 | 170 | SNP | G | b73 | T | mo17 | MRT4577_414575C |
| 18 | Amplicon178833 | 23289 | 251 | 251 | SNP | A | b73 | G | mo17 | MRT4577_199838C |
| 19 | Amplicon179515 | 26314 | 17 | 17 | SNP | A | b73 | G | mo17 | MRT4577_409604C |
| 19 | Amplicon179515 | 26316 | 34 | 34 | IND | * | b73 | A | mo17 | MRT4577_409604C |
| 19 | Amplicon179515 | 26318 | 96 | 96 | SNP | A | b73 | G | mo17 | MRT4577_409604C |
| 19 | Amplicon179515 | 26319 | 133 | 133 | SNP | A | b73 | G | mo17 | MRT4577_409604C |
| 19 | Amplicon179515 | 26321 | 162 | 162 | SNP | C | mo17 | G | b73 | MRT4577_409604C |
| 19 | Amplicon179515 | 26322 | 282 | 284 | IND | *** | b73 | CTG | mo17 | MRT4577_409604C |
| 19 | Amplicon179515 | 26326 | 352 | 352 | SNP | A | mo17 | C | b73 | MRT4577_409604C |
| 19 | Amplicon179515 | 27447 | 311 | 311 | SNP | C | b73 | G | mo17 | MRT4577_409604C |
| 20 | Amplicon235434 | 29819 | 65 | 65 | SNP | C | b73 | T | mo17 | MRT4577_391398C |
| 20 | Amplicon235434 | 29820 | 109 | 109 | SNP | A | b73 | G | mo17 | MRT4577_391398C |
| 20 | Amplicon235434 | 29821 | 121 | 121 | SNP | A | mo17 | G | b73 | MRT4577_391398C |
| 20 | Amplicon235434 | 29822 | 122 | 122 | SNP | A | mo17 | T | b73 | MRT4577_391398C |
| 20 | Amplicon235434 | 29823 | 181 | 181 | SNP | C | mo17 | T | b73 | MRT4577_391398C |
| 20 | Amplicon235434 | 29824 | 187 | 187 | SNP | A | mo17 | G | b73 | MRT4577_391398C |
| 20 | Amplicon235434 | 29825 | 203 | 203 | SNP | A | b73 | C | mo17 | MRT4577_391398C |
| 20 | Amplicon235434 | 29826 | 211 | 211 | SNP | A | mo17 | G | b73 | MRT4577_391398C |
| 20 | Amplicon235434 | 29827 | 216 | 216 | SNP | C | b73 | T | mo17 | MRT4577_391398C |
| 21 | Amplicon235455 | 29867 | 81 | 84 | IND | **** | mo17 | TGAG | b73 | MRT4577_234188C |
| 21 | Amplicon235455 | 29868 | 195 | 196 | IND | ** | mo17 | AA | b73 | MRT4577_234188C |
| 21 | Amplicon235455 | 29869 | 363 | 363 | SNP | A | b73 | G | mo17 | MRT4577_234188C |
| 21 | Amplicon235455 | 29870 | 365 | 365 | SNP | C | mo17 | G | b73 | MRT4577_234188C |
| 21 | Amplicon235455 | 29871 | 375 | 375 | SNP | A | mo17 | C | b73 | MRT4577_234188C |
| 22 | Amplicon236049 | 31050 | 34 | 34 | SNP | A | b73 | C | mo17 | MRT4577_264682C |
| 22 | Amplicon236049 | 31051 | 36 | 36 | SNP | A | b73 | C | mo17 | MRT4577_264682C |
| 22 | Amplicon236049 | 31052 | 38 | 38 | SNP | A | b73 | G | mo17 | MRT4577_264682C |
| 22 | Amplicon236049 | 31053 | 47 | 47 | SNP | A | mo17 | T | b73 | MRT4577_264682C |
| 22 | Amplicon236049 | 31054 | 48 | 48 | SNP | A | mo17 | G | b73 | MRT4577_264682C |
| 22 | Amplicon236049 | 31055 | 49 | 49 | SNP | C | b73 | G | mo17 | MRT4577_264682C |
| 22 | Amplicon236049 | 31056 | 52 | 52 | SNP | A | b73 | T | mo17 | MRT4577_264682C |
| 22 | Amplicon236049 | 31057 | 54 | 54 | SNP | C | b73 | T | mo17 | MRT4577_264682C |
| 22 | Amplicon236049 | 31058 | 55 | 55 | SNP | A | b73 | C | mo17 | MRT4577_264682C |
| 22 | Amplicon236049 | 31059 | 56 | 56 | SNP | A | b73 | C | mo17 | MRT4577_264682C |
| 22 | Amplicon236049 | 31060 | 57 | 57 | SNP | G | b73 | T | mo17 | MRT4577_264682C |
| 22 | Amplicon236049 | 31061 | 59 | 59 | SNP | C | b73 | G | mo17 | MRT4577_264682C |
| 22 | Amplicon236049 | 31062 | 63 | 63 | SNP | C | b73 | T | mo17 | MRT4577_264682C |
| 22 | Amplicon236049 | 31063 | 65 | 66 | IND | ** | mo17 | TC | b73 | MRT4577_264682C |
| 22 | Amplicon236049 | 31064 | 126 | 126 | SNP | A | b73 | C | mo17 | MRT4577_264682C |
| 22 | Amplicon236049 | 31065 | 180 | 180 | SNP | C | mo17 | G | b73 | MRT4577_264682C |
| 22 | Amplicon236049 | 31066 | 540 | 540 | SNP | G | mo17 | T | b73 | MRT4577_264682C |
| 23 | Amplicon236326 | 31684 | 260 | 260 | SNP | A | b73 | T | mo17 | MRT4577_287055C |
| 24 | Amplicon236499 | 32049 | 183 | 183 | SNP | C | b73 | T | mo17 | MRT4577_49099C |
| 24 | Amplicon236499 | 32050 | 402 | 402 | SNP | C | mo17 | T | b73 | MRT4577_49099C |
| 24 | Amplicon236499 | 32051 | 403 | 403 | SNP | A | mo17 | G | b73 | MRT4577_49099C |
| 25 | Amplicon236541 | 32137 | 258 | 258 | IND | * | b73 | A | mo17 | MRT4577_346921C |
| 25 | Amplicon236541 | 32138 | 420 | 430 | IND | *********** | mo17 | CCGATCCATCT | b73 | MRT4577_346921C |
| 26 | Amplicon236590 | 32244 | 27 | 27 | SNP | C | b73 | T | mo17 | MRT4577_257780C |
| 26 | Amplicon236590 | 32245 | 82 | 82 | SNP | A | b73 | G | mo17 | MRT4577_257780C |
| 26 | Amplicon236590 | 32246 | 92 | 98 | IND | ******* | mo17 | AGTGCTG | b73 | MRT4577_257780C |
| 26 | Amplicon236590 | 32247 | 162 | 162 | SNP | C | b73 | T | mo17 | MRT4577_257780C |
| 26 | Amplicon236590 | 32248 | 275 | 275 | SNP | C | b73 | T | mo17 | MRT4577_257780C |
| 27 | Amplicon276497 | 33373 | 96 | 96 | SNP | C | mo17 | T | b73 | MRT4577_410376C |
| 27 | Amplicon276497 | 33374 | 128 | 128 | SNP | C | mo17 | T | b73 | MRT4577_410376C |
| 27 | Amplicon276497 | 33375 | 131 | 131 | SNP | C | mo17 | T | b73 | MRT4577_410376C |
| 27 | Amplicon276497 | 33376 | 363 | 363 | SNP | C | b73 | G | mo17 | MRT4577_410376C |
| 27 | Amplicon276497 | 33377 | 371 | 371 | SNP | G | b73 | T | mo17 | MRT4577_410376C |
| 28 | Amplicon277511 | 34895 | 48 | 48 | SNP | C | b73 | G | mo17 | MRT4577_233403C |
| 28 | Amplicon277511 | 34896 | 49 | 49 | SNP | C | b73 | T | mo17 | MRT4577_233403C |
| 28 | Amplicon277511 | 34897 | 53 | 53 | IND | * | b73 | C | mo17 | MRT4577_233403C |
| 28 | Amplicon277511 | 34898 | 53 | 54 | IND | ** | b73 | C* | mo17 | MRT4577_233403C |
| 28 | Amplicon277511 | 34899 | 76 | 76 | SNP | C | b73 | T | mo17 | MRT4577_233403C |
| 28 | Amplicon277511 | 34900 | 308 | 308 | SNP | A | b73 | C | mo17 | MRT4577_233403C |
| 28 | Amplicon277511 | 34901 | 345 | 345 | SNP | A | mo17 | G | b73 | MRT4577_233403C |
| 28 | Amplicon277511 | 34902 | 348 | 348 | SNP | C | b73 | T | mo17 | MRT4577_233403C |
| 28 | Amplicon277511 | 34903 | 409 | 409 | SNP | C | mo17 | T | b73 | MRT4577_233403C |
| 29 | Amplicon277876 | 35338 | 105 | 105 | SNP | C | mo17 | G | b73 | MRT4577_294774C |
| 29 | Amplicon277876 | 35339 | 330 | 334 | IND | ***** | b73 | CAAAG | mo17 | MRT4577_294774C |
| 29 | Amplicon277876 | 35340 | 368 | 368 | SNP | A | b73 | G | mo17 | MRT4577_294774C |
| 30 | Amplicon277914 | 35377 | 67 | 67 | SNP | C | b73 | G | mo17 | MRT4577_402771C |
| 31 | Amplicon277962 | 35407 | 32 | 32 | SNP | A | mo17 | G | b73 | MRT4577_397598C |
| 31 | Amplicon277962 | 35408 | 221 | 221 | SNP | A | mo17 | C | b73 | MRT4577_397598C |
| 31 | Amplicon277962 | 35409 | 293 | 293 | SNP | A | b73 | C | mo17 | MRT4577_397598C |
| 31 | Amplicon277962 | 35410 | 340 | 340 | SNP | A | mo17 | G | b73 | MRT4577_397598C |
| 32 | Amplicon310739 | 36286 | 336 | 337 | IND | ** | mo17 | AT | b73 | MRT4577_204611C |
| 32 | Amplicon310739 | 36287 | 436 | 437 | IND | ** | b73 | CT | mo17 | MRT4577_204611C |
| 32 | Amplicon310739 | 36288 | 456 | 456 | SNP | A | b73 | G | mo17 | MRT4577_204611C |
| 33 | Amplicon310854 | 36487 | 202 | 204 | IND | *** | b73 | TGG | mo17 | MRT4577_404797C |
| 33 | Amplicon310854 | 36488 | 228 | 229 | IND | ** | b73 | AT | mo17 | MRT4577_404797C |
| 33 | Amplicon310854 | 36489 | 236 | 236 | IND | * | mo17 | T | b73 | MRT4577_404797C |
| 33 | Amplicon310854 | 36490 | 244 | 244 | SNP | G | b73 | T | mo17 | MRT4577_404797C |
| 33 | Amplicon310854 | 36491 | 273 | 275 | IND | *** | b73 | TAG | mo17 | MRT4577_404797C |
| 33 | Amplicon310854 | 36492 | 273 | 276 | IND | **** | b73 | TAGC | mo17 | MRT4577_404797C |
| 33 | Amplicon310854 | 36493 | 316 | 317 | IND | ** | mo17 | GA | b73 | MRT4577_404797C |
| 33 | Amplicon310854 | 36494 | 320 | 320 | SNP | C | b73 | T | mo17 | MRT4577_404797C |
| 34 | Amplicon311738 | 37631 | 272 | 272 | SNP | C | mo17 | G | b73 | MRT4577_32764C |
| 34 | Amplicon311738 | 37632 | 334 | 341 | IND | ******** | mo17 | CGTTCTAA | b73 | MRT4577_32764C |
| 34 | Amplicon311738 | 37633 | 390 | 398 | IND | ********* | b73 | CGTTGGGGG | mo17 | MRT4577_32764C |
| 34 | Amplicon311738 | 37634 | 543 | 543 | SNP | G | mo17 | T | b73 | MRT4577_32764C |
| 35 | Amplicon346472 | 37715 | 393 | 393 | SNP | A | b73 | G | mo17 | MRT4577_284905C |
| 35 | Amplicon346472 | 37716 | 513 | 513 | SNP | C | b73 | T | mo17 | MRT4577_284905C |
| 35 | Amplicon346472 | 37717 | 523 | 523 | IND | * | mo17 | A | b73 | MRT4577_284905C |
| 35 | Amplicon346472 | 37718 | 564 | 564 | SNP | G | mo17 | T | b73 | MRT4577_284905C |
| 35 | Amplicon346472 | 37719 | 574 | 577 | IND | **** | b73 | ACGA | mo17 | MRT4577_284905C |
| 36 | Amplicon347285 | 38909 | 42 | 42 | SNP | A | b73 | T | mo17 | MRT4577_386764C |
| 36 | Amplicon347285 | 38910 | 94 | 97 | IND | **** | mo17 | TGCA | b73 | MRT4577_386764C |
| 36 | Amplicon347285 | 38911 | 100 | 100 | SNP | A | b73 | G | mo17 | MRT4577_386764C |
| 36 | Amplicon347285 | 38912 | 101 | 101 | SNP | C | b73 | T | mo17 | MRT4577_386764C |
| 36 | Amplicon347285 | 38913 | 106 | 106 | SNP | A | mo17 | C | b73 | MRT4577_386764C |
| 36 | Amplicon347285 | 38914 | 129 | 132 | IND | **** | mo17 | ATTA | b73 | MRT4577_386764C |
| 36 | Amplicon347285 | 38915 | 149 | 149 | SNP | A | mo17 | G | b73 | MRT4577_386764C |
| 36 | Amplicon347285 | 38916 | 153 | 153 | SNP | A | mo17 | C | b73 | MRT4577_386764C |
| 36 | Amplicon347285 | 38917 | 159 | 159 | SNP | C | b73 | T | mo17 | MRT4577_386764C |
| 36 | Amplicon347285 | 38918 | 176 | 176 | SNP | A | mo17 | G | b73 | MRT4577_386764C |
| 36 | Amplicon347285 | 38919 | 181 | 181 | IND | * | mo17 | G | b73 | MRT4577_386764C |
| 36 | Amplicon347285 | 38920 | 281 | 281 | SNP | C | b73 | T | mo17 | MRT4577_386764C |
| 36 | Amplicon347285 | 38921 | 376 | 376 | SNP | C | b73 | G | mo17 | MRT4577_386764C |
| 36 | Amplicon347285 | 38922 | 512 | 512 | SNP | G | b73 | T | mo17 | MRT4577_386764C |
| 36 | Amplicon347285 | 38923 | 518 | 518 | SNP | C | mo17 | T | b73 | MRT4577_386764C |
| 37 | Amplicon347598 | 39507 | 138 | 138 | SNP | C | mo17 | T | b73 | MRT4577_417745C |
| 37 | Amplicon347598 | 39508 | 434 | 435 | IND | ** | mo17 | CC | b73 | MRT4577_417745C |
| 37 | Amplicon347598 | 39509 | 478 | 480 | IND | *** | b73 | GCT | mo17 | MRT4577_417745C |
| 37 | Amplicon347598 | 39510 | 501 | 509 | IND | ********* | b73 | ATGGCAGGC | mo17 | MRT4577_417745C |
| 37 | Amplicon347598 | 39511 | 560 | 560 | SNP | C | mo17 | G | b73 | MRT4577_417745C |
| 38 | Amplicon390056 | 40189 | 325 | 325 | SNP | C | mo17 | T | b73 | MRT4577_43098C |
| 39 | Amplicon390137 | 40320 | 320 | 320 | SNP | C | b73 | T | mo17 | MRT4577_222465C |
| 40 | Amplicon391267 | 41850 | 55 | 55 | SNP | C | b73 | T | mo17 | MRT4577_326681C |
| 40 | Amplicon391267 | 41851 | 112 | 112 | SNP | C | b73 | G | mo17 | MRT4577_326681C |
| 40 | Amplicon391267 | 41852 | 120 | 120 | SNP | A | mo17 | T | b73 | MRT4577_326681C |
| 41 | Amplicon391526 | 42161 | 134 | 134 | SNP | G | mo17 | T | b73 | MRT4577_361986C |
| 41 | Amplicon391526 | 42162 | 194 | 194 | SNP | A | b73 | G | mo17 | MRT4577_361986C |
| 41 | Amplicon391526 | 42163 | 254 | 254 | SNP | A | mo17 | G | b73 | MRT4577_361986C |
| 41 | Amplicon391526 | 42164 | 320 | 320 | SNP | A | b73 | G | mo17 | MRT4577_361986C |
| 41 | Amplicon391526 | 42165 | 350 | 350 | SNP | C | mo17 | T | b73 | MRT4577_361986C |
| 41 | Amplicon391526 | 42166 | 374 | 374 | SNP | A | mo17 | G | b73 | MRT4577_361986C |
| 42 | Amplicon437734 | 42930 | 137 | 137 | SNP | A | mo17 | C | b73 | MRT4577_418799C |
| 42 | Amplicon437734 | 42931 | 196 | 196 | SNP | C | b73 | T | mo17 | MRT4577_418799C |
| 42 | Amplicon437734 | 42932 | 298 | 298 | SNP | A | b73 | G | mo17 | MRT4577_418799C |
| 42 | Amplicon437734 | 42933 | 339 | 339 | SNP | A | b73 | G | mo17 | MRT4577_418799C |
| 42 | Amplicon437734 | 42934 | 422 | 422 | SNP | A | b73 | G | mo17 | MRT4577_418799C |
| 42 | Amplicon437734 | 42935 | 428 | 428 | SNP | C | b73 | T | mo17 | MRT4577_418799C |
| 43 | Amplicon438229 | 43576 | 48 | 48 | SNP | A | b73 | T | mo17 | MRT4577_300134C |
| 43 | Amplicon438229 | 43577 | 49 | 49 | SNP | A | b73 | T | mo17 | MRT4577_300134C |
| 43 | Amplicon438229 | 43578 | 72 | 72 | SNP | A | mo17 | T | b73 | MRT4577_300134C |
| 43 | Amplicon438229 | 43579 | 154 | 154 | SNP | C | b73 | T | mo17 | MRT4577_300134C |
| 43 | Amplicon438229 | 43580 | 218 | 218 | SNP | C | b73 | T | mo17 | MRT4577_300134C |
| 43 | Amplicon438229 | 43581 | 275 | 275 | SNP | A | mo17 | C | b73 | MRT4577_300134C |
| 44 | Amplicon558095 | 51419 | 252 | 252 | SNP | C | b73 | T | mo17 | MRT4577_415225C |
| 45 | Amplicon558289 | 52078 | 105 | 105 | IND | * | mo17 | G | b73 | MRT4577_392856C |
| 45 | Amplicon558289 | 52080 | 107 | 107 | IND | * | mo17 | C | b73 | MRT4577_392856C |
| 45 | Amplicon558289 | 52081 | 351 | 351 | SNP | C | b73 | T | mo17 | MRT4577_392856C |
| 46 | Amplicon559759 | 58375 | 494 | 494 | SNP | C | mo17 | T | b73 | MRT4577_56004C |
| 47 | Amplicon559897 | 58904 | 120 | 120 | SNP | C | b73 | G | mo17 | MRT4577_403109C |
| 47 | Amplicon559897 | 58905 | 216 | 216 | SNP | A | mo17 | T | b73 | MRT4577_403109C |
| 47 | Amplicon559897 | 58906 | 314 | 314 | SNP | A | b73 | T | mo17 | MRT4577_403109C |
| 48 | Amplicon559922 | 59006 | 22 | 22 | SNP | A | b73 | T | mo17 | MRT4577_221761C |
| 48 | Amplicon559922 | 59007 | 34 | 34 | SNP | C | b73 | G | mo17 | MRT4577_221761C |
| 48 | Amplicon559922 | 59008 | 83 | 83 | SNP | C | mo17 | T | b73 | MRT4577_221761C |
| 48 | Amplicon559922 | 59009 | 184 | 184 | SNP | A | b73 | C | mo17 | MRT4577_221761C |
| 48 | Amplicon559922 | 59010 | 234 | 234 | SNP | G | b73 | T | mo17 | MRT4577_221761C |
| 48 | Amplicon559922 | 59011 | 261 | 261 | SNP | C | mo17 | T | b73 | MRT4577_221761C |
| 49 | Amplicon560371 | 60751 | 299 | 299 | SNP | A | mo17 | G | b73 | MRT4577_405424C |
| 49 | Amplicon560371 | 60753 | 371 | 371 | SNP | A | mo17 | T | b73 | MRT4577_405424C |
| 49 | Amplicon560371 | 60754 | 376 | 376 | SNP | A | b73 | C | mo17 | MRT4577_405424C |
| 49 | Amplicon560371 | 60755 | 445 | 445 | SNP | A | mo17 | G | b73 | MRT4577_405424C |
| 50 | Amplicon617780 | 69188 | 172 | 172 | SNP | A | mo17 | G | b73 | MRT4577_401949C |
| 51 | Amplicon671043 | 77568 | 250 | 250 | SNP | A | mo17 | G | b73 | MRT4577_417394C |
| 52 | Amplicon671315 | 78437 | 95 | 95 | SNP | C | mo17 | G | b73 | MRT4577_213040C |
| 52 | Amplicon671315 | 78438 | 138 | 138 | SNP | C | b73 | T | mo17 | MRT4577_213040C |
| 53 | Amplicon724218 | 82235 | 507 | 507 | SNP | A | mo17 | C | b73 | MRT4577_394773C |
| 54 | Amplicon993221 | 104389 | 211 | 211 | SNP | C | LH82 | T | 5CM1 | MRT4577_26957C |
| 54 | Amplicon993221 | 104390 | 225 | 225 | SNP | C | LH82 | G | 5CM1 | MRT4577_26957C |
| 54 | Amplicon993221 | 104391 | 226 | 226 | SNP | A | LH82 | G | 5CM1 | MRT4577_26957C |
| 54 | Amplicon993221 | 104392 | 227 | 227 | SNP | C | 5CM1 | T | LH82 | MRT4577_26957C |
| 54 | Amplicon993221 | 104393 | 231 | 231 | SNP | C | 5CM1 | T | LH82 | MRT4577_26957C |
| 54 | Amplicon993221 | 104394 | 233 | 233 | SNP | A | 5CM1 | G | LH82 | MRT4577_26957C |
| 54 | Amplicon993221 | 104395 | 252 | 252 | SNP | C | LH82 | G | 5CM1 | MRT4577_26957C |
| 55 | Amplicon993328 | 104809 | 23 | 23 | SNP | C | LH82 | T | 5CM1 | MRT4577_399958C |
| 55 | Amplicon993328 | 104810 | 24 | 24 | SNP | G | 5CM1 | T | LH82 | MRT4577_399958C |
| 55 | Amplicon993328 | 104811 | 25 | 25 | SNP | A | 5CM1 | G | LH82 | MRT4577_399958C |
| 55 | Amplicon993328 | 104812 | 26 | 26 | SNP | C | LH82 | T | 5CM1 | MRT4577_399958C |
| 55 | Amplicon993328 | 104813 | 27 | 27 | SNP | C | LH82 | T | 5CM1 | MRT4577_399958C |
| 55 | Amplicon993328 | 104814 | 28 | 28 | SNP | A | LH82 | C | 5CM1 | MRT4577_399958C |
| 55 | Amplicon993328 | 104815 | 29 | 29 | SNP | C | 5CM1 | G | LH82 | MRT4577_399958C |
| 55 | Amplicon993328 | 104816 | 30 | 30 | SNP | A | LH82 | G | 5CM1 | MRT4577_399958C |
| 55 | Amplicon993328 | 104817 | 31 | 31 | SNP | A | 5CM1 | G | LH82 | MRT4577_399958C |
| 55 | Amplicon993328 | 104818 | 32 | 32 | SNP | A | LH82 | T | 5CM1 | MRT4577_399958C |
| 55 | Amplicon993328 | 104819 | 34 | 34 | SNP | A | 5CM1 | C | LH82 | MRT4577_399958C |
| 55 | Amplicon993328 | 104820 | 35 | 35 | SNP | A | LH82 | C | 5CM1 | MRT4577_399958C |
| 55 | Amplicon993328 | 104821 | 36 | 36 | SNP | A | LH82 | T | 5CM1 | MRT4577_399958C |
| 55 | Amplicon993328 | 104822 | 46 | 46 | SNP | A | 5CM1 | C | LH82 | MRT4577_399958C |
| 55 | Amplicon993328 | 104823 | 97 | 97 | SNP | A | LH82 | C | 5CM1 | MRT4577_399958C |
| 55 | Amplicon993328 | 104824 | 98 | 100 | IND | *** | 5CM1 | AAA | LH82 | MRT4577_399958C |
| 55 | Amplicon993328 | 104825 | 184 | 184 | SNP | C | LH82 | T | 5CM1 | MRT4577_399958C |
| 55 | Amplicon993328 | 104826 | 213 | 213 | SNP | C | 5CM1 | T | LH82 | MRT4577_399958C |
| 55 | Amplicon993328 | 104827 | 276 | 276 | SNP | A | 5CM1 | G | LH82 | MRT4577_399958C |
| 55 | Amplicon993328 | 104828 | 475 | 475 | SNP | C | 5CM1 | T | LH82 | MRT4577_399958C |
| 56 | Amplicon993333 | 104845 | 33 | 33 | SNP | A | 5CM1 | G | LH82 | MRT4577_401698C |
| 56 | Amplicon993333 | 104846 | 41 | 41 | SNP | C | 5CM1 | T | LH82 | MRT4577_401698C |
| 56 | Amplicon993333 | 104847 | 142 | 142 | SNP | A | 5CM1 | T | LH82 | MRT4577_401698C |
| 56 | Amplicon993333 | 104848 | 324 | 324 | SNP | A | LH82 | C | 5CM1 | MRT4577_401698C |
| 56 | Amplicon993333 | 104849 | 366 | 366 | SNP | G | LH82 | T | 5CM1 | MRT4577_401698C |
| 56 | Amplicon993333 | 104850 | 400 | 400 | SNP | A | 5CM1 | C | LH82 | MRT4577_401698C |
| 56 | Amplicon993333 | 104851 | 432 | 432 | SNP | G | LH82 | T | 5CM1 | MRT4577_401698C |
| 56 | Amplicon993333 | 104852 | 435 | 435 | SNP | C | 5CM1 | T | LH82 | MRT4577_401698C |
| 56 | Amplicon993333 | 104853 | 456 | 456 | SNP | A | LH82 | T | 5CM1 | MRT4577_401698C |
| 56 | Amplicon993333 | 104854 | 457 | 457 | SNP | A | LH82 | T | 5CM1 | MRT4577_401698C |
| 56 | Amplicon993333 | 104855 | 461 | 461 | IND | * | LH82 | C | 5CM1 | MRT4577_401698C |
| 57 | Amplicon993789 | 106844 | 82 | 82 | SNP | A | 5CM1 | G | LH82 | MRT4577_289436C |
| 57 | Amplicon993789 | 106845 | 110 | 110 | SNP | A | LH82 | G | 5CM1 | MRT4577_289436C |
| 58 | Amplicon993841 | 107074 | 181 | 181 | SNP | C | 5CM1 | T | LH82 | MRT4577_221609C |
| 58 | Amplicon993841 | 107075 | 195 | 195 | SNP | C | 5CM1 | T | LH82 | MRT4577_221609C |
| 58 | Amplicon993841 | 107076 | 206 | 206 | SNP | C | LH82 | T | 5CM1 | MRT4577_221609C |
| 58 | Amplicon993841 | 107077 | 381 | 381 | SNP | A | LH82 | G | 5CM1 | MRT4577_221609C |
| 58 | Amplicon993841 | 107078 | 432 | 432 | SNP | C | LH82 | T | 5CM1 | MRT4577_221609C |
| 59 | Amplicon994045 | 107937 | 311 | 311 | SNP | A | 5CM1 | G | LH82 | MRT4577_28967C |
| 59 | Amplicon994045 | 107938 | 332 | 332 | SNP | C | 5CM1 | T | LH82 | MRT4577_28967C |
| 59 | Amplicon994045 | 107939 | 340 | 340 | SNP | G | LH82 | T | 5CM1 | MRT4577_28967C |
| 59 | Amplicon994045 | 107940 | 416 | 416 | SNP | A | 5CM1 | C | LH82 | MRT4577_28967C |
| 60 | Amplicon1017193 | 109396 | 440 | 449 | IND | ********** | LH82 | ACACACACAC | 5CM1 | MRT4577_151195C |
| 60 | Amplicon1017193 | 109397 | 482 | 482 | SNP | C | LH82 | G | 5CM1 | MRT4577_151195C |
| 60 | Amplicon1017193 | 109398 | 488 | 491 | IND | **** | LH82 | CTCA | 5CM1 | MRT4577_151195C |
| 60 | Amplicon1017193 | 109399 | 496 | 496 | SNP | C | LH82 | G | 5CM1 | MRT4577_151195C |
| 60 | Amplicon1017193 | 109400 | 500 | 500 | SNP | C | LH82 | G | 5CM1 | MRT4577_151195C |
| 60 | Amplicon1017193 | 109401 | 504 | 504 | SNP | C | LH82 | G | 5CM1 | MRT4577_151195C |
| 60 | Amplicon1017193 | 109402 | 511 | 511 | SNP | A | 5CM1 | G | LH82 | MRT4577_151195C |
| 60 | Amplicon1017193 | 109403 | 523 | 525 | IND | *** | LH82 | TTC | 5CM1 | MRT4577_151195C |
| 60 | Amplicon1017193 | 109404 | 540 | 540 | SNP | C | LH82 | G | 5CM1 | MRT4577_151195C |
| 61 | Amplicon1017331 | 110063 | 17 | 17 | SNP | G | 5CM1 | T | LH82 | MRT4577_412840C |
| 61 | Amplicon1017331 | 110064 | 21 | 21 | SNP | C | LH82 | G | 5CM1 | MRT4577_412840C |
| 61 | Amplicon1017331 | 110065 | 123 | 123 | SNP | A | 5CM1 | G | LH82 | MRT4577_412840C |
| 61 | Amplicon1017331 | 110066 | 245 | 248 | IND | **** | LH82 | TATA | 5CM1 | MRT4577_412840C |
| 61 | Amplicon1017331 | 110067 | 276 | 276 | SNP | A | 5CM1 | G | LH82 | MRT4577_412840C |
| 61 | Amplicon1017331 | 110068 | 281 | 281 | SNP | C | 5CM1 | G | LH82 | MRT4577_412840C |
| 61 | Amplicon1017331 | 110069 | 314 | 314 | SNP | A | LH82 | G | 5CM1 | MRT4577_412840C |
| 61 | Amplicon1017331 | 110070 | 375 | 375 | SNP | G | 5CM1 | T | LH82 | MRT4577_412840C |
| 62 | Amplicon1017493 | 110780 | 360 | 360 | SNP | A | LH82 | G | 5CM1 | MRT4577_45217C |
| 63 | Amplicon1017519 | 110886 | 94 | 99 | IND | ****** | LH82 | ATCTGC | 5CM1 | MRT4577_420096C |
| 63 | Amplicon1017519 | 110887 | 136 | 136 | SNP | C | LH82 | T | 5CM1 | MRT4577_420096C |
| 63 | Amplicon1017519 | 110888 | 262 | 265 | IND | **** | LH82 | TTAT | 5CM1 | MRT4577_420096C |
| 63 | Amplicon1017519 | 110889 | 356 | 356 | SNP | G | LH82 | T | 5CM1 | MRT4577_420096C |
| 63 | Amplicon1017519 | 110890 | 403 | 403 | IND | * | LH82 | T | 5CM1 | MRT4577_420096C |
| 63 | Amplicon1017519 | 110891 | 405 | 409 | IND | ***** | LH82 | CCTGT | 5CM1 | MRT4577_420096C |
| 63 | Amplicon1017519 | 110892 | 432 | 432 | SNP | A | 5CM1 | T | LH82 | MRT4577_420096C |
| 63 | Amplicon1017519 | 110894 | 465 | 471 | IND | ******* | LH82 | GAACCAA | 5CM1 | MRT4577_420096C |
| 63 | Amplicon1017519 | 110895 | 547 | 547 | SNP | C | 5CM1 | G | LH82 | MRT4577_420096C |
| 63 | Amplicon1017519 | 110896 | 553 | 553 | IND | * | LH82 | A | 5CM1 | MRT4577_420096C |
| 63 | Amplicon1017519 | 110897 | 555 | 557 | IND | *** | LH82 | CAT | 5CM1 | MRT4577_420096C |
| 64 | Amplicon1050237 | 112139 | 94 | 94 | SNP | C | 5CM1 | G | LH82 | MRT4577_220452C |
| 65 | Amplicon1459206 | 143407 | 116 | 116 | SNP | C | LH82 | T | 5CM1 | MRT4577_416979C |
| 65 | Amplicon1459206 | 143408 | 382 | 382 | SNP | G | 5CM1 | T | LH82 | MRT4577_416979C |
| 65 | Amplicon1459206 | 143409 | 517 | 517 | SNP | A | LH82 | C | 5CM1 | MRT4577_416979C |
| 66 | Amplicon1459208 | 143413 | 71 | 71 | SNP | A | 5CM1 | G | LH82 | MRT4577_5002C |
| 66 | Amplicon1459208 | 143418 | 206 | 206 | SNP | A | 5CM1 | T | LH82 | MRT4577_5002C |
| 67 | Amplicon1459269 | 144505 | 46 | 46 | SNP | C | b73 | T | mo17:5CM1:LH82 | MRT4577_400334C |
| 67 | Amplicon1459269 | 144506 | 89 | 92 | IND | **** | b73 | TCTA | mo17:5CM1:LH82 | MRT4577_400334C |
| 68 | Amplicon1459277 | 144731 | 170 | 170 | SNP | A | b73:mo17:5CM1 | G | LH82 | MRT4577_400556C |
| 68 | Amplicon1459277 | 144732 | 239 | 239 | SNP | A | b73:mo17:5CM1 | G | LH82 | MRT4577_400556C |
| 69 | Amplicon1459300 | 145200 | 103 | 103 | SNP | C | b73 | G | mo17:5CM1:LH82 | MRT4577_389607C |
| 69 | Amplicon1459300 | 145202 | 177 | 177 | SNP | A | b73 | G | mo17:5CM1:LH82 | MRT4577_389607C |
| 69 | Amplicon1459300 | 145203 | 178 | 178 | SNP | A | b73 | C | mo17:5CM1:LH82 | MRT4577_389607C |
| 69 | Amplicon1459300 | 145204 | 272 | 272 | SNP | C | b73 | G | mo17:5CM1:LH82 | MRT4577_389607C |
| 69 | Amplicon1459300 | 145205 | 455 | 458 | IND | **** | mo17:LH82 | ACGT | b73:5CM1 | MRT4577_389607C |
| 70 | Amplicon1459304 | 145260 | 159 | 159 | SNP | A | 5CM1 | C | LH82 | MRT4577_405388C |
| 70 | Amplicon1459304 | 145261 | 173 | 173 | SNP | C | LH82 | G | 5CM1 | MRT4577_405388C |
| 70 | Amplicon1459304 | 145263 | 236 | 236 | SNP | C | 5CM1 | T | LH82 | MRT4577_405388C |
| 70 | Amplicon1459304 | 145264 | 526 | 526 | SNP | C | 5CM1 | T | LH82 | MRT4577_405388C |
| 70 | Amplicon1459304 | 145266 | 575 | 575 | SNP | C | 5CM1 | T | LH82 | MRT4577_405388C |
| 71 | Amplicon1459369 | 146410 | 124 | 124 | SNP | G | b73:mo17:LH82 | T | 5CM1 | MRT4577_388272C |
| 71 | Amplicon1459369 | 146411 | 155 | 160 | IND | ****** | 5CM1 | ATCTTC | b73:mo17:LH82 | MRT4577_388272C |
| 71 | Amplicon1459369 | 146412 | 281 | 281 | SNP | C | 5CM1 | T | b73:mo17:LH82 | MRT4577_388272C |
| 71 | Amplicon1459369 | 146413 | 331 | 331 | SNP | A | LH82 | T | b73:mo17:5CM1 | MRT4577_388272C |
| 71 | Amplicon1459369 | 146414 | 332 | 332 | SNP | A | LH82 | C | b73:mo17:5CM1 | MRT4577_388272C |
| 71 | Amplicon1459369 | 146415 | 346 | 346 | SNP | A | b73:LH82 | G | mo17:5CM1 | MRT4577_388272C |
| 71 | Amplicon1459369 | 146416 | 553 | 553 | SNP | G | b73:mo17:LH82 | T | 5CM1 | MRT4577_388272C |
| 71 | Amplicon1459369 | 146417 | 556 | 556 | SNP | C | b73:mo17:LH82 | G | 5CM1 | MRT4577_388272C |
| 71 | Amplicon1459369 | 146418 | 557 | 557 | SNP | G | 5CM1 | T | b73:mo17:LH82 | MRT4577_388272C |
| 71 | Amplicon1459369 | 146419 | 559 | 559 | SNP | G | b73:mo17:LH82 | T | 5CM1 | MRT4577_388272C |
| 71 | Amplicon1459369 | 146420 | 560 | 560 | SNP | A | 5CM1 | T | b73:mo17:LH82 | MRT4577_388272C |
| 71 | Amplicon1459369 | 146421 | 561 | 561 | SNP | A | 5CM1 | T | b73:mo17:LH82 | MRT4577_388272C |
| 71 | Amplicon1459369 | 146422 | 562 | 562 | SNP | A | 5CM1 | T | b73:mo17:LH82 | MRT4577_388272C |
| 71 | Amplicon1459369 | 146423 | 563 | 563 | SNP | G | b73:mo17:LH82 | T | 5CM1 | MRT4577_388272C |
| 71 | Amplicon1459369 | 146424 | 564 | 564 | SNP | A | 5CM1 | G | b73:mo17:LH82 | MRT4577_388272C |
| 71 | Amplicon1459369 | 146425 | 565 | 565 | SNP | A | 5CM1 | G | b73:mo17:LH82 | MRT4577_388272C |
| 71 | Amplicon1459369 | 146426 | 566 | 566 | SNP | A | 5CM1 | T | b73:mo17:LH82 | MRT4577_388272C |
| 71 | Amplicon1459369 | 146427 | 567 | 567 | SNP | A | b73:mo17:LH82 | C | 5CM1 | MRT4577_388272C |
| 71 | Amplicon1459369 | 146428 | 569 | 569 | SNP | C | 5CM1 | T | b73:mo17:LH82 | MRT4577_388272C |
| 71 | Amplicon1459369 | 146429 | 570 | 570 | SNP | C | b73:mo17:LH82 | G | 5CM1 | MRT4577_388272C |
| 71 | Amplicon1459369 | 146430 | 571 | 571 | SNP | A | 5CM1 | T | b73:mo17:LH82 | MRT4577_388272C |
| 71 | Amplicon1459369 | 146431 | 575 | 575 | SNP | A | b73:mo17:LH82 | T | 5CM1 | MRT4577_388272C |
| 71 | Amplicon1459369 | 146432 | 576 | 576 | SNP | A | 5CM1 | T | b73:mo17:LH82 | MRT4577_388272C |
| 71 | Amplicon1459369 | 146433 | 577 | 577 | SNP | G | 5CM1 | T | b73:mo17:LH82 | MRT4577_388272C |
| 71 | Amplicon1459369 | 146434 | 578 | 578 | SNP | A | 5CM1 | G | b73:mo17:LH82 | MRT4577_388272C |
| 71 | Amplicon1459369 | 146435 | 579 | 579 | SNP | A | b73:mo17:LH82 | T | 5CM1 | MRT4577_388272C |
| 71 | Amplicon1459369 | 146436 | 581 | 581 | SNP | C | 5CM1 | G | b73:mo17:LH82 | MRT4577_388272C |
| 71 | Amplicon1459369 | 146437 | 582 | 582 | SNP | A | 5CM1 | T | b73:mo17:LH82 | MRT4577_388272C |
| 71 | Amplicon1459369 | 146438 | 583 | 583 | SNP | A | 5CM1 | G | b73:mo17:LH82 | MRT4577_388272C |
| 71 | Amplicon1459369 | 146439 | 584 | 584 | SNP | A | 5CM1 | G | b73:mo17:LH82 | MRT4577_388272C |
| 71 | Amplicon1459369 | 146441 | 588 | 588 | SNP | A | b73:mo17:LH82 | C | 5CM1 | MRT4577_388272C |
| 71 | Amplicon1459369 | 146442 | 589 | 589 | SNP | A | b73:mo17:LH82 | T | 5CM1 | MRT4577_388272C |
| 71 | Amplicon1459369 | 146443 | 590 | 590 | SNP | G | b73:mo17:LH82 | T | 5CM1 | MRT4577_388272C |
| 71 | Amplicon1459369 | 146444 | 591 | 591 | SNP | C | 5CM1 | G | b73:mo17:LH82 | MRT4577_388272C |
| 71 | Amplicon1459369 | 146445 | 593 | 593 | SNP | C | 5CM1 | G | b73:mo17:LH82 | MRT4577_388272C |
| 71 | Amplicon1459369 | 146446 | 594 | 594 | SNP | C | 5CM1 | T | b73:mo17:LH82 | MRT4577_388272C |
| 71 | Amplicon1459369 | 146447 | 595 | 595 | SNP | A | b73:mo17:LH82 | C | 5CM1 | MRT4577_388272C |
| 71 | Amplicon1459369 | 146448 | 596 | 596 | SNP | C | 5CM1 | T | b73:mo17:LH82 | MRT4577_388272C |
| 71 | Amplicon1459369 | 146449 | 598 | 598 | SNP | A | 5CM1 | G | b73:mo17:LH82 | MRT4577_388272C |
| 71 | Amplicon1459369 | 146450 | 599 | 599 | SNP | A | b73:mo17:LH82 | C | 5CM1 | MRT4577_388272C |
| 72 | Amplicon1460644 | 148039 | 95 | 95 | SNP | C | b73:5CM1 | T | mo17:LH82 | MRT4577_61311C |
| 72 | Amplicon1460644 | 148040 | 116 | 116 | SNP | A | LH82 | C | b73:mo17:5CM1 | MRT4577_61311C |
| 72 | Amplicon1460644 | 148041 | 126 | 126 | SNP | C | 5CM1 | T | b73:mo17:LH82 | MRT4577_61311C |
| 72 | Amplicon1460644 | 148042 | 140 | 140 | IND | * | 5CM1 | C | b73:mo17:LH82 | MRT4577_61311C |
| 72 | Amplicon1460644 | 148043 | 147 | 147 | SNP | A | mo17 | C | b73:5CM1:LH82 | MRT4577_61311C |
| 72 | Amplicon1460644 | 148044 | 172 | 172 | SNP | A | b73:5CM1 | G | mo17:LH82 | MRT4577_61311C |
| 72 | Amplicon1460644 | 148045 | 191 | 191 | SNP | A | b73:mo17:5CM1 | C | LH82 | MRT4577_61311C |
| 72 | Amplicon1460644 | 148046 | 193 | 193 | SNP | A | b73:5CM1 | G | mo17:LH82 | MRT4577_61311C |
| 72 | Amplicon1460644 | 148047 | 210 | 210 | SNP | A | b73:5CM1 | G | mo17:LH82 | MRT4577_61311C |
| 72 | Amplicon1460644 | 148048 | 218 | 218 | SNP | C | b73:mo17:5CM1 | T | LH82 | MRT4577_61311C |
| 72 | Amplicon1460644 | 148049 | 223 | 223 | SNP | A | LH82 | G | b73:mo17:5CM1 | MRT4577_61311C |
| 72 | Amplicon1460644 | 148050 | 253 | 253 | SNP | A | b73:5CM1 | G | mo17:LH82 | MRT4577_61311C |
| 72 | Amplicon1460644 | 148051 | 259 | 259 | SNP | A | b73:5CM1 | G | mo17:LH82 | MRT4577_61311C |
| 72 | Amplicon1460644 | 148052 | 274 | 274 | SNP | A | b73:5CM1:LH82 | G | mo17 | MRT4577_61311C |
| 72 | Amplicon1460644 | 148053 | 291 | 291 | SNP | A | b73 | G | mo17:5CM1:LH82 | MRT4577_61311C |
| 72 | Amplicon1460644 | 148054 | 296 | 296 | SNP | A | mo17:LH82 | G | b73:5CM1 | MRT4577_61311C |
| 72 | Amplicon1460644 | 148055 | 309 | 309 | SNP | C | mo17 | T | b73:5CM1:LH82 | MRT4577_61311C |
| 72 | Amplicon1460644 | 148056 | 326 | 340 | IND | *************** | b73:5CM1 | CCTTCGATGATATG | LH82 | MRT4577_61311C |
| 72 | Amplicon1460644 | 148057 | 343 | 357 | IND | *************** | mo17 | TCGACGATGACGCC | MRT4577_61311C | |
| 72 | Amplicon1460644 | 148058 | 360 | 360 | SNP | C | mo17:LH82 | G | b73:5CM1 | MRT4577_61311C |
| 72 | Amplicon1460644 | 148059 | 361 | 361 | SNP | C | mo17:LH82 | T | b73:5CM1 | MRT4577_61311C |
| 72 | Amplicon1460644 | 148060 | 368 | 368 | SNP | C | b73:5CM1 | T | mo17:LH82 | MRT4577_61311C |
| 72 | Amplicon1460644 | 148061 | 373 | 373 | SNP | A | mo17:LH82 | G | b73:5CM1 | MRT4577_61311C |
| 72 | Amplicon1460644 | 148062 | 376 | 376 | SNP | A | b73:5CM1 | G | mo17:LH82 | MRT4577_61311C |
| 72 | Amplicon1460644 | 148063 | 379 | 379 | SNP | G | b73:5CM1:LH82 | T | mo17 | MRT4577_61311C |
| 72 | Amplicon1460644 | 148064 | 383 | 383 | SNP | A | LH82 | C | b73:mo17:5CM1 | MRT4577_61311C |
| 72 | Amplicon1460644 | 148065 | 385 | 385 | SNP | A | b73:5CM1 | G | mo17:LH82 | MRT4577_61311C |
| 72 | Amplicon1460644 | 148066 | 394 | 394 | SNP | A | b73:5CM1:LH82 | G | mo17 | MRT4577_61311C |
| 72 | Amplicon1460644 | 148067 | 400 | 400 | SNP | A | mo17 | G | b73:5CM1:LH82 | MRT4577_61311C |
| 72 | Amplicon1460644 | 148068 | 425 | 425 | SNP | C | mo17:LH82 | T | b73:5CM1 | MRT4577_61311C |
| 72 | Amplicon1460644 | 148069 | 433 | 433 | SNP | A | b73 | G | mo17:5CM1:LH82 | MRT4577_61311C |
| 73 | Amplicon1461872 | 151382 | 225 | 225 | SNP | A | b73:LH82 | C | 5CM1 | MRT4577_287993C |
| 73 | Amplicon1461872 | 151384 | 419 | 419 | SNP | C | 5CM1 | G | b73:LH82 | MRT4577_287993C |
| 73 | Amplicon1461872 | 151385 | 445 | 445 | SNP | C | b73:LH82 | G | 5CM1 | MRT4577_287993C |
| 73 | Amplicon1461872 | 151386 | 532 | 532 | SNP | A | b73:LH82 | T | 5CM1 | MRT4577_287993C |
| 73 | Amplicon1461872 | 151388 | 535 | 535 | IND | * | b73 | G | 5CM1:LH82 | MRT4577_287993C |
| 73 | Amplicon1461872 | 151389 | 535 | 537 | IND | *** | b73:LH82 | GCG | 5CM1 | MRT4577_287993C |
| 73 | Amplicon1461872 | 151390 | 540 | 544 | IND | ***** | b73:LH82 | TTGCC | 5CM1 | MRT4577_287993C |
| 73 | Amplicon1461872 | 151391 | 559 | 560 | IND | ** | LH82 | *A | | MRT4577_287993C |
| 73 | Amplicon1461872 | 151392 | 563 | 563 | IND | * | b73 | A | 5CM1:LH82 | MRT4577_287993C |
| 73 | Amplicon1461872 | 151393 | 569 | 569 | SNP | C | 5CM1 | G | b73:LH82 | MRT4577_287993C |
| 73 | Amplicon1461872 | 151396 | 639 | 641 | IND | *** | 5CM1 | GGC | b73:LH82 | MRT4577_287993C |
| 73 | Amplicon1461872 | 151397 | 643 | 643 | IND | * | 5CM1 | T | b73:LH82 | MRT4577_287993C |

TABLE 2

| SEQ NUM | Seq ID | Description |
|---|---|---|
| 74 | MRT4577_407583C | gene encoding MRT4577_407583P |
| 75 | MRT4577_37957C | gene encoding MRT4577_37957P |
| 76 | MRT4577_306229C | gene encoding MRT4577_306229P |
| 77 | MRT4577_305583C | gene encoding MRT4577_305583P |
| 78 | MRT4577_189292C | gene encoding MRT4577_189292P |
| 79 | MRT4577_409052C | gene encoding MRT4577_409052P |
| 80 | MRT4577_371170C | gene encoding MRT4577_371170P |
| 81 | MRT4577_169297C | gene encoding MRT4577_169297P |
| 82 | MRT4577_273665C | gene encoding MRT4577_273665P |
| 83 | MRT4577_285101C | gene encoding MRT4577_285101P |
| 84 | MRT4577_284415C | gene encoding MRT4577_284415P |
| 85 | MRT4577_38704C | gene encoding MRT4577_38704P |
| 86 | MRT4577_47332C | gene encoding MRT4577_47332P |
| 87 | MRT4577_386264C | gene encoding MRT4577_386264P |
| 88 | MRT4577_25879C | gene encoding MRT4577_25879P |
| 89 | MRT4577_419574C | gene encoding MRT4577_419574P |
| 90 | MRT4577_414575C | gene encoding MRT4577_414575P |
| 91 | MRT4577_199838C | gene encoding MRT4577_199838P |
| 92 | MRT4577_409604C | gene encoding MRT4577_409604P |
| 93 | MRT4577_391398C | gene encoding MRT4577_391398P |
| 94 | MRT4577_234188C | gene encoding MRT4577_234188P |
| 95 | MRT4577_264682C | gene encoding MRT4577_264682P |
| 96 | MRT4577_287055C | gene encoding MRT4577_287055P |
| 97 | MRT4577_49099C | gene encoding MRT4577_49099P |
| 98 | MRT4577_346921C | gene encoding MRT4577_346921P |
| 99 | MRT4577_257780C | gene encoding MRT4577_257780P |
| 100 | MRT4577_410376C | gene encoding MRT4577_410376P |
| 101 | MRT4577_233403C | gene encoding MRT4577_233403P |
| 102 | MRT4577_294774C | gene encoding MRT4577_294774P |
| 103 | MRT4577_402771C | gene encoding MRT4577_402771P |
| 104 | MRT4577_397598C | gene encoding MRT4577_397598P |
| 105 | MRT4577_204611C | gene encoding MRT4577_204611P |
| 106 | MRT4577_404797C | gene encoding MRT4577_404797P |
| 107 | MRT4577_32764C | gene encoding MRT4577_32764P |
| 108 | MRT4577_284905C | gene encoding MRT4577_284905P |
| 109 | MRT4577_386764C | gene encoding MRT4577_386764P |
| 110 | MRT4577_417745C | gene encoding MRT4577_417745P |
| 111 | MRT4577_43098C | gene encoding MRT4577_43098P |
| 112 | MRT4577_222465C | gene encoding MRT4577_222465P |
| 113 | MRT4577_326681C | gene encoding MRT4577_326681P |
| 114 | MRT4577_361986C | gene encoding MRT4577_361986P |
| 115 | MRT4577_418799C | gene encoding MRT4577_418799P |
| 116 | MRT4577_300134C | gene encoding MRT4577_300134P |
| 117 | MRT4577_415225C | gene encoding MRT4577_415225P |
| 118 | MRT4577_392856C | gene encoding MRT4577_392856P |
| 119 | MRT4577_56004C | gene encoding MRT4577_56004P |
| 120 | MRT4577_403109C | gene encoding MRT4577_403109P |
| 121 | MRT4577_221761C | gene encoding MRT4577_221761P |
| 122 | MRT4577_405424C | gene encoding MRT4577_405424P |
| 123 | MRT4577_401949C | gene encoding MRT4577_401949P |
| 124 | MRT4577_417394C | gene encoding MRT4577_417394P |
| 125 | MRT4577_213040C | gene encoding MRT4577_213040P |
| 126 | MRT4577_394773C | gene encoding MRT4577_394773P |
| 127 | MRT4577_26957C | gene encoding MRT4577_26957P |
| 128 | MRT4577_399958C | gene encoding MRT4577_399958P |
| 129 | MRT4577_401698C | gene encoding MRT4577_401698P |
| 130 | MRT4577_289436C | gene encoding MRT4577_289436P |
| 131 | MRT4577_221609C | gene encoding MRT4577_221609P |
| 132 | MRT4577_28967C | gene encoding MRT4577_28967P |
| 133 | MRT4577_151195C | gene encoding MRT4577_151195P |
| 134 | MRT4577_412840C | gene encoding MRT4577_412840P |
| 135 | MRT4577_45217C | gene encoding MRT4577_45217P |
| 136 | MRT4577_420096C | gene encoding MRT4577_420096P |
| 137 | MRT4577_220452C | gene encoding MRT4577_220452P |
| 138 | MRT4577_416979C | gene encoding MRT4577_416979P |
| 139 | MRT4577_5002C | gene encoding MRT4577_5002P |
| 140 | MRT4577_400334C | gene encoding MRT4577_400334P |
| 141 | MRT4577_400556C | gene encoding MRT4577_400556P |
| 142 | MRT4577_389607C | gene encoding MRT4577_389607P |
| 143 | MRT4577_405388C | gene encoding MRT4577_405388P |
| 144 | MRT4577_388272C | gene encoding MRT4577_388272P |
| 145 | MRT4577_61311C | gene encoding MRT4577_61311P |
| 146 | MRT4577_287993C | gene encoding MRT4577_287993P |

TABLE 3

| Seq Num | Seq ID | Description |
|---|---|---|
| 147 | MRT4577_407583P | /method = simple longest ORF |
| 148 | MRT4577_37957P | gl\|22758323\|gb\|AAN05527.1\|putative glutamine synthetase |
| | | [Oryza sativa (japonica cultivar-group)]/method = extended homology |
| 149 | MRT4577_306229P | gl\|18767126\|gb\|AAL79278.1\|/method = extended homology |
| 150 | MRT4577_305583P | gl\|28566182\|gb\|AAO43227.1\|phosphoethanolamine |
| | | cytidylyltransferase |
| | | [Hordeum vulgare subsp. vulgare]/method = extended homology |
| 151 | MRT4577_189292P | gl\|22094360\|gb\|AAM91887.1\|putative cytokinin oxidase [Oryza sativa |
| | | (japonica cultivar-group)]/method = homology |
| 152 | MRT4577_409052P | gl\|18568267\|gb\|AAL75999.1\|AF466646_7 putative polyprotein |
| | | [Zea mays]/method = extended homology |
| 153 | MRT4577_371170P | gl\|7339715\|dbj\|BAA92920.1\|EST AU057816(S21817) |
| | | corresponds to a reglon |
| | | of the predicted gene. Similar to Arabidopsis |
| | | thaliana chromosome IV BAC T19F06; |
| | | unknown protein. (AC002343) |
| | | [Oryza sativa]/method = extended homology |
| 154 | MRT4577_169297P | gl\|22325962\|ref\|NP_180419.2\|putative vacuolar |
| | | proton-ATPase subunit; protein id: At2g28520.1, |
| | | supported by cDNA: gl_20259418 [Arabidopsis thaliana]/ |
| | | method = extended homology |
| 155 | MRT4577_273665P | gl\|25408357\|pir\|\|C84765 / |
| | | method = extended homology |
| 156 | MRT4577_285101P | gl\|28971970\|dbj\|BAC65371.1\|putative |
| | | cellulose synthase [Oryza sativa (japonica |
| | | cultivar-group)]/method = extended homology |
| 157 | MRT4577_284415P | gl\|18416861\|ref\|NP_568276.1\|/ |
| | | method = extended homology |
| 158 | MRT4577_38704P | gl\|15242264\|ref\|NP_200017.1\|/ |
| | | method = extended homology |
| 159 | MRT4577_47332P | gl\|18404228\|ref\|NP_566752.1\| |
| | | rubisco expression protein -related [Arabidopsis |
| | | thaliana]/method = extended homology |
| 160 | MRT4577_386264P | gl\|6539568\|dbj\|BAA88185.1\|/ |
| | | method = extended homology |
| 161 | MRT4577_25879P | gl\|15236196\|ref\|NP_194375.1\|/ |
| | | method = extended homology |
| 162 | MRT4577_419574P | /method = simple longest ORF |
| 163 | MRT4577_414575P | /method = longest ORF |
| 164 | MRT4577_199838P | gl\|7487920\|pir\|\|T01025 / |
| | | method = extended homology |
| 165 | MRT4577_409604P | /method = simple longest ORF |
| 166 | MRT4577_391398P | gl\|21740740\|emb\|CAD40549.1\|OSJNBa0072K14.5 |
| | | [Oryza sativa]/method = extended homology |
| 167 | MRT4577_234188P | gl\|5679845\|emb\|CAB51838.1\|/ |
| | | method = extended homology |
| 168 | MRT4577_264682P | gl\|28392860\|gb\|AA041867.1\|/ |
| | | method = extended homology |
| 169 | MRT4577_287055P | gl\|20161246\|dbj\|BAB90173.1\| |
| | | putative ATP-dependent Clp protease regulatory |
| | | subunit CLPX [Oryza sativa (japonica cultivar- |
| | | group)]/method = extended homology |
| 170 | MRT4577_49099P | gl\|137460\|sp\|P09469\|VATA_DAUCA |
| | | VACUOLAR ATP SYNTHASE CATALYTIC SUBUNIT A |
| | | (V-ATPASE A SUBUNIT) (VACUOLAR PROTON PUMP |
| | | ALPHA SUBUNIT) (V-ATPASE 69 KDA SUBUNIT). / |
| | | method = extended homology |
| 171 | MRT4577_346921P | gl\|29372756\|emb\|CAD23413.1\| |
| | | m23 [Zea mays]/method = extended homology |
| 172 | MRT4577_257780P | gl\|15232453\|ref\|NP_188116.1\| |
| | | PHD finger transcription factor, putative |
| | | [Arabidopsis thaliana]/method = extended |
| | | homology |
| 173 | MRT4577_410376P | /method = simple longest ORF |
| 174 | MRT4577_233403P | /method = longest ORF |
| 175 | MRT4577_294774P | gl\|22265999\|emb\|CAC82980.1\| |
| | | fatty acid hydroperoxide lyase [Hordeum vulgare]/ |
| | | method = extended homology |
| 176 | MRT4577_402771P | /method = longest ORF |
| 177 | MRT4577_397598P | gl\|13486777\|dbj\|BAB40010.1\| |
| | | putative wall-associated kinase 2 [Oryza sativa |
| | | (japonica cultivar-group)]/method = homology |
| 178 | MRT4577_204611P | gl\|15866696\|emb\|CAC84558.1\|beta-amyrin |
| | | synthase [Avena strigosa]/method = extended homology |
| 179 | MRT4577_404797P | /method = simple longest ORF |
| 180 | MRT4577_32764P | gl\|18396768\|ref\|NP_564307.1\| |
| | | expressed protein [Arabidopsis thaliana]/ |
| | | method = extended homology |
| 181 | MRT4577_284905P | gl\|22748323\|gb\|AAN05325.1\|/ |
| | | method = extended homology |
| 182 | MRT4577_386764P | gl\|22330199\|ref\|NP_683423.1\| |
| | | somatic embryogenesis receptor-like kinase, |
| | | putative; protein id: At1g52540.2, supported |
| | | by cDNA: 21250. [Arabidopsis thaliana]/ |
| | | method = extended homology |
| 183 | MRT4577_417745P | /method = longest ORF |
| 184 | MRT4577_43098P | gl\|29893654\|gb\|AAP06908.1\|/ |
| | | method = extended homology |
| 185 | MRT4577_222465P | gl\|14587221\|db\|BAB61155.1\|/ |
| | | method = extended homology |
| 186 | MRT4577_326681P | gl\|28071332\|db\|BAC56020.1\| |
| | | putative RNA helicase [Oryza sativa |
| | | (japonica cultivar-group)]/ |
| | | method = extended homology |
| 187 | MRT4577_361986P | gl\|15227441\|ref\|NP_181713.1\|/ |
| | | method = homology |
| 188 | MRT4577_418799P | /method = simple longest ORF |
| 189 | MRT4577_300134P | gl\|7489733\|pir\|\|T01171G1/ |
| | | S transition control protein Rb1 - |
| | | maize /method = extended homology |
| 190 | MRT4577_415225P | gl\|15239966\|ref\|NP_196804.1\| |
| | | callose synthase catalytic subunit - |
| | | like protein [Arabidopsis thaliana]/ |
| | | method = extended homology |
| 191 | MRT4577_392856P | gl\|22135459\|gb\|AAM93210.1\| |
| | | AF527609_1 chromdomain-containing |
| | | protein CRD101 [Zea mays]/ |
| | | method = extended homology |
| 192 | MRT4577_56004P | gl\|18415638\|ref\|NP_567620.1\| |
| | | zinc finger and C2 domain protein |
| | | (ZAC) [Arabidopsis thaliana]/ |
| | | method = extended homology |
| 193 | MRT4577_403109P | /method = simple longest ORF |
| 194 | MRT4577_221761P | gl\|22331664\|ref\|NP_190399.2\| |
| | | DP-E2F-like protein 1; protein id: |
| | | At3g48160.1, supported by cDNA: |
| | | gl_20502507 [Arabidopsis thaliana]/ |
| | | method = extended homology |
| 195 | MRT4577_405424P | /method = longest ORF |
| 196 | MRT4577_401949P | gl\|15209148\|gb\|AAK91881.1\| |
| | | AC091665_7 /method = homology |
| 197 | MRT4577_417394P | /method = longest ORF |
| 198 | MRT4577_213040P | /method = longest ORF |
| 199 | MRT4577_394773P | /method = longest ORF |
| 200 | MRT4577_26957P | gl\|18407057\|ref\|NP_566071.1\|/ |
| | | method = extended homology |
| 201 | MRT4577_399958P | /method = simple longest ORF |
| 202 | MRT4577_401698P | /method = longest ORF |
| 203 | MRT4577_289436P | gl\|24413957\|db\|BAC22209.1\|/ |
| | | method = extended homology |
| 204 | MRT4577_221609P | gl\|20160716\|db\|BAB89658.1\|/ |
| | | method = extended homology |
| 205 | MRT4577_28967P | gl\|9663979\|db\|BAB03620.1\|/ |
| | | method = homology |
| 206 | MRT4577_151195P | /method = simple longest ORF |
| 207 | MRT4577_412840P | /method = simple longest ORF |
| 208 | MRT4577_45217P | gl\|19352035\|db\|BAB85911.1\| |
| | | Arabidopsis ETTIN-like protein 2 |
| | | [Oryza sativa]/method = homology |
| 209 | MRT4577_420096P | gl\|20330751\|gb\|AAM19114.1\| |
| | | AC104427_12 Putative bZIP |
| | | transcription factor [Oryza sativa |
| | | (japonica cultivar-group)]/ |
| | | method = extended homology |
| 210 | MRT4577_220452P | gl\|26449867\|db\|BAC42056.1\|/ |
| | | method = extended homology |
| 211 | MRT4577_416979P | /method = longest ORF |
| 212 | MRT4577_5002P | gl\|7489518\|pir\|\|T02745 nucleic |
| | | acid binding protein - |
| | | rice /method = extended homology |
| 213 | MRT4577_400334P | /method = simple longest ORF |
| 214 | MRT4577_400556P | /method = simple longest ORF |
| 215 | MRT4577_389607P | gl\|20161442\|db\|BAB90366.1\|/ |
| | | method = extended homology |
| 216 | MRT4577_405388P | gl\|1703302\|sp\|P55005\|AMYB_MAIZE |
| | | “BETA-AMYLASE (1,4-ALPHA-D-GLUCAN |
| | | MALTOHYDROLASE)./ |
| | | method = extended homology” |
| 217 | MRT4577_388272P | gl\|7488484\|pir\|\|T07980 |
| | | probable choline-phosphate cytidylyltransferase |
| | | (EC 2.7.7.15) (clone CCT2) - |
| | | rape /method = extended homology |
| 218 | MRT4577_61311P | gl\|7489748\|pir\|\|T03381 high |
| | | sulfurzein protein precursor - |
| | | maize /method = homology |
| 219 | MRT4577_287993P | gl\|11993325\|gb\|AAG42687.1\|AF271383_1 Zea |
| | | mays indole-3-glycerol phosphate lyase (Igl) |
| | | gene, complete cds; and putative tryptophan |
| | | synthase alpha (TSAlike) gene, partial cds./ |
| | | method = homology |

TABLE 4

| Seq_Num | Seq_ID | Homolog_ID |
|---|---|---|
| 148 | MRT4577_37957P | gl_21220152 |
| 148 | MRT4577_37957P | gl_21219634 |
| 148 | MRT4577_37957P | gl_21220814 |
| 148 | MRT4577_37957P | gl_17227784 |
| 148 | MRT4577_37957P | gl_17232340 |
| 148 | MRT4577_37957P | gl_22960297 |
| 148 | MRT4577_37957P | gl_22957596 |
| 148 | MRT4577_37957P | gl_22961512 |
| 148 | MRT4577_37957P | gl_22961554 |
| 148 | MRT4577_37957P | gl_22962442 |
| 148 | MRT4577_37957P | gl_22966395 |
| 148 | MRT4577_37957P | gl_16330288 |
| 148 | MRT4577_37957P | gl_32476398 |
| 148 | MRT4577_37957P | gl_22993136 |
| 148 | MRT4577_37957P | gl_22991262 |
| 148 | MRT4577_37957P | gl_22993311 |
| 148 | MRT4577_37957P | gl_15673109 |
| 148 | MRT4577_37957P | gl_15893448 |
| 148 | MRT4577_37957P | gl_15893920 |
| 148 | MRT4577_37957P | gl_26988777 |
| 148 | MRT4577_37957P | gl_16801989 |
| 148 | MRT4577_37957P | gl_2500204 |
| 148 | MRT4577_37957P | gl_16804835 |
| 148 | MRT4577_37957P | gl_27468813 |
| 148 | MRT4577_37957P | gl_15827378 |
| 148 | MRT4577_37957P | gl_15890531 |
| 148 | MRT4577_37957P | gl_23006404 |
| 148 | MRT4577_37957P | gl_23004108 |
| 148 | MRT4577_37957P | gl_16126335 |
| 148 | MRT4577_37957P | gl_16124956 |
| 148 | MRT4577_37957P | gl_18309257 |
| 148 | MRT4577_37957P | gl_19552720 |
| 148 | MRT4577_37957P | gl_19553182 |
| 148 | MRT4577_37957P | gl_23019853 |
| 148 | MRT4577_37957P | gl_23019267 |
| 148 | MRT4577_37957P | gl_23021869 |
| 148 | MRT4577_37957P | gl_23021249 |
| 148 | MRT4577_37957P | gl_23021813 |
| 148 | MRT4577_37957P | gl_23028929 |
| 148 | MRT4577_37957P | gl_23501390 |
| 148 | MRT4577_37957P | gl_23336124 |
| 148 | MRT4577_37957P | gl_23465101 |
| 148 | MRT4577_37957P | gl_23465609 |
| 148 | MRT4577_37957P | gl_23473416 |
| 148 | MRT4577_37957P | gl_22536365 |
| 148 | MRT4577_37957P | gl_15595759 |
| 148 | MRT4577_37957P | gl_15597263 |
| 148 | MRT4577_37957P | gl_15829106 |
| 148 | MRT4577_37957P | gl_28209952 |
| 148 | MRT4577_37957P | gl_28210965 |
| 148 | MRT4577_37957P | gl_27367975 |
| 148 | MRT4577_37957P | gl_28379494 |
| 148 | MRT4577_37957P | gl_23099057 |
| 148 | MRT4577_37957P | gl_28901282 |
| 148 | MRT4577_37957P | gl_16077989 |
| 148 | MRT4577_37957P | gl_17987729 |
| 148 | MRT4577_37957P | gl_29375007 |
| 148 | MRT4577_37957P | gl_29347953 |
| 148 | MRT4577_37957P | gl_29828077 |
| 148 | MRT4577_37957P | gl_29833210 |
| 148 | MRT4577_37957P | gl_15897407 |
| 148 | MRT4577_37957P | gl_27262322 |
| 148 | MRT4577_37957P | gl_23102311 |
| 148 | MRT4577_37957P | gl_23106149 |
| 148 | MRT4577_37957P | gl_30102526 |
| 148 | MRT4577_37957P | gl_15234470 |
| 148 | MRT4577_37957P | gl_13474110 |
| 148 | MRT4577_37957P | gl_15966192 |
| 148 | MRT4577_37957P | MRT3847_53577P.3 |
| 148 | MRT4577_37957P | MRT3847_267642P.1 |
| 148 | MRT4577_37957P | gl_22758323 |
| 148 | MRT4577_37957P | gl_19881629 |
| 148 | MRT4577_37957P | gl_5881832 |
| 148 | MRT4577_37957P | MRT4530_14454P.2 |
| 148 | MRT4577_37957P | MRT4530_14452P.1 |
| 148 | MRT4577_37957P | MRT4565_134443P.1 |
| 148 | MRT4577_37957P | MRT4565_41750P.3 |
| 148 | MRT4577_37957P | gl_19075895 |
| 148 | MRT4577_37957P | gl_6320442 |
| 148 | MRT4577_37957P | gl_23118917 |
| 148 | MRT4577_37957P | gl_32405352 |
| 148 | MRT4577_37957P | gl_23123201 |
| 148 | MRT4577_37957P | gl_15600873 |
| 148 | MRT4577_37957P | gl_23131072 |
| 148 | MRT4577_37957P | gl_15609923 |
| 148 | MRT4577_37957P | gl_15616426 |
| 148 | MRT4577_37957P | gl_16761259 |
| 148 | MRT4577_37957P | gl_23135856 |
| 148 | MRT4577_37957P | gl_16765661 |
| 149 | MRT4577_306229P | MRT3847_254592P.2 |
| 149 | MRT4577_306229P | MRT3847_234305P.2 |
| 149 | MRT4577_306229P | MRT3847_213371P.3 |
| 149 | MRT4577_306229P | MRT3847_223708P.3 |
| 149 | MRT4577_306229P | MRT4565_71415P.2 |
| 150 | MRT4577_305583P | gl_28566182 |
| 150 | MRT4577_305583P | gl_15224925 |
| 150 | MRT4577_305583P | MRT3847_284135P.1 |
| 150 | MRT4577_305583P | MRT3847_52222P.3 |
| 150 | MRT4577_305583P | MRT4530_21638P.2 |
| 150 | MRT4577_305583P | MRT4530_21634P.2 |
| 150 | MRT4577_305583P | MRT4530_21629P.1 |
| 150 | MRT4577_305583P | MRT4565_98294P.2 |
| 151 | MRT4577_189292P | gl_17227820 |
| 151 | MRT4577_189292P | gl_28192488 |
| 151 | MRT4577_189292P | gl_1169648 |
| 151 | MRT4577_189292P | gl_22094360 |
| 151 | MRT4577_189292P | gl_32489847 |
| 151 | MRT4577_189292P | MRT4530_25301P.1 |
| 151 | MRT4577_189292P | MRT4565_4354P.3 |
| 152 | MRT4577_409052P | gl_19881581 |
| 153 | MRT4577_371170P | gl_15233656 |
| 153 | MRT4577_371170P | gl_28973727 |
| 153 | MRT4577_371170P | gl_6633813 |
| 153 | MRT4577_371170P | gl_20259460 |
| 153 | MRT4577_371170P | gl_15217662 |
| 153 | MRT4577_371170P | MRT3847_24864P.2 |
| 153 | MRT4577_371170P | MRT3847_99459P.3 |
| 153 | MRT4577_371170P | gl_7339715 |
| 153 | MRT4577_371170P | MRT4530_100337P.1 |
| 153 | MRT4577_371170P | MRT4530_100340P.1 |
| 153 | MRT4577_371170P | MRT4530_146073P.1 |
| 153 | MRT4577_371170P | MRT4565_66175P.2 |
| 154 | MRT4577_169297P | gl_15027611 |
| 154 | MRT4577_169297P | gl_25956266 |
| 154 | MRT4577_169297P | gl_27125515 |
| 154 | MRT4577_169297P | MRT4530_37728P.2 |
| 154 | MRT4577_169297P | MRT4530_37726P.2 |
| 154 | MRT4577_169297P | gl_18657017 |
| 154 | MRT4577_169297P | MRT4530_71260P.2 |
| 154 | MRT4577_169297P | MRT4530_37730P.2 |
| 154 | MRT4577_169297P | gl_19115131 |
| 154 | MRT4577_169297P | gl_460160 |
| 154 | MRT4577_169297P | gl_6323699 |
| 154 | MRT4577_169297P | gl_264676 |
| 154 | MRT4577_169297P | gl_6324844 |
| 154 | MRT4577_169297P | gl_32404216 |
| 155 | MRT4577_273665P | gl_21741785 |
| 155 | MRT4577_273665P | MRT4565_57148P.3 |
| 156 | MRT4577_285101P | gl_21954719 |
| 156 | MRT4577_285101P | gl_21954721 |
| 156 | MRT4577_285101P | gl_27372782 |
| 156 | MRT4577_285101P | MRT3847_200246P.2 |
| 157 | MRT4577_284415P | gl_14586373 |
| 157 | MRT4577_284415P | gl_30684104 |
| 157 | MRT4577_284415P | gl_15591909 |
| 157 | MRT4577_284415P | gl_30688675 |
| 157 | MRT4577_284415P | gl_17065024 |
| 157 | MRT4577_284415P | gl_7487603 |
| 157 | MRT4577_284415P | MRT3847_26155P.3 |
| 157 | MRT4577_284415P | MRT3847_98076P.3 |
| 157 | MRT4577_284415P | MRT3847_98062P.3 |
| 157 | MRT4577_284415P | MRT3847_11589P.3 |
| 157 | MRT4577_284415P | MRT4530_46211P.2 |
| 157 | MRT4577_284415P | MRT4530_46208P.1 |
| 157 | MRT4577_284415P | MRT4565_9346P.3 |
| 158 | MRT4577_38704P | gl_30696140 |
| 158 | MRT4577_38704P | gl_30696138 |
| 158 | MRT4577_38704P | gl_1707370 |
| 158 | MRT4577_38704P | gl_15235112 |
| 158 | MRT4577_38704P | gl_25386572 |
| 158 | MRT4577_38704P | gl_1667582 |
| 158 | MRT4577_38704P | MRT3847_258276P.2 |
| 158 | MRT4577_38704P | MRT3847_61998P.3 |
| 158 | MRT4577_38704P | MRT3847_63803P.3 |
| 158 | MRT4577_38704P | MRT3847_250868P.2 |
| 158 | MRT4577_38704P | MRT4530_27655P.2 |
| 158 | MRT4577_38704P | gl_6759507 |
| 159 | MRT4577_47332P | gl_21219540 |
| 159 | MRT4577_47332P | gl_21219937 |
| 159 | MRT4577_47332P | gl_17231725 |
| 159 | MRT4577_47332P | gl_22960295 |
| 159 | MRT4577_47332P | gl_3913209 |
| 159 | MRT4577_47332P | gl_22963535 |
| 159 | MRT4577_47332P | gl_32475580 |
| 159 | MRT4577_47332P | gl_20807813 |
| 159 | MRT4577_47332P | gl_14194485 |
| 159 | MRT4577_47332P | gl_22989508 |
| 159 | MRT4577_47332P | gl_2462107 |
| 159 | MRT4577_47332P | gl_2462109 |
| 159 | MRT4577_47332P | gl_6016879 |
| 159 | MRT4577_47332P | gl_6016881 |
| 159 | MRT4577_47332P | gl_98485 |
| 159 | MRT4577_47332P | gl_15828368 |
| 159 | MRT4577_47332P | gl_15826905 |
| 159 | MRT4577_47332P | gl_15827806 |
| 159 | MRT4577_47332P | gl_21401687 |
| 159 | MRT4577_47332P | gl_18310529 |
| 159 | MRT4577_47332P | gl_30263713 |
| 159 | MRT4577_47332P | gl_23017722 |
| 159 | MRT4577_47332P | gl_23043296 |
| 159 | MRT4577_47332P | gl_23099102 |
| 159 | MRT4577_47332P | gl_16078805 |
| 159 | MRT4577_47332P | gl_27377698 |
| 159 | MRT4577_47332P | gl_29827972 |
| 159 | MRT4577_47332P | gl_29828741 |
| 159 | MRT4577_47332P | gl_29833453 |
| 159 | MRT4577_47332P | gl_3913225 |
| 159 | MRT4577_47332P | gl_11465473 |
| 159 | MRT4577_47332P | gl_11465694 |
| 159 | MRT4577_47332P | gl_116144 |
| 159 | MRT4577_47332P | gl_11467528 |
| 159 | MRT4577_47332P | gl_18404228 |
| 159 | MRT4577_47332P | gl_21553510 |
| 159 | MRT4577_47332P | gl_9294047 |
| 159 | MRT4577_47332P | gl_24559828 |
| 159 | MRT4577_47332P | gl_16263937 |
| 159 | MRT4577_47332P | MRT3847_41566P.3 |
| 159 | MRT4577_47332P | MRT3847_25290P.2 |
| 159 | MRT4577_47332P | MRT3847_16287P.3 |
| 159 | MRT4577_47332P | MRT3847_212021P.2 |
| 159 | MRT4577_47332P | MRT3847_218049P.2 |
| 159 | MRT4577_47332P | gl_8489192 |
| 159 | MRT4577_47332P | gl_30468060 |
| 159 | MRT4577_47332P | gl_2541885 |
| 159 | MRT4577_47332P | MRT4530_15443P.1 |
| 159 | MRT4577_47332P | MRT4530_104183P.1 |
| 159 | MRT4577_47332P | MRT4530_143108P.1 |
| 159 | MRT4577_47332P | MRT4530_111094P.1 |
| 159 | MRT4577_47332P | MRT4565_130085P.1 |
| 159 | MRT4577_47332P | MRT4565_8769P.3 |
| 159 | MRT4577_47332P | gl_23112455 |
| 159 | MRT4577_47332P | gl_23111662 |
| 159 | MRT4577_47332P | gl_729237 |
| 159 | MRT4577_47332P | gl_420929 |
| 159 | MRT4577_47332P | gl_729238 |
| 159 | MRT4577_47332P | gl_11467655 |
| 159 | MRT4577_47332P | gl_13812343 |
| 159 | MRT4577_47332P | gl_23131734 |
| 159 | MRT4577_47332P | gl_15839668 |
| 159 | MRT4577_47332P | gl_15843516 |
| 159 | MRT4577_47332P | gl_15607423 |
| 159 | MRT4577_47332P | gl_15611004 |
| 159 | MRT4577_47332P | gl_15611020 |
| 159 | MRT4577_47332P | gl_15608935 |
| 159 | MRT4577_47332P | gl_23134144 |
| 159 | MRT4577_47332P | gl_15614926 |
| 159 | MRT4577_47332P | gl_15614852 |
| 160 | MRT4577_386264P | gl_21223783 |
| 160 | MRT4577_386264P | gl_21220496 |
| 160 | MRT4577_386264P | gl_15616928 |
| 160 | MRT4577_386264P | gl_22968361 |
| 160 | MRT4577_386264P | gl_22970242 |
| 160 | MRT4577_386264P | gl_15921917 |
| 160 | MRT4577_386264P | gl_15618021 |
| 160 | MRT4577_386264P | gl_32476350 |
| 160 | MRT4577_386264P | gl_20808232 |
| 160 | MRT4577_386264P | gl_22980706 |
| 160 | MRT4577_386264P | gl_15676021 |
| 160 | MRT4577_386264P | gl_15793205 |
| 160 | MRT4577_386264P | gl_15807615 |
| 160 | MRT4577_386264P | gl_6708108 |
| 160 | MRT4577_386264P | gl_22988101 |
| 160 | MRT4577_386264P | gl_22990852 |
| 160 | MRT4577_386264P | gl_421428 |
| 160 | MRT4577_386264P | gl_15673314 |
| 160 | MRT4577_386264P | gl_1730064 |
| 160 | MRT4577_386264P | gl_14289139 |
| 160 | MRT4577_386264P | gl_585371 |
| 160 | MRT4577_386264P | gl_282382 |
| 160 | MRT4577_386264P | gl_3041863 |
| 160 | MRT4577_386264P | gl_30724884 |
| 160 | MRT4577_386264P | gl_1730065 |
| 160 | MRT4577_386264P | gl_15894323 |
| 160 | MRT4577_386264P | gl_15893809 |
| 160 | MRT4577_386264P | gl_15802088 |
| 160 | MRT4577_386264P | gl_23000680 |
| 160 | MRT4577_386264P | gl_1346399 |
| 160 | MRT4577_386264P | gl_15924687 |
| 160 | MRT4577_386264P | gl_23002842 |
| 160 | MRT4577_386264P | gl_15675235 |
| 160 | MRT4577_386264P | gl_15837426 |
| 160 | MRT4577_386264P | gl_125607 |
| 160 | MRT4577_386264P | gl_16800673 |
| 160 | MRT4577_386264P | gl_33240373 |
| 160 | MRT4577_386264P | gl_16803610 |
| 160 | MRT4577_386264P | gl_15900780 |
| 160 | MRT4577_386264P | gl_27468291 |
| 160 | MRT4577_386264P | gl_3122320 |
| 160 | MRT4577_386264P | gl_15827659 |
| 160 | MRT4577_386264P | gl_18312204 |
| 160 | MRT4577_386264P | gl_15891188 |
| 160 | MRT4577_386264P | gl_20092686 |
| 160 | MRT4577_386264P | gl_19705084 |
| 160 | MRT4577_386264P | gl_21232615 |
| 160 | MRT4577_386264P | gl_21244070 |
| 160 | MRT4577_386264P | gl_16126292 |
| 160 | MRT4577_386264P | gl_21402641 |
| 160 | MRT4577_386264P | gl_15791759 |
| 160 | MRT4577_386264P | gl_21226817 |
| 160 | MRT4577_386264P | gl_18311131 |
| 160 | MRT4577_386264P | gl_18309344 |
| 160 | MRT4577_386264P | gl_25028545 |
| 160 | MRT4577_386264P | gl_23308892 |
| 160 | MRT4577_386264P | gl_22299818 |
| 160 | MRT4577_386264P | gl_22298059 |
| 160 | MRT4577_386264P | gl_24113065 |
| 160 | MRT4577_386264P | gl_30063190 |
| 160 | MRT4577_386264P | gl_21910448 |
| 160 | MRT4577_386264P | gl_21672587 |
| 160 | MRT4577_386264P | gl_26247926 |
| 160 | MRT4577_386264P | gl_23017104 |
| 160 | MRT4577_386264P | gl_23023645 |
| 160 | MRT4577_386264P | gl_23029594 |
| 160 | MRT4577_386264P | gl_23037947 |
| 160 | MRT4577_386264P | gl_28493257 |
| 160 | MRT4577_386264P | gl_23502605 |
| 160 | MRT4577_386264P | gl_23336674 |
| 160 | MRT4577_386264P | gl_23466988 |
| 160 | MRT4577_386264P | gl_23059426 |
| 160 | MRT4577_386264P | gl_23063854 |
| 160 | MRT4577_386264P | gl_23465557 |
| 160 | MRT4577_386264P | gl_23475131 |
| 160 | MRT4577_386264P | gl_22537102 |
| 160 | MRT4577_386264P | gl_32039540 |
| 160 | MRT4577_386264P | gl_15596695 |
| 160 | MRT4577_386264P | gl_12045070 |
| 160 | MRT4577_386264P | gl_407635 |
| 160 | MRT4577_386264P | gl_27887626 |
| 160 | MRT4577_386264P | gl_24379618 |
| 160 | MRT4577_386264P | gl_13508042 |
| 160 | MRT4577_386264P | gl_15828711 |
| 160 | MRT4577_386264P | gl_25010985 |
| 160 | MRT4577_386264P | gl_24374035 |
| 160 | MRT4577_386264P | gl_28212071 |
| 160 | MRT4577_386264P | gl_13357744 |
| 160 | MRT4577_386264P | gl_16122616 |
| 160 | MRT4577_386264P | gl_16122303 |
| 160 | MRT4577_386264P | gl_27364101 |
| 160 | MRT4577_386264P | gl_27366266 |
| 160 | MRT4577_386264P | gl_28572631 |
| 160 | MRT4577_386264P | gl_15668279 |
| 160 | MRT4577_386264P | gl_28378548 |
| 160 | MRT4577_386264P | gl_23052059 |
| 160 | MRT4577_386264P | gl_23099626 |
| 160 | MRT4577_386264P | gl_28897130 |
| 160 | MRT4577_386264P | gl_28898813 |
| 160 | MRT4577_386264P | gl_28900678 |
| 160 | MRT4577_386264P | gl_16079970 |
| 160 | MRT4577_386264P | gl_15594693 |
| 160 | MRT4577_386264P | gl_17986575 |
| 160 | MRT4577_386264P | gl_27904791 |
| 160 | MRT4577_386264P | gl_29375625 |
| 160 | MRT4577_386264P | gl_29348250 |
| 160 | MRT4577_386264P | gl_30022674 |
| 160 | MRT4577_386264P | gl_6318287 |
| 160 | MRT4577_386264P | gl_29655069 |
| 160 | MRT4577_386264P | gl_29832759 |
| 160 | MRT4577_386264P | gl_29829367 |
| 160 | MRT4577_386264P | gl_32034452 |
| 160 | MRT4577_386264P | gl_32029324 |
| 160 | MRT4577_386264P | gl_15897860 |
| 160 | MRT4577_386264P | gl_16081945 |
| 160 | MRT4577_386264P | gl_155435 |
| 160 | MRT4577_386264P | gl_28564203 |
| 160 | MRT4577_386264P | gl_28564205 |
| 160 | MRT4577_386264P | gl_26553530 |
| 160 | MRT4577_386264P | gl_23055438 |
| 160 | MRT4577_386264P | gl_28565038 |
| 160 | MRT4577_386264P | gl_25005270 |
| 160 | MRT4577_386264P | gl_7861547 |
| 160 | MRT4577_386264P | gl_4433778 |
| 160 | MRT4577_386264P | gl_17549667 |
| 160 | MRT4577_386264P | gl_17545291 |
| 160 | MRT4577_386264P | gl_32490885 |
| 160 | MRT4577_386264P | gl_15241190 |
| 160 | MRT4577_386264P | gl_15236190 |
| 160 | MRT4577_386264P | gl_4033432 |
| 160 | MRT4577_386264P | gl_4033435 |
| 160 | MRT4577_386264P | gl_13473275 |
| 160 | MRT4577_386264P | gl_15966542 |
| 160 | MRT4577_386264P | gl_4586602 |
| 160 | MRT4577_386264P | gl_20465197 |
| 160 | MRT4577_386264P | gl_2497540 |
| 160 | MRT4577_386264P | gl_6539568 |
| 160 | MRT4577_386264P | gl_3122311 |
| 160 | MRT4577_386264P | gl_2497543 |
| 160 | MRT4577_386264P | gl_25814821 |
| 160 | MRT4577_386264P | gl_322787 |
| 160 | MRT4577_386264P | gl_125606 |
| 160 | MRT4577_386264P | MRT4530_57792P.1 |
| 160 | MRT4577_386264P | MRT4530_27060P.2 |
| 160 | MRT4577_386264P | MRT4530_27056P.1 |
| 160 | MRT4577_386264P | MRT4565_39839P.3 |
| 160 | MRT4577_386264P | MRT4565_140767P.1 |
| 160 | MRT4577_386264P | gl_7271955 |
| 160 | MRT4577_386264P | gl_23110381 |
| 160 | MRT4577_386264P | gl_19115258 |
| 160 | MRT4577_386264P | gl_11260405 |
| 160 | MRT4577_386264P | gl_28563985 |
| 160 | MRT4577_386264P | gl_28563989 |
| 160 | MRT4577_386264P | gl_28563987 |
| 160 | MRT4577_386264P | gl_6319279 |
| 160 | MRT4577_386264P | gl_6324923 |
| 160 | MRT4577_386264P | gl_4180 |
| 160 | MRT4577_386264P | gl_23121268 |
| 160 | MRT4577_386264P | gl_28564948 |
| 160 | MRT4577_386264P | gl_101735 |
| 160 | MRT4577_386264P | gl_1170699 |
| 160 | MRT4577_386264P | gl_13541851 |
| 160 | MRT4577_386264P | gl_2497537 |
| 160 | MRT4577_386264P | gl_320885 |
| 160 | MRT4577_386264P | gl_9955873 |
| 160 | MRT4577_386264P | gl_32410899 |
| 160 | MRT4577_386264P | gl_400142 |
| 160 | MRT4577_386264P | gl_5911463 |
| 160 | MRT4577_386264P | gl_12643655 |
| 160 | MRT4577_386264P | gl_3377757 |
| 160 | MRT4577_386264P | gl_147276 |
| 160 | MRT4577_386264P | gl_1310978 |
| 160 | MRT4577_386264P | gl_9955371 |
| 160 | MRT4577_386264P | gl_9955367 |
| 160 | MRT4577_386264P | gl_1805530 |
| 160 | MRT4577_386264P | gl_1742753 |
| 160 | MRT4577_386264P | gl_14600753 |
| 160 | MRT4577_386264P | gl_23122758 |
| 160 | MRT4577_386264P | gl_1526982 |
| 160 | MRT4577_386264P | gl_4033428 |
| 160 | MRT4577_386264P | gl_15640512 |
| 160 | MRT4577_386264P | gl_15642010 |
| 160 | MRT4577_386264P | gl_15601464 |
| 160 | MRT4577_386264P | gl_16273468 |
| 160 | MRT4577_386264P | gl_23131322 |
| 160 | MRT4577_386264P | gl_15602518 |
| 160 | MRT4577_386264P | gl_15605055 |
| 160 | MRT4577_386264P | gl_1791247 |
| 160 | MRT4577_386264P | gl_15608755 |
| 160 | MRT4577_386264P | gl_16129807 |
| 160 | MRT4577_386264P | gl_15831818 |
| 160 | MRT4577_386264P | gl_15835226 |
| 160 | MRT4577_386264P | gl_23133806 |
| 160 | MRT4577_386264P | gl_15615725 |
| 160 | MRT4577_386264P | gl_16760530 |
| 160 | MRT4577_386264P | gl_6691650 |
| 160 | MRT4577_386264P | gl_6729356 |
| 160 | MRT4577_386264P | gl_23135446 |
| 160 | MRT4577_386264P | gl_16764728 |
| 161 | MRT4577_25879P | gl_15236196 |
| 161 | MRT4577_25879P | MRT3847_13189P.3 |
| 161 | MRT4577_25879P | MRT3847_42675P.2 |
| 161 | MRT4577_25879P | gl_32488077 |
| 161 | MRT4577_25879P | MRT4530_10024P.1 |
| 161 | MRT4577_25879P | MRT4530_10021P.1 |
| 161 | MRT4577_25879P | MRT4565_78273P.2 |
| 164 | MRT4577_199838P | MRT3847_233523P.2 |
| 166 | MRT4577_391398P | MRT3847_36848P.3 |
| 166 | MRT4577_391398P | MRT3847_43842P.3 |
| 166 | MRT4577_391398P | MRT3847_250748P.2 |
| 166 | MRT4577_391398P | MRT3847_36849P.2 |
| 167 | MRT4577_234188P | gl_25486627 |
| 167 | MRT4577_234188P | gl_7716952 |
| 167 | MRT4577_234188P | gl_6175246 |
| 167 | MRT4577_234188P | MRT4530_91129P.1 |
| 167 | MRT4577_234188P | MRT4565_77691P.2 |
| 167 | MRT4577_234188P | MRT4565_21523P.3 |
| 167 | MRT4577_234188P | gl_4218537 |
| 168 | MRT4577_264682P | MRT3847_33136P.3 |
| 168 | MRT4577_264682P | gl_32487515 |
| 168 | MRT4577_264682P | gl_24430421 |
| 168 | MRT4577_264682P | gl_7489168 |
| 168 | MRT4577_264682P | gl_7489434 |
| 168 | MRT4577_264682P | gl_7489412 |
| 168 | MRT4577_264682P | MRT4530_101175P.1 |
| 168 | MRT4577_264682P | MRT4565_26905P.2 |
| 169 | MRT4577_287055P | gl_21221074 |
| 169 | MRT4577_287055P | gl_17231176 |
| 169 | MRT4577_287055P | gl_22959136 |
| 169 | MRT4577_287055P | gl_22956679 |
| 169 | MRT4577_287055P | gl_22964886 |
| 169 | MRT4577_287055P | gl_22962301 |
| 169 | MRT4577_287055P | gl_15617074 |
| 169 | MRT4577_287055P | gl_22969349 |
| 169 | MRT4577_287055P | gl_22967579 |
| 169 | MRT4577_287055P | gl_22970179 |
| 169 | MRT4577_287055P | gl_6225171 |
| 169 | MRT4577_287055P | gl_16332067 |
| 169 | MRT4577_287055P | gl_15618755 |
| 169 | MRT4577_287055P | gl_32476155 |
| 169 | MRT4577_287055P | gl_20807120 |
| 169 | MRT4577_287055P | gl_20807894 |
| 169 | MRT4577_287055P | gl_22976982 |
| 169 | MRT4577_287055P | gl_15677237 |
| 169 | MRT4577_287055P | gl_15794478 |
| 169 | MRT4577_287055P | gl_6273581 |
| 169 | MRT4577_287055P | gl_15806971 |
| 169 | MRT4577_287055P | gl_22983077 |
| 169 | MRT4577_287055P | gl_22991721 |
| 169 | MRT4577_287055P | gl_7546983 |
| 169 | MRT4577_287055P | gl_15673133 |
| 169 | MRT4577_287055P | gl_3023975 |
| 169 | MRT4577_287055P | gl_1196314 |
| 169 | MRT4577_287055P | gl_1296452 |
| 169 | MRT4577_287055P | gl_1142616 |
| 169 | MRT4577_287055P | gl_15895897 |
| 169 | MRT4577_287055P | gl_21328719 |
| 169 | MRT4577_287055P | gl_15800168 |
| 169 | MRT4577_287055P | gl_22994398 |
| 169 | MRT4577_287055P | gl_22994632 |
| 169 | MRT4577_287055P | gl_22996222 |
| 169 | MRT4577_287055P | gl_22997796 |
| 169 | MRT4577_287055P | gl_22998791 |
| 169 | MRT4577_287055P | gl_23000020 |
| 169 | MRT4577_287055P | gl_2105144 |
| 169 | MRT4577_287055P | gl_15924664 |
| 169 | MRT4577_287055P | gl_15924244 |
| 169 | MRT4577_287055P | gl_23003622 |
| 169 | MRT4577_287055P | gl_23002438 |
| 169 | MRT4577_287055P | gl_15639499 |
| 169 | MRT4577_287055P | gl_26989025 |
| 169 | MRT4577_287055P | gl_15674910 |
| 169 | MRT4577_287055P | gl_15837790 |
| 169 | MRT4577_287055P | gl_15838086 |
| 169 | MRT4577_287055P | gl_16800375 |
| 169 | MRT4577_287055P | gl_16800386 |
| 169 | MRT4577_287055P | gl_33241266 |
| 169 | MRT4577_287055P | gl_16803308 |
| 169 | MRT4577_287055P | gl_16803319 |
| 169 | MRT4577_287055P | gl_15901412 |
| 169 | MRT4577_287055P | gl_27468267 |
| 169 | MRT4577_287055P | gl_27467848 |
| 169 | MRT4577_287055P | gl_15827775 |
| 169 | MRT4577_287055P | gl_15888589 |
| 169 | MRT4577_287055P | gl_15887403 |
| 169 | MRT4577_287055P | gl_28198387 |
| 169 | MRT4577_287055P | gl_23014985 |
| 169 | MRT4577_287055P | gl_23005242 |
| 169 | MRT4577_287055P | gl_23014725 |
| 169 | MRT4577_287055P | gl_24215258 |
| 169 | MRT4577_287055P | gl_19705311 |
| 169 | MRT4577_287055P | gl_21230440 |
| 169 | MRT4577_287055P | gl_21241839 |
| 169 | MRT4577_287055P | gl_16126204 |
| 169 | MRT4577_287055P | gl_21402518 |
| 169 | MRT4577_287055P | gl_21401812 |
| 169 | MRT4577_287055P | gl_5002358 |
| 169 | MRT4577_287055P | gl_6225163 |
| 169 | MRT4577_287055P | gl_15791646 |
| 169 | MRT4577_287055P | gl_15792017 |
| 169 | MRT4577_287055P | gl_21673243 |
| 169 | MRT4577_287055P | gl_21674017 |
| 169 | MRT4577_287055P | gl_18310374 |
| 169 | MRT4577_287055P | gl_25028847 |
| 169 | MRT4577_287055P | gl_21283347 |
| 169 | MRT4577_287055P | gl_21282866 |
| 169 | MRT4577_287055P | gl_19553586 |
| 169 | MRT4577_287055P | gl_22298053 |
| 169 | MRT4577_287055P | gl_30263833 |
| 169 | MRT4577_287055P | gl_21672725 |
| 169 | MRT4577_287055P | gl_23019058 |
| 169 | MRT4577_287055P | gl_23021744 |
| 169 | MRT4577_287055P | gl_23023390 |
| 169 | MRT4577_287055P | gl_23037705 |
| 169 | MRT4577_287055P | gl_23041315 |
| 169 | MRT4577_287055P | gl_28493446 |
| 169 | MRT4577_287055P | gl_23501986 |
| 169 | MRT4577_287055P | gl_23502927 |
| 169 | MRT4577_287055P | gl_23336808 |
| 169 | MRT4577_287055P | gl_23336272 |
| 169 | MRT4577_287055P | gl_23467432 |
| 169 | MRT4577_287055P | gl_23469166 |
| 169 | MRT4577_287055P | gl_23058851 |
| 169 | MRT4577_287055P | gl_23465516 |
| 169 | MRT4577_287055P | gl_23474551 |
| 169 | MRT4577_287055P | gl_23475994 |
| 169 | MRT4577_287055P | gl_22537459 |
| 169 | MRT4577_287055P | gl_15596999 |
| 169 | MRT4577_287055P | gl_27887595 |
| 169 | MRT4577_287055P | gl_24379392 |
| 169 | MRT4577_287055P | gl_25011425 |
| 169 | MRT4577_287055P | gl_24373361 |
| 169 | MRT4577_287055P | gl_28211966 |
| 169 | MRT4577_287055P | gl_16123318 |
| 169 | MRT4577_287055P | gl_27363511 |
| 169 | MRT4577_287055P | gl_28572441 |
| 169 | MRT4577_287055P | gl_28378738 |
| 169 | MRT4577_287055P | gl_28378504 |
| 169 | MRT4577_287055P | gl_23099532 |
| 169 | MRT4577_287055P | gl_23099005 |
| 169 | MRT4577_287055P | gl_28870880 |
| 169 | MRT4577_287055P | gl_28897692 |
| 169 | MRT4577_287055P | gl_16079874 |
| 169 | MRT4577_287055P | gl_16078679 |
| 169 | MRT4577_287055P | gl_15606540 |
| 169 | MRT4577_287055P | gl_15605757 |
| 169 | MRT4577_287055P | gl_15594957 |
| 169 | MRT4577_287055P | gl_15594640 |
| 169 | MRT4577_287055P | gl_27380054 |
| 169 | MRT4577_287055P | gl_27375757 |
| 169 | MRT4577_287055P | gl_17987158 |
| 169 | MRT4577_287055P | gl_17988331 |
| 169 | MRT4577_287055P | gl_27904899 |
| 169 | MRT4577_287055P | gl_29376445 |
| 169 | MRT4577_287055P | gl_29376200 |
| 169 | MRT4577_287055P | gl_29349251 |
| 169 | MRT4577_287055P | gl_30022560 |
| 169 | MRT4577_287055P | gl_30021917 |
| 169 | MRT4577_287055P | gl_29654073 |
| 169 | MRT4577_287055P | gl_29831992 |
| 169 | MRT4577_287055P | gl_29840676 |
| 169 | MRT4577_287055P | gl_32035049 |
| 169 | MRT4577_287055P | gl_30248063 |
| 169 | MRT4577_287055P | gl_15642920 |
| 169 | MRT4577_287055P | gl_15643288 |
| 169 | MRT4577_287055P | gl_6942107 |
| 169 | MRT4577_287055P | gl_11133033 |
| 169 | MRT4577_287055P | gl_27262354 |
| 169 | MRT4577_287055P | gl_23056436 |
| 169 | MRT4577_287055P | gl_17546431 |
| 169 | MRT4577_287055P | gl_22532109 |
| 169 | MRT4577_287055P | gl_8134368 |
| 169 | MRT4577_287055P | gl_27804891 |
| 169 | MRT4577_287055P | gl_23103564 |
| 169 | MRT4577_287055P | gl_23104278 |
| 169 | MRT4577_287055P | gl_142369 |
| 169 | MRT4577_287055P | gl_28262700 |
| 169 | MRT4577_287055P | gl_28262023 |
| 169 | MRT4577_287055P | gl_32490903 |
| 169 | MRT4577_287055P | gl_13476995 |
| 169 | MRT4577_287055P | gl_13474176 |
| 169 | MRT4577_287055P | gl_22653795 |
| 169 | MRT4577_287055P | gl_15965009 |
| 169 | MRT4577_287055P | MRT4565_98303P.2 |
| 169 | MRT4577_287055P | MRT4565_43124P.2 |
| 169 | MRT4577_287055P | gl_23108079 |
| 169 | MRT4577_287055P | gl_6319704 |
| 169 | MRT4577_287055P | gl_23119424 |
| 169 | MRT4577_287055P | gl_23111737 |
| 169 | MRT4577_287055P | gl_23111624 |
| 169 | MRT4577_287055P | gl_32409603 |
| 169 | MRT4577_287055P | gl_388977 |
| 169 | MRT4577_287055P | gl_23123457 |
| 169 | MRT4577_287055P | gl_7594817 |
| 169 | MRT4577_287055P | gl_6225174 |
| 169 | MRT4577_287055P | gl_23126009 |
| 169 | MRT4577_287055P | gl_15641923 |
| 169 | MRT4577_287055P | gl_1655938 |
| 169 | MRT4577_287055P | gl_16272655 |
| 169 | MRT4577_287055P | gl_23130789 |
| 169 | MRT4577_287055P | gl_15603842 |
| 169 | MRT4577_287055P | gl_15892991 |
| 169 | MRT4577_287055P | gl_15892357 |
| 169 | MRT4577_287055P | gl_15604535 |
| 169 | MRT4577_287055P | gl_15604188 |
| 169 | MRT4577_287055P | gl_15605438 |
| 169 | MRT4577_287055P | gl_15841981 |
| 169 | MRT4577_287055P | gl_15609594 |
| 169 | MRT4577_287055P | gl_15834703 |
| 169 | MRT4577_287055P | gl_23132758 |
| 169 | MRT4577_287055P | gl_15645984 |
| 169 | MRT4577_287055P | gl_15645143 |
| 169 | MRT4577_287055P | gl_15612353 |
| 169 | MRT4577_287055P | gl_15611532 |
| 169 | MRT4577_287055P | gl_15615614 |
| 169 | MRT4577_287055P | gl_15615026 |
| 169 | MRT4577_287055P | gl_16759429 |
| 169 | MRT4577_287055P | gl_23136411 |
| 169 | MRT4577_287055P | gl_23137026 |
| 169 | MRT4577_287055P | gl_16763830 |
| 170 | MRT4577_49099P | gl_5758877 |
| 170 | MRT4577_49099P | gl_5758896 |
| 170 | MRT4577_49099P | gl_5758897 |
| 170 | MRT4577_49099P | gl_5758903 |
| 170 | MRT4577_49099P | gl_5758867 |
| 170 | MRT4577_49099P | gl_5758886 |
| 170 | MRT4577_49099P | gl_5758911 |
| 170 | MRT4577_49099P | gl_6467949 |
| 170 | MRT4577_49099P | gl_6467934 |
| 170 | MRT4577_49099P | gl_14718030 |
| 170 | MRT4577_49099P | gl_12004119 |
| 170 | MRT4577_49099P | gl_12004145 |
| 170 | MRT4577_49099P | gl_12004143 |
| 170 | MRT4577_49099P | gl_12004127 |
| 170 | MRT4577_49099P | gl_12004121 |
| 170 | MRT4577_49099P | gl_12004115 |
| 170 | MRT4577_49099P | gl_12004137 |
| 170 | MRT4577_49099P | gl_12004139 |
| 170 | MRT4577_49099P | gl_12004149 |
| 170 | MRT4577_49099P | gl_12004151 |
| 170 | MRT4577_49099P | gl_12004153 |
| 170 | MRT4577_49099P | gl_12004159 |
| 170 | MRT4577_49099P | gl_12004161 |
| 170 | MRT4577_49099P | gl_12004133 |
| 170 | MRT4577_49099P | gl_12004113 |
| 170 | MRT4577_49099P | gl_15921725 |
| 170 | MRT4577_49099P | gl_15425576 |
| 170 | MRT4577_49099P | gl_21633411 |
| 170 | MRT4577_49099P | gl_11527563 |
| 170 | MRT4577_49099P | gl_14717924 |
| 170 | MRT4577_49099P | gl_14718224 |
| 170 | MRT4577_49099P | gl_14717935 |
| 170 | MRT4577_49099P | gl_14717920 |
| 170 | MRT4577_49099P | gl_14718095 |
| 170 | MRT4577_49099P | gl_14718090 |
| 170 | MRT4577_49099P | gl_14718060 |
| 170 | MRT4577_49099P | gl_14718167 |
| 170 | MRT4577_49099P | gl_14718242 |
| 170 | MRT4577_49099P | gl_14718228 |
| 170 | MRT4577_49099P | gl_24940166 |
| 170 | MRT4577_49099P | gl_24940194 |
| 170 | MRT4577_49099P | gl_17224755 |
| 170 | MRT4577_49099P | gl_27528494 |
| 170 | MRT4577_49099P | gl_27528472 |
| 170 | MRT4577_49099P | gl_16330679 |
| 170 | MRT4577_49099P | gl_15618012 |
| 170 | MRT4577_49099P | gl_22094585 |
| 170 | MRT4577_49099P | gl_14718165 |
| 170 | MRT4577_49099P | gl_21684927 |
| 170 | MRT4577_49099P | gl_18077603 |
| 170 | MRT4577_49099P | gl_18077601 |
| 170 | MRT4577_49099P | gl_20514385 |
| 170 | MRT4577_49099P | gl_15805727 |
| 170 | MRT4577_49099P | gl_584810 |
| 170 | MRT4577_49099P | gl_14718042 |
| 170 | MRT4577_49099P | gl_19033077 |
| 170 | MRT4577_49099P | gl_14718232 |
| 170 | MRT4577_49099P | gl_12005284 |
| 170 | MRT4577_49099P | gl_7687960 |
| 170 | MRT4577_49099P | gl_5001573 |
| 170 | MRT4577_49099P | gl_7708171 |
| 170 | MRT4577_49099P | gl_24940162 |
| 170 | MRT4577_49099P | gl_14717984 |
| 170 | MRT4577_49099P | gl_6687199 |
| 170 | MRT4577_49099P | gl_14717990 |
| 170 | MRT4577_49099P | gl_5001583 |
| 170 | MRT4577_49099P | gl_8517408 |
| 170 | MRT4577_49099P | gl_7708197 |
| 170 | MRT4577_49099P | gl_4063542 |
| 170 | MRT4577_49099P | gl_7687974 |
| 170 | MRT4577_49099P | gl_7708284 |
| 170 | MRT4577_49099P | gl_22992679 |
| 170 | MRT4577_49099P | gl_7687976 |
| 170 | MRT4577_49099P | gl_6687483 |
| 170 | MRT4577_49099P | gl_14718056 |
| 170 | MRT4577_49099P | gl_21684893 |
| 170 | MRT4577_49099P | gl_1171780 |
| 170 | MRT4577_49099P | gl_97924 |
| 170 | MRT4577_49099P | gl_1072369 |
| 170 | MRT4577_49099P | gl_6706178 |
| 170 | MRT4577_49099P | gl_7708570 |
| 170 | MRT4577_49099P | gl_7688039 |
| 170 | MRT4577_49099P | gl_7688411 |
| 170 | MRT4577_49099P | gl_19033051 |
| 170 | MRT4577_49099P | gl_16416760 |
| 170 | MRT4577_49099P | gl_30352098 |
| 170 | MRT4577_49099P | gl_12585416 |
| 170 | MRT4577_49099P | gl_21956014 |
| 170 | MRT4577_49099P | gl_16416758 |
| 170 | MRT4577_49099P | gl_27435896 |
| 170 | MRT4577_49099P | gl_19033069 |
| 170 | MRT4577_49099P | gl_32526541 |
| 170 | MRT4577_49099P | gl_32526543 |
| 170 | MRT4577_49099P | gl_15639519 |
| 170 | MRT4577_49099P | gl_15639417 |
| 170 | MRT4577_49099P | gl_15674362 |
| 170 | MRT4577_49099P | gl_2493121 |
| 170 | MRT4577_49099P | gl_1929027 |
| 170 | MRT4577_49099P | gl_24528335 |
| 170 | MRT4577_49099P | gl_16416730 |
| 170 | MRT4577_49099P | gl_16416738 |
| 170 | MRT4577_49099P | gl_7708329 |
| 170 | MRT4577_49099P | gl_15901171 |
| 170 | MRT4577_49099P | gl_15425574 |
| 170 | MRT4577_49099P | gl_20384961 |
| 170 | MRT4577_49099P | gl_28188331 |
| 170 | MRT4577_49099P | gl_20467373 |
| 170 | MRT4577_49099P | gl_20467387 |
| 170 | MRT4577_49099P | gl_20467383 |
| 170 | MRT4577_49099P | gl_16417186 |
| 170 | MRT4577_49099P | gl_18312083 |
| 170 | MRT4577_49099P | gl_21684909 |
| 170 | MRT4577_49099P | gl_21684891 |
| 170 | MRT4577_49099P | gl_21684869 |
| 170 | MRT4577_49099P | gl_18075919 |
| 170 | MRT4577_49099P | gl_18077607 |
| 170 | MRT4577_49099P | gl_18075921 |
| 170 | MRT4577_49099P | gl_24940196 |
| 170 | MRT4577_49099P | gl_24940262 |
| 170 | MRT4577_49099P | gl_19033091 |
| 170 | MRT4577_49099P | gl_21667292 |
| 170 | MRT4577_49099P | gl_19745323 |
| 170 | MRT4577_49099P | gl_18976554 |
| 170 | MRT4577_49099P | gl_19033067 |
| 170 | MRT4577_49099P | gl_15678973 |
| 170 | MRT4577_49099P | gl_20092951 |
| 170 | MRT4577_49099P | gl_20094453 |
| 170 | MRT4577_49099P | gl_28188341 |
| 170 | MRT4577_49099P | gl_28188339 |
| 170 | MRT4577_49099P | gl_19705056 |
| 170 | MRT4577_49099P | gl_19705057 |
| 170 | MRT4577_49099P | gl_28188329 |
| 170 | MRT4577_49099P | gl_16127677 |
| 170 | MRT4577_49099P | gl_20269434 |
| 170 | MRT4577_49099P | gl_20269410 |
| 170 | MRT4577_49099P | gl_20269418 |
| 170 | MRT4577_49099P | gl_21226882 |
| 170 | MRT4577_49099P | gl_23503627 |
| 170 | MRT4577_49099P | gl_30316239 |
| 170 | MRT4577_49099P | gl_28895034 |
| 170 | MRT4577_49099P | gl_18310620 |
| 170 | MRT4577_49099P | gl_30265987 |
| 170 | MRT4577_49099P | gl_21633339 |
| 170 | MRT4577_49099P | gl_21633397 |
| 170 | MRT4577_49099P | gl_21633375 |
| 170 | MRT4577_49099P | gl_21633369 |
| 170 | MRT4577_49099P | gl_21633323 |
| 170 | MRT4577_49099P | gl_21633359 |
| 170 | MRT4577_49099P | gl_21633435 |
| 170 | MRT4577_49099P | gl_21633371 |
| 170 | MRT4577_49099P | gl_21633423 |
| 170 | MRT4577_49099P | gl_21633419 |
| 170 | MRT4577_49099P | gl_21633441 |
| 170 | MRT4577_49099P | gl_21633405 |
| 170 | MRT4577_49099P | gl_21633433 |
| 170 | MRT4577_49099P | gl_21633431 |
| 170 | MRT4577_49099P | gl_21633365 |
| 170 | MRT4577_49099P | gl_21633355 |
| 170 | MRT4577_49099P | gl_21633349 |
| 170 | MRT4577_49099P | gl_21633343 |
| 170 | MRT4577_49099P | gl_21633399 |
| 170 | MRT4577_49099P | gl_21633415 |
| 170 | MRT4577_49099P | gl_21633417 |
| 170 | MRT4577_49099P | gl_21633425 |
| 170 | MRT4577_49099P | gl_21633427 |
| 170 | MRT4577_49099P | gl_21633301 |
| 170 | MRT4577_49099P | gl_21633437 |
| 170 | MRT4577_49099P | gl_21633361 |
| 170 | MRT4577_49099P | gl_21633379 |
| 170 | MRT4577_49099P | gl_21633383 |
| 170 | MRT4577_49099P | gl_21633381 |
| 170 | MRT4577_49099P | gl_21909656 |
| 170 | MRT4577_49099P | gl_23021511 |
| 170 | MRT4577_49099P | gl_24940164 |
| 170 | MRT4577_49099P | gl_24940168 |
| 170 | MRT4577_49099P | gl_24940174 |
| 170 | MRT4577_49099P | gl_24940176 |
| 170 | MRT4577_49099P | gl_24940246 |
| 170 | MRT4577_49099P | gl_24940248 |
| 170 | MRT4577_49099P | gl_24940256 |
| 170 | MRT4577_49099P | gl_24940266 |
| 170 | MRT4577_49099P | gl_24940204 |
| 170 | MRT4577_49099P | gl_24940264 |
| 170 | MRT4577_49099P | gl_30351915 |
| 170 | MRT4577_49099P | gl_30351917 |
| 170 | MRT4577_49099P | gl_15596894 |
| 170 | MRT4577_49099P | gl_27886806 |
| 170 | MRT4577_49099P | gl_15828737 |
| 170 | MRT4577_49099P | gl_15828707 |
| 170 | MRT4577_49099P | gl_28210705 |
| 170 | MRT4577_49099P | gl_28211923 |
| 170 | MRT4577_49099P | gl_80953 |
| 170 | MRT4577_49099P | gl_15668390 |
| 170 | MRT4577_49099P | gl_12585563 |
| 170 | MRT4577_49099P | gl_114520 |
| 170 | MRT4577_49099P | gl_23051710 |
| 170 | MRT4577_49099P | gl_11267101 |
| 170 | MRT4577_49099P | gl_7708468 |
| 170 | MRT4577_49099P | gl_114516 |
| 170 | MRT4577_49099P | gl_11498766 |
| 170 | MRT4577_49099P | gl_15594440 |
| 170 | MRT4577_49099P | gl_2493099 |
| 170 | MRT4577_49099P | gl_27904521 |
| 170 | MRT4577_49099P | gl_29376065 |
| 170 | MRT4577_49099P | gl_29346709 |
| 170 | MRT4577_49099P | gl_7436320 |
| 170 | MRT4577_49099P | gl_29840442 |
| 170 | MRT4577_49099P | gl_32034348 |
| 170 | MRT4577_49099P | gl_114528 |
| 170 | MRT4577_49099P | gl_15897484 |
| 170 | MRT4577_49099P | gl_14718003 |
| 170 | MRT4577_49099P | gl_16081190 |
| 170 | MRT4577_49099P | gl_9229839 |
| 170 | MRT4577_49099P | gl_18075929 |
| 170 | MRT4577_49099P | gl_6688706 |
| 170 | MRT4577_49099P | gl_21633463 |
| 170 | MRT4577_49099P | gl_6689006 |
| 170 | MRT4577_49099P | gl_7708189 |
| 170 | MRT4577_49099P | gl_6689008 |
| 170 | MRT4577_49099P | gl_14718107 |
| 170 | MRT4577_49099P | gl_24298775 |
| 170 | MRT4577_49099P | gl_4063526 |
| 170 | MRT4577_49099P | gl_1072952 |
| 170 | MRT4577_49099P | gl_29420857 |
| 170 | MRT4577_49099P | gl_27528482 |
| 170 | MRT4577_49099P | gl_29420859 |
| 170 | MRT4577_49099P | gl_27528474 |
| 170 | MRT4577_49099P | gl_29420871 |
| 170 | MRT4577_49099P | gl_29420867 |
| 170 | MRT4577_49099P | gl_29420869 |
| 170 | MRT4577_49099P | gl_27528492 |
| 170 | MRT4577_49099P | gl_27528498 |
| 170 | MRT4577_49099P | gl_29420865 |
| 170 | MRT4577_49099P | gl_27528480 |
| 170 | MRT4577_49099P | gl_32172455 |
| 170 | MRT4577_49099P | gl_1352828 |
| 170 | MRT4577_49099P | gl_23054147 |
| 170 | MRT4577_49099P | gl_11467561 |
| 170 | MRT4577_49099P | gl_15425588 |
| 170 | MRT4577_49099P | gl_7706848 |
| 170 | MRT4577_49099P | gl_6688492 |
| 170 | MRT4577_49099P | gl_6687737 |
| 170 | MRT4577_49099P | gl_4731151 |
| 170 | MRT4577_49099P | gl_14718009 |
| 170 | MRT4577_49099P | gl_14718099 |
| 170 | MRT4577_49099P | gl_14718230 |
| 170 | MRT4577_49099P | gl_7708662 |
| 170 | MRT4577_49099P | gl_2459981 |
| 170 | MRT4577_49099P | gl_7688337 |
| 170 | MRT4577_49099P | gl_4063558 |
| 170 | MRT4577_49099P | gl_7706839 |
| 170 | MRT4577_49099P | gl_29420855 |
| 170 | MRT4577_49099P | gl_14521960 |
| 170 | MRT4577_49099P | gl_27550061 |
| 170 | MRT4577_49099P | gl_21264381 |
| 170 | MRT4577_49099P | gl_7708538 |
| 170 | MRT4577_49099P | gl_7688031 |
| 170 | MRT4577_49099P | gl_17548614 |
| 170 | MRT4577_49099P | gl_19033063 |
| 170 | MRT4577_49099P | gl_28188335 |
| 170 | MRT4577_49099P | gl_28188325 |
| 170 | MRT4577_49099P | gl_19033097 |
| 170 | MRT4577_49099P | gl_19033089 |
| 170 | MRT4577_49099P | gl_5869971 |
| 170 | MRT4577_49099P | gl_11466709 |
| 170 | MRT4577_49099P | gl_24460025 |
| 170 | MRT4577_49099P | gl_14718147 |
| 170 | MRT4577_49099P | gl_4995759 |
| 170 | MRT4577_49099P | gl_4063560 |
| 170 | MRT4577_49099P | gl_3023341 |
| 170 | MRT4577_49099P | gl_12585499 |
| 170 | MRT4577_49099P | gl_27435914 |
| 170 | MRT4577_49099P | gl_11034791 |
| 170 | MRT4577_49099P | gl_14718151 |
| 170 | MRT4577_49099P | gl_5758854 |
| 170 | MRT4577_49099P | gl_18075917 |
| 170 | MRT4577_49099P | gl_24940180 |
| 170 | MRT4577_49099P | gl_24940198 |
| 170 | MRT4577_49099P | gl_6687481 |
| 170 | MRT4577_49099P | gl_29420863 |
| 170 | MRT4577_49099P | gl_29420861 |
| 170 | MRT4577_49099P | gl_27528478 |
| 170 | MRT4577_49099P | gl_7708335 |
| 170 | MRT4577_49099P | gl_14718076 |
| 170 | MRT4577_49099P | gl_7708181 |
| 170 | MRT4577_49099P | gl_7578495 |
| 170 | MRT4577_49099P | gl_1430917 |
| 170 | MRT4577_49099P | gl_6017822 |
| 170 | MRT4577_49099P | gl_7708173 |
| 170 | MRT4577_49099P | gl_13235340 |
| 170 | MRT4577_49099P | gl_1336803 |
| 170 | MRT4577_49099P | gl_11497535 |
| 170 | MRT4577_49099P | gl_67842 |
| 170 | MRT4577_49099P | gl_27529083 |
| 170 | MRT4577_49099P | gl_21684925 |
| 170 | MRT4577_49099P | gl_2493120 |
| 170 | MRT4577_49099P | gl_3334408 |
| 170 | MRT4577_49099P | gl_5758914 |
| 170 | MRT4577_49099P | gl_14718136 |
| 170 | MRT4577_49099P | gl_7708574 |
| 170 | MRT4577_49099P | gl_401322 |
| 170 | MRT4577_49099P | gl_3169287 |
| 170 | MRT4577_49099P | gl_4995846 |
| 170 | MRT4577_49099P | gl_4063538 |
| 170 | MRT4577_49099P | gl_32490757 |
| 170 | MRT4577_49099P | gl_14718222 |
| 170 | MRT4577_49099P | gl_14718189 |
| 170 | MRT4577_49099P | gl_15219234 |
| 170 | MRT4577_49099P | gl_2493122 |
| 170 | MRT4577_49099P | gl_14718140 |
| 170 | MRT4577_49099P | gl_19033053 |
| 170 | MRT4577_49099P | gl_16416736 |
| 170 | MRT4577_49099P | gl_4063562 |
| 170 | MRT4577_49099P | gl_15982954 |
| 170 | MRT4577_49099P | gl_6634078 |
| 170 | MRT4577_49099P | gl_6634488 |
| 170 | MRT4577_49099P | gl_1934688 |
| 170 | MRT4577_49099P | gl_14718046 |
| 170 | MRT4577_49099P | MRT3847_257212P.1 |
| 170 | MRT4577_49099P | MRT3847_70323P.2 |
| 170 | MRT4577_49099P | MRT3847_257209P.2 |
| 170 | MRT4577_49099P | gl_6687375 |
| 170 | MRT4577_49099P | gl_8452718 |
| 170 | MRT4577_49099P | gl_14585885 |
| 170 | MRT4577_49099P | gl_2506211 |
| 170 | MRT4577_49099P | gl_7708139 |
| 170 | MRT4577_49099P | gl_7708327 |
| 170 | MRT4577_49099P | gl_5001589 |
| 170 | MRT4577_49099P | gl_6688704 |
| 170 | MRT4577_49099P | gl_6689056 |
| 170 | MRT4577_49099P | gl_29726150 |
| 170 | MRT4577_49099P | gl_14718007 |
| 170 | MRT4577_49099P | gl_6689562 |
| 170 | MRT4577_49099P | gl_7708177 |
| 170 | MRT4577_49099P | gl_16943664 |
| 170 | MRT4577_49099P | gl_16943662 |
| 170 | MRT4577_49099P | gl_5758894 |
| 170 | MRT4577_49099P | gl_7708568 |
| 170 | MRT4577_49099P | gl_11466794 |
| 170 | MRT4577_49099P | gl_19920171 |
| 170 | MRT4577_49099P | gl_14718072 |
| 170 | MRT4577_49099P | gl_7708333 |
| 170 | MRT4577_49099P | gl_4063522 |
| 170 | MRT4577_49099P | gl_1041768 |
| 170 | MRT4577_49099P | gl_137460 |
| 170 | MRT4577_49099P | gl_553048 |
| 170 | MRT4577_49099P | gl_5001569 |
| 170 | MRT4577_49099P | gl_6706180 |
| 170 | MRT4577_49099P | gl_27884018 |
| 170 | MRT4577_49099P | gl_27883932 |
| 170 | MRT4577_49099P | gl_19033057 |
| 170 | MRT4577_49099P | gl_19033055 |
| 170 | MRT4577_49099P | gl_231596 |
| 170 | MRT4577_49099P | gl_6688901 |
| 170 | MRT4577_49099P | gl_7708153 |
| 170 | MRT4577_49099P | gl_6689307 |
| 170 | MRT4577_49099P | gl_6686963 |
| 170 | MRT4577_49099P | gl_7708634 |
| 170 | MRT4577_49099P | gl_8517661 |
| 170 | MRT4577_49099P | gl_7708308 |
| 170 | MRT4577_49099P | gl_7708444 |
| 170 | MRT4577_49099P | gl_7708215 |
| 170 | MRT4577_49099P | gl_6687550 |
| 170 | MRT4577_49099P | gl_7708464 |
| 170 | MRT4577_49099P | gl_7708630 |
| 170 | MRT4577_49099P | gl_6017838 |
| 170 | MRT4577_49099P | gl_12004131 |
| 170 | MRT4577_49099P | gl_6687278 |
| 170 | MRT4577_49099P | gl_6687548 |
| 170 | MRT4577_49099P | gl_6687660 |
| 170 | MRT4577_49099P | gl_6689113 |
| 170 | MRT4577_49099P | gl_14718013 |
| 170 | MRT4577_49099P | gl_4206564 |
| 170 | MRT4577_49099P | gl_8452749 |
| 170 | MRT4577_49099P | gl_4063566 |
| 170 | MRT4577_49099P | gl_22651734 |
| 170 | MRT4577_49099P | gl_7592738 |
| 170 | MRT4577_49099P | gl_4063564 |
| 170 | MRT4577_49099P | gl_4063524 |
| 170 | MRT4577_49099P | gl_8452756 |
| 170 | MRT4577_49099P | gl_11908164 |
| 170 | MRT4577_49099P | gl_4206610 |
| 170 | MRT4577_49099P | gl_19033085 |
| 170 | MRT4577_49099P | gl_5031147 |
| 170 | MRT4577_49099P | gl_14718153 |
| 170 | MRT4577_49099P | gl_7688029 |
| 170 | MRT4577_49099P | gl_7687964 |
| 170 | MRT4577_49099P | gl_11034787 |
| 170 | MRT4577_49099P | gl_6017814 |
| 170 | MRT4577_49099P | gl_7706835 |
| 170 | MRT4577_49099P | gl_17224761 |
| 170 | MRT4577_49099P | gl_5758895 |
| 170 | MRT4577_49099P | gl_2493123 |
| 170 | MRT4577_49099P | gl_29539348 |
| 170 | MRT4577_49099P | gl_4995717 |
| 170 | MRT4577_49099P | gl_4063552 |
| 170 | MRT4577_49099P | gl_6017792 |
| 170 | MRT4577_49099P | gl_7708321 |
| 170 | MRT4577_49099P | MRT4530_8337P.2 |
| 170 | MRT4577_49099P | MRT4530_87661P.1 |
| 170 | MRT4577_49099P | MRT4530_72752P.2 |
| 170 | MRT4577_49099P | MRT4530_87659P.1 |
| 170 | MRT4577_49099P | MRT4530_87660P.1 |
| 170 | MRT4577_49099P | MRT4530_60814P.1 |
| 170 | MRT4577_49099P | MRT4530_27618P.1 |
| 170 | MRT4577_49099P | gl_227786 |
| 170 | MRT4577_49099P | MRT4565_101762P.1 |
| 170 | MRT4577_49099P | MRT4565_24817P.3 |
| 170 | MRT4577_49099P | MRT4565_59504P.2 |
| 170 | MRT4577_49099P | gl_6689231 |
| 170 | MRT4577_49099P | gl_6017824 |
| 170 | MRT4577_49099P | gl_14717997 |
| 170 | MRT4577_49099P | gl_5758866 |
| 170 | MRT4577_49099P | gl_5758898 |
| 170 | MRT4577_49099P | gl_5758878 |
| 170 | MRT4577_49099P | gl_5758899 |
| 170 | MRT4577_49099P | gl_6688636 |
| 170 | MRT4577_49099P | gl_6689309 |
| 170 | MRT4577_49099P | gl_5758884 |
| 170 | MRT4577_49099P | gl_5758908 |
| 170 | MRT4577_49099P | gl_5758921 |
| 170 | MRT4577_49099P | gl_5758888 |
| 170 | MRT4577_49099P | gl_6692624 |
| 170 | MRT4577_49099P | gl_6601482 |
| 170 | MRT4577_49099P | gl_7708157 |
| 170 | MRT4577_49099P | gl_14718111 |
| 170 | MRT4577_49099P | gl_27528476 |
| 170 | MRT4577_49099P | gl_28202179 |
| 170 | MRT4577_49099P | gl_14717950 |
| 170 | MRT4577_49099P | gl_24940182 |
| 170 | MRT4577_49099P | gl_14717931 |
| 170 | MRT4577_49099P | gl_6687201 |
| 170 | MRT4577_49099P | gl_6689111 |
| 170 | MRT4577_49099P | gl_19114337 |
| 170 | MRT4577_49099P | gl_6689408 |
| 170 | MRT4577_49099P | gl_27526581 |
| 170 | MRT4577_49099P | gl_27528490 |
| 170 | MRT4577_49099P | gl_3417405 |
| 170 | MRT4577_49099P | gl_29420851 |
| 170 | MRT4577_49099P | gl_6320016 |
| 170 | MRT4577_49099P | gl_172907 |
| 170 | MRT4577_49099P | gl_29420847 |
| 170 | MRT4577_49099P | gl_29420835 |
| 170 | MRT4577_49099P | gl_29420833 |
| 170 | MRT4577_49099P | gl_29420837 |
| 170 | MRT4577_49099P | gl_29420849 |
| 170 | MRT4577_49099P | gl_29420843 |
| 170 | MRT4577_49099P | gl_27528502 |
| 170 | MRT4577_49099P | gl_27528500 |
| 170 | MRT4577_49099P | gl_27529077 |
| 170 | MRT4577_49099P | gl_27529081 |
| 170 | MRT4577_49099P | gl_27529079 |
| 170 | MRT4577_49099P | gl_15422204 |
| 170 | MRT4577_49099P | gl_15422208 |
| 170 | MRT4577_49099P | gl_15425560 |
| 170 | MRT4577_49099P | gl_15425564 |
| 170 | MRT4577_49099P | gl_15425590 |
| 170 | MRT4577_49099P | gl_18077605 |
| 170 | MRT4577_49099P | gl_8452620 |
| 170 | MRT4577_49099P | gl_16943741 |
| 170 | MRT4577_49099P | gl_16943745 |
| 170 | MRT4577_49099P | gl_7708256 |
| 170 | MRT4577_49099P | gl_20467381 |
| 170 | MRT4577_49099P | gl_13540883 |
| 170 | MRT4577_49099P | gl_6017840 |
| 170 | MRT4577_49099P | gl_7708658 |
| 170 | MRT4577_49099P | gl_16444949 |
| 170 | MRT4577_49099P | gl_23503623 |
| 170 | MRT4577_49099P | gl_32412440 |
| 170 | MRT4577_49099P | gl_27526583 |
| 170 | MRT4577_49099P | gl_16416748 |
| 170 | MRT4577_49099P | gl_14591712 |
| 170 | MRT4577_49099P | gl_9799472 |
| 170 | MRT4577_49099P | gl_29420853 |
| 170 | MRT4577_49099P | gl_586209 |
| 170 | MRT4577_49099P | gl_3850934 |
| 170 | MRT4577_49099P | gl_3850978 |
| 170 | MRT4577_49099P | gl_6467950 |
| 170 | MRT4577_49099P | gl_12585490 |
| 170 | MRT4577_49099P | gl_7708260 |
| 170 | MRT4577_49099P | gl_14717946 |
| 170 | MRT4577_49099P | gl_6467935 |
| 170 | MRT4577_49099P | gl_6599365 |
| 170 | MRT4577_49099P | gl_11467696 |
| 170 | MRT4577_49099P | gl_14600685 |
| 170 | MRT4577_49099P | gl_18075915 |
| 170 | MRT4577_49099P | gl_6724287 |
| 170 | MRT4577_49099P | gl_14718201 |
| 170 | MRT4577_49099P | gl_7708448 |
| 170 | MRT4577_49099P | gl_16943658 |
| 170 | MRT4577_49099P | gl_5758910 |
| 170 | MRT4577_49099P | gl_8452704 |
| 170 | MRT4577_49099P | gl_16943668 |
| 170 | MRT4577_49099P | gl_7708642 |
| 170 | MRT4577_49099P | gl_7708668 |
| 170 | MRT4577_49099P | gl_12585391 |
| 170 | MRT4577_49099P | gl_12004165 |
| 170 | MRT4577_49099P | gl_12004135 |
| 170 | MRT4577_49099P | gl_12004123 |
| 170 | MRT4577_49099P | gl_12004117 |
| 170 | MRT4577_49099P | gl_12004111 |
| 170 | MRT4577_49099P | gl_12004157 |
| 170 | MRT4577_49099P | gl_12004167 |
| 170 | MRT4577_49099P | gl_11558464 |
| 170 | MRT4577_49099P | gl_7688339 |
| 170 | MRT4577_49099P | gl_7708187 |
| 170 | MRT4577_49099P | gl_7708442 |
| 170 | MRT4577_49099P | gl_10955560 |
| 170 | MRT4577_49099P | gl_14717933 |
| 170 | MRT4577_49099P | gl_7708304 |
| 170 | MRT4577_49099P | gl_7708572 |
| 170 | MRT4577_49099P | gl_14718240 |
| 170 | MRT4577_49099P | gl_732262 |
| 170 | MRT4577_49099P | gl_7687980 |
| 170 | MRT4577_49099P | gl_12229704 |
| 170 | MRT4577_49099P | gl_15790973 |
| 170 | MRT4577_49099P | gl_4063536 |
| 170 | MRT4577_49099P | gl_7688421 |
| 170 | MRT4577_49099P | gl_14718038 |
| 170 | MRT4577_49099P | gl_4995097 |
| 170 | MRT4577_49099P | gl_4063556 |
| 170 | MRT4577_49099P | gl_4063530 |
| 170 | MRT4577_49099P | gl_4206576 |
| 170 | MRT4577_49099P | gl_4206592 |
| 170 | MRT4577_49099P | gl_4995053 |
| 170 | MRT4577_49099P | gl_4995757 |
| 170 | MRT4577_49099P | gl_23503621 |
| 170 | MRT4577_49099P | gl_4063540 |
| 170 | MRT4577_49099P | gl_4063550 |
| 170 | MRT4577_49099P | gl_4995854 |
| 170 | MRT4577_49099P | gl_4063528 |
| 170 | MRT4577_49099P | gl_4063570 |
| 170 | MRT4577_49099P | gl_4063568 |
| 170 | MRT4577_49099P | gl_7708339 |
| 170 | MRT4577_49099P | gl_15425580 |
| 170 | MRT4577_49099P | gl_19033087 |
| 170 | MRT4577_49099P | gl_21684907 |
| 170 | MRT4577_49099P | gl_21684923 |
| 170 | MRT4577_49099P | gl_21684881 |
| 170 | MRT4577_49099P | gl_21684885 |
| 170 | MRT4577_49099P | gl_4206588 |
| 170 | MRT4577_49099P | gl_4206584 |
| 170 | MRT4577_49099P | gl_4206602 |
| 170 | MRT4577_49099P | gl_8388947 |
| 170 | MRT4577_49099P | gl_4206606 |
| 170 | MRT4577_49099P | gl_4206598 |
| 170 | MRT4577_49099P | gl_4206578 |
| 170 | MRT4577_49099P | gl_4206604 |
| 170 | MRT4577_49099P | gl_4206608 |
| 170 | MRT4577_49099P | gl_14718085 |
| 170 | MRT4577_49099P | gl_28188337 |
| 170 | MRT4577_49099P | gl_24940184 |
| 170 | MRT4577_49099P | gl_24940260 |
| 170 | MRT4577_49099P | gl_24940270 |
| 170 | MRT4577_49099P | gl_7708163 |
| 170 | MRT4577_49099P | gl_7708514 |
| 170 | MRT4577_49099P | gl_15605029 |
| 170 | MRT4577_49099P | gl_5758891 |
| 170 | MRT4577_49099P | gl_30351931 |
| 170 | MRT4577_49099P | gl_21633457 |
| 170 | MRT4577_49099P | gl_14717948 |
| 170 | MRT4577_49099P | gl_4995095 |
| 170 | MRT4577_49099P | gl_4995715 |
| 170 | MRT4577_49099P | gl_4995221 |
| 170 | MRT4577_49099P | gl_4995153 |
| 170 | MRT4577_49099P | gl_4995063 |
| 170 | MRT4577_49099P | gl_4995111 |
| 170 | MRT4577_49099P | gl_4995177 |
| 170 | MRT4577_49099P | gl_4995705 |
| 170 | MRT4577_49099P | gl_4995798 |
| 170 | MRT4577_49099P | gl_4995856 |
| 170 | MRT4577_49099P | gl_4995858 |
| 170 | MRT4577_49099P | gl_4995107 |
| 170 | MRT4577_49099P | gl_4995181 |
| 170 | MRT4577_49099P | gl_4995183 |
| 170 | MRT4577_49099P | gl_4995649 |
| 170 | MRT4577_49099P | gl_4995761 |
| 170 | MRT4577_49099P | gl_4995790 |
| 170 | MRT4577_49099P | gl_4995792 |
| 170 | MRT4577_49099P | gl_4995796 |
| 170 | MRT4577_49099P | gl_4995848 |
| 170 | MRT4577_49099P | gl_4995850 |
| 170 | MRT4577_49099P | gl_4995852 |
| 170 | MRT4577_49099P | gl_4995767 |
| 170 | MRT4577_49099P | gl_4995057 |
| 170 | MRT4577_49099P | gl_4995059 |
| 170 | MRT4577_49099P | gl_4995103 |
| 170 | MRT4577_49099P | gl_4995105 |
| 170 | MRT4577_49099P | gl_4995794 |
| 170 | MRT4577_49099P | gl_4995788 |
| 170 | MRT4577_49099P | gl_4995844 |
| 170 | MRT4577_49099P | gl_15608450 |
| 170 | MRT4577_49099P | gl_15835199 |
| 170 | MRT4577_49099P | gl_3850906 |
| 170 | MRT4577_49099P | gl_3850948 |
| 170 | MRT4577_49099P | gl_3850900 |
| 170 | MRT4577_49099P | gl_3850964 |
| 170 | MRT4577_49099P | gl_3850966 |
| 170 | MRT4577_49099P | gl_3850988 |
| 170 | MRT4577_49099P | gl_3850980 |
| 170 | MRT4577_49099P | gl_3850958 |
| 170 | MRT4577_49099P | gl_3850950 |
| 170 | MRT4577_49099P | gl_3850942 |
| 170 | MRT4577_49099P | gl_3850984 |
| 170 | MRT4577_49099P | gl_3850944 |
| 170 | MRT4577_49099P | gl_3850922 |
| 170 | MRT4577_49099P | gl_3850936 |
| 170 | MRT4577_49099P | gl_3850914 |
| 170 | MRT4577_49099P | gl_4731153 |
| 170 | MRT4577_49099P | gl_3850926 |
| 170 | MRT4577_49099P | gl_3850908 |
| 170 | MRT4577_49099P | gl_3850976 |
| 170 | MRT4577_49099P | gl_5001597 |
| 170 | MRT4577_49099P | gl_7708315 |
| 170 | MRT4577_49099P | gl_7708143 |
| 170 | MRT4577_49099P | gl_7708145 |
| 170 | MRT4577_49099P | gl_7708147 |
| 170 | MRT4577_49099P | gl_7708512 |
| 170 | MRT4577_49099P | gl_7708191 |
| 170 | MRT4577_49099P | gl_7708254 |
| 170 | MRT4577_49099P | gl_7708268 |
| 170 | MRT4577_49099P | gl_7708272 |
| 170 | MRT4577_49099P | gl_7708296 |
| 170 | MRT4577_49099P | gl_7708300 |
| 170 | MRT4577_49099P | gl_7708286 |
| 170 | MRT4577_49099P | gl_7708311 |
| 170 | MRT4577_49099P | gl_7708306 |
| 170 | MRT4577_49099P | gl_7708313 |
| 170 | MRT4577_49099P | gl_24940188 |
| 170 | MRT4577_49099P | gl_7708452 |
| 170 | MRT4577_49099P | gl_7708454 |
| 170 | MRT4577_49099P | gl_7708460 |
| 170 | MRT4577_49099P | gl_7708466 |
| 170 | MRT4577_49099P | gl_7708474 |
| 170 | MRT4577_49099P | gl_8517628 |
| 170 | MRT4577_49099P | gl_7708491 |
| 170 | MRT4577_49099P | gl_7708497 |
| 170 | MRT4577_49099P | gl_7708499 |
| 170 | MRT4577_49099P | gl_7708542 |
| 170 | MRT4577_49099P | gl_7708552 |
| 170 | MRT4577_49099P | gl_7708556 |
| 170 | MRT4577_49099P | gl_7708558 |
| 170 | MRT4577_49099P | gl_7708560 |
| 170 | MRT4577_49099P | gl_7708616 |
| 170 | MRT4577_49099P | gl_7708578 |
| 170 | MRT4577_49099P | gl_7708622 |
| 170 | MRT4577_49099P | gl_7708628 |
| 170 | MRT4577_49099P | gl_7708646 |
| 170 | MRT4577_49099P | gl_7708652 |
| 170 | MRT4577_49099P | gl_8452779 |
| 170 | MRT4577_49099P | gl_7708674 |
| 170 | MRT4577_49099P | gl_7688335 |
| 170 | MRT4577_49099P | gl_7708684 |
| 170 | MRT4577_49099P | gl_7708676 |
| 170 | MRT4577_49099P | gl_7688417 |
| 170 | MRT4577_49099P | gl_13518304 |
| 170 | MRT4577_49099P | gl_20269416 |
| 170 | MRT4577_49099P | gl_6687627 |
| 170 | MRT4577_49099P | gl_6706286 |
| 170 | MRT4577_49099P | gl_6687379 |
| 170 | MRT4577_49099P | gl_6688708 |
| 170 | MRT4577_49099P | gl_6687120 |
| 170 | MRT4577_49099P | gl_14717980 |
| 170 | MRT4577_49099P | gl_6687485 |
| 170 | MRT4577_49099P | gl_6687447 |
| 170 | MRT4577_49099P | gl_6688494 |
| 170 | MRT4577_49099P | gl_6689410 |
| 170 | MRT4577_49099P | gl_5001603 |
| 170 | MRT4577_49099P | gl_14587183 |
| 170 | MRT4577_49099P | gl_5001601 |
| 170 | MRT4577_49099P | gl_5758889 |
| 170 | MRT4577_49099P | gl_6017806 |
| 170 | MRT4577_49099P | gl_22406531 |
| 170 | MRT4577_49099P | gl_20384955 |
| 170 | MRT4577_49099P | gl_19033059 |
| 170 | MRT4577_49099P | gl_20384957 |
| 170 | MRT4577_49099P | gl_19033061 |
| 170 | MRT4577_49099P | gl_6689000 |
| 170 | MRT4577_49099P | gl_6017810 |
| 170 | MRT4577_49099P | gl_21684883 |
| 170 | MRT4577_49099P | gl_14718265 |
| 171 | MRT4577_346921P | gl_15810897 |
| 171 | MRT4577_346921P | gl_15810901 |
| 171 | MRT4577_346921P | gl_19698536 |
| 171 | MRT4577_346921P | gl_4096982 |
| 171 | MRT4577_346921P | gl_4103757 |
| 171 | MRT4577_346921P | gl_21667496 |
| 171 | MRT4577_346921P | gl_848999 |
| 171 | MRT4577_346921P | gl_4218162 |
| 171 | MRT4577_346921P | gl_4218160 |
| 171 | MRT4577_346921P | gl_14279306 |
| 171 | MRT4577_346921P | gl_20385590 |
| 171 | MRT4577_346921P | gl_30171291 |
| 171 | MRT4577_346921P | gl_30230270 |
| 171 | MRT4577_346921P | gl_25307920 |
| 171 | MRT4577_346921P | gl_4033721 |
| 171 | MRT4577_346921P | gl_4033725 |
| 171 | MRT4577_346921P | gl_4033710 |
| 171 | MRT4577_346921P | gl_4103486 |
| 171 | MRT4577_346921P | gl_5019431 |
| 171 | MRT4577_346921P | gl_8745072 |
| 171 | MRT4577_346921P | gl_19743774 |
| 171 | MRT4577_346921P | gl_23194453 |
| 171 | MRT4577_346921P | gl_4103346 |
| 171 | MRT4577_346921P | gl_7446520 |
| 171 | MRT4577_346921P | gl_2981131 |
| 171 | MRT4577_346921P | gl_2981133 |
| 171 | MRT4577_346921P | CGPG25.pep |
| 171 | MRT4577_346921P | gl_1345505 |
| 171 | MRT4577_346921P | gl_7446527 |
| 171 | MRT4577_346921P | gl_22328782 |
| 171 | MRT4577_346921P | gl_3915597 |
| 171 | MRT4577_346921P | gl_15231135 |
| 171 | MRT4577_346921P | gl_25307910 |
| 171 | MRT4577_346921P | gl_18406070 |
| 171 | MRT4577_346921P | gl_30689162 |
| 171 | MRT4577_346921P | gl_399096 |
| 171 | MRT4577_346921P | gl_12655901 |
| 171 | MRT4577_346921P | gl_5305232 |
| 171 | MRT4577_346921P | gl_5305242 |
| 171 | MRT4577_346921P | gl_5305260 |
| 171 | MRT4577_346921P | gl_5305244 |
| 171 | MRT4577_346921P | gl_5616513 |
| 171 | MRT4577_346921P | gl_16973298 |
| 171 | MRT4577_346921P | gl_16973296 |
| 171 | MRT4577_346921P | gl_602900 |
| 171 | MRT4577_346921P | MRT3847_56279P.2 |
| 171 | MRT4577_346921P | MRT3847_64872P.3 |
| 171 | MRT4577_346921P | MRT3847_233420P.2 |
| 171 | MRT4577_346921P | MRT3847_64874P.3 |
| 171 | MRT4577_346921P | MRT3847_218209P.1 |
| 171 | MRT4577_346921P | MRT3847_29836P.3 |
| 171 | MRT4577_346921P | MRT3847_225429P.3 |
| 171 | MRT4577_346921P | gl_13161415 |
| 171 | MRT4577_346921P | gl_3913005 |
| 171 | MRT4577_346921P | gl_24967135 |
| 171 | MRT4577_346921P | gl_3913004 |
| 171 | MRT4577_346921P | gl_23428880 |
| 171 | MRT4577_346921P | gl_24967137 |
| 171 | MRT4577_346921P | gl_4097515 |
| 171 | MRT4577_346921P | gl_3913007 |
| 171 | MRT4577_346921P | gl_17827467 |
| 171 | MRT4577_346921P | gl_3913006 |
| 171 | MRT4577_346921P | gl_2129972 |
| 171 | MRT4577_346921P | gl_1067169 |
| 171 | MRT4577_346921P | gl_1568513 |
| 171 | MRT4577_346921P | gl_1364102 |
| 171 | MRT4577_346921P | gl_322801 |
| 171 | MRT4577_346921P | gl_4837612 |
| 171 | MRT4577_346921P | gl_27804365 |
| 171 | MRT4577_346921P | gl_27657747 |
| 171 | MRT4577_346921P | gl_24414622 |
| 171 | MRT4577_346921P | gl_27657745 |
| 171 | MRT4577_346921P | gl_5031217 |
| 171 | MRT4577_346921P | MRT4530_57276P.1 |
| 171 | MRT4577_346921P | gl_2130078 |
| 171 | MRT4577_346921P | MRT4565_47460P.3 |
| 171 | MRT4577_346921P | gl_14041687 |
| 171 | MRT4577_346921P | gl_26517024 |
| 171 | MRT4577_346921P | gl_4101710 |
| 171 | MRT4577_346921P | gl_6970411 |
| 171 | MRT4577_346921P | gl_6970415 |
| 171 | MRT4577_346921P | gl_6970413 |
| 171 | MRT4577_346921P | gl_6970417 |
| 171 | MRT4577_346921P | gl_18650789 |
| 171 | MRT4577_346921P | gl_22091479 |
| 171 | MRT4577_346921P | gl_16549060 |
| 171 | MRT4577_346921P | gl_16549078 |
| 171 | MRT4577_346921P | gl_4887235 |
| 172 | MRT4577_257780P | gl_14626277 |
| 172 | MRT4577_257780P | MRT4530_28144P.1 |
| 172 | MRT4577_257780P | MRT4565_27586P.3 |
| 172 | MRT4577_257780P | MRT4565_9771P.3 |
| 172 | MRT4577_257780P | MRT4565_64073P.2 |
| 172 | MRT4577_257780P | MRT4565_91331P.2 |
| 175 | MRT4577_294774P | gl_7452981 |
| 175 | MRT4577_294774P | gl_7452979 |
| 175 | MRT4577_294774P | gl_13183137 |
| 175 | MRT4577_294774P | gl_29373125 |
| 175 | MRT4577_294774P | gl_25089839 |
| 175 | MRT4577_294774P | gl_21616113 |
| 175 | MRT4577_294774P | gl_11357336 |
| 175 | MRT4577_294774P | gl_25308880 |
| 175 | MRT4577_294774P | gl_15233810 |
| 175 | MRT4577_294774P | MRT3847_39339P.3 |
| 175 | MRT4577_294774P | gl_5830467 |
| 175 | MRT4577_294774P | gl_5830465 |
| 175 | MRT4577_294774P | gl_5830469 |
| 175 | MRT4577_294774P | gl_7446714 |
| 175 | MRT4577_294774P | gl_1272340 |
| 175 | MRT4577_294774P | gl_11278993 |
| 175 | MRT4577_294774P | gl_13506709 |
| 175 | MRT4577_294774P | gl_7677378 |
| 175 | MRT4577_294774P | gl_4850214 |
| 175 | MRT4577_294774P | gl_17646111 |
| 175 | MRT4577_294774P | gl_14627128 |
| 175 | MRT4577_294774P | gl_22265999 |
| 175 | MRT4577_294774P | MRT4530_57126P.1 |
| 175 | MRT4577_294774P | MRT4565_107456P.1 |
| 175 | MRT4577_294774P | gl_15982240 |
| 177 | MRT4577_397598P | MRT3847_29671P.3 |
| 177 | MRT4577_397598P | MRT3847_36085P.3 |
| 177 | MRT4577_397598P | MRT3847_37502P.1 |
| 177 | MRT4577_397598P | gl_10241425 |
| 177 | MRT4577_397598P | MRT4530_77791P.2 |
| 177 | MRT4577_397598P | MRT4530_81676P.1 |
| 177 | MRT4577_397598P | MRT4565_118744P.1 |
| 178 | MRT4577_204611P | gl_28194506 |
| 178 | MRT4577_204611P | gl_28194508 |
| 178 | MRT4577_204611P | gl_15866696 |
| 178 | MRT4577_204611P | gl_27475608 |
| 178 | MRT4577_204611P | gl_28194504 |
| 178 | MRT4577_204611P | gl_7447118 |
| 178 | MRT4577_204611P | gl_8918271 |
| 178 | MRT4577_204611P | gl_8918273 |
| 178 | MRT4577_204611P | gl_30060377 |
| 178 | MRT4577_204611P | MRT4530_76824P.2 |
| 178 | MRT4577_204611P | MRT4530_76823P.2 |
| 178 | MRT4577_204611P | MRT4530_109505P.2 |
| 178 | MRT4577_204611P | MRT4565_29431P.3 |
| 178 | MRT4577_204611P | gl_6456469 |
| 178 | MRT4577_204611P | gl_6456467 |
| 178 | MRT4577_204611P | gl_5922599 |
| 181 | MRT4577_284905P | gl_6822147 |
| 181 | MRT4577_284905P | gl_3702409 |
| 181 | MRT4577_284905P | gl_5834521 |
| 181 | MRT4577_284905P | gl_5834523 |
| 181 | MRT4577_284905P | gl_4584556 |
| 181 | MRT4577_284905P | gl_8980813 |
| 181 | MRT4577_284905P | gl_8980815 |
| 181 | MRT4577_284905P | gl_16225426 |
| 181 | MRT4577_284905P | gl_14329816 |
| 181 | MRT4577_284905P | gl_1469934 |
| 181 | MRT4577_284905P | gl_7447961 |
| 181 | MRT4577_284905P | gl_18087505 |
| 181 | MRT4577_284905P | gl_7447977 |
| 181 | MRT4577_284905P | gl_18379267 |
| 181 | MRT4577_284905P | gl_25410916 |
| 181 | MRT4577_284905P | gl_15219603 |
| 181 | MRT4577_284905P | gl_25402689 |
| 181 | MRT4577_284905P | gl_15220923 |
| 181 | MRT4577_284905P | gl_1352326 |
| 181 | MRT4577_284905P | gl_12655961 |
| 181 | MRT4577_284905P | gl_20149296 |
| 181 | MRT4577_284905P | gl_20149298 |
| 181 | MRT4577_284905P | gl_13548679 |
| 181 | MRT4577_284905P | gl_3900936 |
| 181 | MRT4577_284905P | gl_4883425 |
| 181 | MRT4577_284905P | MRT3847_161472P.3 |
| 181 | MRT4577_284905P | MRT3847_36311P.3 |
| 181 | MRT4577_284905P | gl_7447979 |
| 181 | MRT4577_284905P | gl_119006 |
| 181 | MRT4577_284905P | gl_99998 |
| 181 | MRT4577_284905P | gl_11279328 |
| 181 | MRT4577_284905P | gl_1169445 |
| 181 | MRT4577_284905P | gl_261212 |
| 181 | MRT4577_284905P | gl_20269069 |
| 181 | MRT4577_284905P | gl_32765543 |
| 181 | MRT4577_284905P | gl_1706547 |
| 181 | MRT4577_284905P | gl_4469175 |
| 181 | MRT4577_284905P | gl_10946499 |
| 181 | MRT4577_284905P | gl_29150650 |
| 181 | MRT4577_284905P | gl_22748323 |
| 181 | MRT4577_284905P | gl_6984122 |
| 181 | MRT4577_284905P | gl_11321164 |
| 181 | MRT4577_284905P | gl_461978 |
| 181 | MRT4577_284905P | gl_461979 |
| 181 | MRT4577_284905P | gl_1084399 |
| 181 | MRT4577_284905P | gl_1084400 |
| 181 | MRT4577_284905P | gl_11558184 |
| 181 | MRT4577_284905P | gl_100285 |
| 181 | MRT4577_284905P | gl_100287 |
| 181 | MRT4577_284905P | gl_27529826 |
| 181 | MRT4577_284905P | gl_11071974 |
| 181 | MRT4577_284905P | gl_16903129 |
| 181 | MRT4577_284905P | gl_15150341 |
| 181 | MRT4577_284905P | MRT4530_84009P.2 |
| 181 | MRT4577_284905P | gl_15529115 |
| 181 | MRT4577_284905P | MRT4565_60761P.2 |
| 181 | MRT4577_284905P | gl_14330338 |
| 181 | MRT4577_284905P | gl_20218805 |
| 181 | MRT4577_284905P | gl_688420 |
| 181 | MRT4577_284905P | gl_11279332 |
| 182 | MRT4577_386764P | gl_7573596 |
| 182 | MRT4577_386764P | gl_7573598 |
| 182 | MRT4577_386764P | gl_25287618 |
| 182 | MRT4577_386764P | gl_30695267 |
| 182 | MRT4577_386764P | gl_18400939 |
| 182 | MRT4577_386764P | gl_21593950 |
| 182 | MRT4577_386764P | gl_13447449 |
| 182 | MRT4577_386764P | gl_3668069 |
| 182 | MRT4577_386764P | gl_29427825 |
| 182 | MRT4577_386764P | gl_30421168 |
| 182 | MRT4577_386764P | MRT4530_135930P.1 |
| 182 | MRT4577_386764P | MRT4530_120903P.1 |
| 182 | MRT4577_386764P | gl_21326117 |
| 182 | MRT4577_386764P | MRT4565_103551P.1 |
| 182 | MRT4577_386764P | MRT4565_52855P.3 |
| 182 | MRT4577_386764P | MRT4565_88207P.2 |
| 182 | MRT4577_386764P | gl_28140043 |
| 182 | MRT4577_386764P | gl_28804505 |
| 184 | MRT4577_43098P | gl_27542603 |
| 184 | MRT4577_43098P | gl_22328179 |
| 184 | MRT4577_43098P | MRT3847_52308P.3 |
| 184 | MRT4577_43098P | gl_29893654 |
| 184 | MRT4577_43098P | MRT4530_35848P.1 |
| 184 | MRT4577_43098P | MRT4530_35849P.2 |
| 184 | MRT4577_43098P | MRT4530_121232P.2 |
| 184 | MRT4577_43098P | MRT4530_113489P.2 |
| 184 | MRT4577_43098P | MRT4565_49252P.2 |
| 185 | MRT4577_222465P | MRT3847_10488P.3 |
| 185 | MRT4577_222465P | MRT4530_104720P.2 |
| 185 | MRT4577_222465P | MRT4565_89954P.2 |
| 185 | MRT4577_222465P | MRT4565_71673P.1 |
| 186 | MRT4577_326681P | gl_15240418 |
| 186 | MRT4577_326681P | gl_28071332 |
| 187 | MRT4577_361986P | gl_30385250 |
| 187 | MRT4577_361986P | gl_14495542 |
| 187 | MRT4577_361986P | gl_15227441 |
| 187 | MRT4577_361986P | gl_31540632 |
| 187 | MRT4577_361986P | gl_25004882 |
| 187 | MRT4577_361986P | gl_24935324 |
| 187 | MRT4577_361986P | gl_24940244 |
| 187 | MRT4577_361986P | gl_7488932 |
| 187 | MRT4577_361986P | gl_30421165 |
| 187 | MRT4577_361986P | gl_7434424 |
| 187 | MRT4577_361986P | MRT4530_85948P.1 |
| 187 | MRT4577_361986P | gl_5596996 |
| 187 | MRT4577_361986P | gl_13620169 |
| 189 | MRT4577_300134P | gl_23429044 |
| 189 | MRT4577_300134P | gl_14573437 |
| 189 | MRT4577_300134P | gl_26190149 |
| 189 | MRT4577_300134P | gl_11357139 |
| 189 | MRT4577_300134P | gl_30682129 |
| 189 | MRT4577_300134P | gl_12322049 |
| 189 | MRT4577_300134P | gl_15795149 |
| 189 | MRT4577_300134P | gl_15810509 |
| 189 | MRT4577_300134P | MRT3847_48429P.3 |
| 189 | MRT4577_300134P | gl_6681366 |
| 189 | MRT4577_300134P | gl_6984231 |
| 189 | MRT4577_300134P | gl_4586799 |
| 189 | MRT4577_300134P | MRT4530_81439P.1 |
| 189 | MRT4577_300134P | MRT4530_81446P.2 |
| 189 | MRT4577_300134P | MRT4565_30002P.3 |
| 189 | MRT4577_300134P | gl_7381060 |
| 190 | MRT4577_415225P | gl_32441499 |
| 190 | MRT4577_415225P | gl_4206759 |
| 190 | MRT4577_415225P | gl_28564230 |
| 190 | MRT4577_415225P | gl_28564264 |
| 190 | MRT4577_415225P | gl_32441494 |
| 190 | MRT4577_415225P | MRT3847_52223P.3 |
| 190 | MRT4577_415225P | MRT3847_30045P.3 |
| 190 | MRT4577_415225P | gl_32483423 |
| 190 | MRT4577_415225P | gl_20330757 |
| 190 | MRT4577_415225P | gl_20146358 |
| 190 | MRT4577_415225P | gl_22775591 |
| 190 | MRT4577_415225P | MRT4530_110805P.1 |
| 190 | MRT4577_415225P | MRT4530_87778P.1 |
| 190 | MRT4577_415225P | MRT4530_100513P.2 |
| 190 | MRT4577_415225P | gl_21070389 |
| 190 | MRT4577_415225P | MRT4565_36882P.3 |
| 190 | MRT4577_415225P | MRT4565_110825P.1 |
| 190 | MRT4577_415225P | MRT4565_127690P.1 |
| 190 | MRT4577_415225P | MRT4565_86330P.2 |
| 190 | MRT4577_415225P | MRT4565_40318P.2 |
| 190 | MRT4577_415225P | gl_28564015 |
| 190 | MRT4577_415225P | gl_28564960 |
| 190 | MRT4577_415225P | gl_32441506 |
| 190 | MRT4577_415225P | gl_32441496 |
| 190 | MRT4577_415225P | gl_32441504 |
| 190 | MRT4577_415225P | gl_2274776 |
| 192 | MRT4577_56004P | MRT3847_215323P.2 |
| 192 | MRT4577_56004P | MRT3847_44128P.3 |
| 192 | MRT4577_56004P | MRT4565_57540P.2 |
| 192 | MRT4577_56004P | gl_19112800 |
| 192 | MRT4577_56004P | gl_1808694 |
| 194 | MRT4577_221761P | gl_22331664 |
| 194 | MRT4577_221761P | gl_30692988 |
| 194 | MRT4577_221761P | gl_22330789 |
| 194 | MRT4577_221761P | gl_15242176 |
| 194 | MRT4577_221761P | gl_6094551 |
| 194 | MRT4577_221761P | gl_7487385 |
| 194 | MRT4577_221761P | gl_19578317 |
| 194 | MRT4577_221761P | MRT3847_265345P.2 |
| 194 | MRT4577_221761P | MRT3847_53989P.3 |
| 194 | MRT4577_221761P | MRT3847_6971P.3 |
| 194 | MRT4577_221761P | MRT3847_162726P.3 |
| 194 | MRT4577_221761P | MRT3847_239538P.2 |
| 194 | MRT4577_221761P | MRT3847_253605P.2 |
| 194 | MRT4577_221761P | MRT3847_53988P.3 |
| 194 | MRT4577_221761P | MRT3847_227267P.3 |
| 194 | MRT4577_221761P | MRT3847_30433P.3 |
| 194 | MRT4577_221761P | MRT3847_269768P.1 |
| 194 | MRT4577_221761P | MRT3847_224215P.2 |
| 194 | MRT4577_221761P | MRT3847_272006P.1 |
| 194 | MRT4577_221761P | MRT4530_103360P.1 |
| 194 | MRT4577_221761P | MRT4530_103357P.1 |
| 194 | MRT4577_221761P | MRT4530_103362P.1 |
| 194 | MRT4577_221761P | MRT4530_98210P.1 |
| 194 | MRT4577_221761P | MRT4565_20121P.3 |
| 194 | MRT4577_221761P | MRT4565_90833P.2 |
| 194 | MRT4577_221761P | MRT4565_61922P.2 |
| 194 | MRT4577_221761P | MRT4565_76776P.2 |
| 196 | MRT4577_401949P | MRT3847_241638P.2 |
| 196 | MRT4577_401949P | MRT3847_286535P.1 |
| 196 | MRT4577_401949P | MRT3847_52567P.3 |
| 196 | MRT4577_401949P | MRT3847_255937P.2 |
| 196 | MRT4577_401949P | gl_6782440 |
| 196 | MRT4577_401949P | MRT4530_140459P.1 |
| 196 | MRT4577_401949P | gl_15209148 |
| 196 | MRT4577_401949P | MRT4530_18787P.2 |
| 196 | MRT4577_401949P | MRT4565_19576P.3 |
| 200 | MRT4577_26957P | gl_32477628 |
| 200 | MRT4577_26957P | gl_15896652 |
| 200 | MRT4577_26957P | gl_33113492 |
| 200 | MRT4577_26957P | gl_16610205 |
| 200 | MRT4577_26957P | gl_18407057 |
| 200 | MRT4577_26957P | gl_25345298 |
| 200 | MRT4577_26957P | gl_15292855 |
| 200 | MRT4577_26957P | gl_15236304 |
| 200 | MRT4577_26957P | MRT3847_58239P.2 |
| 200 | MRT4577_26957P | MRT3847_61026P.3 |
| 200 | MRT4577_26957P | MRT3847_249176P.2 |
| 200 | MRT4577_26957P | MRT3847_32267P.3 |
| 200 | MRT4577_26957P | MRT3847_249177P.2 |
| 200 | MRT4577_26957P | gl_8096650 |
| 200 | MRT4577_26957P | MRT4530_97319P.2 |
| 200 | MRT4577_26957P | MRT4530_111084P.2 |
| 200 | MRT4577_26957P | MRT4565_42533P.3 |
| 203 | MRT4577_289436P | gl_7484643 |
| 203 | MRT4577_289436P | gl_7488272 |
| 203 | MRT4577_289436P | MRT4565_141501P.1 |
| 203 | MRT4577_289436P | MRT4565_58034P.2 |
| 204 | MRT4577_221609P | gl_30688566 |
| 204 | MRT4577_221609P | gl_30693084 |
| 204 | MRT4577_221609P | gl_11358184 |
| 204 | MRT4577_221609P | gl_15237549 |
| 204 | MRT4577_221609P | gl_3860313 |
| 204 | MRT4577_221609P | MRT3847_233522P.2 |
| 204 | MRT4577_221609P | MRT3847_286526P.1 |
| 204 | MRT4577_221609P | MRT3847_28679P.3 |
| 204 | MRT4577_221609P | MRT3847_47036P.3 |
| 204 | MRT4577_221609P | MRT4530_7968P.2 |
| 204 | MRT4577_221609P | MRT4565_16821P.3 |
| 204 | MRT4577_221609P | gl_19112558 |
| 204 | MRT4577_221609P | gl_6323275 |
| 204 | MRT4577_221609P | gl_32400328 |
| 204 | MRT4577_221609P | gl_32417454 |
| 204 | MRT4577_221609P | gl_13812075 |
| 204 | MRT4577_221609P | gl_5921507 |
| 205 | MRT4577_28967P | gl_15232517 |
| 205 | MRT4577_28967P | MRT3847_253859P.2 |
| 205 | MRT4577_28967P | MRT3847_198776P.3 |
| 205 | MRT4577_28967P | gl_9663979 |
| 205 | MRT4577_28967P | gl_7489198 |
| 205 | MRT4577_28967P | gl_22128589 |
| 205 | MRT4577_28967P | gl_22128591 |
| 205 | MRT4577_28967P | gl_22128587 |
| 205 | MRT4577_28967P | MRT4530_8279P.1 |
| 205 | MRT4577_28967P | gl_32418640 |
| 208 | MRT4577_45217P | gl_7484972 |
| 208 | MRT4577_45217P | gl_15226178 |
| 208 | MRT4577_45217P | gl_2245390 |
| 208 | MRT4577_45217P | MRT3847_208509P.3 |
| 208 | MRT4577_45217P | gl_20805068 |
| 208 | MRT4577_45217P | gl_19352035 |
| 208 | MRT4577_45217P | MRT4565_34024P.3 |
| 209 | MRT4577_420096P | gl_20330751 |
| 209 | MRT4577_420096P | MRT4530_54698P.1 |
| 209 | MRT4577_420096P | MRT4530_54700P.1 |
| 210 | MRT4577_220452P | MRT3847_2805P.3 |
| 210 | MRT4577_220452P | MRT4530_91499P.1 |
| 212 | MRT4577_5002P | gl_30687843 |
| 212 | MRT4577_5002P | gl_15223786 |
| 212 | MRT4577_5002P | gl_15239624 |
| 212 | MRT4577_5002P | gl_15226967 |
| 212 | MRT4577_5002P | gl_15229157 |
| 212 | MRT4577_5002P | gl_21593407 |
| 212 | MRT4577_5002P | gl_25346630 |
| 212 | MRT4577_5002P | gl_7486722 |
| 212 | MRT4577_5002P | gl_7488751 |
| 212 | MRT4577_5002P | gl_21742732 |
| 212 | MRT4577_5002P | MRT4530_122939P.2 |
| 212 | MRT4577_5002P | MRT4565_43218P.3 |
| 215 | MRT4577_389607P | gl_15223930 |
| 215 | MRT4577_389607P | gl_21553710 |
| 215 | MRT4577_389607P | gl_21536895 |
| 215 | MRT4577_389607P | gl_15242347 |
| 215 | MRT4577_389607P | gl_15237539 |
| 215 | MRT4577_389607P | gl_608671 |
| 215 | MRT4577_389607P | gl_20260650 |
| 215 | MRT4577_389607P | gl_21536979 |
| 215 | MRT4577_389607P | gl_30693784 |
| 215 | MRT4577_389607P | gl_608673 |
| 215 | MRT4577_389607P | gl_25297689 |
| 215 | MRT4577_389607P | MRT3847_30014P.3 |
| 215 | MRT4577_389607P | MRT3847_268909P.1 |
| 215 | MRT4577_389607P | MRT3847_55865P.2 |
| 215 | MRT4577_389607P | MRT3847_35167P.2 |
| 215 | MRT4577_389607P | gl_13676299 |
| 215 | MRT4577_389607P | MRT3847_271867P.1 |
| 215 | MRT4577_389607P | MRT3847_50682P.1 |
| 215 | MRT4577_389607P | MRT3847_85245P.2 |
| 215 | MRT4577_389607P | MRT3847_37580P.3 |
| 215 | MRT4577_389607P | MRT3847_90337P.3 |
| 215 | MRT4577_389607P | gl_6539602 |
| 215 | MRT4577_389607P | gl_4138679 |
| 215 | MRT4577_389607P | gl_15216030 |
| 215 | MRT4577_389607P | gl_15216026 |
| 215 | MRT4577_389607P | gl_15216028 |
| 215 | MRT4577_389607P | gl_7442734 |
| 215 | MRT4577_389607P | gl_7442735 |
| 215 | MRT4577_389607P | gl_4164408 |
| 215 | MRT4577_389607P | gl_20161442 |
| 215 | MRT4577_389607P | gl_27447657 |
| 215 | MRT4577_389607P | gl_27447653 |
| 215 | MRT4577_389607P | gl_7489096 |
| 215 | MRT4577_389607P | gl_7442732 |
| 215 | MRT4577_389607P | gl_4322323 |
| 215 | MRT4577_389607P | gl_4322325 |
| 215 | MRT4577_389607P | MRT4530_114765P.2 |
| 215 | MRT4577_389607P | MRT4565_14138P.3 |
| 216 | MRT4577_405388P | gl_3777497 |
| 216 | MRT4577_405388P | gl_464145 |
| 216 | MRT4577_405388P | gl_13366140 |
| 216 | MRT4577_405388P | gl_10953877 |
| 216 | MRT4577_405388P | gl_29134857 |
| 216 | MRT4577_405388P | gl_10953875 |
| 216 | MRT4577_405388P | gl_3779258 |
| 216 | MRT4577_405388P | gl_30267054 |
| 216 | MRT4577_405388P | gl_30267062 |
| 216 | MRT4577_405388P | gl_30267056 |
| 216 | MRT4577_405388P | gl_30265620 |
| 216 | MRT4577_405388P | gl_30267058 |
| 216 | MRT4577_405388P | gl_25289327 |
| 216 | MRT4577_405388P | gl_30685252 |
| 216 | MRT4577_405388P | gl_7428175 |
| 216 | MRT4577_405388P | gl_18414404 |
| 216 | MRT4577_405388P | gl_602764 |
| 216 | MRT4577_405388P | gl_30683170 |
| 216 | MRT4577_405388P | gl_17224922 |
| 216 | MRT4577_405388P | MRT3847_12543P.1 |
| 216 | MRT4577_405388P | gl_902938 |
| 216 | MRT4577_405388P | gl_231541 |
| 216 | MRT4577_405388P | gl_3913031 |
| 216 | MRT4577_405388P | gl_3913035 |
| 216 | MRT4577_405388P | gl_3913034 |
| 216 | MRT4577_405388P | gl_13489165 |
| 216 | MRT4577_405388P | gl_15082058 |
| 216 | MRT4577_405388P | gl_217936 |
| 216 | MRT4577_405388P | gl_416619 |
| 216 | MRT4577_405388P | gl_10120912 |
| 216 | MRT4577_405388P | gl_217940 |
| 216 | MRT4577_405388P | gl_20530741 |
| 216 | MRT4577_405388P | gl_11322499 |
| 216 | MRT4577_405388P | gl_113786 |
| 216 | MRT4577_405388P | gl_6729696 |
| 216 | MRT4577_405388P | MRT4530_147074P.1 |
| 216 | MRT4577_405388P | gl_169777 |
| 216 | MRT4577_405388P | MRT4530_118075P.1 |
| 216 | MRT4577_405388P | gl_169779 |
| 216 | MRT4577_405388P | gl_478405 |
| 216 | MRT4577_405388P | gl_231540 |
| 216 | MRT4577_405388P | MRT4565_14604P.1 |
| 216 | MRT4577_405388P | gl_3334120 |
| 216 | MRT4577_405388P | MRT4565_106072P.1 |
| 216 | MRT4577_405388P | MRT4565_14599P.3 |
| 216 | MRT4577_405388P | MRT4565_118733P.1 |
| 216 | MRT4577_405388P | MRT4565_58256P.2 |
| 216 | MRT4577_405388P | MRT4565_14593P.3 |
| 216 | MRT4577_405388P | MRT4565_104372P.1 |
| 216 | MRT4577_405388P | MRT4565_118736P.1 |
| 216 | MRT4577_405388P | gl_12006484 |
| 216 | MRT4577_405388P | gl_30267060 |
| 216 | MRT4577_405388P | gl_30267072 |
| 217 | MRT4577_388272P | gl_15225218 |
| 217 | MRT4577_388272P | gl_7488484 |
| 217 | MRT4577_388272P | gl_7488446 |
| 217 | MRT4577_388272P | gl_7488485 |
| 217 | MRT4577_388272P | gl_7488483 |
| 217 | MRT4577_388272P | MRT3847_284959P.1 |
| 217 | MRT4577_388272P | MRT3847_40554P.3 |
| 217 | MRT4577_388272P | MRT3847_7845P.3 |
| 217 | MRT4577_388272P | MRT3847_7846P.2 |
| 217 | MRT4577_388272P | MRT3847_33513P.3 |
| 217 | MRT4577_388272P | MRT3847_249579P.2 |
| 217 | MRT4577_388272P | MRT3847_33514P.2 |
| 217 | MRT4577_388272P | MRT3847_272723P.1 |
| 217 | MRT4577_388272P | gl_7488791 |
| 217 | MRT4577_388272P | gl_12039387 |
| 217 | MRT4577_388272P | MRT4530_114918P.2 |
| 217 | MRT4577_388272P | MRT4565_11213P.3 |
| 218 | MRT4577_61311P | MRT3847_239034P.2 |
| 218 | MRT4577_61311P | MRT3847_199862P.2 |
| 218 | MRT4577_61311P | gl_14906664 |
| 219 | MRT4577_287993P | gl_21220517 |
| 219 | MRT4577_287993P | gl_17227907 |
| 219 | MRT4577_287993P | gl_17232303 |
| 219 | MRT4577_287993P | gl_22959339 |
| 219 | MRT4577_287993P | gl_22962067 |
| 219 | MRT4577_287993P | gl_15616888 |
| 219 | MRT4577_287993P | gl_22965894 |
| 219 | MRT4577_287993P | gl_22972296 |
| 219 | MRT4577_287993P | gl_15921495 |
| 219 | MRT4577_287993P | gl_16329464 |
| 219 | MRT4577_287993P | gl_32473774 |
| 219 | MRT4577_287993P | gl_7674377 |
| 219 | MRT4577_287993P | gl_28380215 |
| 219 | MRT4577_287993P | gl_7674382 |
| 219 | MRT4577_287993P | gl_20808006 |
| 219 | MRT4577_287993P | gl_22977198 |
| 219 | MRT4577_287993P | gl_5764615 |
| 219 | MRT4577_287993P | gl_15676576 |
| 219 | MRT4577_287993P | gl_15793848 |
| 219 | MRT4577_287993P | gl_79917 |
| 219 | MRT4577_287993P | gl_15805966 |
| 219 | MRT4577_287993P | gl_8272441 |
| 219 | MRT4577_287993P | gl_5231208 |
| 219 | MRT4577_287993P | gl_5231187 |
| 219 | MRT4577_287993P | gl_5231184 |
| 219 | MRT4577_287993P | gl_5231202 |
| 219 | MRT4577_287993P | gl_5231181 |
| 219 | MRT4577_287993P | gl_5231193 |
| 219 | MRT4577_287993P | gl_5231190 |
| 219 | MRT4577_287993P | gl_5231205 |
| 219 | MRT4577_287993P | gl_5231196 |
| 219 | MRT4577_287993P | gl_5231199 |
| 219 | MRT4577_287993P | gl_22986693 |
| 219 | MRT4577_287993P | gl_15673444 |
| 219 | MRT4577_287993P | gl_136253 |
| 219 | MRT4577_287993P | gl_18390357 |
| 219 | MRT4577_287993P | gl_7676165 |
| 219 | MRT4577_287993P | gl_15896405 |
| 219 | MRT4577_287993P | gl_15801918 |
| 219 | MRT4577_287993P | gl_22994339 |
| 219 | MRT4577_287993P | gl_22997030 |
| 219 | MRT4577_287993P | gl_22999862 |
| 219 | MRT4577_287993P | gl_136260 |
| 219 | MRT4577_287993P | gl_15924363 |
| 219 | MRT4577_287993P | gl_26986827 |
| 219 | MRT4577_287993P | gl_15837977 |
| 219 | MRT4577_287993P | gl_16800736 |
| 219 | MRT4577_287993P | gl_33240025 |
| 219 | MRT4577_287993P | gl_16803667 |
| 219 | MRT4577_287993P | gl_15901640 |
| 219 | MRT4577_287993P | gl_15903673 |
| 219 | MRT4577_287993P | gl_80601 |
| 219 | MRT4577_287993P | gl_27467972 |
| 219 | MRT4577_287993P | gl_28380195 |
| 219 | MRT4577_287993P | gl_6226270 |
| 219 | MRT4577_287993P | gl_15827655 |
| 219 | MRT4577_287993P | gl_17933944 |
| 219 | MRT4577_287993P | gl_15887378 |
| 219 | MRT4577_287993P | gl_18978077 |
| 219 | MRT4577_287993P | gl_22125938 |
| 219 | MRT4577_287993P | gl_15679655 |
| 219 | MRT4577_287993P | gl_23016131 |
| 219 | MRT4577_287993P | gl_23006623 |
| 219 | MRT4577_287993P | gl_23010914 |
| 219 | MRT4577_287993P | gl_23004962 |
| 219 | MRT4577_287993P | gl_20091808 |
| 219 | MRT4577_287993P | gl_24215987 |
| 219 | MRT4577_287993P | gl_20093841 |
| 219 | MRT4577_287993P | gl_21231972 |
| 219 | MRT4577_287993P | gl_21243443 |
| 219 | MRT4577_287993P | gl_16127773 |
| 219 | MRT4577_287993P | gl_21399162 |
| 219 | MRT4577_287993P | gl_28380210 |
| 219 | MRT4577_287993P | gl_15791717 |
| 219 | MRT4577_287993P | gl_21228923 |
| 219 | MRT4577_287993P | gl_21673370 |
| 219 | MRT4577_287993P | gl_25029429 |
| 219 | MRT4577_287993P | gl_21282989 |
| 219 | MRT4577_287993P | gl_19554226 |
| 219 | MRT4577_287993P | gl_22297982 |
| 219 | MRT4577_287993P | gl_21672546 |
| 219 | MRT4577_287993P | gl_26247590 |
| 219 | MRT4577_287993P | gl_23017117 |
| 219 | MRT4577_287993P | gl_23021827 |
| 219 | MRT4577_287993P | gl_23023764 |
| 219 | MRT4577_287993P | gl_23026706 |
| 219 | MRT4577_287993P | gl_23040075 |
| 219 | MRT4577_287993P | gl_23502956 |
| 219 | MRT4577_287993P | gl_23335097 |
| 219 | MRT4577_287993P | gl_23469383 |
| 219 | MRT4577_287993P | gl_23061852 |
| 219 | MRT4577_287993P | gl_23465333 |
| 219 | MRT4577_287993P | gl_23473439 |
| 219 | MRT4577_287993P | gl_15595233 |
| 219 | MRT4577_287993P | gl_24379020 |
| 219 | MRT4577_287993P | gl_24374549 |
| 219 | MRT4577_287993P | gl_16122431 |
| 219 | MRT4577_287993P | gl_27366338 |
| 219 | MRT4577_287993P | gl_136262 |
| 219 | MRT4577_287993P | gl_15669227 |
| 219 | MRT4577_287993P | gl_7676173 |
| 219 | MRT4577_287993P | gl_28378350 |
| 219 | MRT4577_287993P | gl_23050672 |
| 219 | MRT4577_287993P | gl_23097976 |
| 219 | MRT4577_287993P | gl_28867399 |
| 219 | MRT4577_287993P | gl_28898735 |
| 219 | MRT4577_287993P | gl_16079320 |
| 219 | MRT4577_287993P | gl_15606687 |
| 219 | MRT4577_287993P | gl_11499192 |
| 219 | MRT4577_287993P | gl_136258 |
| 219 | MRT4577_287993P | gl_27375857 |
| 219 | MRT4577_287993P | gl_17988302 |
| 219 | MRT4577_287993P | gl_27904753 |
| 219 | MRT4577_287993P | gl_29345937 |
| 219 | MRT4577_287993P | gl_30019391 |
| 219 | MRT4577_287993P | gl_29654461 |
| 219 | MRT4577_287993P | gl_29832720 |
| 219 | MRT4577_287993P | gl_29840325 |
| 219 | MRT4577_287993P | gl_32034755 |
| 219 | MRT4577_287993P | gl_32029713 |
| 219 | MRT4577_287993P | gl_30248703 |
| 219 | MRT4577_287993P | gl_15897777 |
| 219 | MRT4577_287993P | gl_1004320 |
| 219 | MRT4577_287993P | gl_15642911 |
| 219 | MRT4577_287993P | gl_2120372 |
| 219 | MRT4577_287993P | gl_94733 |
| 219 | MRT4577_287993P | gl_136266 |
| 219 | MRT4577_287993P | gl_401211 |
| 219 | MRT4577_287993P | gl_541528 |
| 219 | MRT4577_287993P | gl_409778 |
| 219 | MRT4577_287993P | gl_3915890 |
| 219 | MRT4577_287993P | gl_11465459 |
| 219 | MRT4577_287993P | gl_11465848 |
| 219 | MRT4577_287993P | gl_27262488 |
| 219 | MRT4577_287993P | gl_28380214 |
| 219 | MRT4577_287993P | gl_23053574 |
| 219 | MRT4577_287993P | gl_136259 |
| 219 | MRT4577_287993P | gl_151617 |
| 219 | MRT4577_287993P | gl_68332 |
| 219 | MRT4577_287993P | gl_14520674 |
| 219 | MRT4577_287993P | gl_5834682 |
| 219 | MRT4577_287993P | gl_28380199 |
| 219 | MRT4577_287993P | gl_136264 |
| 219 | MRT4577_287993P | gl_17546700 |
| 219 | MRT4577_287993P | gl_28380179 |
| 219 | MRT4577_287993P | gl_464911 |
| 219 | MRT4577_287993P | gl_23103063 |
| 219 | MRT4577_287993P | gl_15235430 |
| 219 | MRT4577_287993P | gl_21593559 |
| 219 | MRT4577_287993P | gl_18410104 |
| 219 | MRT4577_287993P | gl_32441888 |
| 219 | MRT4577_287993P | gl_13474231 |
| 219 | MRT4577_287993P | gl_15963782 |
| 219 | MRT4577_287993P | MRT3847_51771P.3 |
| 219 | MRT4577_287993P | MRT3847_243747P.2 |
| 219 | MRT4577_287993P | MRT3847_242965P.2 |
| 219 | MRT4577_287993P | gl_31126752 |
| 219 | MRT4577_287993P | gl_31126747 |
| 219 | MRT4577_287993P | gl_31126749 |
| 219 | MRT4577_287993P | gl_2541878 |
| 219 | MRT4577_287993P | gl_30468052 |
| 219 | MRT4577_287993P | MRT4530_41051P.1 |
| 219 | MRT4577_287993P | MRT4530_19284P.1 |
| 219 | MRT4577_287993P | MRT4530_19282P.1 |
| 219 | MRT4577_287993P | MRT4565_24270P.3 |
| 219 | MRT4577_287993P | MRT4565_3598P.3 |
| 219 | MRT4577_287993P | MRT4565_51329P.3 |
| 219 | MRT4577_287993P | MRT4565_9194P.2 |
| 219 | MRT4577_287993P | MRT4565_6744P.2 |
| 219 | MRT4577_287993P | MRT4565_25946P.3 |
| 219 | MRT4577_287993P | MRT4565_131929P.1 |
| 219 | MRT4577_287993P | MRT4565_28703P.3 |
| 219 | MRT4577_287993P | MRT4565_38061P.3 |
| 219 | MRT4577_287993P | MRT4565_123153P.1 |
| 219 | MRT4577_287993P | MRT4565_118038P.1 |
| 219 | MRT4577_287993P | MRT4565_26535P.2 |
| 219 | MRT4577_287993P | MRT4565_52146P.2 |
| 219 | MRT4577_287993P | MRT4565_115300P.1 |
| 219 | MRT4577_287993P | MRT4565_53782P.2 |
| 219 | MRT4577_287993P | MRT4565_16589P.2 |
| 219 | MRT4577_287993P | MRT4565_113424P.1 |
| 219 | MRT4577_287993P | MRT4565_104502P.1 |
| 219 | MRT4577_287993P | MRT4565_23334P.2 |
| 219 | MRT4577_287993P | gl_23108488 |
| 219 | MRT4577_287993P | gl_23113700 |
| 219 | MRT4577_287993P | gl_23115534 |
| 219 | MRT4577_287993P | gl_775193 |
| 219 | MRT4577_287993P | gl_775168 |
| 219 | MRT4577_287993P | gl_775181 |
| 219 | MRT4577_287993P | gl_775198 |
| 219 | MRT4577_287993P | gl_775174 |
| 219 | MRT4577_287993P | gl_775154 |
| 219 | MRT4577_287993P | gl_20136097 |
| 219 | MRT4577_287993P | gl_20136089 |
| 219 | MRT4577_287993P | gl_20136099 |
| 219 | MRT4577_287993P | gl_20136095 |
| 219 | MRT4577_287993P | gl_20136093 |
| 219 | MRT4577_287993P | gl_20136103 |
| 219 | MRT4577_287993P | gl_14602140 |
| 219 | MRT4577_287993P | gl_68331 |
| 219 | MRT4577_287993P | gl_23122427 |
| 219 | MRT4577_287993P | gl_11513797 |
| 219 | MRT4577_287993P | gl_3212365 |
| 219 | MRT4577_287993P | gl_28373459 |
| 219 | MRT4577_287993P | gl_28373461 |
| 219 | MRT4577_287993P | gl_2098385 |
| 219 | MRT4577_287993P | gl_20135991 |
| 219 | MRT4577_287993P | gl_20135995 |
| 219 | MRT4577_287993P | gl_20135989 |
| 219 | MRT4577_287993P | gl_20136101 |
| 219 | MRT4577_287993P | gl_20136015 |
| 219 | MRT4577_287993P | gl_20136003 |
| 219 | MRT4577_287993P | gl_20135993 |
| 219 | MRT4577_287993P | gl_20136013 |
| 219 | MRT4577_287993P | gl_20136059 |
| 219 | MRT4577_287993P | gl_20136051 |
| 219 | MRT4577_287993P | gl_20136053 |
| 219 | MRT4577_287993P | gl_20136047 |
| 219 | MRT4577_287993P | gl_20136057 |
| 219 | MRT4577_287993P | gl_20136045 |
| 219 | MRT4577_287993P | gl_20136041 |
| 219 | MRT4577_287993P | gl_20136043 |
| 219 | MRT4577_287993P | gl_20136049 |
| 219 | MRT4577_287993P | gl_20136035 |
| 219 | MRT4577_287993P | gl_20136019 |
| 219 | MRT4577_287993P | gl_20136029 |
| 219 | MRT4577_287993P | gl_20136033 |
| 219 | MRT4577_287993P | gl_20136039 |
| 219 | MRT4577_287993P | gl_20136075 |
| 219 | MRT4577_287993P | gl_20136067 |
| 219 | MRT4577_287993P | gl_20136063 |
| 219 | MRT4577_287993P | gl_20136065 |
| 219 | MRT4577_287993P | gl_20136073 |
| 219 | MRT4577_287993P | gl_20136069 |
| 219 | MRT4577_287993P | gl_23128273 |
| 219 | MRT4577_287993P | gl_23124896 |
| 219 | MRT4577_287993P | gl_14574707 |
| 219 | MRT4577_287993P | gl_16554463 |
| 219 | MRT4577_287993P | gl_25409314 |
| 219 | MRT4577_287993P | gl_15641182 |
| 219 | MRT4577_287993P | gl_48491 |
| 219 | MRT4577_287993P | gl_7674396 |
| 219 | MRT4577_287993P | gl_16273337 |
| 219 | MRT4577_287993P | gl_23131139 |
| 219 | MRT4577_287993P | gl_15602442 |
| 219 | MRT4577_287993P | gl_136261 |
| 219 | MRT4577_287993P | gl_20805995 |
| 219 | MRT4577_287993P | gl_15604890 |
| 219 | MRT4577_287993P | gl_20805967 |
| 219 | MRT4577_287993P | gl_20805971 |
| 219 | MRT4577_287993P | gl_20805999 |
| 219 | MRT4577_287993P | gl_20805979 |
| 219 | MRT4577_287993P | gl_6599049 |
| 219 | MRT4577_287993P | gl_6599047 |
| 219 | MRT4577_287993P | gl_15608751 |
| 219 | MRT4577_287993P | gl_16129221 |
| 219 | MRT4577_287993P | gl_23133994 |
| 219 | MRT4577_287993P | gl_15645891 |
| 219 | MRT4577_287993P | gl_15612263 |
| 219 | MRT4577_287993P | gl_32130302 |
| 219 | MRT4577_287993P | gl_15614227 |
| 219 | MRT4577_287993P | gl_28971666 |
| 219 | MRT4577_287993P | gl_16760154 |
| 219 | MRT4577_287993P | gl_32423711 |
| 219 | MRT4577_287993P | gl_23137115 |
| 219 | MRT4577_287993P | gl_16765071 |

TABLE 5

| Seq_Num | Seq_ID | Organism_Name |
|---|---|---|
| 220 | gl_27366338 | Vibrio vulnificus CMCP6 |
| 221 | gl_22991721 | Enterococcus faecium |
| 222 | gl_15425588 | Pentaphragma ellipticum |
| 223 | gl_15897860 | Sulfolobus solfataricus |
| 224 | gl_23037705 | Oenococcus oeni MCW |
| 225 | gl_16081190 | Thermoplasma acidophilum |
| 226 | gl_15888589 | Agrobacterium tumefaciens str. C58 (Cereon) |
| 227 | gl_14718201 | Quiina pteridophylla |
| 228 | MRT4530_27655P.2 | Oryza sativa |
| 229 | gl_4063556 | Ochroma pyramidale |
| 230 | gl_32473774 | Pirellula sp. |
| 231 | gl_15603842 | Pasteurella multocida |
| 232 | gl_5596996 | Sorghum bicolor |
| 233 | gl_14718165 | Pedicularis coronata |
| 234 | gl_23055438 | Geobacter metallireducens |
| 235 | gl_23006404 | Magnetospirillum magnetotacticum |
| 236 | gl_4995103 | Cola nitida |
| 237 | gl_5231187 | Streptococcus pneumoniae |
| 238 | gl_20807813 | Thermoanaerobacter tengcongensis |
| 239 | gl_30230270 | Ginkgo biloba |
| 240 | gl_3850934 | Carnarvonia araliifolia |
| 241 | gl_26517024 | Brassica rapa subsp. pekinensis |
| 242 | gl_15422208 | Argophyllum sp. Telford 5462 |
| 243 | gl_22994339 | Xylella fastidiosa Dixon |
| 244 | MRT3847_12543P.1 | Glycine max |
| 245 | gl_29420859 | Saccharomyces dairenensis |
| 246 | gl_7594817 | Salmonella typhimurium |
| 247 | gl_23099057 | Oceanobacillus iheyensis HTE831 |
| 248 | gl_19553586 | Corynebacterium glutamicum ATCC 13032 |
| 249 | gl_4731151 | Berzelia lanuglnosa |
| 250 | gl_28380179 | Synechococcus sp. PCC 7002 |
| 251 | gl_22961512 | Rhodopseudomonas palustris |
| 252 | gl_11071974 | Nicotiana tabacum |
| 253 | gl_775174 | Escherichia coli |
| 254 | gl_15890531 | Agrobacterium tumefaciens str. C58 (Cereon) |
| 255 | gl_23021869 | Clostridium thermocellum ATCC 27405 |
| 256 | gl_12004151 | Primula gaubaeana |
| 257 | gl_28378548 | Lactobacillus plantarum WCFS1 |
| 258 | gl_10120912 | Ipomoea batatas |
| 259 | gl_11358184 | Arabidopsis thaliana |
| 260 | gl_7339715 | Oryza sativa (japonica cultivar-group) |
| 261 | gl_7676173 | Methanocaldococcus jannaschii |
| 262 | gl_20136063 | Shigella sonnei |
| 263 | gl_7488791 | Pisum sativum |
| 264 | gl_28564960 | Saccharomyces kluyveri |
| 265 | gl_16330679 | Synechocystis sp. PCC 6803 |
| 266 | gl_30263833 | Bacillus anthracis str. Ames |
| 267 | gl_5758908 | Riedelia aff. wrayii SBG 83-203 |
| 268 | gl_18390357 | Bacillus subtilis |
| 269 | gl_1929027 | Beta vulgaris |
| 270 | MRT4530_111084P.2 | Oryza sativa |
| 271 | gl_416619 | Ipomoea batatas |
| 272 | gl_4101710 | Pinus resinosa |
| 273 | gl_4063522 | Acer saccharum |
| 274 | gl_21910448 | Streptococcus pyogenes MGAS315 |
| 275 | gl_28380195 | Agrobacterium tumefaciens str. C58 |
| 276 | gl_17227907 | Nostoc sp. PCC 7120 |
| 277 | gl_15793205 | Neisseria meningltidis Z2491 |
| 278 | gl_25386572 | Arabidopsis thaliana |
| 279 | gl_7708499 | Morus nigra |
| 280 | gl_28564948 | Saccharomyces kluyveri |
| 281 | gl_586209 | Candida tropicalis |
| 282 | gl_23017722 | Thermobifida fusca |
| 283 | gl_22537459 | Streptococcus agalactiae 2603V/R |
| 284 | gl_8918271 | Pisum sativum |
| 285 | gl_27262322 | Heliobacillus mobilis |
| 286 | gl_21536979 | Arabidopsis thaliana |
| 287 | gl_15837426 | Xylella fastidiosa 9a5c |
| 288 | gl_28572441 | Tropheryma whipplei TW08/27 |
| 289 | gl_8452749 | Simarouba glauca |
| 290 | gl_1352828 | Cyanidium caldarium |
| 291 | gl_15810901 | Antirrhinum majus subsp. cirrhigerum |
| 292 | gl_14718111 | Lilium superbum |
| 293 | gl_14627128 | Solanum tuberosum |
| 294 | gl_14717933 | Ancistrocladus korupensis |
| 295 | gl_28373461 | Salmonella typhimurium |
| 296 | gl_1742753 | Escherichia coli |
| 297 | MRT4530_15443P.1 | Oryza sativa |
| 298 | gl_6689056 | Paulownia tomentosa |
| 299 | gl_27435914 | Welwitschia mirabilis |
| 300 | MRT4530_81676P.1 | Oryza sativa |
| 301 | gl_608673 | Arabidopsis thaliana |
| 302 | gl_28493257 | Tropheryma whipplei str. Twist |
| 303 | gl_23026706 | Microbulbifer degradans 2-40 |
| 304 | gl_22994632 | Xylella fastidiosa Dixon |
| 305 | gl_1067169 | Petunia x hybrida |
| 306 | gl_3850936 | Sphalmium racemosum |
| 307 | gl_7447977 | Cucumis sativus |
| 308 | gl_136262 | Methanococcus voltae |
| 309 | gl_21954721 | Mesotaenium caldariorum |
| 310 | gl_6782440 | Nicotiana glauca |
| 311 | gl_22128587 | Petunia x hybrida |
| 312 | gl_15805966 | Deinococcus radiodurans |
| 313 | gl_21402518 | Bacillus anthracis str. A2012 |
| 314 | gl_6634078 | Citrus x paradisi |
| 315 | gl_19033089 | Klebsormidium flaccidum |
| 316 | gl_2462107 | Bacillus cereus |
| 317 | gl_20136101 | Shigella boydii |
| 318 | MRT3847_85245P.2 | Glycine max |
| 319 | gl_401211 | Antithamnion sp. |
| 320 | gl_21220496 | Streptomyces coelicolor A3(2) |
| 321 | MRT4530_41051P.1 | Oryza sativa |
| 322 | gl_21633361 | Seddera hirsuta |
| 323 | gl_23005242 | Magnetospirillum magnetotacticum |
| 324 | MRT3847_36311P.3 | Glycine max |
| 325 | gl_12585416 | Borrelia burgdorferi |
| 326 | gl_7708272 | Dichapetalum brownii |
| 327 | gl_29373125 | Citrus sinensis |
| 328 | gl_322801 | Antirrhinum majus |
| 329 | gl_23099532 | Oceanobacillus iheyensis HTE831 |
| 330 | MRT4565_77691P.2 | Triticum aestivum |
| 331 | gl_2493123 | Hordeum vulgare |
| 332 | gl_7489168 | Nicotiana tabacum |
| 333 | gl_15903673 | Streptococcus pneumoniae R6 |
| 334 | gl_16416730 | Equisetum x ferrissii |
| 335 | gl_16126204 | Caulobacter crescentus CB15 |
| 336 | gl_23108079 | Novosphingobium aromaticivorans |
| 337 | gl_22968361 | Rhodospirillum rubrum |
| 338 | gl_20135995 | Shigella boydii |
| 339 | gl_15828368 | Mycobacterium leprae |
| 340 | gl_4995221 | Hibiscus punaluuensis |
| 341 | gl_4063524 | Aesculus pavia |
| 342 | gl_14718265 | Xanthoceras sorbifolium |
| 343 | gl_19114337 | Schizosaccharomyces pombe |
| 344 | gl_21633433 | Erycibe glomerata |
| 345 | gl_7708284 | Erythroxylum confusum |
| 346 | gl_25308880 | Arabidopsis thaliana |
| 347 | gl_31540632 | Brassica napus |
| 348 | gl_22994398 | Xylella fastidiosa Dixon |
| 349 | gl_21633349 | Hildebrandtia valo |
| 350 | gl_15150341 | Camellia sinensis |
| 351 | gl_20259460 | Arabidopsis thaliana |
| 352 | gl_4995053 | Adansonia rubrostipa |
| 353 | MRT3847_13189P.3 | Glycine max |
| 354 | gl_15237549 | Arabidopsis thaliana |
| 355 | gl_3850966 | Euplassa inaequalis |
| 356 | gl_7708189 | Carpenteria californica |
| 357 | gl_22651734 | Drosophyllum lusitanicum |
| 358 | gl_4995097 | Durio zibethinus |
| 359 | gl_16943668 | Caesia contorta |
| 360 | MRT3847_41566P.3 | Glycine max |
| 361 | gl_15887403 | Agrobacterium tumefaciens str. C58 (Cereon) |
| 362 | gl_28378738 | Lactobacillus plantarum WCFS1 |
| 363 | gl_4586602 | Cicer arietinum |
| 364 | MRT3847_253605P.2 | Glycine max |
| 365 | gl_6970417 | Rosa rugosa |
| 366 | MRT4530_81446P.2 | Oryza sativa |
| 367 | gl_14718232 | Stellaria media |
| 368 | gl_24940162 | Borago officinalis |
| 369 | MRT4565_29431P.3 | Triticum aestivum |
| 370 | MRT4530_97319P.2 | Oryza sativa |
| 371 | gl_16417186 | Saccharomyces sp. DH1-1A |
| 372 | gl_20136095 | Escherichia coli |
| 373 | gl_14717935 | Androstachys johnsonii |
| 374 | gl_23503621 | Carteria cerasiformis |
| 375 | gl_21741785 | Oryza sativa (japonica cultivar-group) |
| 376 | gl_13506709 | Lycopersicon esculentum |
| 377 | gl_27526583 | Kluyveromyces dobzhanskii |
| 378 | gl_21672587 | Buchnera aphidicola str. Sg (Schizaphis graminum) |
| 379 | MRT3847_63803P.3 | Glycine max |
| 380 | gl_14573437 | Chlamydomonas reinhardtii |
| 381 | gl_6822147 | Hieracium piloselloides |
| 382 | gl_22128589 | Petunia x hybrida |
| 383 | gl_15615026 | Bacillus halodurans |
| 384 | gl_3900936 | Cicer arietinum |
| 385 | gl_31126752 | Oryza sativa (japonica cultivar-group) |
| 386 | gl_7708568 | Quisqualis indica |
| 387 | gl_1084399 | Lycopersicon esculentum |
| 388 | MRT4565_98294P.2 | Triticum aestivum |
| 389 | gl_4850214 | Lycopersicon esculentum |
| 390 | gl_19033059 | Nitella opaca |
| 391 | gl_15841981 | Mycobacterium tuberculosis CDC1551 |
| 392 | MRT3847_265345P.2 | Glycine max |
| 393 | gl_15223930 | Arabidopsis thaliana |
| 394 | MRT3847_35167P.2 | Glycine max |
| 395 | gl_23111624 | Desulfitobacterium hafniense |
| 396 | gl_15893920 | Clostridium acetobutylicum |
| 397 | gl_20384961 | Coleochaete sp. 18a1 |
| 398 | gl_22959136 | Rhodobacter sphaeroides |
| 399 | gl_1171780 | Enterococcus hirae |
| 400 | gl_28572631 | Tropheryma whipplei TW08/27 |
| 401 | gl_24940184 | Emmenanthe penduliflora |
| 402 | gl_23063854 | Pseudomonas fluorescens PfO-1 |
| 403 | gl_4995794 | Rulingla sp. Chase 2196 |
| 404 | gl_4033721 | Picea mariana |
| 405 | gl_18312204 | Pyrobaculum aerophilum str. IM2 |
| 406 | gl_21684869 | Anarthria scabra |
| 407 | gl_15831818 | Escherichia coli O157:H7 |
| 408 | gl_388977 | Escherichia coli |
| 409 | gl_6984231 | Euphorbia esula |
| 410 | MRT3847_255937P.2 | Glycine max |
| 411 | MRT3847_284959P.1 | Glycine max |
| 412 | MRT4565_51329P.3 | Triticum aestivum |
| 413 | gl_7488484 | Brassica napus |
| 414 | gl_21231972 | Xanthomonas campestris pv. campestris str. |
| | | ATCC 33913 |
| 415 | gl_23021511 | Clostridium thermocellum ATCC 27405 |
| 416 | MRT4565_24817P.3 | Triticum aestivum |
| 417 | gl_14717997 | Celosia argentea |
| 418 | gl_28188341 | Coleochaete sp. 528a3 |
| 419 | gl_29420865 | Saccharomyces unisporus |
| 420 | gl_22961554 | Rhodopseudomonas palustris |
| 421 | MRT3847_64874P.3 | Glycine max |
| 422 | gl_32475580 | Pirellula sp. |
| 423 | MRT3847_286535P.1 | Glycine max |
| 424 | gl_16800375 | Listeria innocua |
| 425 | gl_217936 | Ipomoea batatas |
| 426 | gl_4731153 | Dillenia retusa |
| 427 | gl_15612353 | Helicobacter pylori J99 |
| 428 | gl_16803610 | Listeria monocytogenes EGD-e |
| 429 | gl_29347953 | Bacteroides thetaiotaomicron VPI-5482 |
| 430 | gl_25004882 | Cicer arietinum |
| 431 | gl_48491 | Vibrio parahaemolyticus |
| 432 | MRT3847_239034P.2 | Glycine max |
| 433 | gl_22406531 | Ferroplasma acidarmanus |
| 434 | MRT4565_107456P.1 | Triticum aestivum |
| 435 | gl_12004153 | Primula palinuri |
| 436 | gl_15810509 | Arabidopsis thaliana |
| 437 | gl_19920171 | Oryza sativa (japonica cultivar-group) |
| 438 | gl_14718042 | Epilobium angustifolium |
| 439 | gl_19115258 | Schizosaccharomyces pombe |
| 440 | gl_7708512 | Planchonella pohlmaniana |
| 441 | gl_23021249 | Clostridium thermocellum ATCC 27405 |
| 442 | MRT3847_26155P.3 | Glycine max |
| 443 | gl_19033091 | Klebsormidium subtilissimum |
| 444 | gl_28188329 | Coleochaete sp. 327d3 |
| 445 | gl_4469175 | Hevea brasiliensis |
| 446 | gl_13548679 | Pyrus pyrifolia |
| 447 | gl_4995788 | Rhopalocarpus sp. Chase 906 |
| 448 | gl_15605757 | Aquifex aeolicus VF5 |
| 449 | gl_17545291 | Ralstonia solanacearum |
| 450 | gl_16803667 | Listeria monocytogenes EGD-e |
| 451 | gl_15216026 | Vicia faba var. minor |
| 452 | MRT3847_200246P.2 | Glycine max |
| 453 | gl_18077607 | Valdivia gayana |
| 454 | gl_15615614 | Bacillus halodurans |
| 455 | gl_23000020 | Magnetococcus sp. MC-1 |
| 456 | gl_14717931 | Allium altaicum |
| 457 | MRT4530_135930P.1 | Oryza sativa |
| 458 | gl_6689562 | Verbascum thapsus |
| 459 | gl_775154 | Escherichia coli |
| 460 | gl_27528500 | Torulaspora delbrueckii |
| 461 | gl_23099102 | Oceanobacillus iheyensis HTE831 |
| 462 | gl_172907 | Saccharomyces cerevisiae |
| 463 | MRT3847_70323P.2 | Glycine max |
| 464 | gl_9955367 | Escherichia coli |
| 465 | gl_7442734 | Ricinus communis |
| 466 | gl_22993136 | Enterococcus faecium |
| 467 | gl_21243443 | Xanthomonas axonopodis pv. citri str. 306 |
| 468 | gl_21221074 | Streptomyces coelicolor A3(2) |
| 469 | gl_15611004 | Mycobacterium tuberculosis H37Rv |
| 470 | gl_6320016 | Saccharomyces cerevisiae |
| 471 | MRT3847_44128P.3 | Glycine max |
| 472 | gl_5869971 | Scherffelia dubia |
| 473 | gl_14718072 | Heteropyxis natalensis |
| 474 | gl_32034755 | Actinobacillus pleuropneumoniae serovar 1 str. 4074 |
| 475 | gl_4033435 | Agrobacterium vitis |
| 476 | gl_9229839 | Thermoplasma acidophilum |
| 477 | gl_21616113 | Cucumis melo |
| 478 | gl_2459981 | Pseudomonas aeruglnosa |
| 479 | gl_15826905 | Mycobacterium leprae |
| 480 | gl_15242176 | Arabidopsis thaliana |
| 481 | MRT3847_267642P.1 | Glycine max |
| 482 | gl_8517408 | Clavija eggersiana |
| 483 | gl_15829106 | Mycoplasma pulmonis |
| 484 | gl_20135993 | Shigella boydii |
| 485 | gl_2497537 | Asperglllus niger |
| 486 | gl_25010985 | Streptococcus agalactiae NEM316 |
| 487 | gl_100287 | Nicotiana sp. |
| 488 | gl_21633431 | Erycibe hellwigli |
| 489 | gl_15614852 | Bacillus halodurans |
| 490 | MRT3847_30045P.3 | Glycine max |
| 491 | MRT4530_8279P.1 | Oryza sativa |
| 492 | gl_6323275 | Saccharomyces cerevisiae |
| 493 | gl_116144 | Xanthobacter flavus |
| 494 | gl_23043296 | Trichodesmium erythraeum IMS101 |
| 495 | gl_3850964 | Cardwellia sublimis |
| 496 | gl_16765661 | Salmonella typhimurium LT2 |
| 497 | gl_3850900 | Bellendena montana |
| 498 | gl_23137026 | Cytophaga hutchinsonii |
| 499 | gl_23103564 | Azotobacter vinelandii |
| 500 | gl_22964886 | Rhodopseudomonas palustris |
| 501 | gl_15924244 | Staphylococcus aureus subsp. aureus Mu50 |
| 502 | gl_14718230 | Spigelia marilandica |
| 503 | gl_7592738 | Nepenthes alata |
| 504 | MRT4530_109505P.2 | Oryza sativa |
| 505 | gl_27447653 | Lycopersicon esculentum |
| 506 | gl_7484972 | Arabidopsis thaliana |
| 507 | gl_32490903 | Wigglesworthia glossinidia endosymbiont |
| | | of Glossina brevipalpis |
| 508 | gl_10241425 | Oryza sativa (indica cultivar-group) |
| 509 | gl_21633419 | Dicranostyles villosus |
| 510 | gl_5758884 | Hedychium flavum |
| 511 | gl_15594640 | Borrelia burgdorferi B31 |
| 512 | gl_24940204 | Hydrolea sp. Chase 3245 |
| 513 | gl_20136093 | Escherichia coli |
| 514 | gl_12585563 | Methanocaldococcus jannaschii |
| 515 | gl_23336124 | Bifidobacterium longum DJO10A |
| 516 | gl_6017814 | Nelumbo lutea |
| 517 | gl_7708308 | Garrya elliptica |
| 518 | gl_15866696 | Avena strigosa |
| 519 | gl_7708339 | Hymenanthera alpina |
| 520 | gl_26553530 | Mycoplasma penetrans |
| 521 | gl_12585391 | Desulfurococcus sp. SY |
| 522 | gl_584810 | Galdieria sulphuraria |
| 523 | gl_15642920 | Thermotoga maritima |
| 524 | gl_23465101 | Bifidobacterium longum NCC2705 |
| 525 | MRT4565_52855P.3 | Triticum aestivum |
| 526 | gl_23058851 | Pseudomonas fluorescens PfO-1 |
| 527 | gl_21223783 | Streptomyces coelicolor A3(2) |
| 528 | gl_4063552 | Muntingla calabura |
| 529 | gl_15924687 | Staphylococcus aureus subsp. aureus Mu50 |
| 530 | gl_136259 | Klebsiella aerogenes |
| 531 | MRT4565_39839P.3 | Triticum aestivum |
| 532 | gl_21672546 | Buchnera aphidicola str. Sg (Schizaphis graminum) |
| 533 | gl_7708181 | Betula pendula |
| 534 | gl_23136411 | Cytophaga hutchinsonii |
| 535 | gl_2541878 | Cyanidioschyzon merolae |
| 536 | gl_7708177 | Brexia madagascariensis |
| 537 | gl_7436320 | Desulfurococcus mobilis |
| 538 | gl_15921725 | Sulfolobus tokodaii |
| 539 | gl_23019853 | Thermobifida fusca |
| 540 | gl_21232615 | Xanthomonas campestris pv. campestris |
| | | str. ATCC 33913 |
| 541 | gl_16127773 | Caulobacter crescentus CB15 |
| 542 | gl_21684925 | Leersia oryzoides |
| 543 | gl_12004121 | Cortusa turkestanica |
| 544 | gl_19705311 | Fusobacterium nucleatum subsp. |
| | | nucleatum ATCC 25586 |
| 545 | gl_7708634 | Sambucus nigra |
| 546 | gl_15425590 | Phyllachne uliglnosa |
| 547 | gl_27375857 | Bradyrhizobium japonicum USDA 110 |
| 548 | gl_17232340 | Nostoc sp. PCC 7120 |
| 549 | gl_22989508 | Burkholderia fungorum |
| 550 | gl_12004143 | Jacquinia keyensis |
| 551 | gl_24940244 | Pisum sativum |
| 552 | gl_27467972 | Staphylococcus epidermidis ATCC 12228 |
| 553 | gl_30351915 | Periboea paucifolia |
| 554 | gl_68332 | Pseudomonas aeruglnosa |
| 555 | gl_8452704 | Nomocharis pardanthina |
| 556 | gl_15892357 | Rickettsia conorii |
| 557 | gl_15609923 | Mycobacterium tuberculosis H37Rv |
| 558 | gl_28897130 | Vibrio parahaemolyticus RIMD 2210633 |
| 559 | gl_4033428 | Photobacterium leiognathi |
| 560 | gl_1730064 | Bacillus licheniformis |
| 561 | gl_7674377 | Buchnera aphidicola (Diuraphis noxia) |
| 562 | gl_15827378 | Mycobacterium leprae |
| 563 | MRT3847_227267P.3 | Glycine max |
| 564 | MRT4530_84009P.2 | Oryza sativa |
| 565 | gl_23023645 | Leuconostoc mesenteroides subsp. |
| | | mesenteroides ATCC 8293 |
| 566 | gl_6688704 | Myoporum mauritianum |
| 567 | MRT4565_91331P.2 | Triticum aestivum |
| 568 | gl_16081945 | Thermoplasma acidophilum |
| 569 | gl_20136047 | Shigella dysenteriae |
| 570 | gl_29420871 | Saccharomyces pastorianus |
| 571 | gl_20091808 | Methanosarcina acetivorans C2A |
| 572 | gl_7708514 | Napoleonaea vogelii |
| 573 | gl_4206588 | Atalantia ceylanica |
| 574 | gl_32488077 | Oryza sativa (japonica cultivar-group) |
| 575 | gl_15837977 | Xylella fastidiosa 9a5c |
| 576 | gl_22330789 | Arabidopsis thaliana |
| 577 | gl_2274776 | Candida albicans |
| 578 | gl_22957596 | Rhodobacter sphaeroides |
| 579 | gl_3122311 | Methylobacterium extorquens |
| 580 | gl_30692988 | Arabidopsis thaliana |
| 581 | gl_12039387 | Oryza sativa (japonica cultivar-group) |
| 582 | gl_24940176 | Echiochilon collenettei |
| 583 | MRT3847_250868P.2 | Glycine max |
| 584 | gl_24414622 | Helianthus annuus |
| 585 | gl_231540 | Secale cereale |
| 586 | gl_21633379 | Stylisma patens |
| 587 | gl_23017104 | Thermobifida fusca |
| 588 | gl_6017810 | Limeum sp. Hoot 983 |
| 589 | gl_22998791 | Magnetococcus sp. MC-1 |
| 590 | gl_264676 | Saccharomyces cerevisiae |
| 591 | gl_1352326 | Brassica rapa |
| 592 | MRT3847_48429P.3 | Glycine max |
| 593 | gl_16416758 | Polytrichum pallidisetum |
| 594 | gl_22298059 | Thermosynechococcus elongatus BP-1 |
| 595 | gl_5231208 | Streptococcus pneumoniae |
| 596 | gl_20807120 | Thermoanaerobacter tengcongensis |
| 597 | gl_23131139 | Prochlorococcus marinus str. MIT 9313 |
| 598 | MRT3847_234305P.2 | Glycine max |
| 599 | MRT4565_115300P.1 | Triticum aestivum |
| 600 | gl_5758889 | Heliconia rostrata |
| 601 | gl_23131322 | Prochlorococcus marinus str. MIT 9313 |
| 602 | gl_23097976 | Oceanobacillus iheyensis HTE831 |
| 603 | gl_7688031 | Peltoboykinia tellimoides |
| 604 | gl_6319279 | Saccharomyces cerevisiae |
| 605 | gl_32418640 | Neurospora crassa |
| 606 | gl_23111737 | Desulfitobacterium hafniense |
| 607 | gl_32490757 | Wigglesworthia glossinidia endosymbiont of |
| | | Glossina brevipalpis |
| 608 | gl_7687974 | Degeneria vitiensis |
| 609 | gl_15676576 | Neisseria meningltidis MC58 |
| 610 | gl_6634488 | Poncirus trifoliata |
| 611 | gl_7452979 | Hordeum vulgare subsp. vulgare |
| 612 | gl_29420851 | Saccharomyces cerevisiae |
| 613 | gl_17827467 | Petunia x hybrida |
| 614 | gl_32476398 | Pirellula sp. |
| 615 | gl_6633813 | Arabidopsis thaliana |
| 616 | gl_26988777 | Pseudomonas putida KT2440 |
| 617 | gl_28209952 | Clostridium tetani E88 |
| 618 | gl_21667496 | Cycas edentata |
| 619 | gl_23014985 | Magnetospirillum magnetotacticum |
| 620 | MRT4530_143108P.1 | Oryza sativa |
| 621 | gl_16903129 | Sambucus nigra |
| 622 | gl_20135991 | Shigella boydii |
| 623 | MRT4530_35848P.1 | Oryza sativa |
| 624 | gl_5758888 | Heliconia paka |
| 625 | gl_15828737 | Mycoplasma pulmonis |
| 626 | gl_16803319 | Listeria monocytogenes EGD-e |
| 627 | gl_15801918 | Escherichia coli O157:H7 EDL933 |
| 628 | gl_15793848 | Neisseria meningltidis Z2491 |
| 629 | gl_29655069 | Coxiella burnetii RSA 493 |
| 630 | gl_20149296 | Malus x domestica |
| 631 | MRT4565_104372P.1 | Triticum aestivum |
| 632 | gl_15233810 | Arabidopsis thaliana |
| 633 | gl_5758854 | Aloe vera |
| 634 | gl_15677237 | Neisseria meningltidis MC58 |
| 635 | gl_20136049 | Shigella dysenteriae |
| 636 | gl_5231190 | Streptococcus pneumoniae |
| 637 | gl_22094360 | Oryza sativa (japonica cultivar-group) |
| 638 | gl_32029324 | Haemophilus somnus 2336 |
| 639 | gl_7488485 | Brassica napus |
| 640 | gl_15675235 | Streptococcus pyogenes M1 GAS |
| 641 | gl_23335097 | Bifidobacterium longum DJO10A |
| 642 | gl_28140043 | Elaeis guineensis |
| 643 | gl_6539602 | Vicia faba |
| 644 | gl_775198 | Escherichia coli |
| 645 | gl_20092686 | Methanosarcina acetivorans C2A |
| 646 | gl_21633417 | Jacquemontia reclinata |
| 647 | gl_15805727 | Deinococcus radiodurans |
| 648 | gl_30468060 | Cyanidioschyzon merolae |
| 649 | gl_18310529 | Clostridium perfringens str. 13 |
| 650 | gl_6681366 | Pisum sativum |
| 651 | gl_28202179 | Anthoceros formosae |
| 652 | gl_29832759 | Streptomyces avermitilis MA-4680 |
| 653 | gl_15640512 | Vibrio cholerae |
| 654 | gl_3377757 | Zymomonas mobilis |
| 655 | gl_15887378 | Agrobacterium tumefaciens str. C58 (Cereon) |
| 656 | MRT3847_37580P.3 | Glycine max |
| 657 | gl_1430917 | Ochrosphaera neapolitana |
| 658 | gl_15606687 | Aquifex aeolicus VF5 |
| 659 | gl_1084400 | Lycopersicon esculentum |
| 660 | gl_2497540 | Ricinus communis |
| 661 | gl_27884018 | Lycopersicon esculentum |
| 662 | gl_8980815 | Castanea sativa |
| 663 | gl_23502605 | Brucella suis 1330 |
| 664 | gl_4063550 | Helianthemum grandiflorum |
| 665 | gl_22977198 | Ralstonia metallidurans |
| 666 | gl_15645891 | Helicobacter pylori 26695 |
| 667 | gl_7688421 | Viscainoa geniculata |
| 668 | gl_15614926 | Bacillus halodurans |
| 669 | gl_1196314 | Borrelia burgdorferi |
| 670 | gl_29654461 | Coxiella burnetii RSA 493 |
| 671 | gl_8918273 | Pisum sativum |
| 672 | gl_19075895 | Schizosaccharomyces pombe |
| 673 | gl_11357139 | Chenopodium rubrum |
| 674 | gl_5758886 | Heliconia irrasa |
| 675 | gl_15673444 | Lactococcus lactis subsp. lactis |
| 676 | gl_6686963 | Barleria prionitis |
| 677 | gl_6016879 | Bacillus sp. |
| 678 | gl_5231199 | Streptococcus pneumoniae |
| 679 | gl_14602140 | Aeropyrum pernix |
| 680 | gl_21220517 | Streptomyces coelicolor A3(2) |
| 681 | gl_29376065 | Enterococcus faecalis V583 |
| 682 | MRT4565_11213P.3 | Triticum aestivum |
| 683 | gl_15642911 | Thermotoga maritima |
| 684 | gl_17546700 | Ralstonia solanacearum |
| 685 | gl_28900678 | Vibrio parahaemolyticus RIMD 2210633 |
| 686 | MRT4565_43124P.2 | Triticum aestivum |
| 687 | gl_24940270 | Wigandia caracasana |
| 688 | gl_14585885 | Pisum sativum |
| 689 | gl_15674910 | Streptococcus pyogenes M1 GAS |
| 690 | gl_4063538 | Carica papaya |
| 691 | gl_7708574 | Rhamnus cathartica |
| 692 | gl_15892991 | Rickettsia conorii |
| 693 | gl_4995854 | Thymelaea hirsuta |
| 694 | gl_11558184 | Lycopersicon esculentum |
| 695 | gl_14718147 | Neurada procumbens |
| 696 | gl_28566182 | Hordeum vulgare subsp. vulgare |
| 697 | gl_23061852 | Pseudomonas fluorescens PfO-1 |
| 698 | MRT3847_47036P.3 | Glycine max |
| 699 | gl_8452756 | Swietenia macrophylla |
| 700 | gl_7708464 | Koelreuteria paniculata |
| 701 | gl_20514385 | Strasburgeria robusta |
| 702 | MRT3847_56279P.2 | Glycine max |
| 703 | gl_28379494 | Lactobacillus plantarum WCFS1 |
| 704 | gl_4995057 | Abroma augustum |
| 705 | gl_19554226 | Corynebacterium glutamicum ATCC 13032 |
| 706 | gl_7708147 | Androsace spinulifera |
| 707 | gl_12004145 | Maesa tenera |
| 708 | gl_23056436 | Geobacter metallireducens |
| 709 | gl_5764615 | Zymomonas mobilis subsp. pomaceae |
| 710 | gl_33240025 | Prochlorococcus marinus subsp. marinus |
| | | str. CCMP1375 |
| 711 | gl_20467387 | Ephedra equisetina |
| 712 | gl_6467934 | Potamogeton berchtoldii |
| 713 | gl_20136045 | Shigella dysenteriae |
| 714 | gl_24967137 | Lycopersicon esculentum |
| 715 | gl_21684881 | Coleochloa abyssinica |
| 716 | gl_80953 | Methanothermococcus thermolithotrophicus |
| 717 | gl_29829367 | Streptomyces avermitilis MA-4680 |
| 718 | gl_28188325 | Coleochaete scutata |
| 719 | gl_23123457 | Prochlorococcus marinus subsp. pastoris |
| | | str. CCMP1378 |
| 720 | gl_3915890 | Cyanidium caldarium |
| 721 | gl_5231184 | Streptococcus pneumoniae |
| 722 | gl_15611532 | Helicobacter pylori J99 |
| 723 | gl_14041687 | Juglans regla |
| 724 | MRT4530_35849P.2 | Oryza sativa |
| 725 | gl_19881629 | Oryza sativa (japonica cultivar-group) |
| 726 | MRT4565_14599P.3 | Triticum aestivum |
| 727 | gl_7447118 | Pisum sativum |
| 728 | MRT4565_14138P.3 | Triticum aestivum |
| 729 | gl_24379618 | Streptococcus mutans UA159 |
| 730 | gl_30689162 | Arabidopsis thaliana |
| 731 | gl_19033067 | Coleochaete irregularis |
| 732 | gl_3334120 | Triticum aestivum |
| 733 | gl_12045070 | Mycoplasma genitalium |
| 734 | gl_14717948 | Balanops vieillardi |
| 735 | MRT3847_52567P.3 | Glycine max |
| 736 | gl_13183137 | Psidium guajava |
| 737 | gl_7708444 | Ilex crenata |
| 738 | gl_5830465 | Medicago sativa |
| 739 | gl_6688901 | Olea europaea |
| 740 | gl_15235430 | Arabidopsis thaliana |
| 741 | gl_4995850 | Triplochiton zambesiacus |
| 742 | gl_30352098 | Adiantum capillus-veneris |
| 743 | gl_23503623 | Carteria radiosa |
| 744 | MRT4565_59504P.2 | Triticum aestivum |
| 745 | gl_28211966 | Clostridium tetani E88 |
| 746 | gl_17231725 | Nostoc sp. PCC 7120 |
| 747 | MRT3847_55865P.2 | Glycine max |
| 748 | gl_12004157 | Primula sieboldii |
| 749 | gl_27364101 | Vibrio vulnificus CMCP6 |
| 750 | gl_14717990 | Carya glabra |
| 751 | gl_6094551 | Arabidopsis thaliana |
| 752 | gl_7447979 | Medicago sativa |
| 753 | gl_14330338 | Schedonorus pratensis |
| 754 | gl_27528492 | Saccharomyces pastorianus |
| 755 | gl_30683170 | Arabidopsis thaliana |
| 756 | gl_7708145 | Anagallis tenella |
| 757 | gl_32035049 | Actinobacillus pleuropneumoniae serovar 1 str. 4074 |
| 758 | gl_19705084 | Fusobacterium nucleatum subsp. nucleatum |
| | | ATCC 25586 |
| 759 | gl_20260650 | Arabidopsis thaliana |
| 760 | gl_688420 | Nicotiana glauca x Nicotiana langsdorffii |
| 761 | gl_4995153 | Fremontodendron californicum x Fremontodendron |
| | | mexicanum |
| 762 | gl_22532109 | Pseudomonas syringae |
| 763 | MRT4565_131929P.1 | Triticum aestivum |
| 764 | gl_14718056 | Flagellaria indica |
| 765 | gl_21633343 | Iseia luxurians |
| 766 | gl_7708300 | Escallonia sp. ‘Chase 2499 K’ |
| 767 | gl_113786 | Hordeum vulgare |
| 768 | gl_23059426 | Pseudomonas fluorescens PfO-1 |
| 769 | gl_17548614 | Ralstonia solanacearum |
| 770 | gl_11499192 | Archaeoglobus fulgldus DSM 4304 |
| 771 | gl_729237 | Ralstonia eutropha |
| 772 | gl_21070389 | Pennisetum glaucum |
| 773 | gl_6984122 | Capsicum annuum |
| 774 | gl_7688417 | Verbena scabrido-glandulosa |
| 775 | gl_28895034 | Streptococcus pyogenes SSI-1 |
| 776 | gl_7708538 | Phytolacca dioica |
| 777 | gl_23194453 | Gossypium hirsutum |
| 778 | MRT3847_258276P.2 | Glycine max |
| 779 | gl_29420867 | Saccharomyces pastorianus |
| 780 | gl_21633415 | Jacquemontia blanchetii |
| 781 | gl_28262023 | Rickettsia sibirica |
| 782 | gl_22969349 | Rhodospirillum rubrum |
| 783 | gl_32034452 | Actinobacillus pleuropneumoniae serovar |
| | | 1 str. 4074 |
| 784 | gl_20149298 | Malus x domestica |
| 785 | gl_8489192 | Lactococcus lactis subsp. lactis bv. |
| | | diacetylactis |
| 786 | gl_6687481 | Euthystachys abbreviata |
| 787 | gl_3850948 | Austromuellera trinervia |
| 788 | gl_114528 | Sulfolobus acidocaldarius |
| 789 | gl_8980813 | Castanea sativa |
| 790 | gl_16079874 | Bacillus subtilis subsp. subtilis str. 168 |
| 791 | gl_28194508 | Lotus japonicus |
| 792 | gl_28210705 | Clostridium tetani E88 |
| 793 | gl_6706178 | Gerbera jamesonii |
| 794 | gl_16943658 | Anemarrhena asphodeloides |
| 795 | gl_21326117 | Sorghum bicolor |
| 796 | gl_15216028 | Vicia faba var. minor |
| 797 | MRT4565_89954P.2 | Triticum aestivum |
| 798 | gl_5758921 | Zinglber gramineum |
| 799 | MRT4565_118733P.1 | Triticum aestivum |
| 800 | gl_27886806 | Fusobacterium nucleatum subsp. |
| | | vincentii ATCC 49256 |
| 801 | gl_3913031 | Medicago sativa |
| 802 | gl_18414404 | Arabidopsis thaliana |
| 803 | MRT4565_134443P.1 | Triticum aestivum |
| 804 | gl_4103757 | Corylus avellana |
| 805 | gl_21228923 | Methanosarcina mazei Goe1 |
| 806 | gl_30688675 | Arabidopsis thaliana |
| 807 | gl_32765543 | Hevea brasiliensis |
| 808 | gl_4063536 | Capparis spinosa |
| 809 | gl_7708313 | Geum sp. ‘Chase 2507 K’ |
| 810 | gl_29420847 | Saccharomyces cerevisiae |
| 811 | gl_1072369 | Enterococcus hirae |
| 812 | gl_23131072 | Prochlorococcus marinus str. MIT 9313 |
| 813 | gl_7708630 | Salacia pallescens |
| 814 | gl_5002358 | Azospirillum brasilense |
| 815 | gl_6017840 | Schisandra chinensis |
| 816 | gl_7861547 | Hydrogenophilus thermoluteolus |
| 817 | gl_23336808 | Bifidobacterium longum DJ010A |
| 818 | gl_1805530 | Escherichia coli |
| 819 | gl_3850914 | Stirlingla latifolia |
| 820 | gl_17231176 | Nostoc sp. PCC 7120 |
| 821 | gl_6687550 | Eremosyne pectinata |
| 822 | gl_21220814 | Streptomyces coelicolor A3(2) |
| 823 | gl_19112800 | Schizosaccharomyces pombe |
| 824 | gl_24374549 | Shewanella oneidensis MR-1 |
| 825 | gl_27467848 | Staphylococcus epidermidis ATCC 12228 |
| 826 | MRT4530_46208P.1 | Oryza sativa |
| 827 | gl_3913034 | Vigna unguiculata |
| 828 | gl_16943741 | Kniphofia uvaria |
| 829 | gl_6687627 | Gustavia superba |
| 830 | MRT4530_21634P.2 | Oryza sativa |
| 831 | gl_19578317 | Arabidopsis thaliana |
| 832 | gl_11034787 | Cabomba caroliniana |
| 833 | gl_18312083 | Pyrobaculum aerophilum str. IM2 |
| 834 | gl_6942107 | Brucella melitensis biovar Abortus |
| 835 | MRT3847_233420P.2 | Glycine max |
| 836 | gl_20136043 | Shigella dysenteriae |
| 837 | gl_24967135 | Lycopersicon esculentum |
| 838 | gl_17224761 | Tacca plantaglnea |
| 839 | gl_16273337 | Haemophilus influenzae Rd |
| 840 | gl_4995181 | Helicteres baruensis |
| 841 | gl_1526982 | Salmonella typhimurium |
| 842 | gl_26247926 | Escherichia coli CFT073 |
| 843 | gl_14906664 | Sorghum bicolor |
| 844 | MRT4565_64073P.2 | Triticum aestivum |
| 845 | gl_4206584 | Chorilaena quercifolia |
| 846 | gl_23099626 | Oceanobacillus iheyensis HTE831 |
| 847 | gl_6691650 | Moritella marina |
| 848 | gl_15791646 | Campylobacter jejuni subsp. jejuni NCTC 11168 |
| 849 | gl_24940246 | Nemophila insignis |
| 850 | gl_11908164 | Swietenia macrophylla |
| 851 | gl_29150650 | Oryza sativa (indica cultivar-group) |
| 852 | gl_22758323 | Oryza sativa (japonica cultivar-group) |
| 853 | MRT4565_42533P.3 | Triticum aestivum |
| 854 | gl_7708321 | Guaiacum sanctum |
| 855 | gl_7708676 | Thunbergla coccinea |
| 856 | gl_7708466 | Krameria ixine |
| 857 | MRT4565_103551P.1 | Triticum aestivum |
| 858 | gl_27377698 | Bradyrhizobium japonicum USDA 110 |
| 859 | gl_30267062 | Ipomoea tabascana |
| 860 | gl_19743774 | Gossypium hirsutum |
| 861 | gl_27657747 | Helianthus annuus |
| 862 | gl_7687980 | Gyrocarpus americanus |
| 863 | gl_7578495 | Quercus rubra |
| 864 | gl_6599047 | Chlamydia trachomatis |
| 865 | gl_732262 | Yersinia pseudotuberculosis |
| 866 | gl_19115131 | Schizosaccharomyces pombe |
| 867 | gl_21633375 | Bonamia spectabilis |
| 868 | gl_7446520 | Cucumis sativus |
| 869 | gl_14717946 | Asteropeia micraster |
| 870 | gl_4206759 | Cryptococcus neoformans var. grubii |
| 871 | gl_17232303 | Nostoc sp. PCC 7120 |
| 872 | gl_21328719 | uncultured proteobacterium |
| 873 | gl_15618755 | Chlamydophila pneumoniae CWL029 |
| 874 | gl_282382 | Geobacillus stearothermophilus |
| 875 | gl_2129972 | Petunia x hybrida |
| 876 | gl_6225171 | Synechococcus sp. PCC 7942 |
| 877 | gl_7688029 | Nymphaea odorata |
| 878 | gl_16943662 | Aspidistra elatior |
| 879 | gl_461978 | Lycopersicon esculentum |
| 880 | MRT4565_40318P.2 | Triticum aestivum |
| 881 | gl_6319704 | Saccharomyces cerevisiae |
| 882 | MRT4565_9194P.2 | Triticum aestivum |
| 883 | MRT3847_90337P.3 | Glycine max |
| 884 | gl_2493122 | Brassica napus |
| 885 | gl_27468291 | Staphylococcus epidermidis ATCC 12228 |
| 886 | gl_19033069 | Coleochaete sieminskiana |
| 887 | MRT4530_91499P.1 | Oryza sativa |
| 888 | gl_7708335 | Humulus lupulus |
| 889 | gl_21402641 | Bacillus anthracis str. A2012 |
| 890 | gl_28563989 | Saccharomyces bayanus |
| 891 | gl_27904791 | Buchnera aphidicola str. Bp (Baizongla pistaciae) |
| 892 | gl_24935324 | Medicago truncatula |
| 893 | gl_5921507 | Mortierella alpina |
| 894 | gl_7708315 | Globularia salicina |
| 895 | gl_114520 | Methanosarcina barkeri |
| 896 | gl_15226178 | Arabidopsis thaliana |
| 897 | gl_1707370 | Arabidopsis thaliana |
| 898 | gl_22997796 | Xylella fastidiosa Ann-1 |
| 899 | gl_16273468 | Haemophilus influenzae Rd |
| 900 | gl_151617 | Pseudomonas aeruglnosa |
| 901 | gl_21219634 | Streptomyces coelicolor A3(2) |
| 902 | MRT4565_76776P.2 | Triticum aestivum |
| 903 | gl_23118917 | Desulfitobacterium hafniense |
| 904 | gl_32130302 | Bacillus subtilis var. natto |
| 905 | MRT3847_52222P.3 | Glycine max |
| 906 | gl_12004159 | Primula veitchiana |
| 907 | gl_6688708 | Mentzelia lindleyi |
| 908 | gl_23021744 | Clostridium thermocellum ATCC 27405 |
| 909 | gl_136258 | Haloferax volcanii |
| 910 | gl_7687960 | Austrobaileya scandens |
| 911 | MRT3847_39339P.3 | Glycine max |
| 912 | gl_32489847 | Oryza sativa (japonica cultivar-group) |
| 913 | gl_30693784 | Arabidopsis thaliana |
| 914 | gl_7381060 | Populus tremula x Populus tremuloides |
| 915 | gl_19033097 | Chlorokybus atmophyticus |
| 916 | gl_27528502 | Saccharomyces kluyveri |
| 917 | MRT4530_27056P.1 | Oryza sativa |
| 918 | MRT3847_30014P.3 | Glycine max |
| 919 | gl_4063568 | Pavonia multiflora |
| 920 | gl_30724884 | Microbispora rosea subsp. aerata |
| 921 | gl_23099005 | Oceanobacillus iheyensis HTE831 |
| 922 | gl_19705056 | Fusobacterium nucleatum subsp. |
| | | nucleatum ATCC 25586 |
| 923 | MRT4565_101762P.1 | Triticum aestivum |
| 924 | MRT3847_225429P.3 | Glycine max |
| 925 | gl_7708542 | Pittosporum fairchildii |
| 926 | gl_7708329 | Helwingla japonica |
| 927 | gl_79917 | Staphylococcus aureus |
| 928 | gl_23016131 | Magnetospirillum magnetotacticum |
| 929 | gl_19352035 | Oryza sativa |
| 930 | MRT4530_60814P.1 | Oryza sativa |
| 931 | gl_4995844 | Sarcolaena sp. Chase 903 |
| 932 | gl_30685252 | Arabidopsis thaliana |
| 933 | gl_7434424 | Oryza longlstaminata |
| 934 | gl_13161415 | Oryza sativa (japonica cultivar-group) |
| 935 | MRT4530_77791P.2 | Oryza sativa |
| 936 | gl_32039540 | Pseudomonas aeruglnosa UCBPP-PA14 |
| 937 | gl_22094585 | Populus tomentosa |
| 938 | gl_6601482 | Allium cepa |
| 939 | gl_136264 | Pseudomonas putida |
| 940 | MRT4565_66175P.2 | Triticum aestivum |
| 941 | gl_27528480 | Saccharomyces unisporus |
| 942 | gl_15669227 | Methanocaldococcus jannaschii |
| 943 | gl_15225218 | Arabidopsis thaliana |
| 944 | gl_18406070 | Arabidopsis thaliana |
| 945 | gl_4837612 | Antirrhinum majus |
| 946 | gl_4995063 | Apeiba tibourbou |
| 947 | gl_16123318 | Yersinia pestis CO92 |
| 948 | gl_31126749 | Oryza sativa (japonica cultivar-group) |
| 949 | gl_7708556 | Polygonum sachalinense |
| 950 | gl_27529081 | Zygosaccharomyces rouxii |
| 951 | gl_20136075 | Shigella sonnei |
| 952 | gl_23124896 | Nostoc punctiforme |
| 953 | gl_29828741 | Streptomyces avermitilis MA-4680 |
| 954 | gl_6688494 | Irvingbaileya sp. Plunkett 1510 |
| 955 | gl_11466709 | Marchantia polymorpha |
| 956 | gl_33113492 | Pringlea antiscorbutica |
| 957 | gl_27529077 | Zygosaccharomyces bailii |
| 958 | gl_15224925 | Arabidopsis thaliana |
| 959 | gl_553048 | Daucus carota |
| 960 | gl_29375007 | Enterococcus faecalis V583 |
| 961 | gl_27887626 | Fusobacterium nucleatum subsp. |
| | | vincentii ATCC 49256 |
| 962 | gl_30421165 | Hordeum vulgare |
| 963 | gl_17546431 | Ralstonia solanacearum |
| 964 | gl_15810897 | Antirrhinum majus subsp. cirrhigerum |
| 965 | gl_15223786 | Arabidopsis thaliana |
| 966 | gl_23465333 | Bifidobacterium longum NCC2705 |
| 967 | MRT4565_26905P.2 | Triticum aestivum |
| 968 | MRT4565_47460P.3 | Triticum aestivum |
| 969 | gl_3779258 | Hordeum vulgare subsp. vulgare |
| 970 | gl_23474551 | Desulfovibrio desulfuricans G20 |
| 971 | gl_7687976 | Eupomatia bennettii |
| 972 | gl_15237539 | Arabidopsis thaliana |
| 973 | gl_16272655 | Haemophilus influenzae Rd |
| 974 | gl_29832720 | Streptomyces avermitilis MA-4680 |
| 975 | gl_15425564 | Crispiloba disperma |
| 976 | gl_11267101 | Methanosarcina mazei |
| 977 | gl_23469383 | Pseudomonas syringae pv. syringae B728a |
| 978 | gl_23104278 | Azotobacter vinelandii |
| 979 | gl_29420853 | Candida glabrata |
| 980 | gl_15828711 | Mycoplasma pulmonis |
| 981 | gl_14718242 | Tapiscia sinensis |
| 982 | gl_7708578 | Rinorea bengalensis |
| 983 | gl_4995757 | Pachira aquatica |
| 984 | gl_14329816 | Atropa belladonna |
| 985 | gl_6688492 | Justicia americana |
| 986 | gl_4995705 | Microcos latistipulata |
| 987 | MRT4565_21523P.3 | Triticum aestivum |
| 988 | gl_23336272 | Bifidobacterium longum DJO10A |
| 989 | gl_20467383 | Ephedra sp. CR08 |
| 990 | gl_7708215 | Corynocarpus laevigatus |
| 991 | gl_23119424 | Desulfitobacterium hafniense |
| 992 | gl_20136041 | Shigella dysenteriae |
| 993 | MRT4565_123153P.1 | Triticum aestivum |
| 994 | MRT3847_243747P.2 | Glycine max |
| 995 | gl_6706286 | Phlox longlfolia |
| 996 | gl_16804835 | Listeria monocytogenes EGD-e |
| 997 | MRT4530_100513P.2 | Oryza sativa |
| 998 | gl_31126747 | Oryza sativa (japonica cultivar-group) |
| 999 | gl_24215258 | Leptospira interrogans serovar lai str. 56601 |
| 1000 | gl_4180 | Saccharomyces cerevisiae |
| 1001 | gl_30385250 | x Citrofortunella mitis |
| 1002 | gl_21226817 | Methanosarcina mazei Goe1 |
| 1003 | MRT4530_54698P.1 | Oryza sativa |
| 1004 | MRT3847_25290P.2 | Glycine max |
| 1005 | MRT4565_104502P.1 | Triticum aestivum |
| 1006 | gl_24940166 | Cerinthe major |
| 1007 | gl_15226967 | Arabidopsis thaliana |
| 1008 | gl_23475994 | Desulfovibrio desulfuricans G20 |
| 1009 | gl_12585490 | Citrus unshiu |
| 1010 | gl_30267060 | Ipomoea setosa |
| 1011 | MRT4530_57126P.1 | Oryza sativa |
| 1012 | MRT3847_52223P.3 | Glycine max |
| 1013 | gl_27657745 | Helianthus annuus |
| 1014 | gl_32400328 | Asperglllus oryzae |
| 1015 | gl_20161442 | Oryza sativa (japonica cultivar-group) |
| 1016 | gl_8388947 | Eriostemon brevifolius |
| 1017 | gl_15897407 | Sulfolobus solfataricus |
| 1018 | gl_30022560 | Bacillus cereus ATCC 14579 |
| 1019 | gl_7708286 | Eucryphia milliganii |
| 1020 | gl_27262488 | Heliobacillus mobilis |
| 1021 | gl_9955371 | Escherichia coli |
| 1022 | gl_6692624 | Allium cepa |
| 1023 | MRT4530_101175P.1 | Oryza sativa |
| 1024 | gl_12004161 | Samolus repens |
| 1025 | gl_94733 | Thermus aquaticus |
| 1026 | gl_3913005 | Panax glnseng |
| 1027 | gl_1169445 | Pisum sativum |
| 1028 | MRT3847_253859P.2 | Glycine max |
| 1029 | gl_18657017 | Oryza sativa |
| 1030 | gl_6320442 | Saccharomyces cerevisiae |
| 1031 | gl_15236190 | Arabidopsis thaliana |
| 1032 | gl_15618012 | Chlamydophila pneumoniae CWL029 |
| 1033 | gl_29420833 | Saccharomyces cerevisiae |
| 1034 | MRT4565_6744P.2 | Triticum aestivum |
| 1035 | gl_14718140 | Moringa oleifera |
| 1036 | gl_15604188 | Rickettsia prowazekii |
| 1037 | gl_12004149 | Omphalogramma delavayi |
| 1038 | gl_775181 | Escherichia coli |
| 1039 | gl_217940 | Ipomoea batatas |
| 1040 | gl_14718085 | Idesia polycarpa |
| 1041 | MRT4530_103357P.1 | Oryza sativa |
| 1042 | gl_27125515 | Mesembryanthemum crystallinum |
| 1043 | gl_25011425 | Streptococcus agalactiae NEM316 |
| 1044 | gl_6456467 | Taraxacum officinale |
| 1045 | gl_7573596 | Populus nigra |
| 1046 | MRT4530_57276P.1 | Oryza sativa |
| 1047 | gl_12004131 | Anagallis arvensis |
| 1048 | gl_15897777 | Sulfolobus solfataricus |
| 1049 | gl_3850978 | Embothrium coccineum |
| 1050 | gl_28563987 | Saccharomyces bayanus |
| 1051 | gl_15901412 | Streptococcus pneumoniae TIGR4 |
| 1052 | gl_21633463 | Montinia caryophyllacea |
| 1053 | gl_20805979 | Chlamydia trachomatis |
| 1054 | gl_7688411 | Utricularia biflora |
| 1055 | gl_27468267 | Staphylococcus epidermidis ATCC 12228 |
| 1056 | gl_25345298 | Arabidopsis thaliana |
| 1057 | gl_16763830 | Salmonella typhimurium LT2 |
| 1058 | gl_28211923 | Clostridium tetani E88 |
| 1059 | gl_17065024 | Arabidopsis thaliana |
| 1060 | gl_22959339 | Rhodobacter sphaeroides |
| 1061 | gl_6759507 | Elaeis guineensis |
| 1062 | gl_28188339 | Coleochaete divergens |
| 1063 | gl_13476995 | Mesorhizobium loti |
| 1064 | gl_7708652 | Spathiphyllum wallisii |
| 1065 | gl_15642010 | Vibrio cholerae |
| 1066 | gl_30695267 | Arabidopsis thaliana |
| 1067 | MRT3847_29671P.3 | Glycine max |
| 1068 | gl_4995796 | Sterculia apetala |
| 1069 | gl_27366266 | Vibrio vulnificus CMCP6 |
| 1070 | gl_1169648 | Rhodococcus fascians |
| 1071 | gl_16122431 | Yersinia pestis CO92 |
| 1072 | gl_25289327 | Arabidopsis thaliana |
| 1073 | gl_4995759 | Neurada procumbens |
| 1074 | gl_30696140 | Arabidopsis thaliana |
| 1075 | gl_7708327 | Heisteria parvifolia |
| 1076 | gl_14289139 | Bacillus sphaericus |
| 1077 | gl_15966192 | Sinorhizobium meliloti |
| 1078 | MRT3847_268909P.1 | Glycine max |
| 1079 | gl_4063566 | Simarouba glauca |
| 1080 | MRT3847_162726P.3 | Glycine max |
| 1081 | gl_28973727 | Arabidopsis thaliana |
| 1082 | gl_16126292 | Caulobacter crescentus CB15 |
| 1083 | gl_602900 | Silene latifolia |
| 1084 | gl_21633411 | Jacquemontia tamnifolia |
| 1085 | gl_5019431 | Gnetum gnemon |
| 1086 | gl_25307920 | Picea abies |
| 1087 | MRT4530_7968P.2 | Oryza sativa |
| 1088 | gl_4206608 | Pleiospermium alatum |
| 1089 | gl_25486627 | Picea mariana |
| 1090 | gl_23122427 | Prochlorococcus marinus subsp. pastoris |
| | | str. CCMP1378 |
| 1091 | MRT4530_85948P.1 | Oryza sativa |
| 1092 | gl_16078679 | Bacillus subtilis subsp. subtilis str. 168 |
| 1093 | gl_15602442 | Pasteurella multocida |
| 1094 | gl_3850944 | Orites lancifolia |
| 1095 | gl_16126335 | Caulobacter crescentus CB15 |
| 1096 | gl_21684883 | Ecdeiocolea monostachya |
| 1097 | gl_23132758 | Synechococcus sp. WH 8102 |
| 1098 | gl_80601 | Corynebacterium glutamicum |
| 1099 | gl_21954719 | Mesotaenium caldariorum |
| 1100 | gl_21536895 | Arabidopsis thaliana |
| 1101 | gl_7442735 | Ricinus communis |
| 1102 | gl_29539348 | Cyanidioschyzon merolae |
| 1103 | gl_2497543 | Nicotiana tabacum |
| 1104 | gl_16800673 | Listeria innocua |
| 1105 | MRT3847_224215P.2 | Glycine max |
| 1106 | gl_23106149 | Azotobacter vinelandii |
| 1107 | gl_125606 | Solanum tuberosum |
| 1108 | gl_15605029 | Chlamydia trachomatis |
| 1109 | gl_7676165 | Methanothermobacter thermautotrophicus |
| 1110 | gl_20136073 | Shigella sonnei |
| 1111 | gl_23135856 | Cytophaga hutchinsonii |
| 1112 | gl_22986693 | Burkholderia fungorum |
| 1113 | gl_11279328 | Pisum sativum |
| 1114 | gl_4586799 | Nicotiana tabacum |
| 1115 | gl_32476350 | Pirellula sp. |
| 1116 | gl_21742732 | Oryza sativa (japonica cultivar-group) |
| 1117 | MRT4565_78273P.2 | Triticum aestivum |
| 1118 | gl_29348250 | Bacteroides thetaiotaomicron VPI-5482 |
| 1119 | gl_30421168 | Hordeum vulgare |
| 1120 | gl_2506211 | Vigna radiata var. radiata |
| 1121 | gl_5830467 | Medicago sativa |
| 1122 | MRT4565_118736P.1 | Triticum aestivum |
| 1123 | gl_8517661 | Silene nutans |
| 1124 | gl_1310978 | Escherichia coli |
| 1125 | gl_21633441 | Dinetus truncatus |
| 1126 | gl_21684927 | Streptochaeta spicata |
| 1127 | gl_15963782 | Sinorhizobium meliloti |
| 1128 | gl_15982240 | Nicotiana attenuata |
| 1129 | MRT4530_98210P.1 | Oryza sativa |
| 1130 | gl_23123201 | Prochlorococcus marinus subsp. pastoris |
| | | str. CCMP1378 |
| 1131 | gl_15617074 | Buchnera aphidicola str. APS |
| | | (Acyrthosiphon pisum) |
| 1132 | gl_15609594 | Mycobacterium tuberculosis H37Rv |
| 1133 | gl_15806971 | Deinococcus radiodurans |
| 1134 | gl_18404228 | Arabidopsis thaliana |
| 1135 | gl_17224755 | Tacca leontopetaloides |
| 1136 | gl_23134144 | Synechococcus sp. WH 8102 |
| 1137 | gl_27528494 | Saccharomyces kudriavzevii |
| 1138 | gl_14718240 | Tamarix pentandra |
| 1139 | gl_22536365 | Streptococcus agalactiae 2603V/R |
| 1140 | gl_17988302 | Brucella melitensis 16M |
| 1141 | gl_20805995 | Chlamydia trachomatis |
| 1142 | gl_21673243 | Chlorobium tepidum TLS |
| 1143 | gl_28897692 | Vibrio parahaemolyticus RIMD 2210633 |
| 1144 | gl_24940188 | Hydrophyllum canadense |
| 1145 | gl_20467381 | Ephedra fragllis |
| 1146 | gl_22970242 | Chloroflexus aurantiacus |
| 1147 | MRT3847_257209P.2 | Glycine max |
| 1148 | gl_7488272 | Arabidopsis thaliana |
| 1149 | gl_22993311 | Enterococcus faecium |
| 1150 | gl_6017824 | Rheum rhaponticum |
| 1151 | gl_13676299 | Glycine max |
| 1152 | gl_15595759 | Pseudomonas aeruglnosa PAO1 |
| 1153 | gl_4033710 | Picea mariana |
| 1154 | gl_7708254 | Celastrus orbiculatus |
| 1155 | gl_15597263 | Pseudomonas aeruglnosa PAO1 |
| 1156 | gl_21672725 | Buchnera aphidicola str. Sg (Schizaphis graminum) |
| 1157 | gl_4063562 | Ruta graveolens |
| 1158 | gl_15802088 | Escherichia coli 0157:H7 EDL933 |
| 1159 | gl_7674396 | Thermococcus kodakaraensis |
| 1160 | gl_32476155 | Pirellula sp. |
| 1161 | MRT3847_32267P.3 | Glycine max |
| 1162 | gl_137460 | Daucus carota |
| 1163 | gl_23029594 | Microbulbifer degradans 2-40 |
| 1164 | gl_23126009 | Nostoc punctiforme |
| 1165 | gl_16078805 | Bacillus subtilis subsp. subtilis str. 168 |
| 1166 | gl_29420869 | Saccharomyces pastorianus |
| 1167 | gl_14718009 | Cleome hassleriana |
| 1168 | gl_21684907 | Mayaca fluviatilis |
| 1169 | gl_16803308 | Listeria monocytogenes EGD-e |
| 1170 | MRT3847_50682P.1 | Glycine max |
| 1171 | gl_21593559 | Arabidopsis thaliana |
| 1172 | gl_21633371 | Cressa truxillensis |
| 1173 | gl_22967579 | Rhodospirillum rubrum |
| 1174 | MRT4565_57148P.3 | Triticum aestivum |
| 1175 | gl_7488446 | Brassica napus |
| 1176 | gl_23002842 | Lactobacillus gasseri |
| 1177 | gl_27528476 | Torulaspora globosa |
| 1178 | gl_15641923 | Vibrio cholerae |
| 1179 | gl_17986575 | Brucella melitensis 16M |
| 1180 | gl_15600873 | Vibrio cholerae |
| 1181 | gl_15606540 | Aquifex aeolicus VF5 |
| 1182 | gl_6687483 | Exacum affine |
| 1183 | gl_32404216 | Neurospora crassa |
| 1184 | gl_15893809 | Clostridium acetobutylicum |
| 1185 | gl_18077601 | Paracryphia alticola |
| 1186 | gl_24298775 | Thermotoga neapolitana |
| 1187 | MRT3847_33136P.3 | Glycine max |
| 1188 | gl_11465694 | Porphyra purpurea |
| 1189 | gl_1346399 | Lactobacillus delbrueckii subsp. bulgaricus |
| 1190 | gl_28870880 | Pseudomonas syringae pv. tomato str. DC3000 |
| 1191 | gl_23130789 | Prochlorococcus marinus str. MIT 9313 |
| 1192 | gl_15837790 | Xylella fastidiosa 9a5c |
| 1193 | gl_32410899 | Neurospora crassa |
| 1194 | gl_21283347 | Staphylococcus aureus subsp. aureus MW2 |
| 1195 | gl_21553710 | Arabidopsis thaliana |
| 1196 | gl_5001601 | Schumacheria sp. SH1999 |
| 1197 | gl_30693084 | Arabidopsis thaliana |
| 1198 | gl_4096982 | Rosa hybrid cultivar |
| 1199 | gl_21633359 | Cladostigma hildebrandtioides |
| 1200 | MRT3847_198776P.3 | Glycine max |
| 1201 | gl_1364102 | Rumex acetosa |
| 1202 | MRT3847_249579P.2 | Glycine max |
| 1203 | gl_15596999 | Pseudomonas aeruglnosa PAO1 |
| 1204 | MRT4565_141501P.1 | Triticum aestivum |
| 1205 | gl_3850976 | Alloxylon wickhamii |
| 1206 | gl_28563985 | Saccharomyces bayanus |
| 1207 | gl_23028929 | Microbulbifer degradans 2-40 |
| 1208 | gl_33240373 | Prochlorococcus marinus subsp. |
| | | marinus str. CCMP1375 |
| 1209 | MRT4530_110805P.1 | Oryza sativa |
| 1210 | gl_22537102 | Streptococcus agalactiae 2603V/R |
| 1211 | gl_3913006 | Petunia x hybrida |
| 1212 | gl_2120372 | Thermotoga maritima |
| 1213 | gl_16079970 | Bacillus subtilis subsp. subtilis str. 168 |
| 1214 | gl_15679655 | Methanothermobacter thermautotrophicus |
| | | str. Delta H |
| 1215 | MRT3847_40554P.3 | Glycine max |
| 1216 | gl_29420849 | Saccharomyces cerevisiae |
| 1217 | gl_28188337 | Coleochaete nitellarum |
| 1218 | MRT4565_86330P.2 | Triticum aestivum |
| 1219 | MRT4565_49252P.2 | Triticum aestivum |
| 1220 | gl_4322325 | Nepenthes alata |
| 1221 | gl_7428175 | Arabidopsis thaliana |
| 1222 | MRT3847_218209P.1 | Glycine max |
| 1223 | gl_7706848 | Amaranthus hypochondriacus |
| 1224 | gl_12004133 | Androsace sp. Anderberg s.n. |
| 1225 | gl_1808694 | Sporobolus stapfianus |
| 1226 | gl_13447449 | Brassica napus |
| 1227 | gl_18410104 | Arabidopsis thaliana |
| 1228 | MRT4530_71260P.2 | Oryza sativa |
| 1229 | gl_30022674 | Bacillus cereus ATCC 14579 |
| 1230 | gl_15827775 | Mycobacterium leprae |
| 1231 | gl_19033085 | Zygnema peliosporum |
| 1232 | gl_4063564 | Schinus molle |
| 1233 | gl_464911 | Pseudomonas syringae pv. syringae |
| 1234 | MRT4565_58034P.2 | Triticum aestivum |
| 1235 | gl_5001597 | Didymeles perrieri |
| 1236 | gl_8096650 | Oryza sativa (japonica cultivar-group) |
| 1237 | gl_20805068 | Oryza sativa (japonica cultivar-group) |
| 1238 | gl_7708143 | Alanglum sp. Chase 2541 |
| 1239 | MRT3847_271867P.1 | Glycine max |
| 1240 | gl_20094453 | Methanopyrus kandleri AV19 |
| 1241 | gl_3023341 | Equisetum arvense |
| 1242 | gl_4206606 | Glycosmis pentaphylla |
| 1243 | gl_7446714 | Capsicum annuum |
| 1244 | gl_22125938 | Yersinia pestis KIM |
| 1245 | gl_18310620 | Clostridium perfringens str. 13 |
| 1246 | gl_1336803 | Mesembryanthemum crystallinum |
| 1247 | gl_7708668 | Symplocos costata |
| 1248 | gl_20136029 | Shigella flexneri |
| 1249 | gl_3850942 | Neorites kevediana |
| 1250 | gl_4995848 | Thomasia solanacea |
| 1251 | gl_1667582 | Arabidopsis thaliana |
| 1252 | gl_11527563 | Hordeum vulgare subsp. vulgare |
| 1253 | gl_68331 | Klebsiella pneumoniae |
| 1254 | MRT4530_121232P.2 | Oryza sativa |
| 1255 | gl_22748323 | Oryza sativa (japonica cultivar-group) |
| 1256 | gl_21956014 | Vitreochlamys aulata |
| 1257 | MRT4565_27586P.3 | Triticum aestivum |
| 1258 | MRT4530_100337P.1 | Oryza sativa |
| 1259 | gl_15240418 | Arabidopsis thaliana |
| 1260 | MRT4530_114765P.2 | Oryza sativa |
| 1261 | gl_16760530 | Salmonella enterica subsp. enterica serovar Typhi |
| 1262 | gl_15645143 | Helicobacter pylori 26695 |
| 1263 | gl_29833210 | Streptomyces avermitilis MA-4680 |
| 1264 | gl_15529115 | Sorghum bicolor |
| 1265 | gl_4995095 | Chorisia speciosa |
| 1266 | MRT4565_71673P.1 | Triticum aestivum |
| 1267 | gl_6729696 | Hordeum vulgare |
| 1268 | gl_15896405 | Clostridium acetobutylicum |
| 1269 | gl_15082058 | Solanum tuberosum |
| 1270 | gl_4995649 | Keraudrenia hermanniifolia |
| 1271 | MRT4530_21638P.2 | Oryza sativa |
| 1272 | gl_3417405 | Saccharomyces cerevisiae |
| 1273 | MRT4565_3598P.3 | Triticum aestivum |
| 1274 | gl_33241266 | Prochlorococcus marinus subsp. |
| | | marinus str. CCMP1375 |
| 1275 | gl_97924 | Enterococcus hirae |
| 1276 | gl_23004108 | Magnetospirillum magnetotacticum |
| 1277 | gl_30316239 | Streptococcus pyogenes SSI-1 |
| 1278 | MRT4565_16589P.2 | Triticum aestivum |
| 1279 | gl_6599049 | Chlamydia trachomatis |
| 1280 | gl_11279332 | Populus x canescens |
| 1281 | gl_29840676 | Chlamydophila caviae GPIC |
| 1282 | gl_32417454 | Neurospora crassa |
| 1283 | gl_5305232 | Brassica napus |
| 1284 | gl_4063530 | Bixa orellana |
| 1285 | gl_26986827 | Pseudomonas putida KT2440 |
| 1286 | gl_32441888 | Brassica oleracea var. capitata |
| 1287 | gl_4218537 | Triticum sp. |
| 1288 | gl_21909656 | Streptococcus pyogenes MGAS315 |
| 1289 | gl_20330757 | Oryza sativa (japonica cultivar-group) |
| 1290 | gl_13474176 | Mesorhizobium loti |
| 1291 | gl_5616513 | Fragaria x ananassa |
| 1292 | gl_16943664 | Calibanus hookeri |
| 1293 | gl_5231202 | Streptococcus pneumoniae |
| 1294 | MRT4530_72752P.2 | Oryza sativa |
| 1295 | gl_15292855 | Arabidopsis thaliana |
| 1296 | gl_20136059 | Shigella dysenteriae |
| 1297 | gl_15209148 | Oryza sativa |
| 1298 | gl_7708452 | Irvingla malayana |
| 1299 | gl_30687843 | Arabidopsis thaliana |
| 1300 | gl_20269434 | Pouteria obovata |
| 1301 | gl_136253 | Geobacillus stearothermophilus |
| 1302 | gl_18976554 | Pyrococcus furiosus DSM 3638 |
| 1303 | gl_14495542 | Ipomoea nil |
| 1304 | gl_16330288 | Synechocystis sp. PCC 6803 |
| 1305 | gl_16800386 | Listeria innocua |
| 1306 | gl_21633427 | Maripa repens |
| 1307 | gl_28380199 | Brucella melitensis |
| 1308 | gl_3913004 | Lycopersicon esculentum |
| 1309 | gl_155435 | unidentified bacterium |
| 1310 | gl_23017117 | Thermobifida fusca |
| 1311 | gl_14718099 | Koeberlinia spinosa |
| 1312 | gl_15674362 | Streptococcus pyogenes M1 GAS |
| 1313 | gl_6017822 | Phytolacca americana |
| 1314 | gl_15807615 | Deinococcus radiodurans |
| 1315 | gl_14521960 | Pyrococcus abyssi |
| 1316 | gl_23111662 | Desulfitobacterium hafniense |
| 1317 | gl_15643288 | Thermotoga maritima |
| 1318 | MRT3847_269768P.1 | Glycine max |
| 1319 | gl_23308892 | Corynebacterium glutamicum ATCC 13032 |
| 1320 | gl_4063560 | Rhus copallina |
| 1321 | gl_7708311 | Hydnocarpus heterophylla |
| 1322 | MRT4565_24270P.3 | Triticum aestivum |
| 1323 | MRT3847_233522P.2 | Glycine max |
| 1324 | MRT4530_87659P.1 | Oryza sativa |
| 1325 | gl_15596894 | Pseudomonas aeruglnosa PAO1 |
| 1326 | gl_25028847 | Corynebacterium efficiens YS-314 |
| 1327 | gl_24379392 | Streptococcus mutans UA159 |
| 1328 | gl_11133033 | Lactobacillus leichmannii |
| 1329 | MRT4565_19576P.3 | Triticum aestivum |
| 1330 | gl_24940194 | Lithodora diffusa |
| 1331 | gl_16610205 | Physcomitrella patens |
| 1332 | MRT3847_212021P.2 | Glycine max |
| 1333 | gl_6970411 | Rosa rugosa |
| 1334 | gl_8745072 | Betula pendula |
| 1335 | gl_16122616 | Yersinia pestis CO92 |
| 1336 | gl_7708684 | Thesium humile |
| 1337 | gl_14718007 | Clarkia xantiana |
| 1338 | gl_7708474 | Lavandula bipinnata |
| 1339 | gl_14718107 | Lepuropetalon spathulatum |
| 1340 | gl_16444949 | Asperglllus oryzae |
| 1341 | gl_27363511 | Vibrio vulnificus CMCP6 |
| 1342 | gl_24559828 | Bradyrhizobium japonicum |
| 1343 | gl_848999 | Petunia integrifolia subsp. inflata |
| 1344 | gl_13489165 | Oryza sativa (japonica cultivar-group) |
| 1345 | gl_6273581 | Oenococcus oeni |
| 1346 | gl_6467949 | Persoonia katerae |
| 1347 | gl_1730065 | Sporosarcina psychrophila |
| 1348 | gl_23102311 | Azotobacter vinelandii |
| 1349 | gl_15639417 | Treponema pallidum |
| 1350 | gl_15235112 | Arabidopsis thaliana |
| 1351 | gl_16077989 | Bacillus subtilis subsp. subtilis str. 168 |
| 1352 | gl_7674382 | Buchnera aphidicola (Schlechtendalia chinensis) |
| 1353 | gl_10946499 | Hevea brasiliensis |
| 1354 | gl_30468052 | Cyanidioschyzon merolae |
| 1355 | gl_23002438 | Lactobacillus gasseri |
| 1356 | gl_29831992 | Streptomyces avermitilis MA-4680 |
| 1357 | gl_21401687 | Bacillus anthracis str. A2012 |
| 1358 | MRT4565_53782P.2 | Triticum aestivum |
| 1359 | gl_21282866 | Staphylococcus aureus subsp. aureus MW2 |
| 1360 | gl_27528482 | Saccharomyces castellii |
| 1361 | gl_21264381 | Vandenboschia davallioides |
| 1362 | gl_12004123 | Coris monspeliensis |
| 1363 | gl_7447961 | Gossypium hirsutum |
| 1364 | gl_22963535 | Rhodopseudomonas palustris |
| 1365 | gl_28804505 | Aster tripolium |
| 1366 | gl_3860313 | Cicer arietinum |
| 1367 | gl_27529083 | Torulaspora pretoriensis |
| 1368 | gl_4995111 | Colona floribunda |
| 1369 | gl_17987158 | Brucella melitensis 16M |
| 1370 | gl_25410916 | Arabidopsis thaliana |
| 1371 | gl_15219234 | Arabidopsis thaliana |
| 1372 | gl_5231193 | Streptococcus pneumoniae |
| 1373 | gl_7487385 | Arabidopsis thaliana |
| 1374 | gl_3913007 | Nicotiana tabacum |
| 1375 | gl_407635 | Mycoplasma genitalium |
| 1376 | gl_27529079 | Zygosaccharomyces bisporus |
| 1377 | gl_27380054 | Bradyrhizobium japonicum USDA 110 |
| 1378 | gl_15219603 | Arabidopsis thaliana |
| 1379 | gl_5001603 | Eucryphia cordifolia |
| 1380 | gl_15839668 | Mycobacterium tuberculosis CDC1551 |
| 1381 | gl_1142616 | Bacillus subtilis |
| 1382 | gl_28188335 | Coleochaete scutata |
| 1383 | gl_21674017 | Chlorobium tepidum TLS |
| 1384 | gl_27375757 | Bradyrhizobium japonicum USDA 110 |
| 1385 | MRT4565_57540P.2 | Triticum aestivum |
| 1386 | gl_4322323 | Nepenthes alata |
| 1387 | MRT4530_46211P.2 | Oryza sativa |
| 1388 | gl_27542603 | Xerophyta humilis |
| 1389 | gl_6687375 | Digltalis grandiflora |
| 1390 | gl_5758878 | Ensete ventricosum |
| 1391 | gl_23041315 | Trichodesmium erythraeum IMS101 |
| 1392 | gl_5001573 | Austrobaileya scandens |
| 1393 | gl_32526541 | Pennantia corymbosa |
| 1394 | gl_14718228 | Sparganium americanum |
| 1395 | gl_29420855 | Kluyveromyces lactis |
| 1396 | gl_15596695 | Pseudomonas aeruglnosa PAO1 |
| 1397 | gl_15616888 | Buchnera aphidicola str. APS |
| | | (Acyrthosiphon pisum) |
| 1398 | MRT4565_140767P.1 | Triticum aestivum |
| 1399 | gl_13812075 | Guillardia theta |
| 1400 | gl_21633323 | Calystegla macrostegla |
| 1401 | gl_23003622 | Lactobacillus gasseri |
| 1402 | gl_4206604 | Ptaeroxylon obliquum |
| 1403 | gl_29346709 | Bacteroides thetaiotaomicron VPI-5482 |
| 1404 | gl_15607423 | Mycobacterium tuberculosis H37Rv |
| 1405 | gl_32487515 | Oryza sativa (japonica cultivar-group) |
| 1406 | gl_20136003 | Shigella boydii |
| 1407 | gl_12004135 | Aeglceras corniculatum |
| 1408 | gl_2493121 | Beta vulgaris |
| 1409 | gl_12229704 | Halobacterium sp. NRC-1 |
| 1410 | gl_15425580 | Forstera bellidifolia |
| 1411 | MRT3847_218049P.2 | Glycine max |
| 1412 | gl_22980706 | Ralstonia metallidurans |
| 1413 | gl_2462109 | Bacillus cereus |
| 1414 | gl_5758877 | Dimerocostus strobilaceus |
| 1415 | gl_21667292 | Adenophorus abietinus |
| 1416 | gl_24940168 | Cordia macrostachya |
| 1417 | gl_18087505 | Cucumis melo |
| 1418 | MRT3847_286526P.1 | Glycine max |
| 1419 | gl_25287618 | Arabidopsis thaliana |
| 1420 | gl_23014725 | Magnetospirillum magnetotacticum |
| 1421 | gl_7708448 | Ipheion dialystemon |
| 1422 | MRT3847_98076P.3 | Glycine max |
| 1423 | MRT4565_130085P.1 | Triticum aestivum |
| 1424 | gl_14600685 | Aeropyrum pernix |
| 1425 | gl_20384957 | Nitella praelonga |
| 1426 | MRT3847_29836P.3 | Glycine max |
| 1427 | gl_461979 | Lycopersicon esculentum |
| 1428 | gl_8517628 | Maesa myrsinoides |
| 1429 | MRT3847_233523P.2 | Glycine max |
| 1430 | gl_6687199 | Callitriche heterophylla |
| 1431 | gl_32172455 | Thermus thermophilus |
| 1432 | MRT4530_113489P.2 | Oryza sativa |
| 1433 | gl_4138679 | Vicia faba |
| 1434 | gl_14586373 | Arabidopsis thaliana |
| 1435 | MRT4530_122939P.2 | Oryza sativa |
| 1436 | gl_7488751 | Medicago sativa |
| 1437 | gl_6687379 | Decumaria barbara |
| 1438 | gl_12585499 | Eremothecium gossypii |
| 1439 | gl_7708260 | Cobaea scandens |
| 1440 | gl_13474110 | Mesorhizobium loti |
| 1441 | gl_29420835 | Saccharomyces cerevisiae |
| 1442 | gl_30171291 | Vitis vinifera |
| 1443 | gl_7688335 | Tetramerista sp. Coode 7925 |
| 1444 | gl_20136057 | Shigella dysenteriae |
| 1445 | gl_23133994 | Synechococcus sp. WH 8102 |
| 1446 | gl_4206598 | Sarcomelicope simplicifolia |
| 1447 | gl_29726150 | Pteridophyllum racemosum |
| 1448 | gl_18075915 | Columellia oblonga |
| 1449 | gl_18400939 | Arabidopsis thaliana |
| 1450 | gl_29840325 | Chlamydophila caviae GPIC |
| 1451 | gl_12004111 | Myrsine africana |
| 1452 | gl_4097515 | Nicotiana tabacum |
| 1453 | gl_15614227 | Bacillus halodurans |
| 1454 | gl_18309344 | Clostridium perfringens str. 13 |
| 1455 | gl_24460025 | Synechococcus sp. PCC 7002 |
| 1456 | gl_6689000 | Proboscidea louisianica |
| 1457 | gl_6456469 | Taraxacum officinale |
| 1458 | gl_27475608 | Medicago truncatula |
| 1459 | gl_4584556 | Beta vulgaris |
| 1460 | gl_30696138 | Arabidopsis thaliana |
| 1461 | gl_21633425 | Maripa glabra |
| 1462 | gl_20805971 | Chlamydia trachomatis |
| 1463 | gl_2541885 | Cyanidioschyzon merolae |
| 1464 | gl_14718189 | Populus tremuloides |
| 1465 | MRT3847_52308P.3 | Glycine max |
| 1466 | gl_22299818 | Thermosynechococcus elongatus BP-1 |
| 1467 | gl_6724287 | Ophioglossum reticulatum |
| 1468 | gl_14718038 | Durio zibethinus |
| 1469 | gl_7708191 | Catalpa bignonioides |
| 1470 | gl_1706547 | Hevea brasiliensis |
| 1471 | gl_400142 | Hypocrea jecorina |
| 1472 | gl_30248703 | Nitrosomonas europaea ATCC 19718 |
| 1473 | gl_4995856 | Sparrmannia ricinocarpa |
| 1474 | gl_6687737 | Hydrolea ovata |
| 1475 | gl_7708491 | Megacarpaea polyandra |
| 1476 | gl_7706839 | Averrhoa carambola |
| 1477 | gl_22972296 | Chloroflexus aurantiacus |
| 1478 | gl_13541851 | Thermoplasma volcanium |
| 1479 | gl_19705057 | Fusobacterium nucleatum subsp. nucleatum ATCC 25586 |
| 1480 | gl_19553182 | Corynebacterium glutamicum ATCC 13032 |
| 1481 | gl_4103346 | Cucumis sativus |
| 1482 | gl_1568513 | Petunia x hybrida |
| 1483 | gl_11465848 | Porphyra purpurea |
| 1484 | MRT3847_61026P.3 | Glycine max |
| 1485 | gl_29827972 | Streptomyces avermitilis MA-4680 |
| 1486 | gl_15234470 | Arabidopsis thaliana |
| 1487 | gl_7708173 | Bougainvillea glabra |
| 1488 | gl_4206564 | Cneorum pulverulentum |
| 1489 | MRT4530_76823P.2 | Oryza sativa |
| 1490 | MRT4565_110825P.1 | Triticum aestivum |
| 1491 | MRT4565_23334P.2 | Triticum aestivum |
| 1492 | gl_11467528 | Odontella sinensis |
| 1493 | gl_26989025 | Pseudomonas putida KT2440 |
| 1494 | gl_409778 | Cyanidium caldarium |
| 1495 | gl_99998 | Phaseolus vulgaris |
| 1496 | gl_11513797 | Salmonella typhimurium |
| 1497 | gl_10953877 | Hordeum vulgare subsp. vulgare |
| 1498 | gl_14587183 | Hanguana malayana |
| 1499 | gl_23502927 | Brucella suis 1330 |
| 1500 | gl_27904521 | Buchnera aphidicola str. Bp (Baizongla pistaciae) |
| 1501 | gl_21684885 | Lachnocaulon anceps |
| 1502 | gl_4033432 | Agrobacterium vitis |
| 1503 | gl_27447657 | Lycopersicon esculentum |
| 1504 | MRT4530_147074P.1 | Oryza sativa |
| 1505 | gl_15891188 | Agrobacterium tumefaciens str. C58 (Cereon) |
| 1506 | gl_23108488 | Novosphingobium aromaticivorans |
| 1507 | MRT4530_111094P.1 | Oryza sativa |
| 1508 | gl_21593407 | Arabidopsis thaliana |
| 1509 | gl_21633355 | Hildebrandtia africana |
| 1510 | gl_902938 | Glycine max |
| 1511 | gl_6467950 | Acorus gramineus |
| 1512 | gl_7708552 | Plumeria obtusa |
| 1513 | gl_24373361 | Shewanella oneidensis MR-1 |
| 1514 | gl_23467432 | Haemophilus somnus 129PT |
| 1515 | gl_15894323 | Clostridium acetobutylicum |
| 1516 | gl_12643655 | Agaricus bisporus |
| 1517 | gl_5758914 | Sparganium eurycarpum |
| 1518 | gl_16416748 | Marsilea drummondii |
| 1519 | gl_4995761 | Paramelhania decaryana |
| 1520 | gl_20385590 | Vitis vinifera |
| 1521 | gl_20530741 | Ipomoea batatas |
| 1522 | gl_6689111 | Rhynchoglossum notonianum |
| 1523 | gl_6689410 | Tagetes sp. Nickrent 3061 |
| 1524 | gl_20135989 | Shigella boydii |
| 1525 | gl_26190149 | Physcomitrella patens |
| 1526 | gl_28898813 | Vibrio parahaemolyticus RIMD 2210633 |
| 1527 | gl_19745323 | Streptococcus pyogenes MGAS8232 |
| 1528 | gl_13620169 | Capsella rubella |
| 1529 | gl_15834703 | Chlamydia muridarum |
| 1530 | MRT3847_6971P.3 | Glycine max |
| 1531 | gl_5830469 | Medicago sativa |
| 1532 | gl_775168 | Escherichia coli |
| 1533 | gl_142369 | Azotobacter vinelandii |
| 1534 | gl_11498766 | Archaeoglobus fulgldus DSM 4304 |
| 1535 | gl_28564015 | Saccharomyces bayanus |
| 1536 | MRT3847_208509P.3 | Glycine max |
| 1537 | gl_27468813 | Staphylococcus epidermidis ATCC 12228 |
| 1538 | gl_24113065 | Shigella flexneri 2a str. 301 |
| 1539 | gl_2130078 | Oryza sativa |
| 1540 | gl_261212 | Pisum sativum |
| 1541 | gl_15901171 | Streptococcus pneumoniae TIGR4 |
| 1542 | gl_19033077 | Cosmocladium perissum |
| 1543 | MRT4530_104183P.1 | Oryza sativa |
| 1544 | gl_5001589 | Kingdonia uniflora |
| 1545 | MRT4565_8769P.3 | Triticum aestivum |
| 1546 | gl_7546983 | Lactococcus lactis |
| 1547 | MRT3847_10488P.3 | Glycine max |
| 1548 | gl_4995715 | Matisia cordata |
| 1549 | gl_16124956 | Caulobacter crescentus CB15 |
| 1550 | gl_27887595 | Fusobacterium nucleatum subsp. vincentii ATCC 49256 |
| 1551 | gl_24940264 | Echiochilon pauciflorum |
| 1552 | gl_4206602 | Eremocitrus glauca |
| 1553 | gl_24940248 | Nonea versicolor |
| 1554 | gl_24215987 | Leptospira interrogans serovar lai str. 56601 |
| 1555 | gl_20136089 | Escherichia coli |
| 1556 | gl_4995105 | Dombeya sp. Chase 273 |
| 1557 | gl_8272441 | Streptococcus mutans |
| 1558 | gl_10955560 | Yersinia enterocolitica |
| 1559 | gl_16759429 | Salmonella enterica subsp. enterica serovar Typhi |
| 1560 | gl_401322 | Gossypium hirsutum |
| 1561 | gl_3777497 | Hordeum vulgare subsp. vulgare |
| 1562 | gl_14194485 | Galdieria sulphuraria |
| 1563 | MRT4565_26535P.2 | Triticum aestivum |
| 1564 | gl_729238 | Ralstonia eutropha |
| 1565 | gl_27435896 | Saglttaria latifolia |
| 1566 | gl_32441504 | Agrocybe aegerita |
| 1567 | MRT4530_81439P.1 | Oryza sativa |
| 1568 | gl_15901640 | Streptococcus pneumoniae TIGR4 |
| 1569 | MRT3847_42675P.2 | Glycine max |
| 1570 | MRT3847_24864P.2 | Glycine max |
| 1571 | gl_3169287 | Gossypium hirsutum |
| 1572 | gl_6324923 | Saccharomyces cerevisiae |
| 1573 | gl_2493099 | Haloferax volcanii |
| 1574 | gl_22983077 | Burkholderia fungorum |
| 1575 | gl_147276 | Escherichia coli |
| 1576 | MRT4530_27060P.2 | Oryza sativa |
| 1577 | gl_27804365 | Chrysanthemum x morifolium |
| 1578 | gl_16127677 | Caulobacter crescentus CB15 |
| 1579 | MRT3847_33513P.3 | Glycine max |
| 1580 | gl_3212365 | Salmonella typhimurium |
| 1581 | MRT4565_38061P.3 | Triticum aestivum |
| 1582 | gl_32409603 | Neurospora crassa |
| 1583 | gl_15924664 | Staphylococcus aureus subsp. aureus Mu50 |
| 1584 | gl_21684909 | Pharus parvifolius |
| 1585 | gl_23501986 | Brucella suis 1330 |
| 1586 | gl_20384955 | Chara rusbyana |
| 1587 | gl_15835199 | Chlamydia muridarum |
| 1588 | gl_3850926 | Isopogon buxifolius |
| 1589 | gl_12004137 | Lysimachia maxima |
| 1590 | MRT4530_146073P.1 | Oryza sativa |
| 1591 | gl_27804891 | Myxococcus xanthus |
| 1592 | gl_13540883 | Thermoplasma volcanium |
| 1593 | gl_7708468 | Lactoris fernandeziana |
| 1594 | gl_15645984 | Helicobacter pylori 26695 |
| 1595 | gl_15618021 | Chlamydophila pneumoniae CWL029 |
| 1596 | gl_29134857 | Hordeum vulgare subsp. vulgare |
| 1597 | gl_30021917 | Bacillus cereus ATCC 14579 |
| 1598 | gl_4995858 | Tilia platyphyllos |
| 1599 | gl_27528478 | Saccharomyces exiguus |
| 1600 | gl_2493120 | Acetabularia acetabulum |
| 1601 | MRT4530_103360P.1 | Oryza sativa |
| 1602 | gl_5758911 | Sansevieria socotrana |
| 1603 | gl_12005284 | Amborella trichopoda |
| 1604 | gl_18077603 | Polyosma cunninghamii |
| 1605 | gl_16973296 | Malus x domestica |
| 1606 | MRT4530_76824P.2 | Oryza sativa |
| 1607 | gl_2098385 | Salmonella typhimurium |
| 1608 | gl_20269069 | Sesbania rostrata |
| 1609 | MRT4565_41750P.3 | Triticum aestivum |
| 1610 | gl_3668069 | Lycopersicon esculentum |
| 1611 | gl_21633423 | Dicranostyles mildbraediana |
| 1612 | gl_11466794 | Oryza sativa (japonica cultivar-group) |
| 1613 | gl_5758910 | Ruscus aculeatus |
| 1614 | MRT4530_18787P.2 | Oryza sativa |
| 1615 | gl_14718095 | Kiggelaria africana |
| 1616 | gl_23051710 | Methanosarcina barkeri |
| 1617 | gl_7716952 | Medicago truncatula |
| 1618 | MRT4530_37726P.2 | Oryza sativa |
| 1619 | gl_27367975 | Vibrio vulnificus CMCP6 |
| 1620 | gl_5834682 | Rhizobium etli |
| 1621 | gl_15231135 | Arabidopsis thaliana |
| 1622 | MRT4530_14452P.1 | Oryza sativa |
| 1623 | gl_21226882 | Methanosarcina mazei Goe1 |
| 1624 | gl_15595233 | Pseudomonas aeruglnosa PAO1 |
| 1625 | gl_29654073 | Coxiella burnetii RSA 493 |
| 1626 | gl_12004113 | Grammadenia sp. Stahl 1579 |
| 1627 | gl_16329464 | Synechocystis sp. PCC 6803 |
| 1628 | gl_20269418 | Heliamphora sp. Anderberg s.n. |
| 1629 | gl_14718222 | Shepherdia canadensis |
| 1630 | gl_22128591 | Petunia x hybrida |
| 1631 | gl_23054147 | Geobacter metallireducens |
| 1632 | gl_24528335 | Emericella nidulans |
| 1633 | gl_32441506 | Pleurotus ostreatus |
| 1634 | gl_29345937 | Bacteroides thetaiotaomicron VPI-5482 |
| 1635 | gl_7489434 | Hordeum vulgare |
| 1636 | gl_23006623 | Magnetospirillum magnetotacticum |
| 1637 | gl_15794478 | Neisseria meningltidis Z2491 |
| 1638 | gl_1791247 | Chlamydia trachomatis |
| 1639 | gl_14718003 | Chrysobalanus icaco |
| 1640 | gl_6017806 | Itea ilicifolia |
| 1641 | gl_7489096 | Nicotiana sylvestris |
| 1642 | gl_19552720 | Corynebacterium glutamicum ATCC 13032 |
| 1643 | MRT3847_223708P.3 | Glycine max |
| 1644 | MRT4565_4354P.3 | Triticum aestivum |
| 1645 | gl_4433778 | Hydrogenophilus thermoluteolus |
| 1646 | gl_119006 | Phaseolus vulgaris |
| 1647 | gl_15605438 | Chlamydia trachomatis |
| 1648 | gl_15795149 | Arabidopsis thaliana |
| 1649 | gl_67842 | Spinacia oleracea |
| 1650 | gl_10953875 | Hordeum vulgare subsp. vulgare |
| 1651 | gl_1041768 | Acer pseudoplatanus |
| 1652 | gl_15966542 | Sinorhizobium meliloti |
| 1653 | gl_22960295 | Rhodobacter sphaeroides |
| 1654 | gl_16761259 | Salmonella enterica subsp. enterica serovar Typhi |
| 1655 | MRT4530_87660P.1 | Oryza sativa |
| 1656 | gl_24940196 | Buglossoides arvensis |
| 1657 | MRT3847_37502P.1 | Glycine max |
| 1658 | gl_23429044 | Cocos nucifera |
| 1659 | gl_14718153 | Nuphar variegata |
| 1660 | gl_18379267 | Arabidopsis thaliana |
| 1661 | gl_20807894 | Thermoanaerobacter tengcongensis |
| 1662 | gl_23010914 | Magnetospirillum magnetotacticum |
| 1663 | gl_28212071 | Clostridium tetani E88 |
| 1664 | MRT4565_106072P.1 | Triticum aestivum |
| 1665 | gl_23053574 | Geobacter metallireducens |
| 1666 | gl_18978077 | Pyrococcus furiosus DSM 3638 |
| 1667 | gl_30248063 | Nitrosomonas europaea ATCC 19718 |
| 1668 | gl_12006484 | Calystegla sepium |
| 1669 | gl_11465459 | Cyanidium caldarium |
| 1670 | gl_7708256 | Cinchona pubescens |
| 1671 | gl_19112558 | Schizosaccharomyces pombe |
| 1672 | gl_22328782 | Arabidopsis thaliana |
| 1673 | gl_15639519 | Treponema pallidum |
| 1674 | gl_7573598 | Populus nigra |
| 1675 | gl_136266 | Thermus thermophilus |
| 1676 | gl_3915597 | Arabidopsis thaliana |
| 1677 | gl_29840442 | Chlamydophila caviae GPIC |
| 1678 | MRT3847_33514P.2 | Glycine max |
| 1679 | gl_22991262 | Enterococcus faecium |
| 1680 | gl_4206592 | Lunasia amara |
| 1681 | gl_28188331 | Coleochaete sp. 18b3 |
| 1682 | gl_7708163 | Barringtonia asiatica |
| 1683 | MRT4565_61922P.2 | Triticum aestivum |
| 1684 | gl_15828707 | Mycoplasma pulmonis |
| 1685 | gl_23000680 | Magnetococcus sp. MC-1 |
| 1686 | gl_3850958 | Macadamia jansenii |
| 1687 | gl_8452718 | Parnassia palustris |
| 1688 | gl_32441499 | Stropharia aeruglnosa |
| 1689 | MRT3847_257212P.1 | Glycine max |
| 1690 | gl_14718224 | Siphonodon celastrineus |
| 1691 | MRT3847_213371P.3 | Glycine max |
| 1692 | gl_15800168 | Escherichia coli O157:H7 EDL933 |
| 1693 | gl_15921917 | Sulfolobus tokodaii |
| 1694 | gl_28565038 | Kluyveromyces lactis |
| 1695 | gl_21633383 | Wilsonia backhousei |
| 1696 | gl_25814821 | Stigmatella aurantiaca |
| 1697 | gl_16801989 | Listeria innocua |
| 1698 | gl_28194504 | Medicago truncatula |
| 1699 | gl_12004127 | Ardisiandra wettsteinii |
| 1700 | gl_23466988 | Haemophilus somnus 129PT |
| 1701 | gl_6729356 | Selenomonas ruminantium |
| 1702 | gl_25346630 | Arabidopsis thaliana |
| 1703 | gl_14520674 | Pyrococcus abyssi |
| 1704 | MRT4565_118038P.1 | Triticum aestivum |
| 1705 | gl_7688339 | Trigonobalanus verticillata |
| 1706 | gl_22298053 | Thermosynechococcus elongatus BP-1 |
| 1707 | gl_5911463 | Agaricus bisporus |
| 1708 | gl_23131734 | Prochlorococcus marinus str. MIT 9313 |
| 1709 | gl_7687964 | Brasenia schreberi |
| 1710 | gl_4883425 | Cicer arietinum |
| 1711 | MRT4530_28144P.1 | Oryza sativa |
| 1712 | gl_3913035 | Trifolium repens |
| 1713 | gl_19033061 | Tolypella prolifera |
| 1714 | gl_6970413 | Rosa rugosa |
| 1715 | gl_22962067 | Rhodopseudomonas palustris |
| 1716 | MRT3847_11589P.3 | Glycine max |
| 1717 | gl_28901282 | Vibrio parahaemolyticus RIMD 2210633 |
| 1718 | gl_3850988 | Grevillea baileyana |
| 1719 | gl_17988331 | Brucella melitensis 16M |
| 1720 | gl_7708153 | Antirrhinum majus |
| 1721 | gl_136261 | Methanothermobacter marburgensis str. Marburg |
| 1722 | gl_32490885 | Wigglesworthia glossinidia endosymbiont of Glossina brevipalpis |
| 1723 | gl_27528472 | Saccharomyces cariocanus |
| 1724 | gl_21399162 | Bacillus anthracis str. A2012 |
| 1725 | gl_28867399 | Pseudomonas syringae pv. tomato str. DC3000 |
| 1726 | gl_15425576 | Escallonia rubra |
| 1727 | gl_1004320 | Sulfolobus solfataricus |
| 1728 | gl_23465516 | Bifidobacterium longum NCC2705 |
| 1729 | gl_11357336 | Arabidopsis thaliana |
| 1730 | MRT4530_104720P.2 | Oryza sativa |
| 1731 | gl_20136053 | Shigella dysenteriae |
| 1732 | MRT4530_120903P.1 | Oryza sativa |
| 1733 | gl_22966395 | Rhodospirillum rubrum |
| 1734 | gl_9799472 | Mytilaria laosensis |
| 1735 | gl_23503627 | Pseudocarteria mucosa |
| 1736 | gl_2500204 | Corynebacterium ammoniagenes |
| 1737 | gl_6017838 | Heuchera sanguinea |
| 1738 | gl_6225174 | Yersinia enterocolitica |
| 1739 | MRT3847_36848P.3 | Glycine max |
| 1740 | gl_30019391 | Bacillus cereus ATCC 14579 |
| 1741 | gl_17987729 | Brucella melitensis 16M |
| 1742 | gl_12004139 | Lysimachia minoricensis |
| 1743 | MRT4530_87661P.1 | Oryza sativa |
| 1744 | gl_18075929 | Escallonia resinosa |
| 1745 | gl_22091479 | Daucus carota subsp. sativus |
| 1746 | gl_29893654 | Oryza sativa (japonica cultivar-group) |
| 1747 | gl_24940180 | Echium vulgare |
| 1748 | gl_30267072 | Ipomoea umbraticola |
| 1749 | gl_3702409 | Cichorium intybus x Cichorium endivia |
| 1750 | MRT3847_215323P.2 | Glycine max |
| 1751 | gl_15965009 | Sinorhizobium meliloti |
| 1752 | gl_14717924 | Agave ghiesbreghtii |
| 1753 | gl_16416736 | Isoetes engelmannii |
| 1754 | gl_6318287 | Thermoproteus tenax |
| 1755 | gl_7708658 | Stackhousia minima |
| 1756 | gl_28071332 | Oryza sativa (japonica cultivar-group) |
| 1757 | gl_14574707 | Nostoc punctiforme |
| 1758 | gl_17646111 | Nicotiana tabacum |
| 1759 | gl_25089839 | Parthenium argentatum |
| 1760 | gl_15668390 | Methanocaldococcus jannaschii |
| 1761 | gl_11497535 | Spinacia oleracea |
| 1762 | gl_16549060 | Magnolia praecocissima |
| 1763 | gl_22265999 | Hordeum vulgare |
| 1764 | gl_20269416 | Halesia carolina |
| 1765 | gl_15673109 | Lactococcus lactis subsp. lactis |
| 1766 | gl_6688636 | Melanophylla alnifolia |
| 1767 | gl_28380210 | Azospirillum brasilense |
| 1768 | MRT4530_25301P.1 | Oryza sativa |
| 1769 | gl_29420861 | Saccharomyces exiguus |
| 1770 | MRT3847_199862P.2 | Glycine max |
| 1771 | gl_6689307 | Sesamum indicum |
| 1772 | gl_4887235 | Hyacinthus orientalis |
| 1773 | MRT4530_10021P.1 | Oryza sativa |
| 1774 | gl_15893448 | Clostridium acetobutylicum |
| 1775 | gl_32526543 | Pennantia cunninghamii |
| 1776 | gl_7708268 | Dicella nucifera |
| 1777 | gl_4995183 | Hermannia erodioides |
| 1778 | gl_7489198 | Nicotiana tabacum |
| 1779 | MRT4530_100340P.1 | Oryza sativa |
| 1780 | MRT4565_9771P.3 | Triticum aestivum |
| 1781 | MRT4530_19282P.1 | Oryza sativa |
| 1782 | gl_15425560 | Brunonia australis |
| 1783 | MRT4530_103362P.1 | Oryza sativa |
| 1784 | gl_12004115 | Douglasia nivalis |
| 1785 | gl_4063542 | Cupaniopsis anacardioides |
| 1786 | gl_4995798 | Schoutenia glomerata |
| 1787 | gl_19698536 | Hordeum vulgare subsp. vulgare |
| 1788 | gl_30265620 | Ipomoea cordatotriloba |
| 1789 | gl_20146358 | Oryza sativa (japonica cultivar-group) |
| 1790 | gl_29349251 | Bacteroides thetaiotaomicron VPI-5482 |
| 1791 | gl_24940174 | Cystostemon heliocharis |
| 1792 | gl_23465557 | Bifidobacterium longum NCC2705 |
| 1793 | gl_14718151 | Nolina recurvata |
| 1794 | gl_13508042 | Mycoplasma pneumoniae |
| 1795 | gl_22960297 | Rhodobacter sphaeroides |
| 1796 | gl_6689231 | Scrophularia californica |
| 1797 | MRT3847_53577P.3 | Glycine max |
| 1798 | MRT3847_58239P.2 | Glycine max |
| 1799 | gl_15236304 | Arabidopsis thaliana |
| 1800 | MRT4530_140459P.1 | Oryza sativa |
| 1801 | gl_6226270 | Mycobacterium intracellulare |
| 1802 | gl_23050672 | Methanosarcina barkeri |
| 1803 | gl_3334408 | Acetabularia acetabulum |
| 1804 | gl_25409314 | Halobacterium sp. NRC-1 |
| 1805 | gl_30263713 | Bacillus anthracis str. Ames |
| 1806 | gl_5305242 | Brassica rapa |
| 1807 | gl_18407057 | Arabidopsis thaliana |
| 1808 | gl_22970179 | Chloroflexus aurantiacus |
| 1809 | gl_15608751 | Mycobacterium tuberculosis H37Rv |
| 1810 | gl_27904899 | Buchnera aphidicola str. Bp (Baizongla pistaciae) |
| 1811 | MRT4530_37728P.2 | Oryza sativa |
| 1812 | MRT4530_87778P.1 | Oryza sativa |
| 1813 | gl_14279306 | Vitis vinifera |
| 1814 | MRT4530_14454P.2 | Oryza sativa |
| 1815 | gl_6689408 | Titanotrichum oldhamii |
| 1816 | gl_23137115 | Cytophaga hutchinsonii |
| 1817 | gl_17227784 | Nostoc sp. PCC 7120 |
| 1818 | gl_21633339 | Aniseia cernua |
| 1819 | gl_13473275 | Mesorhizobium loti |
| 1820 | gl_7688337 | Trema micrantha |
| 1821 | gl_20136019 | Shigella flexneri |
| 1822 | gl_15791717 | Campylobacter jejuni subsp. jejuni NCTC 11168 |
| 1823 | gl_7708622 | Rourea minor |
| 1824 | gl_15639499 | Treponema pallidum |
| 1825 | gl_7708442 | Fouquieria columnaris |
| 1826 | gl_11558464 | Deutzia rubens |
| 1827 | gl_4995792 | Ruizia cordata |
| 1828 | gl_6706180 | Gilia capitata |
| 1829 | gl_18075917 | Desfontainia spinosa |
| 1830 | gl_12655901 | Brassica napus |
| 1831 | gl_15217662 | Arabidopsis thaliana |
| 1832 | gl_11321164 | Capsicum annuum |
| 1833 | gl_14718030 | Dialypetalanthus fuscescens |
| 1834 | gl_7271955 | Lilium longlflorum |
| 1835 | MRT4565_9346P.3 | Triticum aestivum |
| 1836 | MRT3847_242965P.2 | Glycine max |
| 1837 | gl_16760154 | Salmonella enterica subsp. enterica serovar Typhi |
| 1838 | gl_21633381 | Wilsonia humilis |
| 1839 | gl_19033051 | Chara connivens |
| 1840 | gl_23135446 | Cytophaga hutchinsonii |
| 1841 | MRT3847_36849P.2 | Glycine max |
| 1842 | gl_20093841 | Methanopyrus kandleri AV19 |
| 1843 | gl_15608935 | Mycobacterium tuberculosis H37Rv |
| 1844 | gl_460160 | Saccharomyces cerevisiae |
| 1845 | gl_15227441 | Arabidopsis thaliana |
| 1846 | gl_15616426 | Bacillus halodurans |
| 1847 | gl_6689309 | Sollya heterophylla |
| 1848 | gl_7708558 | Pouteria macrantha |
| 1849 | gl_5922599 | Allium macrostemon |
| 1850 | gl_13474231 | Mesorhizobium loti |
| 1851 | gl_11467561 | Odontella sinensis |
| 1852 | gl_29427825 | Lycopersicon peruvianum |
| 1853 | gl_1469934 | Nicotiana glutinosa |
| 1854 | gl_23475131 | Desulfovibrio desulfuricans G20 |
| 1855 | gl_11278993 | Lycopersicon esculentum |
| 1856 | gl_125607 | Emericella nidulans |
| 1857 | gl_6467935 | Triglochin maritimum |
| 1858 | gl_21633369 | Breweria rotundifolia |
| 1859 | gl_28194506 | Lotus japonicus |
| 1860 | MRT3847_272723P.1 | Glycine max |
| 1861 | gl_5758895 | Maranta bicolor |
| 1862 | gl_15673314 | Lactococcus lactis subsp. lactis |
| 1863 | gl_5031147 | Trochodendron aralioides |
| 1864 | gl_3850922 | Petrophile circinata |
| 1865 | gl_16332067 | Synechocystis sp. PCC 6803 |
| 1866 | gl_25028545 | Corynebacterium efficiens YS-314 |
| 1867 | gl_21684891 | Paepalanthus fasciculatus |
| 1868 | gl_15602518 | Pasteurella multocida |
| 1869 | gl_24940260 | Phacelia grandiflora |
| 1870 | gl_22997030 | Xylella fastidiosa Ann-1 |
| 1871 | gl_20805999 | Chlamydia trachomatis |
| 1872 | gl_15604535 | Rickettsia prowazekii |
| 1873 | gl_7489412 | Hordeum vulgare |
| 1874 | gl_6687660 | Guettarda uruguensis |
| 1875 | gl_6687201 | Cyrtandra hawaiensis |
| 1876 | gl_23052059 | Methanosarcina barkeri |
| 1877 | MRT4565_52146P.2 | Triticum aestivum |
| 1878 | gl_21244070 | Xanthomonas axonopodis pv. citri str. 306 |
| 1879 | gl_19033063 | Coleochaete orbicularis |
| 1880 | gl_20808006 | Thermoanaerobacter tengcongensis |
| 1881 | gl_20136051 | Shigella dysenteriae |
| 1882 | gl_5758894 | Liriope muscari |
| 1883 | gl_7708454 | Jasminum polyanthum |
| 1884 | gl_5834521 | Cichorium intybus x Cichorium endivia |
| 1885 | gl_30682129 | Arabidopsis thaliana |
| 1886 | gl_16122303 | Yersinia pestis CO92 |
| 1887 | MRT4565_88207P.2 | Triticum aestivum |
| 1888 | gl_23023390 | Leuconostoc mesenteroides subsp. mesenteroides ATCC 8293 |
| 1889 | gl_6689006 | Phyllonoma laticuspis |
| 1890 | gl_11465473 | Cyanidium caldarium |
| 1891 | MRT3847_284135P.1 | Glycine max |
| 1892 | gl_15611020 | Mycobacterium tuberculosis H37Rv |
| 1893 | gl_23103063 | Azotobacter vinelandii |
| 1894 | gl_322787 | Solanum tuberosum |
| 1895 | gl_30351931 | Brimeura amethystina |
| 1896 | gl_231596 | Cuscuta reflexa |
| 1897 | gl_14626277 | Oryza sativa (japonica cultivar-group) |
| 1898 | gl_20136039 | Shigella flexneri |
| 1899 | MRT3847_98062P.3 | Glycine max |
| 1900 | gl_24940164 | Buglossoides purpurocaerulea |
| 1901 | gl_7487603 | Arabidopsis thaliana |
| 1902 | gl_29828077 | Streptomyces avermitilis MA-4680 |
| 1903 | gl_1072952 | Thermus aquaticus |
| 1904 | gl_320885 | Asperglllus niger |
| 1905 | gl_20465197 | Bartonella henselae |
| 1906 | gl_28971666 | Burkholderia multivorans |
| 1907 | MRT4565_34024P.3 | Triticum aestivum |
| 1908 | gl_4218160 | Gerbera hybrid cv. [Terra Reglna] |
| 1909 | gl_136260 | Lactobacillus casei |
| 1910 | gl_29375625 | Enterococcus faecalis V583 |
| 1911 | gl_30267058 | Ipomoea nil |
| 1912 | gl_1345505 | Arabidopsis thaliana |
| 1913 | gl_28373459 | Salmonella typhimurium |
| 1914 | MRT4565_60761P.2 | Triticum aestivum |
| 1915 | gl_16416738 | Tmesipteris obliqua |
| 1916 | gl_27528498 | Saccharomyces servazzii |
| 1917 | gl_4995717 | Muntingla calabura |
| 1918 | gl_15827655 | Mycobacterium leprae |
| 1919 | gl_27550061 | Photorhabdus luminescens |
| 1920 | gl_24940266 | Tiquilia plicata |
| 1921 | gl_21219540 | Streptomyces coelicolor A3(2) |
| 1922 | gl_16416760 | Sphagnum palustre |
| 1923 | gl_4063540 | Cistus revolii |
| 1924 | MRT4565_127690P.1 | Triticum aestivum |
| 1925 | gl_6687548 | Erithalis fruticosa |
| 1926 | gl_17933944 | Agrobacterium tumefaciens str. C58 (U. Washington) |
| 1927 | gl_15678973 | Methanothermobacter thermautotrophicus str. Delta H |
| 1928 | gl_21593950 | Arabidopsis thaliana |
| 1929 | gl_602764 | Arabidopsis thaliana |
| 1930 | gl_14717984 | Callitriche heterophylla |
| 1931 | gl_3913209 | Rhodobacter sphaeroides |
| 1932 | MRT3847_61998P.3 | Glycine max |
| 1933 | gl_15229157 | Arabidopsis thaliana |
| 1934 | gl_7484643 | Beta vulgaris |
| 1935 | gl_22331664 | Arabidopsis thaliana |
| 1936 | gl_22962301 | Rhodopseudomonas palustris |
| 1937 | MRT4565_25946P.3 | Triticum aestivum |
| 1938 | gl_15027611 | Cryptococcus neoformans var. grubii |
| 1939 | gl_5758891 | Hemerocallis lilioasphodelus |
| 1940 | gl_15616928 | Buchnera aphidicola str. APS (Acyrthosiphon pisum) |
| 1941 | gl_12004117 | Dodecatheon meadia |
| 1942 | gl_15604890 | Chlamydia trachomatis |
| 1943 | gl_4103486 | Pinus radiata |
| 1944 | gl_32441496 | Trametes versicolor |
| 1945 | gl_541528 | Cyanidium caldarium |
| 1946 | gl_6225163 | Azospirillum brasilense |
| 1947 | gl_23019267 | Thermobifida fusca |
| 1948 | gl_22328179 | Arabidopsis thaliana |
| 1949 | gl_11034791 | Gnetum gnemon |
| 1950 | gl_21673370 | Chlorobium tepidum TLS |
| 1951 | gl_23473416 | Desulfovibrio desulfuricans G20 |
| 1952 | gl_27904753 | Buchnera aphidicola str. Bp (Baizongla pistaciae) |
| 1953 | gl_15425574 | Echinops bannaticus |
| 1954 | gl_15827806 | Mycobacterium leprae |
| 1955 | gl_7708560 | Prostanthera ovalifolia |
| 1956 | MRT4565_113424P.1 | Triticum aestivum |
| 1957 | gl_7486722 | Arabidopsis thaliana |
| 1958 | gl_23469166 | Pseudomonas syringae pv. syringae B728a |
| 1959 | gl_16800736 | Listeria innocua |
| 1960 | gl_23128273 | Nostoc punctiforme |
| 1961 | gl_18077605 | Quintinia verdonii |
| 1962 | gl_16129807 | Escherichia coli K12 |
| 1963 | gl_21220152 | Streptomyces coelicolor A3(2) |
| 1964 | gl_16973298 | Malus x domestica |
| 1965 | gl_464145 | Hordeum vulgare subsp. vulgare |
| 1966 | gl_28564205 | Saccharomyces castellii |
| 1967 | gl_7708304 | Frankenia pulverulenta |
| 1968 | gl_19881581 | Oryza sativa (japonica cultivar-group) |
| 1969 | gl_5305244 | Brassica oleracea |
| 1970 | gl_22990852 | Enterococcus faecium |
| 1971 | gl_21553510 | Arabidopsis thaliana |
| 1972 | gl_18310374 | Clostridium perfringens str. 13 |
| 1973 | gl_16263937 | Sinorhizobium meliloti |
| 1974 | gl_5758899 | Musa acuminata |
| 1975 | MRT4530_27618P.1 | Oryza sativa |
| 1976 | gl_15594693 | Borrelia burgdorferi B31 |
| 1977 | gl_14717950 | Barbeya oleoides |
| 1978 | MRT4530_19284P.1 | Oryza sativa |
| 1979 | MRT3847_16287P.3 | Glycine max |
| 1980 | gl_16764728 | Salmonella typhimurium LT2 |
| 1981 | gl_7708187 | Carallia brachiata |
| 1982 | gl_4206576 | Calodendrum capense |
| 1983 | gl_6687447 | Donatia sp. Morgan 2142 |
| 1984 | gl_15792017 | Campylobacter jejuni subsp. jejuni NCTC 11168 |
| 1985 | gl_6687485 | Eucnide bartonioides |
| 1986 | gl_23040075 | Trichodesmium erythraeum IMS101 |
| 1987 | gl_29420857 | Saccharomyces castellii |
| 1988 | gl_30063190 | Shigella flexneri 2a str. 2457T |
| 1989 | MRT4530_8337P.2 | Oryza sativa |
| 1990 | gl_15924363 | Staphylococcus aureus subsp. aureus Mu50 |
| 1991 | gl_14591712 | Pyrococcus horikoshii |
| 1992 | gl_15422204 | Acicarpha tribuloides |
| 1993 | gl_22956679 | Rhodobacter sphaeroides |
| 1994 | gl_21241839 | Xanthomonas axonopodis pv. citri str. 306 |
| 1995 | gl_7708157 | Asparagus officinalis |
| 1996 | gl_28493446 | Tropheryma whipplei str. Twist |
| 1997 | gl_15608755 | Mycobacterium tuberculosis H37Rv |
| 1998 | gl_19033053 | Lamprothamnium macropogon |
| 1999 | gl_15921495 | Sulfolobus tokodaii |
| 2000 | gl_32405352 | Neurospora crassa |
| 2001 | gl_5758898 | Monocostus uniflorus |
| 2002 | gl_23004962 | Magnetospirillum magnetotacticum |
| 2003 | gl_30102526 | Arabidopsis thaliana |
| 2004 | gl_28210965 | Clostridium tetani E88 |
| 2005 | gl_20808232 | Thermoanaerobacter tengcongensis |
| 2006 | gl_7706835 | Acorus calamus |
| 2007 | gl_23501390 | Brucella suis 1330 |
| 2008 | gl_15605055 | Chlamydia trachomatis |
| 2009 | gl_5231205 | Streptococcus pneumoniae |
| 2010 | gl_27526581 | Kluyveromyces thermotolerans |
| 2011 | gl_3850984 | Opisthiolepis heterophylla |
| 2012 | MRT3847_53988P.3 | Glycine max |
| 2013 | gl_7708497 | Metrosideros nervulosa |
| 2014 | MRT3847_161472P.3 | Glycine max |
| 2015 | gl_169779 | Oryza sativa |
| 2016 | gl_3122320 | Mycobacterium intracellulare |
| 2017 | MRT3847_249176P.2 | Glycine max |
| 2018 | gl_15239624 | Arabidopsis thaliana |
| 2019 | gl_14718090 | Ixonanthes icosandra |
| 2020 | gl_25956266 | Lotus japonicus |
| 2021 | gl_5758897 | Mayaca aubletii |
| 2022 | gl_29376200 | Enterococcus faecalis V583 |
| 2023 | gl_32029713 | Haemophilus somnus 2336 |
| 2024 | gl_15608450 | Mycobacterium tuberculosis H37Rv |
| 2025 | gl_3850908 | Symphionema montanum |
| 2026 | gl_29420837 | Saccharomyces cerevisiae |
| 2027 | gl_12004165 | Soldanella montana |
| 2028 | gl_27883932 | Lycopersicon esculentum |
| 2029 | gl_15900780 | Streptococcus pneumoniae TIGR4 |
| 2030 | gl_15232517 | Arabidopsis thaliana |
| 2031 | gl_13235340 | Mesembryanthemum crystallinum |
| 2032 | gl_7708646 | Sloanea berteriana |
| 2033 | gl_29833453 | Streptomyces avermitilis MA-4680 |
| 2034 | gl_14717920 | Abatia parviflora |
| 2035 | gl_608671 | Arabidopsis thaliana |
| 2036 | gl_15615725 | Bacillus halodurans |
| 2037 | gl_23021827 | Clostridium thermocellum ATCC 27405 |
| 2038 | gl_98485 | Bacillus subtilis |
| 2039 | gl_7688039 | Schisandra sphenanthera |
| 2040 | MRT3847_7845P.3 | Glycine max |
| 2041 | MRT3847_51771P.3 | Glycine max |
| 2042 | gl_23502956 | Brucella suis 1330 |
| 2043 | gl_5758896 | Marantochloa atropurpurea |
| 2044 | gl_20330751 | Oryza sativa (japonica cultivar-group) |
| 2045 | gl_3850950 | Musgravea heterophylla |
| 2046 | gl_7442732 | Solanum tuberosum |
| 2047 | gl_15676021 | Neisseria meningltidis MC58 |
| 2048 | gl_4206578 | Severinia buxifolia |
| 2049 | MRT4565_28703P.3 | Triticum aestivum |
| 2050 | gl_15843516 | Mycobacterium tuberculosis CDC1551 |
| 2051 | gl_4995767 | Pterospermum celebicum |
| 2052 | gl_22976982 | Ralstonia metallidurans |
| 2053 | gl_11467696 | Guillardia theta |
| 2054 | gl_21633405 | Dipteropeltis poranoides |
| 2055 | gl_6599365 | Pistacia vera |
| 2056 | gl_30267056 | Ipomoea littoralis |
| 2057 | gl_16225426 | Castanea sativa |
| 2058 | gl_15594440 | Borrelia burgdorferi B31 |
| 2059 | MRT3847_28679P.3 | Glycine max |
| 2060 | gl_3041863 | Bacillus subtilis |
| 2061 | MRT4530_10024P.1 | Oryza sativa |
| 2062 | MRT4565_71415P.2 | Triticum aestivum |
| 2063 | gl_14718136 | Mollugo verticillata |
| 2064 | gl_5231196 | Streptococcus pneumoniae |
| 2065 | gl_14718060 | Galphimia gracilis |
| 2066 | gl_16079320 | Bacillus subtilis subsp. subtilis str. 168 |
| 2067 | MRT4565_30002P.3 | Triticum aestivum |
| 2068 | gl_1296452 | Bacillus subtilis |
| 2069 | gl_20136067 | Shigella sonnei |
| 2070 | gl_22992679 | Enterococcus faecium |
| 2071 | MRT3847_239538P.2 | Glycine max |
| 2072 | gl_23473439 | Desulfovibrio desulfuricans G20 |
| 2073 | gl_24940256 | Patagonula americana |
| 2074 | gl_22775591 | Cryptococcus neoformans var. neoformans |
| 2075 | gl_22996222 | Xylella fastidiosa Ann-1 |
| 2076 | gl_22653795 | Mesorhizobium loti |
| 2077 | gl_15982954 | Prunus persica |
| 2078 | gl_6970415 | Rosa rugosa |
| 2079 | gl_32441494 | Auricularia auricula-judae |
| 2080 | gl_15790973 | Halobacterium sp. NRC-1 |
| 2081 | gl_9955873 | Asperglllus oryzae |
| 2082 | gl_478405 | Secale cereale |
| 2083 | gl_23115534 | Desulfitobacterium hafniense |
| 2084 | MRT4565_20121P.3 | Triticum aestivum |
| 2085 | gl_227786 | Sorghum bicolor |
| 2086 | gl_15601464 | Vibrio cholerae |
| 2087 | gl_21633399 | Itzaea sericea |
| 2088 | gl_14600753 | Aeropyrum pernix |
| 2089 | gl_1170699 | Yarrowia lipolytica |
| 2090 | gl_28378350 | Lactobacillus plantarum WCFS1 |
| 2091 | MRT3847_53989P.3 | Glycine max |
| 2092 | gl_20136015 | Shigella boydii |
| 2093 | gl_15827659 | Mycobacterium leprae |
| 2094 | MRT3847_241638P.2 | Glycine max |
| 2095 | gl_28564203 | Saccharomyces castellii |
| 2096 | gl_32423711 | primary endosymbiont of Bemisia tabaci |
| 2097 | gl_12004119 | Diospyros digyna |
| 2098 | gl_23428880 | Lycopersicon esculentum |
| 2099 | gl_8134368 | Myxococcus xanthus |
| 2100 | gl_21401812 | Bacillus anthracis str. A2012 |
| 2101 | gl_2981133 | Populus balsamifera subsp. trichocarpa |
| 2102 | MRT3847_249177P.2 | Glycine max |
| 2103 | MRT3847_250748P.2 | Glycine max |
| 2104 | gl_28564230 | Saccharomyces castellii |
| 2105 | gl_7708171 | Borago officinalis |
| 2106 | gl_25402689 | Arabidopsis thaliana |
| 2107 | gl_22988101 | Burkholderia fungorum |
| 2108 | gl_100285 | Nicotiana sp. |
| 2109 | gl_4995852 | Trochetiopsis erythroxylon |
| 2110 | gl_25005270 | Lactobacillus delbrueckii subsp. lactis |
| 2111 | gl_21219937 | Streptomyces coelicolor A3(2) |
| 2112 | gl_24940182 | Ehretia cymosa |
| 2113 | MRT3847_43842P.3 | Glycine max |
| 2114 | gl_15897484 | Sulfolobus solfataricus |
| 2115 | MRT4530_54700P.1 | Oryza sativa |
| 2116 | gl_5231181 | Streptococcus pneumoniae |
| 2117 | gl_25307910 | Arabidopsis thaliana |
| 2118 | MRT4530_118075P.1 | Oryza sativa |
| 2119 | gl_4995107 | Eriolaena spectabilis |
| 2120 | gl_7452981 | Hordeum vulgare subsp. vulgare |
| 2121 | gl_585371 | Geobacillus stearothermophilus |
| 2122 | gl_26247590 | Escherichia coli CFT073 |
| 2123 | gl_399096 | Brassica napus |
| 2124 | gl_4164408 | Ricinus communis |
| 2125 | gl_32034348 | Actinobacillus pleuropneumoniae serovar 1 str. 4074 |
| 2126 | gl_20092951 | Methanosarcina acetivorans C2A |
| 2127 | MRT4565_14604P.1 | Triticum aestivum |
| 2128 | MRT3847_7846P.2 | Glycine max |
| 2129 | gl_21633365 | Evolvulus glomeratus |
| 2130 | gl_29420863 | Saccharomyces exiguus |
| 2131 | gl_6688706 | Montinia caryophyllacea |
| 2132 | gl_21633301 | Merremia aegyptia |
| 2133 | gl_23023764 | Leuconostoc mesenteroides subsp. mesenteroides ATCC 8293 |
| 2134 | gl_7708306 | Fuchsia procumbens |
| 2135 | gl_8452779 | Staphylea trifolia |
| 2136 | gl_4995790 | Reevesia thyrsoidea |
| 2137 | gl_15242347 | Arabidopsis thaliana |
| 2138 | gl_30684104 | Arabidopsis thaliana |
| 2139 | gl_20136069 | Shigella sonnei |
| 2140 | gl_5758867 | Costus malortieanus |
| 2141 | gl_16129221 | Escherichia coli K12 |
| 2142 | gl_3023975 | Borrelia burgdorferi |
| 2143 | MRT4565_118744P.1 | Triticum aestivum |
| 2144 | gl_15220923 | Arabidopsis thaliana |
| 2145 | MRT4530_91129P.1 | Oryza sativa |
| 2146 | gl_27372782 | Populus tremuloides |
| 2147 | gl_23122758 | Prochlorococcus marinus subsp. pastoris str. CCMP1378 |
| 2148 | gl_169777 | Oryza sativa |
| 2149 | gl_4063528 | Berrya javanica |
| 2150 | gl_32483423 | Oryza sativa (japonica cultivar-group) |
| 2151 | gl_9663979 | Oryza sativa (japonica cultivar-group) |
| 2152 | gl_21633437 | Porana paniculata |
| 2153 | gl_4995846 | Theobroma cacao |
| 2154 | gl_19033055 | Lychnothamnus barbatus |
| 2155 | gl_20218805 | Pinus pinaster |
| 2156 | gl_20805967 | Chlamydia trachomatis |
| 2157 | gl_28378504 | Lactobacillus plantarum WCFS1 |
| 2158 | gl_15591909 | Arabidopsis thaliana |
| 2159 | gl_15241190 | Arabidopsis thaliana |
| 2160 | gl_3850906 | Agastachys odorata |
| 2161 | gl_775193 | Escherichia coli |
| 2162 | gl_17227820 | Nostoc sp. PCC 7120 |
| 2163 | gl_16765071 | Salmonella typhimurium LT2 |
| 2164 | gl_420929 | Ralstonia eutropha |
| 2165 | gl_5758866 | Costus barbatus |
| 2166 | gl_7708572 | Rhabdodendron amazonicum |
| 2167 | gl_4063570 | Tropaeolum tricolor |
| 2168 | gl_18311131 | Clostridium perfringens str. 13 |
| 2169 | MRT3847_272006P.1 | Glycine max |
| 2170 | gl_17224922 | Brassica napus |
| 2171 | MRT3847_30433P.3 | Glycine max |
| 2172 | gl_7488932 | Daucus carota |
| 2173 | gl_15612263 | Helicobacter pylori J99 |
| 2174 | gl_25297689 | Arabidopsis thaliana |
| 2175 | gl_20136099 | Escherichia coli |
| 2176 | gl_15791759 | Campylobacter jejuni subsp. jejuni NCTC 11168 |
| 2177 | gl_29420843 | Saccharomyces cerevisiae |
| 2178 | gl_231541 | Glycine max |
| 2179 | gl_20136035 | Shigella flexneri |
| 2180 | gl_6016881 | Bacillus sp. |
| 2181 | gl_7708628 | Saintpaulia ionantha |
| 2182 | MRT3847_254592P.2 | Glycine max |
| 2183 | gl_12004167 | Theophrasta americana |
| 2184 | MRT4565_98303P.2 | Triticum aestivum |
| 2185 | gl_15896652 | Clostridium acetobutylicum |
| 2186 | gl_20269410 | Eurya sp. Chung & Anderberg 1406 |
| 2187 | gl_23021813 | Clostridium thermocellum ATCC 27405 |
| 2188 | gl_7708570 | Reinwardtia indica |
| 2189 | gl_4063558 | Pelargonium cotyledonis |
| 2190 | gl_6324844 | Saccharomyces cerevisiae |
| 2191 | gl_13812343 | Guillardia theta |
| 2192 | gl_24940198 | Lobostemon fruticosus |
| 2193 | gl_7708197 | Coffea arabica |
| 2194 | gl_27528474 | Saccharomyces dairenensis |
| 2195 | gl_28898735 | Vibrio parahaemolyticus RIMD 2210633 |
| 2196 | gl_5881832 | Gluconobacter oxydans |
| 2197 | CGPG25.pep | Arabidopsis thaliana |
| 2198 | gl_30267054 | Ipomoea ramosissima |
| 2199 | gl_14718167 | Pelliciera rhizophorae |
| 2200 | gl_11467655 | Guillardia theta |
| 2201 | MRT3847_99459P.3 | Glycine max |
| 2202 | MRT4565_90833P.2 | Triticum aestivum |
| 2203 | MRT3847_36085P.3 | Glycine max |
| 2204 | gl_18075919 | Forgesia racemosa |
| 2205 | gl_8452620 | Bulbine succulenta |
| 2206 | gl_2245390 | Arabidopsis thaliana |
| 2207 | gl_6323699 | Saccharomyces cerevisiae |
| 2208 | gl_30688566 | Arabidopsis thaliana |
| 2209 | gl_15895897 | Clostridium acetobutylicum |
| 2210 | gl_4995059 | Byttneria filipes |
| 2211 | gl_4033725 | Picea mariana |
| 2212 | gl_24430421 | Nicotiana sylvestris |
| 2213 | MRT4530_37730P.2 | Oryza sativa |
| 2214 | gl_16554463 | Halobacterium sp. NRC-1 |
| 2215 | gl_28380215 | Buchnera aphidicola (Melaphis rhois) |
| 2216 | gl_6687278 | Cephalanthus occidentalis |
| 2217 | gl_7677378 | Lycopersicon esculentum |
| 2218 | gl_5031217 | Liquidambar styraciflua |
| 2219 | gl_32477628 | Pirellula sp. |
| 2220 | gl_27529826 | Nicotiana tabacum |
| 2221 | gl_22965894 | Rhodospirillum rubrum |
| 2222 | gl_20136065 | Shigella sonnei |
| 2223 | gl_11322499 | Hordeum vulgare |
| 2224 | gl_14717980 | Cajophora acuminata |
| 2225 | gl_1934688 | Tmesipteris tannensis |
| 2226 | gl_12655961 | Brassica rapa |
| 2227 | gl_5001583 | Cercidiphyllum japonicum |
| 2228 | MRT4565_43218P.3 | Triticum aestivum |
| 2229 | gl_23037947 | Oenococcus oeni MCW |
| 2230 | gl_23113700 | Desulfitobacterium hafniense |
| 2231 | gl_101735 | Yarrowia lipolytica |
| 2232 | gl_21282989 | Staphylococcus aureus subsp. aureus MW2 |
| 2233 | gl_23110381 | Novosphingobium aromaticivorans |
| 2234 | gl_15838086 | Xylella fastidiosa 9a5c |
| 2235 | gl_21633457 | Cuscuta japonica |
| 2236 | MRT4565_14593P.3 | Triticum aestivum |
| 2237 | gl_23336674 | Bifidobacterium longum DJO10A |
| 2238 | gl_16943745 | Polygonatum hookeri |
| 2239 | gl_24379020 | Streptococcus mutans UA159 |
| 2240 | gl_21684893 | Flagellaria indica |
| 2241 | gl_24940262 | Saccellium lanceolatum |
| 2242 | gl_14718046 | Eucryphia lucida |
| 2243 | MRT4530_57792P.1 | Oryza sativa |
| 2244 | MRT4565_36882P.3 | Triticum aestivum |
| 2245 | gl_28564264 | Saccharomyces castellii |
| 2246 | gl_21633397 | Bonamia media |
| 2247 | gl_7446527 | Arabidopsis thaliana |
| 2248 | gl_7708139 | Aextoxicon punctatum |
| 2249 | gl_9294047 | Arabidopsis thaliana |
| 2250 | gl_30060377 | Oryza sativa (japonica cultivar-group) |
| 2251 | gl_13518304 | Oenothera elata subsp. hookeri |
| 2252 | gl_421428 | Lactococcus lactis subsp. lactis |
| 2253 | gl_20136013 | Shigella boydii |
| 2254 | gl_15668279 | Methanocaldococcus jannaschii |
| 2255 | MRT4530_21629P.1 | Oryza sativa |
| 2256 | gl_7708642 | Schima superba |
| 2257 | gl_15641182 | Vibrio cholerae |
| 2258 | gl_6017792 | Haloragls erecta |
| 2259 | gl_6539568 | Oryza sativa (japonica cultivar-group) |
| 2260 | gl_15594957 | Borrelia burgdorferi B31 |
| 2261 | gl_6687120 | Cajophora acuminata |
| 2262 | gl_5834523 | Cichorium intybus x Cichorium endivia |
| 2263 | MRT3847_2805P.3 | Glycine max |
| 2264 | gl_2981131 | Populus balsamifera subsp. trichocarpa |
| 2265 | gl_19033087 | Mougeotia sp. UTEX LB 758 |
| 2266 | gl_2105144 | Treponema denticola |
| 2267 | gl_14718013 | Cneorum pulverulentum |
| 2268 | gl_6708108 | Streptococcus thermophilus |
| 2269 | gl_6689008 | Philadelphus lewisii |
| 2270 | gl_20467373 | Ephedra intermedia |
| 2271 | gl_23019058 | Thermobifida fusca |
| 2272 | gl_7708296 | Ercilla volubilis |
| 2273 | gl_21684923 | Xyris involucrata |
| 2274 | gl_23133806 | Synechococcus sp. WH 8102 |
| 2275 | gl_28198387 | Xylella fastidiosa Temecula1 |
| 2276 | gl_7708616 | Rheum pinchonii |
| 2277 | gl_5758903 | Ornithogalum caudatum |
| 2278 | MRT4565_58256P.2 | Triticum aestivum |
| 2279 | gl_28380214 | Vibrio metschnikovii |
| 2280 | gl_28262700 | Rickettsia sibirica |
| 2281 | gl_22962442 | Rhodopseudomonas palustris |
| 2282 | gl_15233656 | Arabidopsis thaliana |
| 2283 | gl_30351917 | Polyxena ensifolia |
| 2284 | gl_23121268 | Desulfitobacterium hafniense |
| 2285 | MRT4530_114918P.2 | Oryza sativa |
| 2286 | gl_7708460 | Kedrostis nana |
| 2287 | gl_15673133 | Lactococcus lactis subsp. lactis |
| 2288 | gl_7488483 | Brassica napus |
| 2289 | gl_4995177 | Grewia occidentalis |
| 2290 | gl_1272340 | Capsicum annuum |
| 2291 | gl_12322049 | Arabidopsis thaliana |
| 2292 | gl_27528490 | Saccharomyces bayanus |
| 2293 | gl_1655938 | Vibrio parahaemolyticus |
| 2294 | gl_17549667 | Ralstonia solanacearum |
| 2295 | gl_15236196 | Arabidopsis thaliana |
| 2296 | gl_20136103 | Escherichia fergusonii |
| 2297 | gl_14718076 | Humulus lupulus |
| 2298 | gl_4218162 | Gerbera hybrid cv. [Terra Reglna] |
| 2299 | gl_7708662 | Strychnos nux-vomica |
| 2300 | gl_25029429 | Corynebacterium efficiens YS-314 |
| 2301 | gl_5305260 | Brassica rapa |
| 2302 | gl_3913225 | Cyanidium caldarium |
| 2303 | gl_6689113 | Roglera suffrutescens |
| 2304 | gl_3850980 | Lomatia myricoides |
| 2305 | gl_22999862 | Magnetococcus sp. MC-1 |
| 2306 | gl_21230440 | Xanthomonas campestris pv. campestris str. ATCC 33913 |
| 2307 | gl_18309257 | Clostridium perfringens str. 13 |
| 2308 | gl_13357744 | Ureaplasma urealyticum |
| 2309 | gl_27262354 | Heliobacillus mobilis |
| 2310 | gl_7708333 | Humiria balsamifera |
| 2311 | gl_29376445 | Enterococcus faecalis V583 |
| 2312 | gl_4063526 | Ailanthus altissima |
| 2313 | gl_15835226 | Chlamydia muridarum |
| 2314 | gl_28192488 | Streptomyces carzinostaticus subsp. neocarzinostaticus |
| 2315 | gl_21633435 | Cordisepalum phalanthopetalum |
| 2316 | gl_114516 | Halobacterium salinarum |
| 2317 | gl_32412440 | Neurospora crassa |
| 2318 | gl_7708674 | Tetracera asiatica |
| 2319 | gl_16549078 | Magnolia praecocissima |
| 2320 | gl_30265987 | Coleochaete sp. 489a1 |
| 2321 | gl_23465609 | Bifidobacterium longum NCC2705 |
| 2322 | gl_6175246 | Lycopersicon esculentum |
| 2323 | gl_18650789 | Phalaenopsis equestris |
| 2324 | MRT4565_16821P.3 | Triticum aestivum |
| 2325 | gl_18075921 | Escallonia calcottiae |
| 2326 | gl_23112455 | Desulfitobacterium hafniense |
| 2327 | MRT3847_64872P.3 | Glycine max |
| 2328 | gl_22297982 | Thermosynechococcus elongatus BP-1 |
| 2329 | gl_13366140 | Hordeum vulgare subsp. vulgare |
| 2330 | gl_19033057 | Nitellopsis obtusa |
| 2331 | gl_4206610 | Trichilia emetica |
| 2332 | gl_24374035 | Shewanella oneidensis MR-1 |
| 2333 | gl_15216030 | Vicia faba var. minor |
| 2334 | gl_20136097 | Escherichia coli |
| 2335 | gl_20136033 | Shigella flexneri |
| 2336 | gl_5001569 | Hedera helix |
| 2337 | gl_11260405 | Schizosaccharomyces pombe |

What is claimed is:

1-4. (canceled)

5. Hybrid maize seed which is produced by crossing two parental maize lines where at least one of said parental maize lines is a transgenic maize line which has in its genome a recombinant DNA construct comprising at least one oil-associated gene operably linked to a promoter which is functional in said plant to transcribe said oil-associated gene.

6-9. (canceled)

10. A method of breeding maize comprising selecting from a breeding population of maize plants a selected maize plant with higher oil than other maize plants in said breeding population based on allelic polymorphisms associated by linkage disequilibrium to a higher seed oil-related trait, wherein the selected maize plant has 1 or more higher oil alleles linked to a maize oil marker.

11. (canceled)

12. A method of breeding maize according to claim 10 wherein said selected maize plant has 2 or more higher oil alleles linked to a maize oil marker.

13. A method of breeding maize according to claim 10 wherein said selected maize plant has 3 or more higher oil alleles linked to a maize oil marker.

14-20. (canceled)

21. A method of associating a seed oil-related trait to a genotype in maize comprising
(a) identifying a set of one or more seed oil level traits characterizing said maize plants,
(b) selecting tissue from at least two maize plants having allelic DNA and assaying DNA or mRNA from said tissue to identify the presence or absence of a set of distinct polymorphisms comprising at least one polymorphism linked to a polymorphic maize DNA locus which comprises at least 20 consecutive nucleotides which include or are adjacent to a maize oil marker, and
(c) identifying associations between said set of polymorphisms and said set of traits.
